(19) United States
(12) Patent Application Publication
(10) Pub. No.: US 2024/0197879 A1
(43) Pub. Date: Jun. 20, 2024

(54) ANTIGEN RECOGNIZING RECEPTORS TARGETING CD33 AND USES THEREOF

(71) Applicants: MEMORIAL SLOAN-KETTERING CANCER CENTER, New York, NY (US); SLOAN-KETTERING INSTITUTE FOR CANCER RESEARCH, New York, NY (US); MEMORIAL HOSPITAL FOR CANCER AND ALLIED DISEASES, New York, NY (US); TRI-INSTITUTIONAL THERAPEUTICS DISCOVERY INSTITUTE, INC., New York, NY (US)

(72) Inventors: Anthony Daniyan, Somers, NY (US); Renier J. BRENTJENS, Short Hills, NJ (US); Ivo C. LORENZ, New York, NY (US); Abdul KHAN, Cedar Park, TX (US)

(73) Assignees: MEMORIAL SLOAN-KETTERING CANCER CENTER, New York, NY (US); SLOAN-KETTERING INSTITUTE FOR CANCER RESEARCH, New York, NY (US); MEMORIAL HOSPITAL FOR CANCER AND ALLIED DISEASES, New York, NY (US); TRI-INSTITUTIONAL THERAPEUTICS DISCOVERY INSTITUTE, INC., New York, NY (US)

(21) Appl. No.: 18/591,191

(22) Filed: Feb. 29, 2024

Related U.S. Application Data
(63) Continuation of application No. PCT/US2022/042444, filed on Sep. 2, 2022.
(60) Provisional application No. 63/306,395, filed on Feb. 3, 2022.
(60) Provisional application No. 63/240,196, filed on Sep. 2, 2021.

(51) Int. Cl. A61K 39/00; A61P 35/02; C07K 14/705; C07K 14/725; C07K 14/73; C07K 16/28; C12N 5/0783

(52) U.S. Cl. CPC A61K 39/464411; A61K 39/4611; A61K 39/4631; A61P 35/02; C07K 14/70503; C07K 14/7051; C07K 14/70514; C07K 14/70517; C07K 14/70521; C07K 14/70532; C07K 14/70578; C07K 16/2803; C12N 5/0636; C07K 2317/622; C07K 2319/02; C07K 2319/03

(57) ABSTRACT

The presently disclosed subject matter provides for antigen-recognizing receptors that specifically target CD33 and cells comprising such CD33-targeted antigen-recognizing receptors. The presently disclosed subject matter further provides uses of the CD33-targeted antigen-recognizing receptors for treatment.

### CROSS REFERENCE TO RELATED APPLICATIONS

The present application is a Continuation Application of International Patent Application No. PCT/US2022/042444, filed on Sep. 2, 2022, which claims priority to U.S. Provisional Patent Application No. 63/306,395, filed on Feb. 3, 2022, and U.S. Provisional Patent Application No. 63/240,196, filed on Sep. 2, 2021, the contents of each of which are incorporated by reference in their entireties, and to each of which priority is claimed.

### SEQUENCE LISTING

The present application contains a Sequence Listing which has been electronically submitted and is hereby incorporated by reference in its entirety. Said Sequence Listing, created on Feb. 29, 2024, is named 072734_1555.xml and is 164,037 bytes in size.

### 1. INTRODUCTION

The presently disclosed subject matter provides methods and compositions for immunotherapies. It relates to antigen-recognizing receptors (e.g., chimeric antigen receptors (CARs)) that specifically target CD33, cells comprising such receptors, and methods of using such cells for treatments.

### 2. BACKGROUND OF THE INVENTION

Acute myeloid leukemia (AML) is the most common and most lethal form of acute leukemia in the United States. For the last four decades, the standard of care has been chemotherapy with the recent addition of a CD33 antibody-drug conjugate (ADC) and targeted small molecules to the armamentarium. Despite these new additions, AML continues to be a devastating disease with a 5-year survival of less than 30%. Hence, there is a critical need for novel AML interventions.

Over the last decade, CAR T cells have emerged as one of the most potent forms of immunotherapy. Although many groups have deployed this technology to the treatment of AML via the targeting of CD33, one major obstacle remains: the heterogeneous density of this antigen within and between patient populations. This is of great importance as it has been demonstrated that CAR T cell efficacy correlates with antigen density and that a reduction, rather than a total loss of antigen surface expression, is a mechanism of CAR T cell immune escape. To overcome this, membrane-proximal epitope targeting has emerged as a strategy to bolster CAR T cell efficacy, leading to increasing overall CAR T cell potency, and the ability of effector cells to recognize low antigen density variants. As a result, developing a CAR T cell that targets a membrane-proximal epitope of CD33 would allow for enhanced elimination of disease with varying CD33 expression including splice variants that may lack the membrane-distal domain, a target of all currently known antibodies.

### 3. SUMMARY OF THE INVENTION

The presently disclosed subject matter provides antigen-recognizing receptors that specifically target CD33 and cells comprising such CD33-targeted antigen-recognizing receptors. The presently disclosed subject matter further provides uses of the CD33-targeted antigen-recognizing receptors for treatment.

The presently disclosed subject matter provides an antigen-recognizing receptor, comprising an extracellular antigen-binding domain, a transmembrane domain, and an intracellular signaling domain, wherein the extracellular antigen-binding domain specifically binds to CD33. In certain embodiments, the extracellular antigen-binding domain comprises a heavy chain variable region and a light chain variable region. In certain embodiments, the extracellular antigen-binding domain is a single-chain variable fragment (scFv). In certain embodiments, the variable regions are positioned from the N- to the C-terminus: V_{H}-V_{L}. In certain embodiments, the extracellular antigen-binding domain is a human scFv. In certain embodiments, the extracellular antigen-binding domain is a Fab, which is optionally crosslinked. In certain embodiments, the extracellular antigen-binding domain is a F(ab)₂. In certain embodiments, one or more of the scFv, Fab and F(ab)₂ are comprised in a fusion protein with a heterologous sequence to form the extracellular antigen-binding domain.

In certain embodiments, the heavy chain variable region comprises
(a) a CDR1 comprising the amino acid sequence set forth in SEQ ID NO: 2 or a conservative modification thereof, a CDR2 comprising the amino acid sequence set forth in SEQ ID NO: 3 or a conservative modification thereof, and a CDR3 comprising the amino acid sequence set forth in SEQ ID NO: 4 or a conservative modification thereof;
(b) a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof;
(c) a CDR1 comprising the amino acid sequence set forth in SEQ ID NO: 22 or a conservative modification thereof, a CDR2 comprising the amino acid sequence set forth in SEQ ID NO: 23 or a conservative modification thereof, and a CDR3 comprising the amino acid sequence set forth in SEQ ID NO: 24 or a conservative modification thereof;
(d) a CDR1 comprising the amino acid sequence set forth in SEQ ID NO: 32 or a conservative modification thereof, a CDR2 comprising the amino acid sequence set forth in SEQ ID NO: 33 or a conservative modification thereof, and a CDR3 comprising the amino acid sequence set forth in SEQ ID NO: 34 or a conservative modification thereof;
(e) a CDR1 comprising the amino acid sequence set forth in SEQ ID NO: 37 or a conservative modification thereof, a CDR2 comprising the amino acid sequence set forth in SEQ ID NO: 38 or a conservative modification thereof, and a CDR3 comprising the amino acid sequence set forth in SEQ ID NO: 39 or a conservative modification thereof;
(f) a CDR1 comprising the amino acid sequence set forth in SEQ ID NO: 47 or a conservative modification thereof, a CDR2 comprising the amino acid sequence set forth in SEQ ID NO: 38 or a conservative modification thereof, and a CDR3 comprising the amino acid sequence set forth in SEQ ID NO: 48 or a conservative modification thereof;
(g) a CDR1 comprising the amino acid sequence set forth in SEQ ID NO: 56 or a conservative modification thereof, a CDR2 comprising the amino acid sequence set forth in SEQ ID NO: 57 or a conservative modification thereof, and a CDR3 comprising the amino acid sequence set forth in SEQ ID NO: 58 or a conservative modification thereof;
(h) a CDR1 comprising the amino acid sequence set forth in SEQ ID NO: 66 or a conservative modification thereof, a CDR2 comprising the amino acid sequence set forth in SEQ ID NO: 67 or a conservative modification thereof, and a CDR3 comprising the amino acid sequence set forth in SEQ ID NO: 68 or a conservative modification thereof;
(i) a CDR1 comprising the amino acid sequence set forth in SEQ ID NO: 76 or a conservative modification thereof, a CDR2 comprising the amino acid sequence set forth in SEQ ID NO: 57 or a conservative modification thereof, and a CDR3 comprising the amino acid sequence set forth in SEQ ID NO: 77 or a conservative modification thereof; or
(j) a CDR1 comprising the amino acid sequence set forth in SEQ ID NO: 85 or a conservative modification thereof, a CDR2 comprising the amino acid sequence set forth in SEQ ID NO: 86 or a conservative modification thereof, and a CDR3 comprising the amino acid sequence set forth in SEQ ID NO: 87 or a conservative modification thereof.

In certain embodiments, the heavy chain variable region comprises:
(a) a CDR1 comprising the amino acid sequence set forth in SEQ ID NO: 2 or a conservative modification thereof, a CDR2 comprising the amino acid sequence set forth in SEQ ID NO: 3 or a conservative modification thereof, and a CDR3 comprising the amino acid sequence set forth in SEQ ID NO: 4 or a conservative modification thereof;
(b) a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof; or
(c) a CDR1 comprising the amino acid sequence set forth in SEQ ID NO: 22 or a conservative modification thereof, a CDR2 comprising the amino acid sequence set forth in SEQ ID NO: 23 or a conservative modification thereof, and a CDR3 comprising the amino acid sequence set forth in SEQ ID NO: 24 or a conservative modification thereof.

In certain embodiments, the light chain variable region comprises:
(a) a CDR1 comprising the amino acid sequence set forth in SEQ ID NO: 5 or a conservative modification thereof, a CDR2 comprising the amino acid sequence set forth in SEQ ID NO: 6 or a conservative modification thereof, and a CDR3 comprising the amino acid sequence set forth in SEQ ID NO: 7 or a conservative modification thereof;
(b) a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising SEQ ID NO: 17 or a conservative modification thereof;
(c) a CDR1 comprising the amino acid sequence set forth in SEQ ID NO: 25 or a conservative modification thereof, a CDR2 comprising the amino acid sequence set forth in SEQ ID NO: 26 or a conservative modification thereof, and a CDR3 comprising the amino acid sequence set forth in SEQ ID NO: 27 or a conservative modification thereof;
(d) a CDR1 comprising the amino acid sequence set forth in SEQ ID NO: 40 or a conservative modification thereof, a CDR2 comprising SEQ ID NO: 41 or a conservative modification thereof, and a CDR3 comprising the amino acid sequence set forth in SEQ ID NO: 42 or a conservative modification thereof;
(e) a CDR1 comprising the amino acid sequence set forth in SEQ ID NO: 49 or a conservative modification thereof, a CDR2 comprising the amino acid sequence set forth in SEQ ID NO: 50 or a conservative modification thereof, and a CDR3 comprising the amino acid sequence set forth in SEQ ID NO: 51 or a conservative modification thereof;
(f) a CDR1 comprising the amino acid sequence set forth in SEQ ID NO: 59 or a conservative modification thereof, a CDR2 comprising the amino acid sequence set forth in SEQ ID NO: 60 or a conservative modification thereof, and a CDR3 comprising the amino acid sequence set forth in SEQ ID NO: 61 or a conservative modification thereof;
(g) a CDR1 comprising the amino acid sequence set forth in SEQ ID NO: 69 or a conservative modification thereof, a CDR2 comprising the amino acid sequence set forth in SEQ ID NO: 70 or a conservative modification thereof, and a CDR3 comprising the amino acid sequence set forth in SEQ ID NO: 71 or a conservative modification thereof;
(h) a CDR1 comprising the amino acid sequence set forth in SEQ ID NO: 78 or a conservative modification thereof, a CDR2 comprising the amino acid sequence set forth in SEQ ID NO: 79 or a conservative modification thereof, and a CDR3 comprising the amino acid sequence set forth in SEQ ID NO: 80 or a conservative modification thereof; or
(i) a CDR1 comprising the amino acid sequence set forth in SEQ ID NO: 5 or a conservative modification thereof, a CDR2 comprising the amino acid sequence set forth in SEQ ID NO: 41 or a conservative modification thereof, and a CDR3 comprising the amino acid sequence set forth in SEQ ID NO: 42 or a conservative modification thereof.

In certain embodiments, the light chain variable region comprises:
(a) a CDR1 comprising the amino acid sequence set forth in SEQ ID NO: 5 or a conservative modification thereof, a CDR2 comprising the amino acid sequence set forth in SEQ ID NO: 6 or a conservative modification thereof, and a CDR3 comprising the amino acid sequence set forth in SEQ ID NO: 7 or a conservative modification thereof;
(b) a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising SEQ ID NO: 17 or a conservative modification thereof;
(c) a CDR1 comprising the amino acid sequence set forth in SEQ ID NO: 25 or a conservative modification thereof, a CDR2 comprising the amino acid sequence set forth in SEQ ID NO: 26 or a conservative modification thereof, and a CDR3 comprising the amino acid sequence set forth in SEQ ID NO: 27 or a conservative modification thereof.

In certain embodiments,
(a) the heavy chain variable region comprises a CDR1 comprising the amino acid sequence set forth in SEQ ID NO: 2, a CDR2 comprising the amino acid sequence set forth in SEQ ID NO: 3, and a CDR3 comprising the amino acid sequence set forth in SEQ ID NO: 4; and the light chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 7;
(b) the heavy chain variable region comprises a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 14; and the light chain variable region comprises a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17;
(c) the heavy chain variable region comprises a CDR1 comprising the amino acid sequence set forth in SEQ ID NO: 22, a CDR2 comprising the amino acid sequence set forth in SEQ ID NO: 23, and a CDR3 comprising the amino acid sequence set forth in SEQ ID NO: 24; and the light chain variable region comprises a CDR1 comprising the amino acid sequence set forth in SEQ ID NO: 25, a CDR2 comprising the amino acid sequence set forth in SEQ ID NO: 26, and a CDR3 comprising the amino acid sequence set forth in SEQ ID NO: 27;
(d) the heavy chain variable region comprises a CDR1 comprising the amino acid sequence set forth in SEQ ID NO: 32, a CDR2 comprising the amino acid sequence set forth in SEQ ID NO: 33, and a CDR3 comprising the amino acid sequence set forth in SEQ ID NO: 34; and the light chain variable region comprises a CDR1 comprising the amino acid sequence set forth in SEQ ID NO: 25, a CDR2 comprising the amino acid sequence set forth in SEQ ID NO: 26, and a CDR3 comprising the amino acid sequence set forth in SEQ ID NO: 27;
(e) the heavy chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 39; and the light chain variable region comprises a CDR1 comprising the amino acid sequence set forth in SEQ ID NO: 40, a CDR2 comprising the amino acid sequence set forth in SEQ ID NO: 41, and a CDR3 comprising the amino acid sequence set forth in SEQ ID NO: 42;
(f) the heavy chain variable region comprises a CDR1 comprising the amino acid sequence set forth in SEQ ID NO: 47, a CDR2 comprising the amino acid sequence set forth in SEQ ID NO: 38, and a CDR3 comprising the amino acid sequence set forth in SEQ ID NO: 48; and the light chain variable region comprises a CDR1 comprising the amino acid sequence set forth in SEQ ID NO: 49, a CDR2 comprising the amino acid sequence set forth in SEQ ID NO: 50, and a CDR3 comprising the amino acid sequence set forth in SEQ ID NO: 51;
(g) the heavy chain variable region comprises a CDR1 comprising the amino acid sequence set forth in SEQ ID NO: 56, a CDR2 comprising the amino acid sequence set forth in SEQ ID NO: 57, and a CDR3 comprising the amino acid sequence set forth in SEQ ID NO: 58; and the light chain variable region comprises a CDR1 comprising the amino acid sequence set forth in SEQ ID NO: 59, a CDR2 comprising the amino acid sequence set forth in SEQ ID NO: 60, and a CDR3 comprising the amino acid sequence set forth in SEQ ID NO: 61;
(h) the heavy chain variable region comprises a CDR1 comprising the amino acid sequence set forth in SEQ ID NO: 66, a CDR2 comprising the amino acid sequence set forth in SEQ ID NO: 67, and a CDR3 comprising the amino acid sequence set forth in SEQ ID NO: 68; and the light chain variable region comprises a CDR1 comprising the amino acid sequence set forth in SEQ ID NO: 69, a CDR2 comprising the amino acid sequence set forth in SEQ ID NO: 70, and a CDR3 comprising the amino acid sequence set forth in SEQ ID NO: 71;
(i) the heavy chain variable region comprises a CDR1 comprising the amino acid sequence set forth in SEQ ID NO: 76, a CDR2 comprising the amino acid sequence set forth in SEQ ID NO: 57, and a CDR3 comprising the amino acid sequence set forth in SEQ ID NO: 77; and the light chain variable region comprises a CDR1 comprising the amino acid sequence set forth in SEQ ID NO: 78, a CDR2 comprising the amino acid sequence set forth in SEQ ID NO: 79, and a CDR3 comprising the amino acid sequence set forth in SEQ ID NO: 80; or
(j) the heavy chain variable region comprises a CDR1 comprising the amino acid sequence set forth in SEQ ID NO: 85, a CDR2 comprising the amino acid sequence set forth in SEQ ID NO: 86, and a CDR3 comprising the amino acid sequence set forth in SEQ ID NO: 87; and the light chain variable region comprises a CDR1 comprising the amino acid sequence set forth in SEQ ID NO: 5, a CDR2 comprising the amino acid sequence set forth in SEQ ID NO: 41, and a CDR3 comprising the amino acid sequence set forth in SEQ ID NO: 42.

In certain embodiments,
(a) the heavy chain variable region comprises a CDR1 comprising the amino acid sequence set forth in SEQ ID NO: 2, a CDR2 comprising the amino acid sequence set forth in SEQ ID NO: 3, and a CDR3 comprising the amino acid sequence set forth in SEQ ID NO: 4; and the light chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 7;
(b) the heavy chain variable region comprises a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 14; and the light chain variable region comprises a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17; or
(c) the heavy chain variable region comprises a CDR1 comprising the amino acid sequence set forth in SEQ ID NO: 22, a CDR2 comprising the amino acid sequence set forth in SEQ ID NO: 23, and a CDR3 comprising the amino acid sequence set forth in SEQ ID NO: 24; and the light chain variable region comprises a CDR1 comprising the amino acid sequence set forth in SEQ ID NO: 25, a CDR2 comprising the amino acid sequence set forth in SEQ ID NO: 26, and a CDR3 comprising the amino acid sequence set forth in SEQ ID NO: 27.

In certain embodiments, the heavy chain variable region comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence set forth in SEQ ID NO: 8, SEQ ID NO: 18, SEQ ID NO: 28, SEQ ID NO: 35, SEQ ID NO: 43, SEQ ID NO: 52, SEQ ID NO: 62, SEQ ID NO: 72, SEQ ID NO: 81, or SEQ ID NO: 88. In certain embodiments, the heavy chain variable region comprises the amino acid sequence set forth in SEQ ID NO: 8, SEQ ID NO: 18, SEQ ID NO: 28, SEQ ID NO: 35, SEQ ID NO: 43, SEQ ID NO: 52, SEQ ID NO: 62, SEQ ID NO: 72, SEQ ID NO: 81, or SEQ ID NO: 88. In certain embodiments, the heavy chain variable region comprises the amino acid sequence set forth in SEQ ID NO: 8, SEQ ID NO: 18, or SEQ ID NO: 28.

In certain embodiments, the light chain variable region comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence set forth in SEQ ID NO: 9, SEQ ID NO: 19, SEQ ID NO: 29, SEQ ID NO: 44, SEQ ID NO: 53, SEQ ID NO: 63, SEQ ID NO: 73, SEQ ID NO: 82, SEQ ID NO: 89, or SEQ ID NO: 92. In certain embodiments, the light chain variable region comprises the amino acid sequence set forth in SEQ ID NO: 9, SEQ ID NO: 19, SEQ ID NO: 29, SEQ ID NO: 44, SEQ ID NO: 53, SEQ ID NO: 63, SEQ ID NO: 73, SEQ ID NO: 82, SEQ ID NO: 89, or SEQ ID NO: 92. In certain embodiments, the light chain variable region comprises the amino acid sequence set forth in SEQ ID NO: 9, SEQ ID NO: 19, or SEQ ID NO: 29.

In certain embodiments, (a) the heavy chain variable region comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence selected set forth in SEQ ID NO: 8, SEQ ID NO: 18, SEQ ID NO: 28, SEQ ID NO: 35, SEQ ID NO: 43, SEQ ID NO: 52, SEQ ID NO: 62, SEQ ID NO: 72, SEQ ID NO: 81, or SEQ ID NO: 88; and (b) the light chain variable region comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence set forth in SEQ ID NO: 9, SEQ ID NO: 19, SEQ ID NO: 29, SEQ ID NO: 44, SEQ ID NO: 53, SEQ ID NO: 63, SEQ ID NO: 73, SEQ ID NO: 82, SEQ ID NO: 89, or SEQ ID NO: 92.

In certain embodiments, (a) the heavy chain variable region comprises the amino acid sequence set forth in SEQ ID NO: 8, SEQ ID NO: 18, SEQ ID NO: 28, SEQ ID NO: 35, SEQ ID NO: 43, SEQ ID NO: 52, SEQ ID NO: 62, SEQ ID NO: 72, SEQ ID NO: 81, or SEQ ID NO: 88; and (b) the light chain variable region comprises the amino acid sequence set forth in SEQ ID NO: 9, SEQ ID NO: 19, SEQ ID NO: 29, SEQ ID NO: 44, SEQ ID NO: 53, SEQ ID NO: 63, SEQ ID NO: 73, SEQ ID NO: 82, SEQ ID NO: 89, or SEQ ID NO: 92. In certain embodiments, (a) the heavy chain variable region comprises the amino acid sequence set forth in SEQ ID NO: 8, SEQ ID NO: 18, or SEQ ID NO: 28; and (b) the light chain variable region comprises the amino acid sequence set forth in SEQ ID NO: 9, SEQ ID NO: 19, or SEQ ID NO: 29.

In certain embodiments,
(a) the heavy chain variable region comprises the amino acid sequence set forth in SEQ ID NO: 8, and the light chain variable region comprises the amino acid sequence set forth in SEQ ID NO: 9;
(b) the heavy chain variable region comprises the amino acid sequence set forth in SEQ ID NO: 18, and the light chain variable region comprises the amino acid sequence set forth in SEQ ID NO: 19;
(c) the heavy chain variable region comprises the amino acid sequence set forth in SEQ ID NO: 28, and the light chain variable region comprises the amino acid sequence set forth in SEQ ID NO: 29;
(d) the heavy chain variable region comprises the amino acid sequence set forth in SEQ ID NO: 35, and the light chain variable region comprises the amino acid sequence set forth in SEQ ID NO: 29;
(e) the heavy chain variable region comprises the amino acid sequence set forth in SEQ ID NO: 43, and the light chain variable region comprises the amino acid sequence set forth in SEQ ID NO: 44;
(f) the heavy chain variable region comprises the amino acid sequence set forth in SEQ ID NO: 52, and the light chain variable region comprises the amino acid sequence set forth in SEQ ID NO: 53;
(g) the heavy chain variable region comprises the amino acid sequence set forth in SEQ ID NO: 62, and the light chain variable region comprises the amino acid sequence set forth in SEQ ID NO: 63;
(h) the heavy chain variable region comprises the amino acid sequence set forth in SEQ ID NO: 72, and the light chain variable region comprises the amino acid sequence set forth in SEQ ID NO: 73;
(i) the heavy chain variable region comprises the amino acid sequence set forth in SEQ ID NO: 81, and the light chain variable region comprises the amino acid sequence set forth in SEQ ID NO: 82;
j) the heavy chain variable region comprises the amino acid sequence set forth in SEQ ID NO: 88, and the light chain variable region comprises the amino acid sequence set forth in SEQ ID NO: 89; or
(k) the heavy chain variable region comprises the amino acid sequence set forth in SEQ ID NO: 8, and the light chain variable region comprises the amino acid sequence set forth in SEQ ID NO: 93.

In certain embodiments,
(a) the heavy chain variable region comprises the amino acid sequence set forth in SEQ ID NO: 8, and the light chain variable region comprises the amino acid sequence set forth in SEQ ID NO: 9;
(b) the heavy chain variable region comprises the amino acid sequence set forth in SEQ ID NO: 18, and the light chain variable region comprises the amino acid sequence set forth in SEQ ID NO: 19; or
(c) the heavy chain variable region comprises the amino acid sequence set forth in SEQ ID NO: 28, and the light chain variable region comprises the amino acid sequence set forth in SEQ ID NO: 29.

In certain embodiments, the extracellular antigen-binding domain comprises a linker between the heavy chain variable region and the light chain variable region. In certain embodiments, the linker comprises or consists of the amino acid sequence set forth in SEQ ID NO: 95, SEQ ID NO: 96, SEQ ID NO: 97, SEQ ID NO: 98, SEQ ID NO: 99, or SEQ ID NO: 100. In certain embodiments, a signal peptide is covalently joined to the 5′ terminus of the extracellular antigen-binding domain.

In certain embodiments, the transmembrane domain comprises a CD8 polypeptide, a CD28 polypeptide, a CD3ζ polypeptide, a CD4 polypeptide, a 4-1BB polypeptide, an OX40 polypeptide, an ICOS polypeptide, a CTLA-4 polypeptide, a PD-1 polypeptide, a LAG-3 polypeptide, a 2B4 polypeptide, a BTLA polypeptide, or a combination thereof.

In certain embodiments, the intracellular signaling domain comprises a CD3ζ polypeptide. In certain embodiments, the intracellular signaling domain further comprises at least one co-stimulatory signaling region. In certain embodiments, the at least one co-stimulatory signaling region comprises a CD28 polypeptide, a 4-1BB polypeptide, an OX40 polypeptide, an ICOS polypeptide, a DAP-10 polypeptide, or a combination thereof.

In certain embodiments, the antigen-recognizing receptor is a chimeric antigen receptor (CAR), or a T-cell like fusion protein. In certain embodiments, the antigen-recognizing receptor is a CAR.

In certain embodiments, the antigen-recognizing receptor is recombinantly expressed. In certain embodiments, the antigen-recognizing receptor is expressed from a vector. In certain embodiments, the vector is a γ-retroviral vector.

The presently disclosed subject matter provides cells comprises a presently disclosed antigen-recognizing receptor. In certain embodiments, the cell is transduced with the antigen-recognizing receptor. In certain embodiment, the antigen-recognizing receptor is constitutively expressed on the surface of the cell.

In certain embodiments, the cell is an immunoresponsive cell. In certain embodiments, the cell is a cell of the lymphoid lineage or a cell of the myeloid lineage. In certain embodiments, the cell is selected from the group consisting of a T cell, a Natural Killer (NK) cell, and a stem cell from which lymphoid cells may be differentiated. In certain embodiments, the cell is a T cell. In certain embodiments, the T cell is a cytotoxic T lymphocyte (CTL) or a regulatory T cell. In certain embodiments, the stem cell is a pluripotent stem cell. In certain embodiments, the pluripotent stem cell is an embryoid stem cell or an induced pluripotent stem cell.

The presently disclosed subject matter further provides nucleic acid that encode a presently disclosed antigen-recognizing receptor. The presently disclosed subject matter further provides vectors comprising the presently disclosed nucleic acid molecules. In certain embodiments, the vector is a viral vector. In certain embodiments, the vector is a γ-retroviral vector.

In addition, the presently disclosed subject matter provides host cells expressing the nucleic acid molecule disclosed herein. In certain embodiments, the host cell is a T cell.

The presently disclosed subject matter further provides compositions comprising the cells disclosed herein. In certain embodiments, the composition is a pharmaceutical composition further comprising a pharmaceutically acceptable carrier.

The presently disclosed subject matter also provides lipid nanoparticles comprising the nucleic acids disclosed herein. Also, the presently disclosed subject matter provides compositions comprising the lipid nanoparticles disclosed herein. In certain embodiments, the composition is a pharmaceutical composition further comprising a pharmaceutically acceptable carrier.

The presently disclosed subject matter further provides methods of treating or ameliorating a disease or disorder in a subject. In certain embodiments, the method comprises administering to the subject the presently disclosed cells, or the compositions.

The presently disclosed subject matter also provides methods of reducing tumor burden in a subject. In certain embodiments, the methods comprise administering to the subject the presently disclosed cells, or the compositions. In certain embodiments, the methods reduce the number of the tumor cells, reduce the tumor size, and/or eradicate the tumor in the subject.

The presently disclosed subject matter provides methods of treating and/or preventing a tumor in a subject. In certain embodiments, the methods comprise administering to the subject the presently disclosed cells, or the compositions. The presently disclosed subject matter provides methods of increasing or lengthening survival of a subject having a tumor. In certain embodiments, the methods comprise administering to the subject the presently disclosed cells, or the compositions. In certain embodiments, the methods reduce or eradicate tumor burden in the subject.

In certain embodiments, the disease or disorder is a tumor. In certain embodiments, the tumor is hematological cancer or solid tissue cancer. In certain embodiments, the tumor is selected from the group consisting of acute myeloid leukemia (AML), myelodysplastic syndromes (MDS), acute lymphoblastic leukemia (ALL), chronic lymphocytic leukemia (CLL), myeloproliferative neoplasms (MPNs), and chronic myeloid neoplasms. In certain embodiments, the tumor is acute myeloid leukemia (AML). In certain embodiments, the subject is a human.

The presently disclosed subject matter further provides kits for treating or ameliorating a disease or disorder in a subject, reducing tumor burden in a subject, treating and/or preventing a tumor in a subject, and/or increasing or lengthening survival of a subject having a tumor comprising the presently disclosed cells, the nucleic acids, or the compositions. In certain embodiments, the kit further comprises written instructions for using the presently disclosed cell or composition for treating or ameliorating a disease or disorder in a subject, reducing tumor burden in a subject, treating and/or preventing a tumor in a subject, and/or increasing or lengthening survival of a subject having a tumor.

In addition, the presently disclosed subject matter provides methods of producing a CD33-targeted antigen-recognizing receptor, comprising introducing into the cell a nucleic acid that encodes the antigen-recognizing receptor.

Finally, the presently disclosed subject matter provides cells or compositions disclosed herein for use in treating or ameliorating a disease or disorder in a subject. In certain embodiments, the disease or disorder is a tumor. In certain embodiments, the tumor is cancer. In certain embodiments, the disease or disorder is selected from the group consisting of neuroendocrine tumors of the lung, extrapulmonary neuroendocrine carcinomas, melanoma, neuroendocrine prostate cancer, breast cancer, neuroendocrine tumors of the gastrointestinal tract, pancreatic cancer, medullary thyroid cancer, small cell bladder cancer, ovarian small cell carcinoma, low-grade glioma, glioblastoma and neuroblastoma. In certain embodiments, the neuroendocrine tumors of the lung are selected from the group consisting of pulmonary neuroendocrine cancer, large cell neuroendocrine carcinoma, and small-cell lung cancer. In certain embodiments, the tumor is small-cell lung cancer. In certain embodiments, the subject is a human

### 4. BRIEF DESCRIPTION OF THE FIGURES

The following Detailed Description, given by way of example, but not intended to limit the invention to specific embodiments described, may be understood in conjunction with the accompanying drawings.

FIGS. 1A and 1B depict CD33 domain architecture. FIG. 1A shows full-length CD33 (CD33M) having two Ig-like domains (IgV and IgC2) in the extracellular region, a single transmembrane segment, an immunoreceptor tyrosine-based inhibitory motif (ITIM) and an ITIM-like motif in the cytosolic region. In a common CD33 variant (CD33m), the IgV domain is missing due to alternate splicing. FIG. 1B depicts CD33 gene structure with and without alternate splicing. Removal of exon 2 results in the loss of the IgV domain. Several antibodies that have been advanced to the clinic with various formats (CAR, BiTE, radiotherapy) target the IgV domain. “GO” represents gemtuzumab ozogamicin.

FIG. 2 depicts the structure of an exemplified CD33-targeted CAR in accordance with the presently disclosed subject matter. The CD33-targeted CAR comprises a truncated EGFR (EGFRt), a anti-CD33 scFv, a Myc tag, a transmembrane domain comprising a CD28 polypeptide, and an intracellular signaling domain comprising a CD3ζ polypeptide and a co-stimulatory signaling region that comprises a CD28 polypeptide. T2A is a cleavage site.

FIG. 3 depicts surface expression levels of and flow cytometry analysis of transduced PBMCs indicating the transduction efficiency and CAR surface expression of each construct detected by anti-Myc tag antibody.

FIGS. 4A and 4B depict cytotoxicity of membrane proximal CARs. FIG. 4A shows 3P14 and 4B2 along with the H195 reference CAR were tested with the heavy and light chain variable regions of each antibody in V_{H}-V_{L} (HL) or V_{L}-V_{H} (LH) orientation on U937 cell lines. FIG. 4B shows 3P14 and 4B2 along with the H195 reference CAR were tested on OCi-AML3 cell lines.

FIGS. 5A and 5B depict cytotoxicity of membrane distal CARs. FIG. 5A shows in vitro killing of U937 cells by CAR T cells generated from the 5 antibodies targeting a membrane-distal epitope in the CD33 IgV domain using cells from Donor 2. FIG. 5B shows in vitro killing of U937 cells by CAR T cells generated from the 5 antibodies targeting a membrane-distal epitope in the CD33 IgV domain using cells from Donor 3.

FIGS. 6A-6F depict in vitro cytotoxicity of TDI-Y-006 and TDI-Y-007 CARs. FIGS. 6A-6C show cytotoxicity of 3 AML target lines containing GFP-firefly luciferase reporter (gL) with donor 1 at increasing effector to target ratio as indicated. FIGS. 6D-6F show cytotoxicity of 3 AML target lines containing GFP-firefly luciferase reporter (gL) with donor 2 at increasing effector to target ratio as indicated. Killing was evaluated using luciferase assay. A potent killing with TDI-Y-006 and TDI-Y-007 was observed in various CD33+ target cells and PBMCs from different donors.

FIGS. 7A-7D depict cytotoxicity of 1J19 CAR. FIG. 7A shows cytotoxicity as percent killing of HL60 cell lines containing GFP-firefly luciferase reporter (gL) with Donor 2 at increasing effector to target ratio as indicated. FIG. 7B shows cytotoxicity as percent killing of HL60 cell lines containing GFP-firefly luciferase reporter (gL) with Donor 3 at increasing effector to target ratio as indicated. FIG. 7C shows cytotoxicity as percent killing of U937 cell lines containing GFP-firefly luciferase reporter (gL) with Donor 2 at increasing effector to target ratio as indicated. FIG. 7D shows cytotoxicity as percent killing of OCI-AML3 cell lines containing GFP-firefly luciferase reporter (gL) with Donor 1 at increasing effector to target ratio as indicated. Killing was evaluated using luciferase assay. Overall a potent killing with 1J19 was observed in comparison to H195 reference CAR.

FIGS. 8A-8D depict CD33 CAR T cell proliferation upon binding to target cells. FIGS. 8A and 8B show human T cells from two donors transduced with lead CARs were co-cultured with U937 cells (CD33high). FIGS. 8C and 8D show human T cells from two donors transduced with lead CARs were co-cultured with OCi-AML3 (CD33low) cells. At 7, 14 and 21 days post-co-culture, the total number of remaining target and T cells was detected using flow cytometry. Total remaining human T cells per mL is shown over time in days.

FIGS. 9A-9D depict cytokine secretion profile of lead CD33 CAR T cells. A panel of 4 cytokines was analyzed with three different donors. FIG. 9A shows levels of GMCSF in the supernatant collected after co-culture of target and effector cells at 24h were measured using human 12-plex Luminex panel kit. FIG. 9B shows levels of INF-7. FIG. 9C shows levels of TNF-α. FIG. 9D shows levels of IL-2. Data from representative donor 1 are shown.

FIGS. 10A and 10B depict in vivo efficacy of lead CAR T in U937 AML xenograft mouse model. FIG. 10A shows imaging and quantification over time of U937 cells (CD33high) tagged with GFP-Firefly Luciferase in NCG mice treated IV with lead CAR T cells. FIG. 10B shows survival curve.

FIGS. 11A and 11B depict in vivo efficacy of lead CAR T with titrated dosing. FIG. 11A shows data of animals injected with 5·10⁵ CAR T cells and monitored for tumor growth and survival. FIG. 11B shows data of animals injected with 2.5·10⁵ CAR T cells.

FIGS. 12A and 12B depict in vivo efficacy of lead CAR T in OCi-AML3 xenograft mouse model. Imaging and quantification over time of OCi-AML3 cells (CD33low) tagged with firefly luciferase in NCG mice treated IV with lead CAR T cells. FIG. 12A shows average survival for 5 animals per group. FIG. 12B shows individual survival curves for each animal.

FIG. 13 depicts direct comparison of in vivo potency of two lead and one backup CAR in U937 AML xenograft mouse model. Imaging and quantification over time of U937 cells (CD33high) tagged with Firefly Luciferase in NCG mice treated IV with lead and backup 1J19 CAR T cells.

FIG. 14 depicts in vivo efficacy of TDI-Y-006 CAR T with patient-derived AML xenograft. Patient-derived tumor cells were infused intravenously and allowed to grow for 10 days. TDI-Y-006 CAR T were injected at day 14. Seven days later, the number of CD2− CD33+ cells were determined by flow cytometry. Average data from five mice for H195_Del and four mice for TDI-Y-006 CAR T shown with p value of <0.05*.

FIGS. 15A and 15B depict pro-inflammatory cytokine levels in PDX model. FIG. 15A shows levels of IFN-gamma measured using human 12-plex Luminex panel kit from mice treated with TDI-Y-006 CAR T after patient-derived xenograft implant. FIG. 15B shows levels of TNF-alpha. *p value<0.05.

FIGS. 16A and 16B depict in vitro assessment of toxicities of CD33 CAR T. FIG. 16A shows hematopoietic stem cells isolated from umbilical cord tested by colony forming unit with and without CD33 lead CAR T. FIG. 16B shows total number of colonies to assess the toxicity of experiment agents. Gemtuzumab ozogamicin (GO, Anti-CD33 ADC) and Del CAR were used as control.

FIG. 17 depicts a heat map showing relative binding of selected antibodies to human full-length CD33, CD33-IgC, and U937 CD33-expressing tumor lines.

FIGS. 18A-18D depict selection of a membrane-proximal CD33-IgC targeting scFv. FIG. 18A shows quantification of hybridoma-derived monoclonal antibody cross-reactivity as measured by His-tag binding. FIG. 18B shows quantification of hybridoma-derived monoclonal antibody binding kinetics to CD33 in solution. FIG. 18C shows quantification of EC50 of lead hybridoma-derived monoclonal antibodies on overexpressing (3T3) and AML cell line (U937). FIG. 18D shows quantification of epitope binning of selected hybridoma-derived monoclonal antibodies.

FIG. 19 depicts representative flow cytometry plot demonstrating comparable retroviral transduction efficiency as determined by flow cytometry. P-values determined by repeated measures one-way ANOVA. Data are a mean±SEM of three independent experiments; ns, non-significant.

FIGS. 20A-20G depict membrane-proximal targeting CAR T cells enhancing functionality and proliferative capacity in vitro. FIG. 20A shows flow cytometry histograms and quantitative geometric mean fluorescence (MFI) depicting expression of CD33 on wildtype or CD33-knockout (CD33KO) AML cells detected with fluorescently labeled anti-CD33 antibody. FIG. 20B shows 24-hour D-luciferin assay demonstrating lysis of U937-CD33highgfpLuc⁺ tumor cells (n=4; ****, P<0.0001; ***, P<0.001; *, P<0.05 by two-way ANOVA). Data are a mean±SEM of four independent experiments. FIG. 20C shows a 24-hour D-luciferin assay demonstrating lysis of OCiAML3-CD33lowgfpLuc⁺ tumor cells (n=4; *, P<0.05 by two-way ANOVA). Data are a mean±SEM of four independent experiments. FIG. 20D shows a 138-hour (6 day) assay demonstrating lysis of U937-CD33highgfpLuc⁺ tumor cells at low effector-to-target ratios (n=2; **, P<0.01; *, P<0.05 by two-way ANOVA). Data are representative of four independent experiments. FIG. 20E shows a 138-hour assay demonstrating lysis of OCiAML3-CD33lowgfpLuc⁺ tumor cells at low effector-to-target ratios (n=2; **, P<0.01; *, P<0.05 by two-way ANOVA). Data are representative of four independent experiments. FIG. 20F shows quantification of flow cytometric analysis demonstrating enhanced proliferation by membrane-proximal CD33 targeting CAR T cell in the presence of U937-CD33high tumor cells (n=9; ****, P<0.0001 by two-way ANOVA). Data are a mean±SEM of three independent experiments. FIG. 20G shows quantification of flow cytometric analysis demonstrating enhanced proliferation by membrane-proximal CD33 targeting CAR T cell in the presence of OCiAML3-CD33lowgfpLuc⁺ tumor cells (n=9; ****, P<0.0001 by two-way ANOVA). Data are a mean±SEM of three independent experiments; ns, non-significant.

FIGS. 21A-21D depict specificity of membrane-proximal targeting CAR T cells. FIG. 21A shows flow cytometry histograms depicting FLAG expression on transduced U937-CD33IgCgfpLuc⁺ tumor cells. FIG. 21B shows a 24-hour D-luciferin assay demonstrating lysis of U937-CD33IgCgfpLuc⁺ tumor cells. Data representative of two independent experiments. FIG. 21C shows a 24-hour D-luciferin assay demonstrating lysis of U937-CD33KOgfpLuc⁺ tumor cells. Data representative of two independent experiments. FIG. 21D shows quantification of total colonies produced using a colony-forming unit (CFU) assay to measure hematopoietic killing, where total colony count is a composite of BFU-E, GEMM-CFU, and GM-CFU (n=9; ****, P<0.0001; ***, P<0.001 by ordinary one-way ANOVA; ns, non-significant). Data shown are a composite of mean±SEM of three independent experiments.

FIG. 22A-22G depict membrane-proximal targeting CAR T cells characterized by a unique activation profile in vitro. FIG. 22A shows a 24-hour cytokine secretion profile of Tc1/Th1 cytokines when co-cultured with U937-CD33highgfpLuc⁺, OCiAML3-CD33lowgfpLuc⁺, U937-CD33IgCgfpLuc⁺ tumor, and U937-CD33KOgfpLuc⁺ as detected by human 12-plex Luminex panel (n=3; ****, P<0.0001; *, P<0.05 by Dunnett's multiple comparison tests against H195DEL control). Data are representative of three independent experiments. FIG. 22B shows quantification of flow cytometric analysis showing CAR T cell intracellular production of Tc1/Th1 activation cytokines (n=4; ****, P<0.0001; **, P<0.01; *, P<0.05 by two-way ANOVA; ns, non-significant). Data are a mean±SEM of four independent experiments. FIG. 22C shows qualitative representation of CD4⁺CAR⁺ and CD8⁺CAR⁺ Tc1/Th1 activation cytokines secretion. FIG. 22D shows quantification of flow cytometric analysis showing CD4⁺ CAR T cell intracellular production of Tc1/Th1 activation cytokines (n=4; ****, P<0.0001, *, P<0.05 by two-way ANOVA). Data are a mean±SEM of four independent experiments. FIG. 22E shows quantification of flow cytometric analysis showing CD8⁺ CAR T cell intracellular production of Tc1/Th1 activation cytokines (n=4; ***, P<0.001; **, P<0.01; *, P<0.05 by two-way ANOVA). Data are a mean±SEM of four independent experiments. FIG. 22F shows quantification of flow cytometric analysis demonstrating CAR T cell activation status 7 days post-antigen stimulation (n=4; ****, P<0.0001 by two-way ANOVA). Data are a mean±SEM of three independent experiments. FIG. 22G shows qualitative representation of flow cytometric analysis comparing CAR T cell activation profiles. Data are pooled from three independent experiments.

FIGS. 23A-23K depict membrane-proximal CD33-targeting CAR T cells enhancing survival in xenograft mouse model. FIG. 23A shows schematic diagram of in vivo experimental setup. NCG mice were inoculated with U937-CD33highgfpLuc⁺ tumor and subsequently treated with CAR T cells. FIG. 23B shows survival of NCG mice bearing U937-CD33highgfpLuc⁺ tumors and treated with 5.0×10⁵ CAR T cells (n=5; **, P<0.01). P values for survival determined by log-rank Mantel-Cox test, with 95% confidence interval. In vivo data shown are pooled from 5 mice. FIG. 23C shows survival of NCG mice bearing U937-CD33highgfpLuc⁺ tumors and treated with 2.5×10⁵ CAR T cells (n=5; **, P<0.01; *, P<0.05). P values for survival determined by log-rank Mantel-Cox test, with 95% confidence interval. In vivo data shown are pooled from 5 mice. FIG. 23D shows survival of NCG mice bearing U937-CD33highgfpLuc⁺ tumors and treated with 1.0×10⁵ CAR T cells (n=5; **, P<0.01; ns, non-significant). P values for survival determined by log-rank Mantel-Cox test, with 95% confidence interval. FIG. 23E shows survival of NCG mice bearing U937-CD33highgfpLuc⁺ tumors and treated with 5.0×10⁴ CAR T cells (n=5; *, P<0.05). P values for survival determined by log-rank Mantel-Cox test, with 95% confidence interval. FIG. 23F shows imaging over time of U937-CD33highgfpLuc⁺ in tumor-bearing NCG mice treated with 5.0×10⁵ CAR T cells. FIG. 23G shows tumor regression of NCG mice inoculated with U937-CD33highgfpLuc⁺ tumor and subsequently treated with 5.0×10⁵ of CAR T cells. FIG. 23H shows schematic diagram of in vivo experimental setup. NCG mice were inoculated with OCiAML3-CD33lowgfpLuc⁺ tumor and subsequently treated with CAR T cells. FIG. 23I shows survival of NCG mice inoculated with OCiAML3-CD33lowgfpLuc⁺ tumors and treated with 5.0×10⁵ CAR T cells (n=5; *, P<0.05). P values for survival determined by log-rank Mantel-Cox test, with 95% confidence interval. FIG. 23J shows imaging over time of OCiAML3-CD33lowgfpLuc⁺ in tumor-bearing NCG mice treated with 5.0×10⁵ CAR T cells. FIG. 23K shows tumor regression of NCG mice bearing OCiAML3-CD33lowgfpLuc⁺ tumors and treated with 5.0×10⁵ CAR T cells.

FIGS. 24A-24D depict membrane-proximal CD33-targeting CAR T cells decreasing tumor burden in patient-derived AML xenograft model. FIG. 24A shows flow cytometry histograms and geometric median fluorescence intensity (MFI) of CD33 expression on AML60B patient sample detected with fluorescently labeled CD33-specific antibody and isotype control. FIG. 24B shows schematic diagram of experimental setup of a patient-derived xenograft model. NCG mice were inoculated with patient-derived AML blasts and treated with allogenic CAR T cells. Bone marrow aspirates were analyzed 28 days post tumor inoculation. FIG. 24C shows quantification of flow cytometric analysis demonstrating decreased tumor burden in mice treated with membrane-proximal CD33-targeting CAR T cells (n=6; *, P<0.05 by ordinary one-way ANOVA; ns, non-significant). FIG. 24D shows survival of NCG mice bearing AML60B patient-derived tumor and treated with membrane-distal or membrane-proximal CAR T cells (n=6; **, P<0.01; *, P<0.05). P values for survival determined by log-rank Mantel-Cox test, with 95% confidence interval. Data shown are pooled from two independent experiments using two different healthy donors.

### 5. DETAILED DESCRIPTION OF THE INVENTION

The presently disclosed subject matter provides antigen-recognizing receptors (e.g., chimeric antigen receptors (CARs)) that specifically target CD33. The presently disclosed subject matter further provides cells comprising such receptors. The cells can be immunoresponsive cells, e.g., genetically modified immunoresponsive cells (e.g., T cells or Natural Killer (NK) cells). The presently disclosed subject matter also provides methods of using such cells for treatments, e.g., for treating and or ameliorating a disease or disorder associated with CD33 (e.g., AML).

Non-limiting embodiments of the present disclosure are described by the present specification and Examples.

For purposes of clarity of disclosure and not by way of limitation, the detailed description is divided into the following subsections:
5.1. Definitions;
5.2. CD33;
5.3. Antigen-Recognizing Receptors;
5.4. Cells;
5.5. Compositions and Vectors;
5.6. Polypeptides;
5.7. Formulations and Administration;
5.8. Methods of Treatment;
5.9. Kits; and
5.10. Exemplary Embodiments.

### 5.1. Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

By “immunoresponsive cell” is meant a cell that functions in an immune response or a progenitor, or progeny thereof. In certain embodiments, the immunoresponsive cell is a cell of lymphoid lineage. Non-limiting examples of cells of lymphoid lineage include T cells, Natural Killer (NK) cells, B cells, and stem cells from which lymphoid cells may be differentiated. In certain embodiments, the immunoresponsive cell is a cell of myeloid lineage.

By “activates an immunoresponsive cell” is meant induction of signal transduction or changes in protein expression in the cell resulting in initiation of an immune response. For example, when CD3 Chains cluster in response to ligand binding and immunoreceptor tyrosine-based inhibition motifs (ITAMs) a signal transduction cascade is produced. In certain embodiments, when an endogenous TCR or an exogenous CAR binds to an antigen, a formation of an immunological synapse occurs that includes clustering of many molecules near the bound receptor (e.g. CD4 or CD8, CD3γ/δ/ε/ζ, etc.). This clustering of membrane bound signaling molecules allows for ITAM motifs contained within the CD3 chains to become phosphorylated. This phosphorylation in turn initiates a T cell activation pathway ultimately activating transcription factors, such as NF-κB and AP-1. These transcription factors induce global gene expression of the T cell to increase IL-2 production for proliferation and expression of master regulator T cell proteins in order to initiate a T cell mediated immune response.

By “stimulates an immunoresponsive cell” is meant a signal that results in a robust and sustained immune response. In various embodiments, this occurs after immune cell (e.g., T-cell) activation or concomitantly mediated through receptors including, but not limited to, CD28, CD137 (4-1BB), OX40, CD40 and ICOS. Receiving multiple stimulatory signals can be important to mount a robust and long-term T cell mediated immune response. T cells can quickly become inhibited and unresponsive to antigen. While the effects of these co-stimulatory signals may vary, they generally result in increased gene expression in order to generate long lived, proliferative, and anti-apoptotic T cells that robustly respond to antigen for complete and sustained eradication.

The term “antigen-recognizing receptor” as used herein refers to a receptor that is capable of recognizing a target antigen (e.g., CD33). In certain embodiments, the antigen-recognizing receptor is capable of activating an immune or immunoresponsive cell (e.g., a T cell) upon its binding to the target antigen.

As used herein, the term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments F(ab′)₂, and Fab. F(ab′)₂, and Fab fragments that lack the Fe fragment of intact antibody, clear more rapidly from the circulation, and may have less non-specific tissue binding of an intact antibody (Wahl et al., Nucl Med (1983); 24:316-325). As used herein, include whole native antibodies, bispecific antibodies; chimeric antibodies; Fab, Fab′, single chain V region fragments (scFv), fusion polypeptides, and unconventional antibodies. In certain embodiments, an antibody is a glycoprotein comprising at least two heavy (H) chains and two light (L) chains inter-connected by disulfide bonds. Each heavy chain is comprised of a heavy chain variable region (abbreviated herein as V_{H}) and a heavy chain constant (C_{H}) region. The heavy chain constant region is comprised of three domains, CH1, CH2 and CH3. Each light chain is comprised of a light chain variable region (abbreviated herein as V_{L}) and a light chain constant C_{L} region. The light chain constant region is comprised of one domain, C_{L}. The V_{H} and V_{L}regions can be further sub-divided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

As used herein, “CDRs” are defined as the complementarity determining region amino acid sequences of an antibody which are the hypervariable regions of immunoglobulin heavy and light chains. See, e.g., Kabat et al., Sequences of Proteins of Immunological Interest, 4th U. S. Department of Health and Human Services, National Institutes of Health (1987), or IMGT numbering system (Lefranc, The Immunologist (1999); 7:132-136; Lefranc et al., Dev. Comp. Immunol. (2003); 27:55-77). Generally, antibodies comprise three heavy chain and three light chain CDRs or CDR regions in the variable region. CDRs provide the majority of contact residues for the binding of the antibody to the antigen or epitope. In certain embodiments, the CDRs regions are delineated using the IMGT numbering system. In certain embodiments, the CDR regions are delineated using the IMGT numbering system accessible at http://www.imgt.org/IMGT_vquest/input.

As used herein, the term “single-chain variable fragment” or “scFv” is a fusion protein of the variable regions of the heavy (V_{H}) and light chains (V_{L}) of an immunoglobulin (e.g., mouse or human) covalently linked to form a V_{H}::VL heterodimer. The heavy (V_{H}) and light chains (V_{L}) are either joined directly or joined by a peptide-encoding linker (e.g., 10, 15, 20, 25 amino acids), which connects the N-terminus of the V_{H} with the C-terminus of the V_{L}, or the C-terminus of the V_{H} with the N-terminus of the V_{L}. The linker is usually rich in glycine for flexibility, as well as serine or threonine for solubility. The linker can link the heavy chain variable region and the light chain variable region of the extracellular antigen-binding domain. Non-limiting examples of linkers are disclosed in Shen et al., Anal. Chem. 80(6):1910-1917 (2008) and WO 2014/087010, the contents of which are hereby incorporated by reference in their entireties. In certain embodiments, the linker is a G4S linker.

In certain embodiments, the linker comprises or consists of the amino acid sequence set forth in SEQ ID NO: 95, which is provided below:

[SEQ ID NO: 95]
GGGGSGGGGSGGGSGGGGS

In certain embodiments, the linker comprise or consists of the amino acid sequence set forth in SEQ ID NO: 96, which is provided below:

[SEQ ID NO: 96]
GGGGSGGGGSGGGGS

In certain embodiments, the linker comprises or consists of the amino acid sequence set forth in SEQ ID NO: 97, which is provided below:

[SEQ ID NO: 97]
GGGGSGGGGSGGGGSGGGSGGGGS

In certain embodiments, the linker comprises or consists of the amino acid sequence set forth in SEQ ID NO: 98, which is provided below:

[SEQ ID NO: 98]
GGGGSGGGGSGGGGSGGGGSGGGSGGGGS

In certain embodiments, the linker comprises or consists of the amino acid sequence set forth in SEQ ID NO: 99, which is provided below:

[SEQ ID NO: 99]
GGGGS

In certain embodiments, the linker comprises or consists of the amino acid sequence set forth in SEQ ID NO: 100, which is provided below:

[SEQ ID NO: 100]
GGGGSGGGGS

Despite removal of the constant regions and the introduction of a linker, scFv proteins retain the specificity of the original immunoglobulin. Single chain Fv polypeptide antibodies can be expressed from a nucleic acid comprising V_{H}- and V_{L}-encoding sequences as described by Huston, et al. Proc. Nat. Acad. Sci. USA, (1988); 85:5879-5883; U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hyrbidoma (Larchmt) (2008); 27(6):455-51; Peter et al., J Cachexia Sarcopenia Muscle (2012); August 12; Shieh et al., J Imunol (2009); 183(4):2277-85; Giomarelli et al., Thromb Haemost (2007); 97(6):955-63; Fife eta., J Clin Invst (2006); 116(8):2252-61; Brocks et al., Immunotechnology 1997 3(3):173-84; Moosmayer et al., Ther Immunol 1995 2(10:31-40). Agonistic scFvs having stimulatory activity have been described (Peter et al., J Biol Chern (2003); 25278(38):36740-7; Xie et al., Nat Biotech 1997 15(8):768-71; Ledbetter et al., Crit Rev Immunol (1997); 17(5-6):427-55; Ho et al., BioChim Biophys Acta (2003); 1638(3):257-66).

The term “chimeric antigen receptor” or “CAR” as used herein refers to a molecule comprising an extracellular antigen-binding domain that is fused to an intracellular signaling domain that is capable of activating or stimulating an immunoresponsive cell, and a transmembrane domain. In certain embodiments, the extracellular antigen-binding domain of a CAR comprises a scFv. The scFv can be derived from fusing the variable heavy and light regions of an antibody. Alternatively or additionally, the scFv may be derived from Fab's (instead of from an antibody, e.g., obtained from Fab libraries). In certain embodiments, the scFv is fused to the transmembrane domain and then to the intracellular signaling domain. By “substantially identical” or “substantially homologous” is meant a polypeptide or nucleic acid molecule exhibiting at least about 50% homologous or identical to a reference amino acid sequence (for example, any of the amino acid sequences described herein) or a reference nucleotide sequence (for example, any of the nucleotide sequences described herein). In certain embodiments, such a sequence is at least about 60%, at least about 65%, at least about 70%, at least about 75%, at least about 80%, at least about 85%, at least about 90%, at least about 95%, at least about 99%, or at least about 100% homologous or identical to the sequence of the amino acid or nucleic acid used for comparison.

Sequence identity can be measured by using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-100 indicating a closely related sequence.

As used herein, the percent homology between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm.

The percent homology between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the amino acids sequences of the presently disclosed subject matter can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the specified sequences (e.g., heavy and light chain variable region sequences of scFv m903, m904, m905, m906, and m900) disclosed herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used.

An “effective amount” is an amount sufficient to affect a beneficial or desired clinical result upon treatment. An effective amount can be administered to a subject in one or more doses. In certain embodiments, an effective amount can be an amount that is sufficient to palliate, ameliorate, stabilize, reverse or slow the progression of the disease, or otherwise reduce the pathological consequences of the disease. The effective amount can be determined by a physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the cells administered.

As used herein, the term “a conservative sequence modification” refers to an amino acid modification that does not significantly affect or alter the binding characteristics of the presently disclosed CD33-targeted CAR (e.g., the extracellular antigen-binding domain) comprising the amino acid sequence. Conservative modifications can include amino acid substitutions, additions and deletions. Modifications can be introduced into the extracellular antigen-binding domain of the presently disclosed CAR by standard techniques known in the art, such as site-directed mutagenesis and PCR-mediated mutagenesis. Amino acids can be classified into groups according to their physicochemical properties such as charge and polarity. Conservative amino acid substitutions are ones in which the amino acid residue is replaced with an amino acid within the same group. For example, amino acids can be classified by charge: positively-charged amino acids include lysine, arginine, histidine, negatively-charged amino acids include aspartic acid, glutamic acid, neutral charge amino acids include alanine, asparagine, cysteine, glutamine, glycine, isoleucine, leucine, methionine, phenylalanine, proline, serine, threonine, tryptophan, tyrosine, and valine. In addition, amino acids can be classified by polarity: polar amino acids include arginine (basic polar), asparagine, aspartic acid (acidic polar), glutamic acid (acidic polar), glutamine, histidine (basic polar), lysine (basic polar), serine, threonine, and tyrosine; non-polar amino acids include alanine, cysteine, glycine, isoleucine, leucine, methionine, phenylalanine, proline, tryptophan, and valine. Thus, one or more amino acid residues within a CDR region can be replaced with other amino acid residues from the same group and the altered antibody can be tested for retained function (i.e., the functions set forth in (c) through (1) above) using the functional assays described herein. In certain embodiments, no more than one, no more than two, no more than three, no more than four, no more than five residues within a specified sequence or a CDR region are altered.

As used herein, the term “endogenous” refers to a nucleic acid molecule or polypeptide that is normally expressed in a cell or tissue.

As used herein, the term “exogenous” refers to a nucleic acid molecule or polypeptide that is not endogenously present in a cell. The term “exogenous” would therefore encompass any recombinant nucleic acid molecule or polypeptide expressed in a cell, such as foreign, heterologous, and over-expressed nucleic acid molecules and polypeptides. By “exogenous” nucleic acid is meant a nucleic acid not present in a native wild-type cell; for example, an exogenous nucleic acid may vary from an endogenous counterpart by sequence, by position/location, or both. For clarity, an exogenous nucleic acid may have the same or different sequence relative to its native endogenous counterpart; it may be introduced by genetic engineering into the cell itself or a progenitor thereof, and may optionally be linked to alternative control sequences, such as a non-native promoter or secretory sequence.

By a “heterologous nucleic acid molecule or polypeptide” is meant a nucleic acid molecule (e.g., a cDNA, DNA or RNA molecule) or polypeptide that is not normally present in a cell or sample obtained from a cell. This nucleic acid may be from another organism, or it may be, for example, an mRNA molecule that is not normally expressed in a cell or sample.

By “increase” is meant to alter positively by at least about 5%. An alteration may be by about 5%, about 10%, about 25%, about 30%, about 50%, about 75%, about 100% or more.

By “reduce” is meant to alter negatively by at least about 5%. An alteration may be by about 5%, about 10%, about 25%, about 30%, about 50%, about 75%, or even by about 100%.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel.

For a protein that can be subjected to modifications, for example, phosphorylation or glycosylation, different modifications may give rise to different isolated proteins, which can be separately purified.

By “isolated cell” is meant a cell that is separated from the molecular and/or cellular components that naturally accompany the cell.

The term “antigen-binding domain” as used herein refers to a domain capable of specifically binding a particular antigenic determinant or set of antigenic determinants present on a cell.

By “recognize” is meant selectively binds to a target. A T cell that recognizes a tumor can expresses a receptor (e.g., a CAR) that binds to a tumor antigen.

By “signal sequence” or “leader sequence” is meant a peptide sequence (e.g., 5, 10, 15, 20, 25 or 30 amino acids) present at the N-terminus of newly synthesized proteins that directs their entry to the secretory pathway

By “specifically binds” or “specifically binds to” or “specifically target” is meant a polypeptide or a fragment thereof that recognizes and/or binds to a biological molecule of interest (e.g., a polypeptide, e.g., a CD33 polypeptide), but which does not substantially recognize and/or bind other molecules in a sample, for example, a biological sample, which naturally includes a presently disclosed polypeptide (e.g., a CD33 polypeptide). In certain embodiments, the that the presently disclosed antigen recognizing receptor binds to CD33 (e.g., human CD33) with a dissociation constant (K_{D}) of about 1×10−8 M or less, about 5×10⁻⁹ M or less, about 1×10⁻⁹ M or less, about 5×10⁻¹⁰ M or less, about 1×10⁻¹⁰ M or less, about 5×10⁻¹¹ M or less, or about 1×10⁻¹¹ M or less.

As used herein, the term “derivative” refers to a compound that is derived from some other compound and maintains its general structure. For example, but without any limitation, trichloromethane (chloroform) is a derivative of methane.

The terms “comprises”, “comprising”, and are intended to have the broad meaning ascribed to them in U.S. Patent Law and can mean “includes”, “including” and the like.

As used herein, “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

An “individual” or “subject” herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

Other aspects of the presently disclosed subject matter are described in the following disclosure and are within the ambit of the presently disclosed subject matter.

### 5.2. CD33

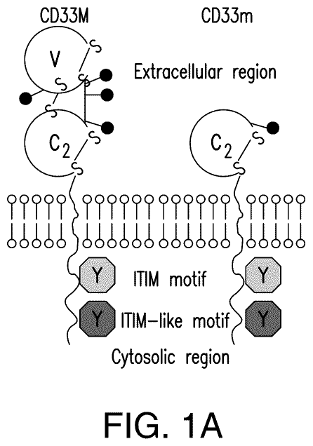

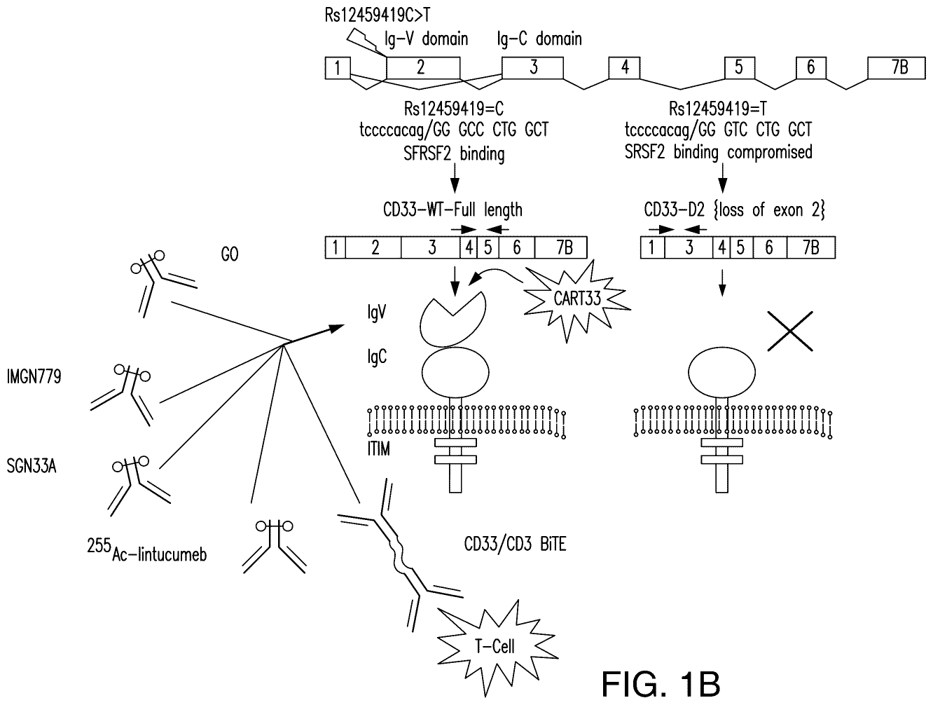

CD33 is a single pass transmembrane molecule and a member of the sialic acid-binding immunoglobulin (Ig)-like lectin (Siglec) family. CD33 consists of two extracellular domains with immunoglobulin-like folds, IgV and IgC2 (see FIG. 1A). CD33 has 3 isoforms produced via alternate splicing, with isoform 3 missing the IgV domain (Ehninger et al., Blood Cancer J 4, e218 (2014); Sanford et al., Leuk Lymphoma 57, 1965-1968 (2016); and Haubner et al., Leukemia 33, 64-74 (2019)) Recent studies showed that about 50% of AML patients have a CD33 single-nucleotide polymorphism (SNP) (rs12459419 C>T) that leads to the expression of an alternatively spliced CD33 isoform lacking exon 2, resulting in the elimination of the IgV domain (Bakker et al., Cancer Res 64, 8443-8450 (2004)). In these patients, gemtuzumab ozogamicin (GO), an antibody-drug conjugate (ADC) targeting CD33, had no impact and increased relapse risk likely due to the inability of this ADC to kill AML cells expressing this IgV-lacking CD33 isoform. This issue is bound to be encountered by all the currently clinically available CD33-targeting products given their epitopes in the IgV domain (see FIG. 1B) (Perna et al., Cancer Cell 32, 506-519 e505 (2017)).

In certain embodiments, the antigen recognizing receptor binds to human CD33. In certain embodiments, the human CD33 comprises or consists of the amino acid sequence with a UniProt Reference No: P20138-1 (SEQ ID NO: 1) or a fragment thereof. SEQ ID NO: 1 is provided below. In certain embodiments, the CD33 comprises an extracellular domain, a transmembrane domain, and a cytoplasmic domain. In certain embodiments, the extracellular domain comprises or consists of amino acids 18 to 259 of SEQ ID NO: 1. In certain embodiments, the transmembrane domain comprises or consists of amino acids 260 to 282 of SEQ ID NO: 1. In certain embodiments, the cytoplasmic domain comprises or consists of amino acids 283 to 364 of SEQ ID NO: 1.

[SEQ ID NO: 1]
MPLLLLLPLLWAGALAMDPNFWLQVQESVT
VQEGLCVLVPCTFFHPIPYYDKNSPVHGYW
FREGAIISRDSPVATNKLDQEVQEETQGRF
RLLGDPSRNNCSLSIVDARRRDNGSYFFRM
ERGSTKYSYKSPQLSVHVTDLTHRPKILIP
GTLEPGHSKNLTCSVSWACEQGTPPIFSWL
SAAPTSLGPRTTHSSVLIITPRPQDHGTNL
TCQVKFAGAGVTTERTIQLNVTYVPQNPTT
GIFPGDGSGKQETRAGVVHGAIGGAGVTAL
LALCLCLIFFIVKTHRRKAARTAVGRNDTH
PTTGSASPKHQKKSKLHGPTETSSCSGAAP
TVEMDEELHYASLNFHGMNPSKDTSTEYSE
VRTQ

In certain embodiments, the CD33 comprises or consists of an amino acid sequence that is at least about 80%, at least about 85%, at least about 90%, at least about 95%, at least about 96%, at least about 97%, at least about 98%, or at least about 99%, at least about 100% identical to the amino acid sequence set forth in SEQ ID NO: 1 or a fragment thereof.

In certain embodiments, the antigen recognizing receptor binds to a portion of human CD33. In certain embodiments, the antigen recognizing receptor binds to the extracellular domain of CD33. In certain embodiments, the extracellular domain of CD33 comprises an Ig-like V-type domain and an Ig-like C2-type. In certain embodiments, the extracellular domain of CD33 comprises an Ig-like C2-type. In certain embodiments, the Ig-like V-type domain comprises or consists of amino acids 19 to 135 of SEQ ID NO: 1. In certain embodiments, the Ig-like C2-type domain comprises or consists of amino acids 145 to 228 of SEQ ID NO: 1.

### 5.3. Antigen-Recognizing Receptors

The presently disclosed antigen-recognizing receptors specifically target or bind to CD33. In certain embodiments, the antigen-recognizing receptor is a chimeric antigen receptor (CAR). In certain embodiments, the antigen-recognizing receptor is a TCR like fusion molecule.

The presently disclosed subject matter also provides nucleic acid molecules that encode the presently disclosed antigen-recognizing receptors. In certain embodiments, the nucleic acid molecule comprises a nucleotide sequence that encodes a polypeptide of a CD33-targeted antigen recognizing receptor disclosed herein.

### 5.3.1. Extracellular Antigen-Binding Domains

In certain embodiments, the extracellular antigen-binding domain of the antigen-recognizing receptor binds to CD33.

In certain embodiments, the extracellular antigen-binding domain is an scFv. In certain embodiments, the scFv is a human scFv. In certain embodiments, the scFv is a humanized scFv. In certain embodiments, the scFv is a murine scFv. In certain embodiments, the scFv is identified by screening scFv phage library with an antigen-Fc fusion protein.

In certain embodiments, the extracellular antigen-binding domain is a Fab. In certain embodiments, the Fab is crosslinked. In certain embodiments, the extracellular antigen-binding domain is a F(ab)₂.

Any of the foregoing molecules may be comprised in a fusion protein with a heterologous sequence to form the extracellular antigen-binding domain. In certain non-limiting embodiments, the extracellular antigen-binding domain (embodied, for example, an scFv) binds to CD33 (e.g., human CD33) with a binding affinity, for example with a dissociation constant (K_{D}) of 1×10−8 M or less, e.g., about 1×10⁻⁸ M or less, about 5×10⁻⁹ M or less, about 1×10⁻⁹ M or less, about 5×10⁻¹⁰ M or less, about 1×10⁻¹⁰ M or less, or about 1×10⁻¹¹ M or less. In certain embodiments, the presently disclosed extracellular antigen-binding domain binds to CD33 (e.g., human CD33, e.g. soluble human CD33) with a dissociation constant (K_{D}) of about 5×10⁻⁹ M or less. In certain embodiments, the presently disclosed extracellular antigen-binding domain binds to CD33 (e.g., human CD33, e.g. soluble human CD33) with a dissociation constant (K_{D}) of about 5×10⁻⁹ M. In certain embodiments, the presently disclosed extracellular antigen-binding domain binds to CD33 (e.g., human CD33, e.g. soluble human CD33) with a dissociation constant (K_{D}) of about 1×10⁻⁹ M. In certain embodiments, the presently disclosed extracellular antigen-binding domain binds to CD33 (e.g., human CD33, e.g. soluble human CD33) with a dissociation constant (K_{D}) of between about 1×10⁻⁹ M and about 5×10⁻⁹ M. In certain embodiments, the presently disclosed extracellular antigen-binding domain binds to CD33 (e.g., human CD33, e.g. soluble human CD33) with a dissociation constant (K_{D}) of between about 1×10⁻⁹ M and about 2×10⁻⁹ M. In certain embodiments, the presently disclosed extracellular antigen-binding domain binds to CD33 (e.g., human CD33, e.g. soluble human CD33) with a dissociation constant (K_{D}) of about 5×10⁻⁹ M. In certain embodiments, the presently disclosed extracellular antigen-binding domain binds to CD33 (e.g., human CD33, e.g. soluble human CD33) with a dissociation constant (K_{D}) of about 1×10⁻⁹ M.

In certain embodiments, a presently disclosed extracellular antigen-binding domain binds to a cell expressing CD33 (e.g., an AML cell expressing CD33) with a Half maximal Effective Concentration (EC50) value of from about 1 nM to about 50 nM, from about 5 nM to about 50 nM, from about 10 nM to about 50 nM, from about 20 nM to about 50 nM, from about 30 nM to about 50 nM, from about 40 nM to about 50 nM, or greater than about 50 nM. In certain embodiments, a presently disclosed extracellular antigen-binding domain binds to a cell expressing CD33 (e.g., an AML cell expressing CD33) with an EC50 value from about 1 nM to about 5 nM. In certain embodiments, a presently disclosed extracellular antigen-binding domain binds to a cell expressing CD33 (e.g., an AML cell expressing CD33) with an EC50 value of about 2 nM. In certain embodiments, a presently disclosed extracellular antigen-binding domain binds to a cell expressing CD33 (e.g., an AML cell expressing CD33) with an EC50 value of about 2.16 nM. In certain embodiments, a presently disclosed extracellular antigen-binding domain binds to a cell expressing CD33 (e.g., an AML cell expressing CD33) with an EC50 value from about 5 nM to about 10 nM. In certain embodiments, a presently disclosed extracellular antigen-binding domain binds to a cell expressing CD33 (e.g., an AML cell expressing CD33) with an EC50 value of about 10 nM. In certain embodiments, a presently disclosed extracellular antigen-binding domain binds to a cell expressing CD33 (e.g., an AML cell expressing CD33) with an EC50 value of about 8.5 nM. In certain embodiments, a presently disclosed extracellular antigen-binding domain binds to a cell expressing CD33 (e.g., an AML cell expressing CD33) with an EC50 value from about 40 nM to about 50 nM. In certain embodiments, a presently disclosed extracellular antigen-binding domain binds to a cell expressing CD33 (e.g., an AML cell expressing CD33) with an EC50 value of about 45 nM. In certain embodiments, a presently disclosed extracellular antigen-binding domain binds to a cell expressing CD33 (e.g., an AML cell expressing CD33) with an EC50 value of about 45 nM.

Binding of the extracellular antigen-binding domain can be confirmed by, for example, enzyme-linked immunosorbent assay (ELISA), radioimmunoassay (RIA), FACS analysis, bioassay (e.g., growth inhibition), or Western Blot assay. Each of these assays generally detect the presence of protein-antibody complexes of particular interest by employing a labeled reagent (e.g., an antibody, or a scFv) specific for the complex of interest. For example, the scFv can be radioactively labeled and used in a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a γ counter or a scintillation counter or by autoradiography. In certain embodiments, the CD33-targeted extracellular antigen-binding domain is labeled with a fluorescent marker. Non-limiting examples of fluorescent markers include green fluorescent protein (GFP), blue fluorescent protein (e.g., EBFP, EBFP2, Azurite, and mKalama1), cyan fluorescent protein (e.g., ECFP, Cerulean, and CyPet), and yellow fluorescent protein (e.g., YFP, Citrine, Venus, and YPet). In certain embodiments, the CD33-targeted human scFv is labeled with GFP.

In certain embodiments, the CDRs are identified according to the IMGT numbering system.

In certain embodiments, the extracellular antigen-binding domain (e.g., an scFv) comprises a V_{H} comprising a CDR1 comprising the amino acid sequence set forth in SEQ ID NO: 2 or a conservative modification thereof, a CDR2 comprising the amino acid sequence set forth in SEQ ID NO: 3 or a conservative modification thereof, and a CDR3 comprising the amino acid sequence set forth in SEQ ID NO: 4 or a conservative modification thereof. SEQ ID NOs: 2-4 are provided in Table 1.

In certain embodiments, the extracellular antigen-binding domain (e.g., an scFv) comprises a V_{L} comprising a CDR1 comprising the amino acid sequence set forth in SEQ ID NO: 5 or a conservative modification thereof, a CDR2 comprising the amino acid sequence set forth in SEQ ID NO: 6 or a conservative modification thereof, and a CDR3 comprising the amino acid sequence set forth in SEQ ID NO: 7 or a conservative modification thereof. SEQ ID NOs: 5-7 are provided in Table 1.

In certain embodiments, the extracellular antigen-binding domain (e.g., an scFv) comprises a V_{H} comprising a CDR1 comprising the amino acid sequence set forth in SEQ ID NO: 2 or a conservative modification thereof, a CDR2 comprising the amino acid sequence set forth in SEQ ID NO: 3 or a conservative modification thereof, and a CDR3 comprising the amino acid sequence set forth in SEQ ID NO: 4 or a conservative modification thereof; and a V_{L} comprising a CDR1 comprising the amino acid sequence set forth in SEQ ID NO: 5 or a conservative modification thereof, a CDR2 comprising the amino acid sequence set forth in SEQ ID NO: 6 or a conservative modification, and a CDR3 comprising the amino acid sequence set forth in SEQ ID NO: 7 or a conservative modification thereof.

In certain embodiments, the extracellular antigen-binding domain (e.g., an scFv) comprises a V_{H} comprising a CDR1 comprising the amino acid sequence set forth in SEQ ID NO: 2, a CDR2 comprising the amino acid sequence set forth in SEQ ID NO: 3, and a CDR3 comprising the amino acid sequence set forth in SEQ ID NO: 4; and a V_{L} comprising a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 7.

In certain embodiments, the extracellular antigen-binding domain (e.g., an scFv) comprises a V_{H} comprising an amino acid sequence that is at least about 80% (e.g., at least about 85%, at least about 90%, or at least about 95%) homologous or identical to the amino sequence set forth in SEQ ID NO: 8. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 8. In certain embodiments, the extracellular antigen-binding domain comprises a V_{H} comprising the amino sequence set forth in SEQ ID NO: 8. An exemplary nucleotide sequence encoding the amino acid sequence of SEQ ID NO: 8 is set forth in SEQ ID NO: 10. SEQ ID NOs: 8 and 10 are provided in Table 1 below.

In certain embodiments, the extracellular antigen-binding domain (e.g., an scFv) comprises a V_{L} comprising an amino acid sequence that is at least about 80% (e.g., at least about 85%, at least about 90%, or at least about 95%) homologous or identical to the amino sequence set forth in SEQ ID NO: 9. For example, the extracellular antigen-binding domain (e.g., an scFv) comprises a V_{L}comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 9. In certain embodiments, the extracellular antigen-binding domain comprises a V_{L} comprising the amino sequence set forth in SEQ ID NO: 9. An exemplary nucleotide sequence encoding the amino acid sequence of SEQ ID NO: 9 is set forth in SEQ ID NO: 11. SEQ ID NOs: 9 and 11 are provided in Table 1 below.

In certain embodiments, the extracellular antigen-binding domain (e.g., an scFv) comprises a V_{H} comprising the amino acid sequence set forth in SEQ ID NO: 8, and a V_{L} Comprising the amino acid sequence set forth in SEQ ID NO: 9. In certain embodiments, the extracellular antigen-binding domain is an scFv. In certain embodiments, the scFv is designated as “3-P14”. In certain embodiments, the V_{H}- and V_{L} are linked via a linker. In certain embodiments, the linker comprises the amino acid sequence set forth in SEQ ID NO: 96.

In certain embodiments, the variable regions within the extracellular antigen-binding domain have to be linked one after another such that at the N-terminus of the extracellular antigen-binding domain, a heavy chain variable region (V_{H}) is positioned. In certain embodiments, if the extracellular antigen-binding domain is an scFv, the variable regions are positioned from the N- to the C-terminus: V_{H}-V_{L}.

In certain embodiments, the variable regions within the extracellular antigen-binding domain have to be linked one after another such that at the N-terminus of the extracellular antigen-binding domain, a light chain variable region (V_{L}) is positioned. In certain embodiments, the extracellular antigen-binding domain is an scFv, the variable regions are positioned from the N- to the C-terminus: V_{L}-V_{H}.

TABLE 1

| CDRs | 1 | 2 | 3 |
|---|---|---|---|
| V_{H} | GFTESTYA | ISGRGGST | AGRGDYYYYYGMDV |
| | [SEQ ID NO: 2] | [SEQ ID NO: 3] | [SEQ ID NO: 4] |
| V_{L} | QSLVYSDGNTY | KIS | MQSTQFPHT |
| | [SEQ ID NO: 5] | [SEQ ID NO: 6] | [SEQ ID NO: 7] |

| | |
|---|---|
| Full V_{H} | EVQLLESGGGLVQPGGSLRLSCAASGFTFSTYAMSWVRQAPGKGLEWVSAISGRGGSTY |
| | YTDSVKGRFTI SRDNSKNTVSLQMNSLRAEDTAVYYCAGRGDYYYYYGMDVWGQGTTVT |
| | VSA [SEQ ID NO: 8] |
| Full V_{L} | DIVMTQSPLSSPVTLGQPASFSCRSSQSLVYSDGNTYLSWLQQRPGQPPRLLIYKISNR |
| | FSGVPDRESGSGAGTDFTLKI SRVEAEDVGVYYCMQSTQFPHTFGQGTKLEIK [SEQ |
| | ID NO: 9] |
| DNA for | GAGGTGCAGCTGTTGGAGTCTGGGGGAGGCTTGGTCCAGCCTGGGGGGTCCCTGAGACT |
| Full V_{H} | CTCCTGTGCAGCCTCTGGATTCACCTTTAGCACCTATGCCATGAGCTGGGTCCGCCAGG |
| | CTCCAGGGAAGGGGCTGGAGTGGGTCTCAGCTATTAGTGGTCGTGGTGGTAGCACATAC |
| | TACACAGACTCCGTGAAGGGCCGGTTCACCATCTCCAGAGACAATTCCAAGAACACGGT |
| | GTCTCTGCAAATGAACAGCCTGAGAGCCGAGGACACGGCCGTATATTACTGTGCGGGCC |
| | GGGGAGATTACTACTACTACTACGGTATGGACGTCTGGGGCCAAGGGACCACGGTCACC |
| | GTCTCCGCA [SEQ ID NO: 10] |
| DNA for | GATATTGTGATGACCCAGAGTCCACTCTCCTCACCTGTCACCCTTGGACAGCCGGCCTC |
| Full V_{L} | CTTCTCCTGCAGGTCTAGTCAAAGCCTCGTATACAGTGATGGAAACACCTACTTGAGTT |
| | GGCTTCAGCAGAGGCCAGGCCAGCCTCCAAGACTCCTAATTTATAAGATTTCTAACCGG |
| | TTCTCTGGGGTCCCAGACAGATTCAGTGGCAGTGGGGCAGGGACAGATTTCACACTGAA |
| | AATCAGCAGGGTGGAAGCTGAGGATGTCGGGGTTTATTACTGCATGCAATCTACACAAT |
| | TTCCTCACACTTTTGGCCAGGGGACCAAGCTGGAGATCAAA [SEQ ID NO: 11] |

In certain embodiments, the extracellular antigen-binding domain (e.g., an scFv) comprises a V_{H} comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof. SEQ ID NOs: 12-14 are provided in Table 2.

In certain embodiments, the extracellular antigen-binding domain (e.g., an scFv) comprises a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 2.

In certain embodiments, the extracellular antigen-binding domain (e.g., an scFv) comprises a V_{H} comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, a CDR3 comprising the amino acid sequence set forth in SEQ ID NO: 14 or a conservative modification thereof; and a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof.

In certain embodiments, the extracellular antigen-binding domain (e.g., a scFv) comprises a V_{H} comprising a CDR1 comprising the amino acid sequence set forth in SEQ ID NO: 12, a CDR2 comprising the amino acid sequence set forth in SEQ ID NO: 13, a CDR3 comprising the amino acid sequence set forth in SEQ ID NO: 14; and a V_{L} comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the extracellular antigen-binding domain (e.g., an scFv) comprises a V_{H} comprising an amino acid sequence that is at least about 80% (e.g., at least about 85%, at least about 90%, or at least about 95%) homologous or identical to the amino sequence set forth in SEQ ID NO: 18. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 18. In certain embodiments, the extracellular antigen-binding domain comprises a V_{H} comprising the amino sequence set forth in SEQ ID NO: 18. An exemplary nucleotide sequence encoding the amino acid sequence of SEQ ID NO: 18 is set forth in SEQ ID NO: 20. SEQ ID NO: 18 and 20 are provided in Table 2 below.

In certain embodiments, the extracellular antigen-binding domain (e.g., an scFv) comprises a V_{L} comprising an amino acid sequence that is at least about 80% (e.g., at least about 85%, at least about 90%, or at least about 95%) homologous or identical to the amino sequence set forth in SEQ ID NO: 19. For example, the extracellular antigen-binding domain (e.g., an scFv) comprises a V_{L}comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 19. In certain embodiments, the extracellular antigen-binding domain comprises a V_{L} comprising the amino sequence set forth in SEQ ID NO: 19. An exemplary nucleotide sequence encoding the amino acid sequence of SEQ ID NO: 19 is set forth in SEQ ID NO: 21. SEQ ID NO: 19 and 21 are provided in Table 2 below.

In certain embodiments, the extracellular antigen-binding domain (e.g., an scFv) comprises a V_{H} comprising the amino acid sequence set forth in SEQ ID NO: 18, and a V_{L} comprising the amino acid sequence set forth in SEQ ID NO: 19. In certain embodiments, the extracellular antigen-binding domain is an scFv. In certain embodiments, the scFv is designated as “4-B2”. In certain embodiments, the V_{H} and V_{L} are linked via a linker. In certain embodiments, the linker comprises the amino acid sequence set forth in SEQ ID NO: 96.

In certain embodiments, the variable regions within the extracellular antigen-binding domain have to be linked one after another such that at the N-terminus of the extracellular antigen-binding domain, a heavy chain variable region (V_{H}) is positioned. In certain embodiments, if the extracellular antigen-binding domain is an scFv, the variable regions are positioned from the N- to the C-terminus: V_{H}-V_{L}.

In certain embodiments, the variable regions within the extracellular antigen-binding domain have to be linked one after another such that at the N-terminus of the extracellular antigen-binding domain, a light chain variable region (V_{L}) is positioned. In certain embodiments, if the extracellular antigen-binding domain is an scFv, the variable regions are positioned from the N- to the C-terminus: V_{L}-V_{H}.

TABLE 2

| CDRs | 1 | 2 | 3 |
|---|---|---|---|
| V_{H} | GFIFSSNA | ISGYGGNT | AKWGTYIVGATGDY |
| | [SEQ ID NO: 12] | [SEQ ID NO: 13] | [SEQ ID NO: 14] |
| V_{L} | SNDVGGYNY | EVS | SSYAGSNNWV |
| | [SEQ ID NO: 15] | [SEQ ID NO: 16] | [SEQ ID NO: 17] |

| | |
|---|---|
| Full V_{H} | EVHLLESGGGLVQPGGSLRLSCAASGFIFSSNAMSWVRQAPGKGLEWVSAISGYGGNTY |
| | YADSVKGRFTISRDNSKNTLYLQMNSLRAEDTAVYYCAKWGTYIVGATGDYWGQGTLVT |
| | VSS [SEQ ID NO: 18] |
| Full V_{L} | QSALTQPPSASGSPGQSVTISCTGTSNDVGGYNYVSWYQQHPGKAPKLLIYEVSKRPSG |
| | VPDRESGSQSGNTASLTVSGLQAEDEADYYCSSYAGSNNWVFGGGTKLTVL [SEQ ID |
| | NO: 19] |
| DNA for | GAGGTGCACCTGTTGGAGTCTGGGGGAGGCTTGGTACAGCCTGGGGGGTCCCTGAGACT |
| Full V_{H} | CTCCTGTGCAGCCTCTGGATTCATCTTTAGCAGCAATGCCATGAGCTGGGTCCGCCAGG |
| | CTCCAGGGAAGGGACTGGAGTGGGTCTCAGCTATTAGTGGTTATGGTGGTAACACATAC |
| | TACGCAGACTCCGTGAAGGGCCGGTTCACCATCTCCAGAGACAATTCCAAGAACACGCT |
| | ATATCTGCAAATGAACAGCCTGAGAGCCGAGGACACGGCCGTATATTACTGTGCGAAAT |
| | GGGGGACTTATATAGTGGGAGCTACGGGTGACTACTGGGGCCAGGGAACTCTGGTCACC |
| | GTCTCCTCA [SEQ ID NO: 20] |
| DNA for | CAGTCTGCCCTGACTCAGCCTCCCTCCGCGTCCGGGTCTCCTGGACAGTCAGTCACCAT |
| Full V_{L} | CTCCTGCACTGGAACCAGCAATGACGTTGGTGGTTATAACTATGTCTCCTGGTACCAAC |
| | AGCACCCAGGCAAAGCCCCCAAACTCTTGATTTATGAGGTCAGTAAGCGGCCCTCAGGG |
| | GTCCCTGATCGCTTCTCTGGCTCCCAGTCTGGCAACACGGCCTCCCTGACCGTCTCTGG |
| | GCTCCAGGCTGAGGATGAGGCTGATTATTACTGCAGCTCATATGCAGGCAGCAACAATT |
| | GGGTGTTCGGCGGAGGGACCAAGCTGACCGTCCTA [SEQ ID NO: 21] |

In certain embodiments, the extracellular antigen-binding domain (e.g., an scFv) comprises a V_{H} comprising a CDR1 comprising the amino acid sequence set forth in SEQ ID NO: 22 or a conservative modification thereof, a CDR2 comprising the amino acid sequence set forth in SEQ ID NO: 23 or a conservative modification thereof, and a CDR3 comprising the amino acid sequence set forth in SEQ ID NO: 24 or a conservative modification thereof. SEQ ID NOS: 22-24 are provided in Table 3.

In certain embodiments, the extracellular antigen-binding domain (e.g., an scFv) comprises a V_{L} comprising a CDR1 comprising the amino acid sequence set forth in SEQ ID NO: 25 or a conservative modification thereof, a CDR2 comprising the amino acid sequence set forth in SEQ ID NO: 26 or a conservative modification thereof, and a CDR3 comprising the amino acid sequence set forth in SEQ ID NO: 27 or a conservative modification thereof. SEQ ID NOs: 25-27 are provided in Table 3.

In certain embodiments, the extracellular antigen-binding domain (e.g., an scFv) comprises a V_{H} comprising a CDR1 comprising the amino acid sequence set forth in SEQ ID NO: 22 or a conservative modification thereof, a CDR2 comprising the amino acid sequence set forth in SEQ ID NO: 23 or a conservative modification thereof, a CDR3 comprising the amino acid sequence set forth in SEQ ID NO: 24 or a conservative modification thereof; and a V_{L} Comprising a CDR1 comprising the amino acid sequence set forth in SEQ ID NO: 25 or a conservative modification thereof, a CDR2 comprising the amino acid sequence set forth in SEQ ID NO: 26 or a conservative modification thereof, and a CDR3 comprising the amino acid sequence set forth in SEQ ID NO: 27 or a conservative modification thereof.

In certain embodiments, the extracellular antigen-binding domain (e.g., an scFv) comprises a V_{H} comprising a CDR1 comprising the amino acid sequence set forth in SEQ ID NO: 22, a CDR2 comprising the amino acid sequence set forth in SEQ ID NO: 23, a CDR3 comprising the amino acid sequence set forth in SEQ ID NO: 24; and a V_{L} comprising a CDR1 comprising the amino acid sequence set forth in SEQ ID NO: 25, a CDR2 comprising the amino acid sequence set forth in SEQ ID NO: 26, and a CDR3 comprising the amino acid sequence set forth in SEQ ID NO: 27.

In certain embodiments, the extracellular antigen-binding domain (e.g., an scFv) comprises a V_{H} comprising an amino acid sequence that is at least about 80% (e.g., at least about 85%, at least about 90%, or at least about 95%) homologous or identical to the amino sequence set forth in SEQ ID NO: 28. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 28. In certain embodiments, the extracellular antigen-binding domain comprises a V_{H} comprising the amino sequence set forth in SEQ ID NO: 28. An exemplary nucleotide sequence encoding the amino acid sequence of SEQ ID NO: 28 is set forth in SEQ ID NO: 30. SEQ ID NO: 28 and 30 are provided in Table 3 below.

In certain embodiments, the extracellular antigen-binding domain (e.g., an scFv) comprises a V_{L} comprising an amino acid sequence that is at least about 80% (e.g., at least about 85%, at least about 90%, or at least about 95%) homologous or identical to the amino sequence set forth in SEQ ID NO: 29. For example, the extracellular antigen-binding domain (e.g., an scFv) comprises a V_{L}comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 29. In certain embodiments, the extracellular antigen-binding domain comprises a V_{L} comprising the amino sequence set forth in SEQ ID NO: 29. An exemplary nucleotide sequence encoding the amino acid sequence of SEQ ID NO: 29 is set forth in SEQ ID NO: 31. SEQ ID NO: 29 and 31 are provided in Table 3 below.

In certain embodiments, the extracellular antigen-binding domain (e.g., an scFv) comprises a V_{H} comprising the amino acid sequence set forth in SEQ ID NO: 28, and a V_{L} comprising the amino acid sequence set forth in SEQ ID NO: 29. In certain embodiments, the extracellular antigen-binding domain is an scFv. In certain embodiments, the scFv is designated as “1-J19”. In certain embodiments, the V_{H} and V_{L} are linked via a linker. In certain embodiments, the linker comprises the amino acid sequence set forth in SEQ ID NO: 96.

In certain embodiments, the variable regions within the extracellular antigen-binding domain have to be linked one after another such that at the N-terminus of the extracellular antigen-binding domain, a heavy chain variable region (V_{H}) is positioned. In certain embodiments, if the extracellular antigen-binding domain is an scFv, the variable regions are positioned from the N- to the C-terminus: V_{H}-V_{L}.

In certain embodiments, the variable regions within the extracellular antigen-binding domain have to be linked one after another such that at the N-terminus of the extracellular antigen-binding domain, a light chain variable region (V_{L}) is positioned. In certain embodiments, if the extracellular antigen-binding domain is an scFv, the variable regions are positioned from the N- to the C-terminus: V_{L}-V_{H}.

TABLE 3

| CDRs | 1 | 2 | 3 |
|---|---|---|---|
| V_{H} | GDSVSSNSAA | TYFRSKWYN | ASEGGSYYDH |
| | [SEQ ID NO: 22] | [SEQ ID NO: 23] | [SEQ ID NO: 24] |
| V_{L} | QGISNW | AAS | QQADSFPFT |
| | [SEQ ID NO: 25] | [SEQ ID NO: 26] | [SEQ ID NO: 27] |

| | |
|---|---|
| Full V_{H} | QVQLQQSGPGLVKPSQTLSLTCAISGDSVSSNSAAWNWIRQSPSRGLEWLGRTYFRSKW |
| | YNVYAVSVKSRITINPDTSKNQFSLQLNSVTPEDTAVYYCASEGGSYYDHWGQGTLVTV |
| | SS [SEQ ID NO: 28] |
| Full V_{L} | DIQMTQSPSSVSASVGDRVTITCRASQGISNWLTWYQQKPGKAPKLLIYAASSLQSGVP |
| | SRFSGSGSGTDFTLTISSLQPEDFATYYCQQADSFPFTFGPGTKVDIK [SEQ ID |
| | NO: 29] |
| DNA for | CAGGTACAGCTGCAGCAGTCAGGTCCAGGACTGGTGAAGCCCTCGCAGACCCTCTCACT |
| Full V_{H} | CACCTGTGCCATCTCCGGGGACAGTGTCTCTAGCAACAGTGCTGCTTGGAACTGGATCA |
| | GGCAGTCCCCATCGAGAGGCCTTGAGTGGCTGGGAAGGACATACTTCAGGTCCAAGTGG |
| | TATAATGTTTATGCAGTGTCTGTGAAGAGTCGAATAACCATCAACCCAGACACATCCAA |
| | GAACCAGTTCTCCCTGCAGCTGAACTCTGTGACTCCCGAGGACACGGCTGTGTATTATT |
| | GTGCAAGCGAGGGTGGGAGCTATTATGACCACTGGGGCCAGGGAACCCTGGTCACCGTC |
| | TCCTCA [SEQ ID NO: 30] |
| DNA for | GACATCCAGATGACCCAGTCTCCATCTTCCGTGTCTGCATCTGTAGGAGACAGAGTCAC |
| Full V_{L} | CATCACTTGTCGGGCGAGTCAGGGTATTAGTAATTGGTTAACCTGGTATCAGCAGAAAC |
| | CAGGGAAAGCCCCTAAGCTCCTGATCTATGCTGCATCCAGTTTGCAAAGTGGGGTCCCA |
| | TCAAGGTTCAGCGGCAGTGGATCTGGGACAGATTTCACTCTCACCATCAGCAGCCTGCA |
| | GCCTGAAGATTTTGCAACTTACTATTGTCAACAGGCTGACAGTTTCCCATTCACTTTCG |
| | GCCCTGGGACCAAAGTGGATATCAAA [SEQ ID NO: 31] |

In certain embodiments, the extracellular antigen-binding domain (e.g., an scFv) comprises a V_{H} comprising a CDR1 comprising the amino acid sequence set forth in SEQ ID NO: 32 or a conservative modification thereof, a CDR2 comprising the amino acid sequence set forth in SEQ ID NO: 33 or a conservative modification thereof, and a CDR3 comprising the amino acid sequence set forth in SEQ ID NO: 34 or a conservative modification thereof. SEQ ID NOs: 32-34 are provided in Table 4.

In certain embodiments, the extracellular antigen-binding domain (e.g., an scFv) comprises a V_{L} comprising a CDR1 comprising the amino acid sequence set forth in SEQ ID NO: 25 or a conservative modification thereof, a CDR2 comprising the amino acid sequence set forth in SEQ ID NO: 26 or a conservative modification thereof, and a CDR3 comprising the amino acid sequence set forth in SEQ ID NO: 27 or a conservative modification thereof. SEQ ID NOs: 25-27 are provided in Table 4.

In certain embodiments, the extracellular antigen-binding domain (e.g., an scFv) comprises a V_{H} comprising a CDR1 comprising the amino acid sequence set forth in SEQ ID NO: 32 or a conservative modification thereof, a CDR2 comprising the amino acid sequence set forth in SEQ ID NO: 33 or a conservative modification thereof, a CDR3 comprising the amino acid sequence set forth in SEQ ID NO: 34 or a conservative modification thereof; and a V_{L} Comprising a CDR1 comprising the amino acid sequence set forth in SEQ ID NO: 25 or a conservative modification thereof, a CDR2 comprising the amino acid sequence set forth in SEQ ID NO: 26 or a conservative modification thereof, and a CDR3 comprising the amino acid sequence set forth in SEQ ID NO: 27 or a conservative modification thereof.

In certain embodiments, the extracellular antigen-binding domain (e.g., an scFv) comprises a V_{H} comprising a CDR1 comprising the amino acid sequence set forth in SEQ ID NO: 32, a CDR2 comprising the amino acid sequence set forth in SEQ ID NO: 33, a CDR3 comprising the amino acid sequence set forth in SEQ ID NO: 34; and a V_{L} comprising a CDR1 comprising the amino acid sequence set forth in SEQ ID NO: 25, a CDR2 comprising the amino acid sequence set forth in SEQ ID NO: 26, and a CDR3 comprising the amino acid sequence set forth in SEQ ID NO: 27.

In certain embodiments, the extracellular antigen-binding domain (e.g., an scFv) comprises a V_{H} comprising an amino acid sequence that is at least about 80% (e.g., at least about 85%, at least about 90%, or at least about 95%) homologous or identical to the amino sequence set forth in SEQ ID NO: 35. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 35. In certain embodiments, the extracellular antigen-binding domain comprises a V_{H} comprising the amino sequence set forth in SEQ ID NO: 35. An exemplary nucleotide sequence encoding the amino acid sequence of SEQ ID NO: 35 is set forth in SEQ ID NO: 36. SEQ ID NO: 35 and 36 are provided in Table 4 below.

In certain embodiments, the extracellular antigen-binding domain (e.g., an scFv) comprises a V_{L} comprising an amino acid sequence that is at least about 80% (e.g., at least about 85%, at least about 90%, or at least about 95%) homologous or identical to the amino sequence set forth in SEQ ID NO: 29. For example, the extracellular antigen-binding domain (e.g., an scFv) comprises a V_{L}comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 29. In certain embodiments, the extracellular antigen-binding domain comprises a V_{L} comprising the amino sequence set forth in SEQ ID NO: 29. An exemplary nucleotide sequence encoding the amino acid sequence of SEQ ID NO: 29 is set forth in SEQ ID NO: 31. SEQ ID NO: 29 and 31 are provided in Table 4 below.

In certain embodiments, the extracellular antigen-binding domain (e.g., an scFv) comprises a V_{H} comprising the amino acid sequence set forth in SEQ ID NO: 35, and a V_{L} comprising the amino acid sequence set forth in SEQ ID NO: 29. In certain embodiments, the extracellular antigen-binding domain is an scFv. In certain embodiments, the scFv is designated as “1-J19-2”. In certain embodiments, the V_{H} and V_{L} are linked via a linker. In certain embodiments, the linker comprises the amino acid sequence set forth in SEQ ID NO: 96.

In certain embodiments, the variable regions within the extracellular antigen-binding domain have to be linked one after another such that at the N-terminus of the extracellular antigen-binding domain, a heavy chain variable region (V_{H}) is positioned. In certain embodiments, if the extracellular antigen-binding domain is an scFv, the variable regions are positioned from the N- to the C-terminus: V_{H}-V_{L}.

In certain embodiments, the variable regions within the extracellular antigen-binding domain have to be linked one after another such that at the N-terminus of the extracellular antigen-binding domain, a light chain variable region (V_{L}) is positioned. In certain embodiments, if the extracellular antigen-binding domain is an scFv, the variable regions are positioned from the N- to the C-terminus: V_{L}-V_{H}.

TABLE 4

| CDRs | 1 | 2 | 3 |
|---|---|---|---|
| V_{H} | GFSLSTSGMC | IDWDDDK | ARTPYSGSYNWFDP |
| | [SEQ ID NO: 32] | [SEQ ID NO: 33] | [SEQ ID NO: 34] |
| V_{L} | QGISNW | AAS | QQADSFPFT |
| | [SEQ ID NO: 25] | [SEQ ID NO: 26] | [SEQ ID NO: 27] |

| | |
|---|---|
| Full V_{H} | QVTLRESGPALVKPTQTLTLTCTFSGFSLSTSGMCVSWIRQPPGKALEWLALIDWDDDK |
| | YYSTSLKTRLTISKDTSKNQVVLTMTNMDPVDTATYYCARTPYSGSYNWEDPWGQGTLV |
| | TVSS [SEQ ID NO: 35] |
| Full V_{L} | DIQMTQSPSSVSASVGDRVTITCRASQGISNWLTWYQQKPGKAPKLLIYAASSLQSGVP |
| | SRFSGSGSGTDFTLTISSLQPEDFATYYCQQADSFPFTFGPGTKVDIK [SEQ ID |
| | NO: 29] |
| DNA for | CAGGTCACCTTGAGGGAGTCTGGTCCTGCGCTGGTGAAACCCACACAGACCCTCACACT |
| Full V_{H} | GACCTGCACCTTCTCTGGGTTCTCACTCAGCACTAGTGGAATGTGTGTGAGCTGGATCC |
| | GTCAGCCCCCAGGGAAGGCCCTGGAGTGGCTTGCACTCATTGATTGGGATGATGATAAA |
| | TACTACAGCACATCTCTGAAGACCAGGCTCACCATCTCCAAGGACACCTCCAAAAACCA |
| | GGTGGTCCTTACAATGACCAACATGGACCCTGTGGACACAGCCACGTATTATTGTGCAC |
| | GGACCCCCTATAGTGGGAGCTACAACTGGTTCGACCCCTGGGGCCAGGGAACCCTGGTC |
| | ACCGTCTCCTCA [SEQ ID NO: 36] |
| DNA for | GACATCCAGATGACCCAGTCTCCATCTTCCGTGTCTGCATCTGTAGGAGACAGAGTCAC |
| Full V_{L} | CATCACTTGTCGGGCGAGTCAGGGTATTAGTAATTGGTTAACCTGGTATCAGCAGAAAC |
| | CAGGGAAAGCCCCTAAGCTCCTGATCTATGCTGCATCCAGTTTGCAAAGTGGGGTCCCA |
| | TCAAGGTTCAGCGGCAGTGGATCTGGGACAGATTTCACTCTCACCATCAGCAGCCTGCA |
| | GCCTGAAGATTTTGCAACTTACTATTGTCAACAGGCTGACAGTTTCCCATTCACTTTCG |
| | GCCCTGGGACCAAAGTGGATATCAAA [SEQ ID NO: 31] |

In certain embodiments, the extracellular antigen-binding domain (e.g., an scFv) comprises a V_{H} comprising a CDR1 comprising the amino acid sequence set forth in SEQ ID NO: 37 or a conservative modification thereof, a CDR2 comprising the amino acid sequence set forth in SEQ ID NO: 38 or a conservative modification thereof, and a CDR3 comprising the amino acid sequence set forth in SEQ ID NO: 39 or a conservative modification thereof. SEQ ID NOs: 37-39 are provided in Table 5.

In certain embodiments, the extracellular antigen-binding domain (e.g., an scFv) comprises a V_{L} comprising a CDR1 comprising the amino acid sequence set forth in SEQ ID NO: 40 or a conservative modification thereof, a CDR2 comprising the amino acid sequence set forth in SEQ ID NO: 41 or a conservative modification thereof, and a CDR3 comprising the amino acid sequence set forth in SEQ ID NO: 42 or a conservative modification thereof. SEQ ID NOs: 40-42 are provided in Table 5.

In certain embodiments, the extracellular antigen-binding domain (e.g., an scFv) comprises a V_{H} comprising a CDR1 comprising the amino acid sequence set forth in SEQ ID NO: 37 or a conservative modification thereof, a CDR2 comprising the amino acid sequence set forth in SEQ ID NO: 38 or a conservative modification thereof, a CDR3 comprising the amino acid sequence set forth in SEQ ID NO: 39 or a conservative modification thereof; and a V_{L} Comprising a CDR1 comprising the amino acid sequence set forth in SEQ ID NO: 40 or a conservative modification thereof, a CDR2 comprising the amino acid sequence set forth in SEQ ID NO: 41 or a conservative modification thereof, and a CDR3 comprising the amino acid sequence set forth in SEQ ID NO: 42 or a conservative modification thereof.

In certain embodiments, the extracellular antigen-binding domain (e.g., an scFv) comprises a V_{H} comprising a CDR1 comprising the amino acid sequence set forth in SEQ ID NO: 37, a CDR2 comprising the amino acid sequence set forth in SEQ ID NO: 38, a CDR3 comprising the amino acid sequence set forth in SEQ ID NO: 39; and a V_{L} comprising a CDR1 comprising the amino acid sequence set forth in SEQ ID NO: 40, a CDR2 comprising the amino acid sequence set forth in SEQ ID NO: 41, and a CDR3 comprising the amino acid sequence set forth in SEQ ID NO: 42.

In certain embodiments, the extracellular antigen-binding domain (e.g., an scFv) comprises a V_{H} comprising an amino acid sequence that is at least about 80% (e.g., at least about 85%, at least about 90%, or at least about 95%) homologous or identical to the amino sequence set forth in SEQ ID NO: 43. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 43. In certain embodiments, the extracellular antigen-binding domain comprises a V_{H} comprising the amino sequence set forth in SEQ ID NO: 43. An exemplary nucleotide sequence encoding the amino acid sequence of SEQ ID NO: 43 is set forth in SEQ ID NO: 45. SEQ ID NO: 43 and 45 is provided in Table 5 below.

In certain embodiments, the extracellular antigen-binding domain (e.g., an scFv) comprises a V_{L} comprising an amino acid sequence that is at least about 80% (e.g., at least about 85%, at least about 90%, or at least about 95%) homologous or identical to the amino sequence set forth in SEQ ID NO: 44. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 44. In certain embodiments, the extracellular antigen-binding domain comprises a V_{L} comprising the amino sequence set forth in SEQ ID NO: 44. An exemplary nucleotide sequence encoding the amino acid sequence of SEQ ID NO: 44 is set forth in SEQ ID NO: 46. SEQ ID NO: 44 and 46 are provided in Table 5 below.

In certain embodiments, the extracellular antigen-binding domain (e.g., an scFv) comprises a V_{H} comprising the amino acid sequence set forth in SEQ ID NO: 43, and a V_{L} comprising the amino acid sequence set forth in SEQ ID NO: 44. In certain embodiments, the extracellular antigen-binding domain is an scFv. In certain embodiments, the scFv is designated as “1-P13”. In certain embodiments, the V_{H} and V_{L} are linked via a linker. In certain embodiments, the linker comprises the amino acid sequence set forth in SEQ ID NO: 96.

In certain embodiments, the variable regions within the extracellular antigen-binding domain have to be linked one after another such that at the N-terminus of the extracellular antigen-binding domain, a heavy chain variable region (V_{H}) is positioned. In certain embodiments, if the extracellular antigen-binding domain is an scFv, the variable regions are positioned from the N- to the C-terminus: V_{H}-V_{L}.

In certain embodiments, the variable regions within the extracellular antigen-binding domain have to be linked one after another such that at the N-terminus of the extracellular antigen-binding domain, a light chain variable region (V_{L}) is positioned. In certain embodiments, if the extracellular antigen-binding domain is an scFv, the variable regions are positioned from the N- to the C-terminus: V_{L}-V_{H}.

TABLE 5

| CDRs | 1 | 2 | 3 |
|---|---|---|---|
| V_{H} | GFTFSTYG | ISYDGSNK | ARGRVGTLDY |
| | [SEQ ID NO: 37] | [SEQ ID NO: 38] | [SEQ ID NO: 39] |
| V_{L} | RGLVYSDVNTN | KVS | MQGTHWPWT |
| | [SEQ ID NO: 40] | [SEQ ID NO: 41] | [SEQ ID NO: 42] |

| | |
|---|---|
| Full V_{H} | QVQLVESGGGVVQPGRSLRLSCAASGFTFSTYGMHWVRQAPGKGLEWVAVISYDGSNKY |
| | HGDAVKGRFTISRDNSKNTLYLQMNSLRAEDTAVYYCARGRVGTLDYWGQGTLVTVSS |
| | [SEQ ID NO: 43] |
| Full V_{L} | DVVMTQSPLSLPVSLGQPASISCRSSRGLVYSDVNTNLNWFQQRPGQSPRRLIYKVSNR |
| | DSGVPDRFSGSGSGTDFTLKISRVEAEDVGVYYCMQGTHWPWTFGQGTKVEIK |
| | [SEQ ID NO: 44] |
| DNA for | CAGGTGCAGCTGGTGGAGTCTGGGGGAGGCGTGGTCCAGCCTGGGAGGTCCCTGAGACT |
| Full V_{H} | CTCCTGTGCAGCCTCTGGATTTACCTTCAGTACCTATGGCATGCACTGGGTCCGCCAGG |
| | CTCCAGGCAAGGGGCTGGAGTGGGTGGCAGTCATATCATATGATGGAAGTAATAAATAT |
| | CATGGAGACGCCGTGAAGGGCCGGTTCACCATCTCCAGAGACAATTCCAAGAACACGCT |
| | GTATCTGCAAATGAACAGCCTGAGAGCTGAGGACACGGCTGTGTATTACTGTGCGAGGG |
| | GGAGAGTGGGAACTCTTGACTATTGGGGCCAGGGAACCCTGGTCACCGTCTCCTCA |
| | [SEQ ID NO: 45] |
| DNA for | GATGTTGTGATGACTCAGTCTCCACTCTCCCTGCCCGTCAGCCTTGGACAGCCGGCCTC |
| Full V_{L} | CATCTCCTGCAGGTCTAGTCGAGGCCTCGTATACAGTGATGTAAACACCAACTTGAATT |
| | GGTTTCAGCAGAGGCCAGGCCAATCTCCAAGGCGCCTAATTTATAAGGTTTCTAACCGG |
| | GACTCTGGGGTCCCAGACAGATTCAGCGGCAGTGGGTCAGGCACTGATTTCACACTGAA |
| | AATCAGCAGGGTGGAGGCTGAGGATGTTGGGGTTTATTACTGCATGCAAGGTACACACT |
| | GGCCTTGGACGTTCGGCCAAGGGACCAAGGTGGAAATCAAA [SEQ ID NO: 46] |

In certain embodiments, the extracellular antigen-binding domain (e.g., an scFv) comprises a V_{H} comprising a CDR1 comprising the amino acid sequence set forth in SEQ ID NO: 47 or a conservative modification thereof, a CDR2 comprising the amino acid sequence set forth in SEQ ID NO: 38 or a conservative modification thereof, and a CDR3 comprising the amino acid sequence set forth in SEQ ID NO: 48 or a conservative modification thereof. SEQ ID NOs: 38, 47, and 48 are provided in Table 6.

In certain embodiments, the extracellular antigen-binding domain (e.g., an scFv) comprises a V_{L} comprising a CDR1 comprising the amino acid sequence set forth in SEQ ID NO: 49 or a conservative modification thereof, a CDR2 comprising the amino acid sequence set forth in SEQ ID NO: 50 or a conservative modification thereof, and a CDR3 comprising the amino acid sequence set forth in SEQ ID NO: 51 or a conservative modification thereof. SEQ ID NOs: 49-51 are provided in Table 6.

In certain embodiments, the extracellular antigen-binding domain (e.g., an scFv) comprises a V_{H} comprising a CDR1 comprising the amino acid sequence set forth in SEQ ID NO: 47 or a conservative modification thereof, a CDR2 comprising the amino acid sequence set forth in SEQ ID NO: 38 or a conservative modification thereof, a CDR3 comprising the amino acid sequence set forth in SEQ ID NO: 48 or a conservative modification thereof; and a V_{L} comprising a CDR1 comprising the amino acid sequence set forth in SEQ ID NO: 49 or a conservative modification thereof, a CDR2 comprising the amino acid sequence set forth in SEQ ID NO: 50 or a conservative modification thereof, and a CDR3 comprising the amino acid sequence set forth in SEQ ID NO: 51 or a conservative modification thereof.

In certain embodiments, the extracellular antigen-binding domain (e.g., an scFv) comprises a V_{H} comprising a CDR1 comprising the amino acid sequence set forth in SEQ ID NO: 47, a CDR2 comprising the amino acid sequence set forth in SEQ ID NO: 38, a CDR3 comprising the amino acid sequence set forth in SEQ ID NO: 48; and a V_{L} comprising a CDR1 comprising the amino acid sequence set forth in SEQ ID NO: 49, a CDR2 comprising the amino acid sequence set forth in SEQ ID NO: 50, and a CDR3 comprising the amino acid sequence set forth in SEQ ID NO: 51.

In certain embodiments, the extracellular antigen-binding domain (e.g., an scFv) comprises a V_{H} comprising an amino acid sequence that is at least about 80% (e.g., at least about 85%, at least about 90%, or at least about 95%) homologous or identical to the amino sequence set forth in SEQ ID NO: 52. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 52. In certain embodiments, the extracellular antigen-binding domain comprises a V_{H} comprising the amino sequence set forth in SEQ ID NO: 52. An exemplary nucleotide sequence encoding the amino acid sequence of SEQ ID NO: 52 is set forth in SEQ ID NO: 54. SEQ ID NO: 52 and 54 are provided in Table 6 below.

In certain embodiments, the extracellular antigen-binding domain (e.g., an scFv) comprises a V_{L} comprising an amino acid sequence that is at least about 80% (e.g., at least about 85%, at least about 90%, or at least about 95%) homologous or identical to the amino sequence set forth in SEQ ID NO: 53. For example, the extracellular antigen-binding domain (e.g., an scFv) comprises a V_{L}comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 53. In certain embodiments, the extracellular antigen-binding domain comprises a V_{L} comprising the amino sequence set forth in SEQ ID NO: 53. An exemplary nucleotide sequence encoding the amino acid sequence of SEQ ID NO: 53 is set forth in SEQ ID NO: 55. SEQ ID NO: 53 and 55 are provided in Table 6 below.

In certain embodiments, the extracellular antigen-binding domain (e.g., an scFv) comprises a V_{H} comprising the amino acid sequence set forth in SEQ ID NO: 52, and a V_{L} comprising the amino acid sequence set forth in SEQ ID NO: 53. In certain embodiments, the extracellular antigen-binding domain is an scFv. In certain embodiments, the scFv is designated as “1-P23”. In certain embodiments, the linker comprises the amino acid sequence set forth in SEQ ID NO: 96.

In certain embodiments, the variable regions within the extracellular antigen-binding domain have to be linked one after another such that at the N-terminus of the extracellular antigen-binding domain, a heavy chain variable region (V_{H}) is positioned. In certain embodiments, if the extracellular antigen-binding domain is an scFv, the variable regions are positioned from the N- to the C-terminus: V_{H}-V_{L}.

In certain embodiments, the variable regions within the extracellular antigen-binding domain have to be linked one after another such that at the N-terminus of the extracellular antigen-binding domain, a light chain variable region (V_{L}) is positioned. In certain embodiments, if the extracellular antigen-binding domain is an scFv, the variable regions are positioned from the N- to the C-terminus: V_{L}-V_{H}.

TABLE 6

| CDRs | 1 | 2 | 3 |
|---|---|---|---|
| V_{H} | GFTENSYG | ISYDGSNK | ARDNYDSSGYNWYFDL |
| | [SEQ ID NO: 47] | [SEQ ID NO: 38] | [SEQ ID NO: 48] |
| V_{L} | SLRSYY | GKN | NSRDSSGNLWV |
| | [SEQ ID NO: 49] | [SEQ ID NO: 50] | [SEQ ID NO: 51] |

| | |
|---|---|
| Full V_{H} | QVQLVESGGGVVQPGRSLRLSCAASGFTENSYGMHWVRQAPGKGLEWVAIISYDGSNKY |
| | YADSVKGRFTISRDNSKNTLYLQMNSLRAEDTAVYYCARDNYDSSGYNWYFDLWGRGTL |
| | VTVSS [SEQ ID NO: 52] |
| Full V_{L} | SSELTQDPAVSVALGQTVRITCQGDSLRSYYASWYQQKPGQAPVLVIYGKNNRPSGIPD |
| | RFSGSSSGNTASLTITGAQAEDEADYYCNSRDSSGNLWVFGGGTKLTVL [SEQ ID |
| | NO: 53] |
| DNA for | CAGGTGCAGCTGGTGGAGTCTGGGGGAGGCGTGGTCCAGCCTGGGAGGTCCCTGAGACT |
| Full V_{H} | CTCCTGTGCAGCCTCTGGATTCACCTTCAACAGCTATGGCATGCACTGGGTCCGCCAGG |
| | CTCCAGGCAAGGGGCTGGAGTGGGTGGCAATTATATCATATGATGGAAGTAATAAATAC |
| | TATGCAGACTCCGTGAAGGGCCGATTCACCATCTCCAGAGACAATTCCAAGAACACGCT |
| | GTATCTGCAAATGAACAGCCTGAGAGCTGAGGACACGGCTGTGTATTATTGTGCGAGAG |
| | ATAATTATGATAGTAGTGGTTATAACTGGTACTTCGATCTCTGGGGCCGTGGCACCCTG |
| | GTCACTGTCTCCTCA [SEQ ID NO: 54] |
| DNA for | TCTTCTGAGCTGACTCAGGACCCTGCTGTGTCTGTGGCCTTGGGACAGACAGTCAGGAT |
| Full V_{L} | CACATGCCAAGGAGACAGCCTCAGAAGCTATTATGCAAGCTGGTACCAGCAGAAGCCAG |
| | GACAGGCCCCTGTACTTGTCATCTATGGTAAAAACAACCGGCCCTCAGGGATCCCAGAC |
| | CGATTCTCTGGCTCCAGCTCAGGAAACACAGCTTCCTTGACCATCACTGGGGCTCAGGC |
| | GGAAGATGAGGCTGACTATTACTGTAACTCCCGGGACAGCAGTGGTAACCTCTGGGTGT |
| | TCGGCGGAGGGACCAAGCTGACCGTCCTA [SEQ ID NO: 55] |

In certain embodiments, the extracellular antigen-binding domain (e.g., an scFv) comprises a V_{H} comprising a CDR1 comprising the amino acid sequence set forth in SEQ ID NO: 56 or a conservative modification thereof, a CDR2 comprising the amino acid sequence set forth in SEQ ID NO: 57 or a conservative modification thereof, and a CDR3 comprising the amino acid sequence set forth in SEQ ID NO: 58 or a conservative modification thereof. SEQ ID NOs: 56-58 are provided in Table 7.

In certain embodiments, the extracellular antigen-binding domain (e.g., an scFv) comprises a V_{L} comprising a CDR1 comprising the amino acid sequence set forth in SEQ ID NO: 59 or a conservative modification thereof, a CDR2 comprising the amino acid sequence set forth in SEQ ID NO: 60 or a conservative modification thereof, and a CDR3 comprising the amino acid sequence set forth in SEQ ID NO: 61 or a conservative modification thereof. SEQ ID NOs: 59-61 are provided in Table 7.

In certain embodiments, the extracellular antigen-binding domain (e.g., an scFv) comprises a V_{H} comprising a CDR1 comprising the amino acid sequence set forth in SEQ ID NO: 56 or a conservative modification thereof, a CDR2 comprising the amino acid sequence set forth in SEQ ID NO: 57 or a conservative modification thereof, a CDR3 comprising the amino acid sequence set forth in SEQ ID NO: 58 or a conservative modification thereof; and a V_{L} comprising a CDR1 comprising the amino acid sequence set forth in SEQ ID NO: 59 or a conservative modification thereof, a CDR2 comprising the amino acid sequence set forth in SEQ ID NO: 60 or a conservative modification thereof, and a CDR3 comprising the amino acid sequence set forth in SEQ ID NO: 61 or a conservative modification thereof.

In certain embodiments, the extracellular antigen-binding domain (e.g., an scFv) comprises a V_{H} comprising a CDR1 comprising the amino acid sequence set forth in SEQ ID NO: 56, a CDR2 comprising the amino acid sequence set forth in SEQ ID NO: 57, a CDR3 comprising the amino acid sequence set forth in SEQ ID NO: 58; and a V_{L} comprising a CDR1 comprising the amino acid sequence set forth in SEQ ID NO: 59, a CDR2 comprising the amino acid sequence set forth in SEQ ID NO: 60, and a CDR3 comprising the amino acid sequence set forth in SEQ ID NO: 61.

In certain embodiments, the extracellular antigen-binding domain (e.g., an scFv) comprises a V_{H} comprising an amino acid sequence that is at least about 80% (e.g., at least about 85%, at least about 90%, or at least about 95%) homologous or identical to the amino sequence set forth in SEQ ID NO: 62. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 62. In certain embodiments, the extracellular antigen-binding domain comprises a V_{H} comprising the amino sequence set forth in SEQ ID NO: 62. An exemplary nucleotide sequence encoding the amino acid sequence of SEQ ID NO: 62 is set forth in SEQ ID NO: 64. SEQ ID NO: 62 and 64 are provided in Table 7 below.

In certain embodiments, the extracellular antigen-binding domain (e.g., an scFv) comprises a V_{L} comprising an amino acid sequence that is at least about 80% (e.g., at least about 85%, at least about 90%, or at least about 95%) homologous or identical to the amino sequence set forth in SEQ ID NO: 63. For example, the extracellular antigen-binding domain (e.g., an scFv) comprises a V_{L}comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 63. In certain embodiments, the extracellular antigen-binding domain comprises a V_{L} comprising the amino sequence set forth in SEQ ID NO: 63. An exemplary nucleotide sequence encoding the amino acid sequence of SEQ ID NO: 63 is set forth in SEQ ID NO: 65. SEQ ID NO: 63 and 65 are provided in Table 7 below.

In certain embodiments, the extracellular antigen-binding domain (e.g., an scFv) comprises a V_{H} comprising the amino acid sequence set forth in SEQ ID NO: 62, and a V_{L} comprising the amino acid sequence set forth in SEQ ID NO: 63. In certain embodiments, the extracellular antigen-binding domain is an scFv. In certain embodiments, the scFv is designated as “1-A20”. In certain embodiments, the V_{H} and V_{L} are linked via a linker. In certain embodiments, the linker comprises the amino acid sequence set forth in SEQ ID NO: 96.

In certain embodiments, the variable regions within the extracellular antigen-binding domain have to be linked one after another such that at the N-terminus of the extracellular antigen-binding domain, a heavy chain variable region (V_{H}) is positioned. In certain embodiments, if the extracellular antigen-binding domain is an scFv, the variable regions are positioned from the N- to the C-terminus: V_{H}-V_{L}.

In certain embodiments, the variable regions within the extracellular antigen-binding domain have to be linked one after another such that at the N-terminus of the extracellular antigen-binding domain, a light chain variable region (V_{L}) is positioned. In certain embodiments, if the extracellular antigen-binding domain is an scFv, the variable regions are positioned from the N- to the C-terminus: V_{L}-V_{H}.

TABLE 7

| CDRs | 1 | 2 | 3 |
|---|---|---|---|
| V_{H} | GDSISSYY | IYTSGNT | ARDGDNRDSDAFDI |
| | [SEQ ID NO: 56] | [SEQ ID NO: 57] | [SEQ ID NO: 58] |
| V_{L} | QNISSSY | GTS | QQYGSSPLT |
| | [SEQ ID NO: 59] | [SEQ ID NO: 60] | [SEQ ID NO: 61] |

| | |
|---|---|
| Full V_{H} | QVQLQESGPGLVKPSETLSLTCTVSGDSISSYYWSWIRQPAGKGLEWIGRIYTSGNTNY |
| | NPSLKSRVTMSVDKSKNQFSLKLRSVTAADTAVYYCARDGDNRDSDAFDIWGQGTMVTV |
| | SS [SEQ ID NO: 62] |
| Full V_{L} | SPGTLSLSPGERATLSCRASQNISSSYLAWCQQKPGQAPRLFIYGTSRRATGIPDRESG |
| | SGSGTDFTLTISRLEPEDFAVYYCQQYGSSPLTFGGGTKVEIK [SEQ ID NO: 63] |
| DNA for | CAGGTGCAGCTGCAGGAGTCGGGCCCAGGACTGGTGAAGCCTTCGGAGACCCTGTCCCT |
| Full V_{H} | CACCTGCACTGTCTCTGGTGACTCCATCAGTAGTTATTACTGGAGCTGGATCCGGCAGC |
| | CCGCCGGGAAGGGACTGGAGTGGATTGGACGTATCTATACCAGTGGGAACACCAACTAC |
| | AACCCCTCCCTCAAGAGTCGAGTCACCATGTCAGTAGACAAGTCCAAGAACCAGTTCTC |
| | CCTGAAGCTGAGGTCTGTGACCGCCGCGGACACGGCCGTGTATTACTGTGCGAGAGATG |
| | GTGATAACCGGGACTCTGATGCTTTTGATATCTGGGGCCAAGGGACAATGGTCACCGTC |
| | TCTTCA [SEQ ID NO: 64] |
| DNA for | GAAATTGTGTTGACGCAGTCTCCAGGCACCCTGTCTTTGTCTCCAGGGGAAAGAGCCAC |
| Full V_{L} | CCTCTCCTGCAGGGCCAGTCAGAATATTAGCAGCAGCTACTTAGCCTGGTGCCAGCAGA |
| | AACCTGGCCAGGCTCCCAGGCTCTTCATCTATGGTACATCCCGCAGGGCCACTGGCATC |
| | CCAGACAGGTTCAGTGGCAGTGGGTCTGGGACAGACTTCACTCTCACCATCAGCAGACT |
| | GGAGCCTGAAGATTTTGCAGTGTATTACTGTCAGCAGTATGGTAGTTCACCTCTCACTT |
| | TCGGCGGAGGGACCAAGGTGGAGATCAAA [SEQ ID NO: 65] |

In certain embodiments, the extracellular antigen-binding domain (e.g., an scFv) comprises a V_{H} comprising a CDR1 comprising the amino acid sequence set forth in SEQ ID NO: 66 or a conservative modification thereof, a CDR2 comprising the amino acid sequence set forth in SEQ ID NO: 67 or a conservative modification thereof, and a CDR3 comprising the amino acid sequence set forth in SEQ ID NO: 68 or a conservative modification thereof. SEQ ID NOs: 66-68 are provided in Table 8.

In certain embodiments, the extracellular antigen-binding domain (e.g., an scFv) comprises a V_{L} comprising a CDR1 comprising the amino acid sequence set forth in SEQ ID NO: 69 or a conservative modification thereof, a CDR2 comprising the amino acid sequence set forth in SEQ ID NO: 70 or a conservative modification thereof, and a CDR3 comprising the amino acid sequence set forth in SEQ ID NO: 71 or a conservative modification thereof. SEQ ID NOs: 69-71are provided in Table 8.

In certain embodiments, the extracellular antigen-binding domain (e.g., an scFv) comprises a V_{H} comprising a CDR1 comprising the amino acid sequence set forth in SEQ ID NO: 66 or a conservative modification thereof, a CDR2 comprising the amino acid sequence set forth in SEQ ID NO: 67 or a conservative modification thereof, a CDR3 comprising the amino acid sequence set forth in SEQ ID NO: 68 or a conservative modification thereof; and a V_{L} comprising a CDR1 comprising the amino acid sequence set forth in SEQ ID NO: 69 or a conservative modification thereof, a CDR2 comprising the amino acid sequence set forth in SEQ ID NO: 70 or a conservative modification thereof, and a CDR3 comprising the amino acid sequence set forth in SEQ ID NO: 71 or a conservative modification thereof.

In certain embodiments, the extracellular antigen-binding domain (e.g., an scFv) comprises a V_{H} comprising a CDR1 comprising the amino acid sequence set forth in SEQ ID NO: 66, a CDR2 comprising the amino acid sequence set forth in SEQ ID NO: 67, a CDR3 comprising the amino acid sequence set forth in SEQ ID NO: 68; and a V_{L} comprising a CDR1 comprising the amino acid sequence set forth in SEQ ID NO: 69, a CDR2 comprising the amino acid sequence set forth in SEQ ID NO: 70, and a CDR3 comprising the amino acid sequence set forth in SEQ ID NO: 71.

In certain embodiments, the extracellular antigen-binding domain (e.g., an scFv) comprises a V_{H} comprising an amino acid sequence that is at least about 80% (e.g., at least about 85%, at least about 90%, or at least about 95%) homologous or identical to the amino sequence set forth in SEQ ID NO: 72. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 72. In certain embodiments, the extracellular antigen-binding domain comprises a V_{H} comprising the amino sequence set forth in SEQ ID NO: 72. An exemplary nucleotide sequence encoding the amino acid sequence of SEQ ID NO: 72 is set forth in SEQ ID NO: 74. SEQ ID NOs: 72 and 74 are provided in Table 8 below.

In certain embodiments, the extracellular antigen-binding domain (e.g., an scFv) comprises a V_{L} comprising an amino acid sequence that is at least about 80% (e.g., at least about 85%, at least about 90%, or at least about 95%) homologous or identical to the amino sequence set forth in SEQ ID NO: 73. For example, the extracellular antigen-binding domain (e.g., an scFv) comprises a V_{L}comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 73. In certain embodiments, the extracellular antigen-binding domain comprises a V_{L} comprising the amino sequence set forth in SEQ ID NO: 73. An exemplary nucleotide sequence encoding the amino acid sequence of SEQ ID NO: 73 is set forth in SEQ ID NO: 75. SEQ ID NOs: 73 and 75 are provided in Table 8 below.

In certain embodiments, the extracellular antigen-binding domain (e.g., an scFv) comprises a V_{H} comprising the amino acid sequence set forth in SEQ ID NO: 72, and a V_{L} comprising the amino acid sequence set forth in SEQ ID NO: 73. In certain embodiments, the extracellular antigen-binding domain is an scFv. In certain embodiments, the scFv is designated as “1-N3”. In certain embodiments, the V_{H} and V_{L} are linked via a linker. In certain embodiments, the linker comprises the amino acid sequence set forth in SEQ ID NO: 96.

In certain embodiments, the variable regions within the extracellular antigen-binding domain have to be linked one after another such that at the N-terminus of the extracellular antigen-binding domain, a heavy chain variable region (V_{H}) is positioned. In certain embodiments, if the extracellular antigen-binding domain is an scFv, the variable regions are positioned from the N- to the C-terminus: V_{H}-V_{L}.

In certain embodiments, the variable regions within the extracellular antigen-binding domain have to be linked one after another such that at the N-terminus of the extracellular antigen-binding domain, a light chain variable region (V_{L}) is positioned. In certain embodiments, if the extracellular antigen-binding domain is an scFv, the variable regions are positioned from the N- to the C-terminus: V_{L}-V_{H}.

TABLE 8

| CDRs | 1 | 2 | 3 |
|---|---|---|---|
| V_{H} | GFSFTSHW | IYPGDSDT | ARHETGNGYSYGMDV |
| | [SEQ ID | [SEQ ID | [SEQ ID |
| | NO: 66] | NO: 67] | NO: 68] |
| V_{L} | QSLLHSNGYNY | LGS | MQALQPPLT |
| | [SEQ ID | [SEQ ID | [SEQ ID |
| | NO: 69] | NO: 70] | NO: 71] |

| | |
|---|---|
| Full | EVQLVQSGADVKKPGESLKISCKGSGFSFTSHWIAWVRQMP |
| V_{H} | GKGLEWMGIIYPGDSDTRYSPSLQGRVTISADKSISTAYLQ |
| | WSSLKASDVTAMYFCARHETGNGYSYGMDVWGQGTTTVSS |
| | [SEQ ID NO: 72] |
| Full | DIVMTQSPLSLPVTPGEPASISCRSSQSLLHSNGYNYLDWY |
| V_{L} | LQKPGQSPQLLFSLGSNRASGVPDRFSGSGSGTDFSLKISR |
| | VEAEDVGLYYCMQALQPPLTFGGGTKVEIK |
| | [SEQ ID NO: 73] |
| DNA | GAGGTGCAGCTGGTGCAGTCTGGAGCAGACGTGAAAAAGCC |
| for | CGGGGAGTCTCTGAAGATCTCCTGTAAGGGTTCTGGATTCA |
| Full | GTTTTACCAGCCACTGGATCGCCTGGGTGCGCCAGATGCCC |
| V_{H} | GGGAAAGGCCTGGAGTGGATGGGGATAATCTATCCTGGTGA |
| | CTCTGATACCAGATACAGCCCGTCCTTACAAGGCCGGGTCA |
| | CCATCTCAGCCGACAAGTCCATCAGCACCGCCTACCTGCAG |
| | TGGAGCAGCCTGAAGGCCTCGGACACCGCCATGTATTTCTG |
| | TGCGAGGCATGAAACTGGGAATGGCTACTCCTACGGAATGG |
| | ACGTCTGGGGCCAAGGGACCACGGTCACCGTCTCCTCA |
| | [SEQ ID NO: 74] |
| DNA | GATATTGTGATGACTCAGTCTCCACTCTCCCTGCCCGTCAC |
| for | CCCTGGAGAGCCGGCCTCCATCTCCTGCAGGTCTAGTCAGA |
| Full | GCCTCCTGCATAGTAATGGATACAACTATTTGGATTGGTAC |
| V_{L} | CTGCAGAAGCCAGGGCAGTCTCCACAGCTCCTGTTCTCTTT |
| | GGGTTCTAATCGGGCCTCCGGGGTCCCTGACAGGTTCAGTG |
| | GCAGTGGATCAGGCACAGATTTTTCACTGAAAATCAGCAGA |
| | GTGGAGGCTGAGGATGTTGGGCTTTATTACTGCATGCAAGC |
| | TCTACAACCTCCGCTCACTTTCGGCGGAGGGACCAAGGTGG |
| | AGATCAAA |
| | [SEQ ID NO: 75] |

In certain embodiments, the extracellular antigen-binding domain (e.g., an scFv) comprises a V_{H} comprising a CDR1 comprising the amino acid sequence set forth in SEQ ID NO: 76 or a conservative modification thereof, a CDR2 comprising the amino acid sequence set forth in SEQ ID NO: 57 or a conservative modification thereof, and a CDR3 comprising the amino acid sequence set forth in SEQ ID NO: 77 or a conservative modification thereof. SEQ ID NOs: 57, 76, and 77 are provided in Table 9.

In certain embodiments, the extracellular antigen-binding domain (e.g., an scFv) comprises a V_{L} comprising a CDR1 comprising the amino acid sequence set forth in SEQ ID NO: 78 or a conservative modification thereof, a CDR2 comprising the amino acid sequence set forth in SEQ ID NO: 79 or a conservative modification thereof, and a CDR3 comprising the amino acid sequence set forth in SEQ ID NO: 80 or a conservative modification thereof. SEQ ID NOs: 78-80 are provided in Table 9.

In certain embodiments, the extracellular antigen-binding domain (e.g., an scFv) comprises a V_{H} comprising a CDR1 comprising the amino acid sequence set forth in SEQ ID NO: 76 or a conservative modification thereof, a CDR2 comprising the amino acid sequence set forth in SEQ ID NO: 57 or a conservative modification thereof, a CDR3 comprising the amino acid sequence set forth in SEQ ID NO: 77 or a conservative modification thereof; and a V_{L} comprising a CDR1 comprising the amino acid sequence set forth in SEQ ID NO: 78 or a conservative modification thereof, a CDR2 comprising the amino acid sequence set forth in SEQ ID NO: 79 or a conservative modification thereof, and a CDR3 comprising the amino acid sequence set forth in SEQ ID NO: 80 or a conservative modification thereof.

In certain embodiments, the extracellular antigen-binding domain (e.g., an scFv) comprises a V_{H} comprising a CDR1 comprising the amino acid sequence set forth in SEQ ID NO: 76, a CDR2 comprising the amino acid sequence set forth in SEQ ID NO: 57, a CDR3 comprising the amino acid sequence set forth in SEQ ID NO: 77; and a V_{L} comprising a CDR1 comprising the amino acid sequence set forth in SEQ ID NO: 78, a CDR2 comprising the amino acid sequence set forth in SEQ ID NO: 79, and a CDR3 comprising the amino acid sequence set forth in SEQ ID NO: 80.

In certain embodiments, the extracellular antigen-binding domain (e.g., an scFv) comprises a V_{H} comprising an amino acid sequence that is at least about 80% (e.g., at least about 85%, at least about 90%, or at least about 95%) homologous or identical to the amino sequence set forth in SEQ ID NO: 81. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 81. In certain embodiments, the extracellular antigen-binding domain comprises a V_{H} comprising the amino sequence set forth in SEQ ID NO: 81. An exemplary nucleotide sequence encoding the amino acid sequence of SEQ ID NO: 81 is set forth in SEQ ID NO: 83. SEQ ID NOs: 81 and 83 are provided in Table 9 below.

In certain embodiments, the extracellular antigen-binding domain (e.g., an scFv) comprises a V_{L} comprising an amino acid sequence that is at least about 80% (e.g., at least about 85%, at least about 90%, or at least about 95%) homologous or identical to the amino sequence set forth in SEQ ID NO: 82. For example, the extracellular antigen-binding domain (e.g., an scFv) comprises a V_{L}comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 82. In certain embodiments, the extracellular antigen-binding domain comprises a V_{L} comprising the amino sequence set forth in SEQ ID NO: 82. An exemplary nucleotide sequence encoding the amino acid sequence of SEQ ID NO: 82 is set forth in SEQ ID NO: 84. SEQ ID NO: 82 and 84 are provided in Table 9 below.

In certain embodiments, the extracellular antigen-binding domain (e.g., an scFv) comprises a V_{H} comprising the amino acid sequence set forth in SEQ ID NO: 81, and a V_{L} comprising the amino acid sequence set forth in SEQ ID NO: 82. In certain embodiments, the extracellular antigen-binding domain is an scFv. In certain embodiments, the scFv is designated as “1-H19”. In certain embodiments, the V_{H} and V_{L} are linked via a linker. In certain embodiments, the linker comprises the amino acid sequence set forth in SEQ ID NO: 96.

In certain embodiments, the variable regions within the extracellular antigen-binding domain have to be linked one after another such that at the N-terminus of the extracellular antigen-binding domain, a heavy chain variable region (V_{H}) is positioned. In certain embodiments, if the extracellular antigen-binding domain is an scFv, the variable regions are positioned from the N- to the C-terminus: V_{H}-V_{L}.

In certain embodiments, the variable regions within the extracellular antigen-binding domain have to be linked one after another such that at the N-terminus of the extracellular antigen-binding domain, a light chain variable region (V_{L}) is positioned. In certain embodiments, if the extracellular antigen-binding domain is an scFv, the variable regions are positioned from the N- to the C-terminus: V_{L}-V_{H}.

TABLE 9

| CDRs | 1 | 2 | 3 |
|---|---|---|---|
| V_{H} | GGSISTYY | IYTSGNT | ARDGDNRNSDAFDI |
| | [SEQ ID | [SEQ ID | [SEQ ID |
| | NO: 76] | NO: 57] | NO: 77] |
| V_{L} | SSNIGAGYD | GNS | QSYDRSLSGWV |
| | [SEQ ID | [SEQ ID | [SEQ ID |
| | NO: 78] | NO: 79] | NO: 80] |

| | |
|---|---|
| Full | QVQLQESGPGLVKPSETLSLTCTVSGGSISTYYWSWIRQPA |
| V_{H} | GKGLEWIGRIYTSGNTNYNPSLKSRVTMSVDTSKNQLSLKL |
| | RSVTAADTAVYYCARDGDNRNSDAFDIWGQGTMVTVSS |
| | [SEQ ID NO: 81] |
| Full | QSVLTQPPSVSGAPGQRVTISCTGSSSNIGAGYDVHWYQQL |
| V_{L} | PGTAPKLLIYGNSNRPSGVPDRFSGSKSGTSASLAITGLQA |
| | EDEADYYCQSYDRSLSGWVFGGGTKLTVL |
| | [SEQ ID NO: 82] |
| DNA | CAGGTGCAGCTGCAGGAGTCGGGCCCAGGACTGGTGAAGCC |
| for | TTCGGAGACCCTGTCCCTCACCTGCACTGTCTCTGGTGGCT |
| Full | CCATCAGTACTTATTACTGGAGCTGGATCCGGCAGCCCGCC |
| V_{H} | GGGAAGGGACTGGAGTGGATTGGGCGTATCTATACCAGTGG |
| | GAACACCAACTACAACCCCTCCCTCAAGAGTCGAGTCACCA |
| | TGTCAGTAGACACGTCCAAGAATCAGCTCTCCCTGAAGCTG |
| | AGGTCTGTGACCGCCGCGGACACGGCCGTGTATTACTGTGC |
| | GAGAGATGGGGATAACCGGAACTCTGATGCTTTTGATATTT |
| | GGGGCCAAGGGACAATGGTCACCGTCTCTTCA |
| | [SEQ ID NO: 83] |
| DNA | CAGTCTGTGCTGACGCAGCCGCCCTCAGTGTCTGGGGCCCC |
| for | AGGGCAGAGGGTCACCATCTCCTGCACTGGGAGCAGCTCCA |
| Full | ACATCGGGGCAGGTTATGATGTACACTGGTACCAGCAGCTT |
| V_{L} | CCAGGAACAGCCCCCAAACTCCTCATCTATGGTAACAGCAA |
| | TCGGCCCTCAGGGGTCCCTGACCGATTCTCTGGCTCCAAGT |
| | CTGGCACCTCAGCCTCCCTGGCCATCACTGGGCTCCAGGCT |
| | GAGGATGAGGCTGATTATTACTGCCAGTCCTATGACCGCAG |
| | CCTGAGTGGTTGGGTGTTCGGCGGAGGGACCAAGCTGACCG |
| | TCCTA |
| | [SEQ ID NO: 84] |

In certain embodiments, the extracellular antigen-binding domain (e.g., an scFv) comprises a V_{H} comprising a CDR1 comprising the amino acid sequence set forth in SEQ ID NO: 85 or a conservative modification thereof, a CDR2 comprising the amino acid sequence set forth in SEQ ID NO: 86 or a conservative modification thereof, and a CDR3 comprising the amino acid sequence set forth in SEQ ID NO: 87 or a conservative modification thereof. SEQ ID NOs: 85-87 are provided in Table 10.

In certain embodiments, the extracellular antigen-binding domain (e.g., an scFv) comprises a V_{L} comprising a CDR1 comprising the amino acid sequence set forth in SEQ ID NO: 5 or a conservative modification thereof, a CDR2 comprising the amino acid sequence set forth in SEQ ID NO: 41 or a conservative modification thereof, and a CDR3 comprising the amino acid sequence set forth in SEQ ID NO: 42 or a conservative modification thereof. SEQ ID NOs: 5, 41, and 42 are provided in Table 10.

In certain embodiments, the extracellular antigen-binding domain (e.g., an scFv) comprises a V_{H} comprising a CDR1 comprising the amino acid sequence set forth in SEQ ID NO: 85 or a conservative modification thereof, a V_{H} comprising a CDR2 comprising the amino acid sequence set forth in SEQ ID NO: 86 or a conservative modification thereof, a V_{H} comprising a CDR3 comprising the amino acid sequence set forth in SEQ ID NO: 87 or a conservative modification thereof; and a V_{L}comprising a CDR1 comprising the amino acid sequence set forth in SEQ ID NO: 5 or a conservative modification thereof, a CDR2 comprising the amino acid sequence set forth in SEQ ID NO: 41 or a conservative modification thereof, and a CDR3 comprising the amino acid sequence set forth in SEQ ID NO: 42 or a conservative modification thereof.

In certain embodiments, the extracellular antigen-binding domain (e.g., an scFv) comprises a V_{H} comprising a CDR1 comprising the amino acid sequence set forth in SEQ ID NO: 85, a CDR2 comprising the amino acid sequence set forth in SEQ ID NO: 86, a CDR3 comprising the amino acid sequence set forth in SEQ ID NO: 87; and a V_{L} comprising a CDR1 comprising the amino acid sequence set forth in SEQ ID NO: 5, a CDR2 comprising the amino acid sequence set forth in SEQ ID NO: 41, and a CDR3 comprising the amino acid sequence set forth in SEQ ID NO: 42.

In certain embodiments, the extracellular antigen-binding domain (e.g., an scFv) comprises a V_{H} comprising an amino acid sequence that is at least about 80% (e.g., at least about 85%, at least about 90%, or at least about 95%) homologous or identical to the amino sequence set forth in SEQ ID NO: 88. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 88. In certain embodiments, the extracellular antigen-binding domain comprises a V_{H} comprising the amino sequence set forth in SEQ ID NO: 88. An exemplary nucleotide sequence encoding the amino acid sequence of SEQ ID NO: 88 is set forth in SEQ ID NO: 90. SEQ ID NOs: 88 and 90 are provided in Table 10 below.

In certain embodiments, the extracellular antigen-binding domain (e.g., an scFv) comprises a V_{L} comprising an amino acid sequence that is at least about 80% (e.g., at least about 85%, at least about 90%, or at least about 95%) homologous or identical to the amino sequence set forth in SEQ ID NO: 89. For example, the extracellular antigen-binding domain (e.g., an scFv) comprises a V_{L}comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 89. In certain embodiments, the extracellular antigen-binding domain comprises a V_{L} comprising the amino sequence set forth in SEQ ID NO: 89. An exemplary nucleotide sequence encoding the amino acid sequence of SEQ ID NO: 89 is set forth in SEQ ID NO: 91. SEQ ID NOs: 89 and 91 are provided in Table 10 below.

In certain embodiments, the extracellular antigen-binding domain (e.g., an scFv) comprises a V_{H} comprising the amino acid sequence set forth in SEQ ID NO: 88, and a V_{L} comprising the amino acid sequence set forth in SEQ ID NO: 89. In certain embodiments, the extracellular antigen-binding domain is an scFv. In certain embodiments, the scFv is designated as “2-F18”. In certain embodiments, the V_{H} and V_{L} are linked via a linker. In certain embodiments, the linker comprises the amino acid sequence set forth in SEQ ID NO: 96.

In certain embodiments, the variable regions within the extracellular antigen-binding domain have to be linked one after another such that at the N-terminus of the extracellular antigen-binding domain, a heavy chain variable region (V_{H}) is positioned. In certain embodiments, if the extracellular antigen-binding domain is an scFv, the variable regions are positioned from the N- to the C-terminus: V_{H}-V_{L}.

In certain embodiments, the variable regions within the extracellular antigen-binding domain have to be linked one after another such that at the N-terminus of the extracellular antigen-binding domain, a light chain variable region (V_{L}) is positioned. In certain embodiments, if the extracellular antigen-binding domain is an scFv, the variable regions are positioned from the N- to the C-terminus: V_{L}-V_{H}.

TABLE 10

| CDRs | 1 | 2 | 3 |
|---|---|---|---|
| V_{H} | GFTFSAYP | ISYDGSNN | ARGKVGTLDF |
| | [SEQ ID | [SEQ ID | [SEQ ID |
| | NO: 85] | NO: 86] | NO: 87] |
| V_{L} | QSLVYSDGNTY | KVS | MQGTHWPWT |
| | [SEQ ID | [SEQ ID | [SEQ ID |
| | NO: 5] | NO: 41] | NO: 42] |

| | |
|---|---|
| Full | QIQLVESGGGVVQPGRSLRLSCAASGFTFSAYPMHWVRQAP |
| V_{H} | GKGLEWVAIISYDGSNNYYADSVKGRFTISRDNSKNTMYLQ |
| | INSLRAEDTGVYYCARGKVGTLDFWGQGTLVTVSS |
| | [SEQ ID NO: 88] |
| Full | DVVLTQSPLSLPVTLGQPASISCRSSQSLVYSDGNTYLNWF |
| V_{L} | QQRPGQSPRRLIYKVSNRDSGVPDRFSGSGSGTDFTLKISR |
| | VEAEDVGVYYCMQGTHWPWTFGQGTKVEIK |
| | [SEQ ID NO: 89] |
| DNA | CAGATACAGCTGGTGGAGTCTGGGGGAGGCGTGGTCCAGCC |
| for | TGGGAGGTCCCTGAGACTCTCCTGTGCAGCCTCTGGATTCA |
| Full | CCTTCAGTGCCTATCCCATGCACTGGGTCCGCCAGGCTCCA |
| V_{H} | GGCAAGGGGCTGGAGTGGGTGGCAATTATATCATATGATGG |
| | AAGTAATAACTACTATGCAGACTCCGTGAAGGGCCGATTCA |
| | CCATCTCCAGAGACAATTCCAAGAACACGATGTATCTGCAA |
| | ATTAACAGCCTGAGAGCTGAGGACACGGGTGTGTATTACTG |
| | TGCGCGAGGGAAAGTGGGAACCCTTGACTTCTGGGGCCAGG |
| | GAACCCTGGTCACCGTCTCCTCA |
| | [SEQ ID NO: 90] |
| DNA | GATGTTGTGCTGACTCAGTCTCCACTCTCCCTGCCCGTCAC |
| for | CCTTGGACAGCCGGCCTCCATCTCCTGCAGGTCTAGTCAAA |
| Full | GCCTCGTATACAGTGATGGAAACACCTACTTGAATTGGTTT |
| V_{L} | CAGCAGAGGCCAGGCCAATCTCCAAGGCGCCTAATTTATAA |
| | GGTTTCTAACCGGGACTCTGGGGTCCCAGACAGATTCAGCG |
| | GCAGTGGGTCAGGCACTGATTTCACACTGAAAATCAGCAGG |
| | GTGGAGGCTGAGGATGTTGGGGTTTATTACTGCATGCAAGG |
| | TACACACTGGCCGTGGACGTTCGGCCAAGGGACCAAGGTGG |
| | AAATCAAA |
| | [SEQ ID NO: 91] |

In certain embodiments, the extracellular antigen-binding domain (e.g., an scFv) comprises a V_{H} comprising a CDR1 comprising the amino acid sequence set forth in SEQ ID NO: 2 or a conservative modification thereof, a CDR2 comprising the amino acid sequence set forth in SEQ ID NO: 3 or a conservative modification thereof, and a CDR3 comprising the amino acid sequence set forth in SEQ ID NO: 4 or a conservative modification thereof. SEQ ID NOs:2-4 are provided in Table 11.

In certain embodiments, the extracellular antigen-binding domain (e.g., an scFv) comprises a V_{L} comprising a CDR1 comprising the amino acid sequence set forth in SEQ ID NO: 5 or a conservative modification thereof, a CDR2 comprising the amino acid sequence set forth in SEQ ID NO: 6 or a conservative modification thereof, and a CDR3 comprising the amino acid sequence set forth in SEQ ID NO: 7 or a conservative modification thereof. SEQ ID NOs: 5-7 are provided in Table 11.

In certain embodiments, the extracellular antigen-binding domain (e.g., an scFv) comprises a V_{H} comprising a CDR1 comprising the amino acid sequence set forth in SEQ ID NO: 2 or a conservative modification thereof, a CDR2 comprising the amino acid sequence set forth in SEQ ID NO: 3 or a conservative modification thereof, a CDR3 comprising the amino acid sequence set forth in SEQ ID NO: 4 or a conservative modification thereof; and a V_{L} comprising a CDR1 comprising the amino acid sequence set forth in SEQ ID NO: 5 or a conservative modification thereof, a CDR2 comprising the amino acid sequence set forth in SEQ ID NO: 6 or a conservative modification thereof, and a CDR3 comprising the amino acid sequence set forth in SEQ ID NO: 7 or a conservative modification thereof.

In certain embodiments, the extracellular antigen-binding domain (e.g., an scFv) comprises a V_{H} comprising a CDR1 comprising the amino acid sequence set forth in SEQ ID NO: 2, a CDR2 comprising the amino acid sequence set forth in SEQ ID NO: 3, a CDR3 comprising the amino acid sequence set forth in SEQ ID NO: 4; and a V_{L} comprising a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 7.

In certain embodiments, the extracellular antigen-binding domain (e.g., an scFv) comprises a V_{H} comprising an amino acid sequence that is at least about 80% (e.g., at least about 85%, at least about 90%, or at least about 95%) homologous or identical to the amino sequence set forth in SEQ ID NO: 8. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 8. In certain embodiments, the extracellular antigen-binding domain comprises a V_{H} comprising the amino sequence set forth in SEQ ID NO: 8. An exemplary nucleotide sequence encoding the amino acid sequence of SEQ ID NO: 8 is set forth in SEQ ID NO: 93. SEQ ID NO: 8 and 93 are provided in Table 11 below.

In certain embodiments, the extracellular antigen-binding domain (e.g., an scFv) comprises a V_{L} comprising an amino acid sequence that is at least about 80% (e.g., at least about 85%, at least about 90%, or at least about 95%) homologous or identical to the amino sequence set forth in SEQ ID NO: 92. For example, the extracellular antigen-binding domain (e.g., an scFv) comprises a V_{L}comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 92. In certain embodiments, the extracellular antigen-binding domain comprises a V_{L} comprising the amino sequence set forth in SEQ ID NO: 92. An exemplary nucleotide sequence encoding the amino acid sequence of SEQ ID NO: 92 is set forth in SEQ ID NO: 94. SEQ ID NO: 92 and 94 are provided in Table 11 below.

In certain embodiments, the extracellular antigen-binding domain (e.g., an scFv) comprises a V_{H} comprising the amino acid sequence set forth in SEQ ID NO: 8, and a V_{L} comprising the amino acid sequence set forth in SEQ ID NO: 92. In certain embodiments, the extracellular antigen-binding domain is an scFv. In certain embodiments, the scFv is designated as “4-P3”. In certain embodiments, the V_{H} and V_{L} are linked via a linker. In certain embodiments, the linker comprises the amino acid sequence set forth in SEQ ID NO: 96.

In certain embodiments, the variable regions within the extracellular antigen-binding domain have to be linked one after another such that at the N-terminus of the extracellular antigen-binding domain, a heavy chain variable region (V_{H}) is positioned. In certain embodiments, if the extracellular antigen-binding domain is an scFv, the variable regions are positioned from the N- to the C-terminus: V_{H}-V_{L} G In certain embodiments, the variable regions within the extracellular antigen-binding domain have to be linked one after another such that at the N-terminus of the extracellular antigen-binding domain, a light chain variable region (V_{L}) is positioned. In certain embodiments, if the extracellular antigen-binding domain is an scFv, the variable regions are positioned from the N- to the C-terminus: V_{L}-V_{H}.

TABLE 11

| CDRs | 1 | 2 | 3 |
|---|---|---|---|
| V_{H} | GFTFSTYA | ISGRGGST | AGRGDYYYYYGMDV |
| | [SEQ ID | [SEQ ID | [SEQ ID |
| | NO: 2] | NO: 3] | NO: 4] |
| V_{L} | QSLVYSDGNTY | KIS | MQSTQFPHT |
| | [SEQ ID | [SEQ ID | [SEQ ID |
| | NO: 5] | NO: 6] | NO: 7] |

| | |
|---|---|
| Full | EVQLLESGGGLVQPGGSLRLSCAASGFTFSTYAMSWVRQAP |
| V_{H} | GKGLEWVSAISGRGGSTYYTDSVKGRFTISRDNSKNTVSLQ |
| | MNSLRAEDTAVYYCAGRGDYYYYYGMDVWGQGTTVTVSA |
| | [SEQ ID NO: 8] |
| Full | DIVMTQSPLSSPVTLGQPASISCRSSQSLVYSDGNTYLSWL |
| V_{L} | QQRPGQPPRLLIYKISNRFSGVPDRFSGSGAGTDFTLKISR |
| | VEAEDVGVYYCMQSTQFPHTFGQGTKLEIK |
| | [SEQ ID NO: 92] |
| DNA | GAGGTGCAGCTGTTGGAGTCTGGGGGAGGCTTGGTCCAGCC |
| for | TGGGGGGTCCCTGAGACTCTCCTGTGCAGCCTCTGGATTCA |
| Full | CCTTTAGCACCTATGCCATGAGCTGGGTCCGCCAGGCTCCA |
| V_{H} | GGGAAGGGGCTGGAGTGGGTCTCAGCTATTAGTGGTCGTGG |
| | TGGTAGCACATACTACACAGACTCCGTGAAGGGCCGGTTCA |
| | CCATCTCCAGAGACAATTCCAAGAATACGGTGTCTCTGCAA |
| | ATGAACAGCCTGAGAGCCGAGGACACGGCCGTATATTACTG |
| | TGCGGGCCGGGGAGATTACTACTACTACTACGGTATGGACG |
| | TCTGGGGCCAAGGGACCACGGTCACCGTCTCCGCA |
| | [SEQ ID NO: 93] |
| DNA | GATATTGTGATGACCCAGAGTCCACTCTCCTCACCTGTCAC |
| for | CCTTGGACAGCCGGCCTCCATCTCCTGCAGGTCTAGTCAAA |
| Full | GCCTCGTATACAGTGATGGAAACACCTACTTGAGTTGGCTT |
| V_{L} | CAGCAGAGGCCAGGCCAGCCTCCAAGACTCCTAATTTATAA |
| | GATTTCTAACCGGTTCTCTGGGGTCCCAGACAGATTCAGTG |
| | GCAGTGGGGCAGGGACAGATTTCACACTGAAAATCAGCAGG |
| | GTGGAAGCTGAGGATGTCGGGGTTTATTACTGCATGCAATC |
| | TACACAATTTCCTCACACTTTTGGCCAGGGGACCAAGCTGG |
| | AGATCAAA |
| | [SEQ ID NO: 94] |

The V_{H} and/or V_{L} amino acid sequences having at least about 80%, at least about 80%, at least about 85%, at least about 90%, or at least about 95% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homology or identity to a specific sequence (e.g., SEQ ID NO: 8, SEQ ID NO: 9, SEQ ID NO: 18, SEQ ID NO: 19, SEQ ID NO: 28, SEQ ID NO: 29, SEQ ID NO: 35, SEQ ID NO: 43, SEQ ID NO: 44, SEQ ID NO: 52, SEQ ID NO: 53, SEQ ID NO: 62, SEQ ID NO: 63, SEQ ID NO: 72, SEQ ID NO: 73, SEQ ID NO: 81, SEQ ID NO: 82, SEQ ID NO: 88, SEQ ID NO: 89, or SEQ ID NO: 92) may contain substitutions (e.g., conservative substitutions), insertions, or deletions relative to the specified sequence(s), but retain the ability to bind to CD33.

In certain embodiments, a total of 1 to 10 amino acids are substituted, inserted and/or deleted in a specific sequence (e.g., SEQ ID NO: 8, SEQ ID NO: 9, SEQ ID NO: 18, SEQ ID NO: 19, SEQ ID NO: 28, SEQ ID NO: 29, SEQ ID NO: 35, SEQ ID NO: 43, SEQ ID NO: 44, SEQ ID NO: 52, SEQ ID NO: 53, SEQ ID NO: 62, SEQ ID NO: 63, SEQ ID NO: 72, SEQ ID NO: 73, SEQ ID NO: 81, SEQ ID NO: 82, SEQ ID NO: 88, SEQ ID NO: 89, or SEQ ID NO: 92). In certain embodiments, substitutions, insertions, or deletions occur in regions outside the CDRs (e.g., in the FRs) of the extracellular antigen-binding domain. In certain embodiments, the extracellular antigen-binding domain comprises V_{H} and/or V_{L} sequence selected from SEQ ID NOs: 8, 9, 18, 19, 28, 29, 35, 43, 44, 52, 53, 62, 63, 72, 73, 81, 82, 88, 89, or 92 including post-translational modifications of that sequence (SEQ ID NO: 8, 9, 18, 19, 28, 29, 35, 43, 44, 52, 53, 62, 63, 72, 73, 81, 82, 88, 89, or 92).

In certain embodiments, the extracellular antigen-binding domain of a presently disclosed antigen recognizing receptor (e.g., a CAR) cross-competes for binding to CD33 (e.g., human CD33) with a reference antibody or an antigen-binding fragment thereof comprising the V_{H} CDR1, CDR2, and CDR3 sequences and the V_{L} CDR1, CDR2, and CDR3 sequences of, for example, any one of the presently disclosed scFvs (e.g. 3-P14, 4-B2, 1-J19, 1-J19-2, 1-P13, 1-P23, 1-A20, 2-N3, 1-H19, 2-F18, and 4-P3). In certain embodiments, the extracellular antigen-binding domain of a presently disclosed CAR cross-competes for binding to CD33 (e.g., human CD33) with a reference antibody or an antigen-binding portion thereof comprising the V_{H} and V_{L} sequences of, for example, any one of the presently disclosed scFvs (e.g., 3-P14, 4-B2, 1-J19, 1-J19-2, 1-P13, 1-P23, 1-A20, 2-N3, 1-H19, 2-F18, and 4-P3).

In certain embodiments, the extracellular antigen-binding domain of a presently disclosed antigen recognizing receptor (e.g., a CAR) cross-competes for binding to CD33 (e.g., human CD33) with a reference antibody or an antigen-binding portion thereof comprising the V_{H} CDR1, CDR2, and CDR3 sequences and the V_{L} CDR1, CDR2, and CDR3 sequences of scFv 3-P14. For example, the extracellular antigen-binding domain of a presently disclosed antigen recognizing receptor (e.g., a CAR) cross-competes for binding to CD33 (e.g., human CD33) with a reference antibody or an antigen-binding portion thereof comprising a V_{H} CDR1 comprising the amino acid sequence set forth in SEQ ID NO: 2, a V_{H} CDR2 comprising the amino acid sequence set forth in SEQ ID NO: 3; a V_{H} CDR3 comprising the amino acid sequence set forth in SEQ ID NO: 4; a V_{L} CDR1 comprising the amino acid sequence set forth in SEQ ID NO: 5; a V_{L} CDR2 comprising amino acids having the sequence set forth in SEQ ID NO: 6; and a V_{L} CDR3 comprising amino acids having the sequence set forth in SEQ ID NO: 7. In certain embodiments, the extracellular antigen-binding domain of a presently disclosed antigen recognizing receptor (e.g., a CAR) cross-competes for binding to CD33 (e.g., human CD33) with a reference antibody or an antigen-binding portion thereof comprising the V_{H} and V_{L} sequences of scFv 3-P14. For example, the extracellular antigen-binding domain of a presently disclosed antigen recognizing receptor (e.g., a CAR) cross-competes for binding to CD33 (e.g., human CD33) with a reference antibody or an antigen-binding portion thereof comprising a V_{H} comprising amino acids having the sequence set forth in SEQ ID NO: 8, and a V_{L} Comprising amino acids having the sequence set forth in SEQ ID NO: 9.

In certain embodiments, the extracellular antigen-binding domain of a presently disclosed antigen recognizing receptor (e.g., a CAR) binds to the same epitope region on CD33 (e.g., human CD33) as the reference antibody or antigen-binding portion thereof. For example, the extracellular antigen-binding domain of a presently disclosed CAR binds to the same epitope region on CD33 (e.g., human CD33) as a reference antibody or an antigen-binding portion thereof comprising the V_{H} CDR1, CDR2, and CDR3 sequences and the V_{L} CDR1, CDR2, and CDR3 sequences of, for example, any one of the presently disclosed scFvs (e.g., 3-P14, 4-B2, 1-J19, 1-J19-2, 1-P13, 1-P23, 1-A20, 2-N3, 1-H19, 2-F18, and 4-P3). In certain embodiments, the extracellular antigen-binding domain of a presently disclosed antigen recognizing receptor (e.g., a CAR) binds to the same epitope region on CD33 (e.g., human CD33) as a reference antibody or an antigen-binding portion thereof comprising the V_{H} and V_{L} sequences of, for example, any one of the presently disclosed scFvs (e.g., 3-P14, 4-B2, 1-J19, 1-J19-2, 1-P13, 1-P23, 1-A20, 2-N3, 1-H19, 2-F18, and 4-P3).

In certain embodiments, the extracellular antigen-binding domain of a presently disclosed antigen recognizing receptor (e.g., a CAR) cross-competes for binding to CD33 (e.g., human CD33) with a reference antibody or an antigen-binding portion thereof. For example, the extracellular antigen-binding domain of a presently disclosed antigen recognizing receptor (e.g., a CAR) cross-competes for binding to CD33 (e.g., human CD33) as a reference antibody or an antigen-binding portion thereof comprising the V_{H} CDR1, CDR2, and CDR3 sequences and the V_{L} CDR1, CDR2, and CDR3 sequences of, for example, any one of the presently disclosed scFvs (e.g., 3-P14, 4-B2, 1-J19, 1-J19-2, 1-P13, 1-P23, 1-A20, 2-N3, 1-H19, 2-F18, and 4-P3). In certain embodiments, the extracellular antigen-binding domain of a presently disclosed antigen recognizing receptor (e.g., a CAR) binds to the same epitope region on CD33 (e.g., human CD33) as a reference antibody or an antigen-binding portion thereof comprising the V_{H} and V_{L} sequences of, for example, any one of the presently disclosed scFvs (e.g., 3-P14, 4-B2, 1-J19, 1-J19-2, 1-P13, 1-P23, 1-A20, 2-N3, 1-H19, 2-F18, and 4-P3).

Extracellular antigen-binding domains that cross-compete or compete with the reference antibody or antigen-binding portions thereof for binding to CD33 (e.g., human CD33) can be identified by using routine methods known in the art, including, but not limited to, ELISAs, radioimmunoassays (RIAs), Biacore, flow cytometry, Western blotting, and any other suitable quantitative or qualitative antibody-binding assays. Competition ELISA is described in Morris, “Epitope Mapping of Protein Antigens by Competition ELISA”, The Protein Protocols Handbook (1996), pp 595-600, edited by J. Walker, which is incorporated by reference in its entirety. In certain embodiments, the antibody-binding assay comprises measuring an initial binding of a reference antibody to a CD33 polypeptide, admixing the reference antibody with a test extracellular antigen-binding domain, measuring a second binding of the reference antibody to the CD33 polypeptide in the presence of the test extracellular antigen-binding domain, and comparing the initial binding with the second binding of the reference antibody, wherein a decreased second binding of the reference antibody to the CD33 polypeptide in comparison to the initial binding indicates that the test extracellular antigen-binding domain cross-competes with the reference antibody for binding to CD33, e.g., one that recognizes the same or substantially the same epitope, an overlapping epitope, or an adjacent epitope. In certain embodiments, the reference antibody is labeled, e.g., with a fluorochrome, biotin, or peroxidase. In certain embodiments, the CD33 polypeptide is expressed in cells, e.g., in a flow cytometry test. In certain embodiments, the CD33 polypeptide is immobilized onto a surface, including a Biacore ship (e.g., in a Biacore test), or other media suitable for surface plasmon resonance analysis. The binding of the reference antibody in the presence of a completely irrelevant antibody (that does not bind to CD33) can serve as the control high value. The control low value can be obtained by incubating a labeled reference antibody with an unlabeled reference antibody, where competition and reduced binding of the labeled reference antibody would occur. In certain embodiments, a test extracellular antigen-binding domain that reduces the binding of the reference antibody to a CD33polypeptide by at least about 20%, at least about 30%, at least about 40%, at least about 50%, at least about 60%, at least about 70%, at least about 80%, at least about 90%, or at least about 95% is considered to be an extracellular antigen-binding domain that cross-competes with the reference antibody for binding to CD33. In certain embodiments, the assays are performed at room temperature.

In certain embodiments, the antibody-binding assay comprises measuring an initial binding of a test extracellular antigen-binding domain to a CD33 polypeptide, admixing the test extracellular antigen-binding domain with a reference antibody, measuring a second binding of the test extracellular antigen-binding domain to the CD33 polypeptide in the presence of the reference antibody, and comparing the initial binding with the second binding of the test extracellular antigen-binding domain, where a decreased second binding of the test extracellular antigen-binding domain to the CD33 polypeptide in comparison to the initial binding indicates that the test extracellular antigen-binding domain cross-competes with the reference antibody for binding to CD33, e.g., one that recognizes the same or substantially the same epitope, an overlapping epitope, or an adjacent epitope. In certain embodiments, the test extracellular antigen-binding domain is labeled, e.g., with a fluorochrome, biotin, or peroxidase. In certain embodiments, the CD33 polypeptide is expressed in cells, e.g., in a flow cytometry test. In certain embodiments, the CD33polypeptide is immobilized onto a surface, including a Biacore ship (e.g., in a Biacore test), or other media suitable for surface plasmon resonance analysis. The binding of the test extracellular antigen-binding domain in the presence of a completely irrelevant antibody (that does not bind to CD33) can serve as the control high value. The control low value can be obtained by incubating a labeled test extracellular antigen-binding domain with an unlabeled test extracellular antigen-binding domain, where competition and reduced binding of the labeled test extracellular antigen-binding domain would occur. In certain embodiments, a test extracellular antigen-binding domain, whose binding to a CD33 polypeptide is decreased by at least about 20%, at least about 30%, at least about 40%, at least about 50%, at least about 60%, at least about 70%, at least about 80%, at least about 90%, or at least about 95% in the presence of a reference antibody, is considered to be an extracellular antigen-binding domain that cross-competes with the reference antibody for binding to CD33. In certain embodiments, the assays are performed at room temperature.

In certain embodiments, the extracellular antigen-binding domain of the presently disclosed antigen recognizing receptor (e.g., a CAR) comprises a linker connecting the heavy chain variable region and light chain variable region of the extracellular antigen-binding domain. In certain embodiments, the linker comprises or consists of the amino acid sequence set forth in SEQ ID NO: 95. In certain embodiments, the linker comprises or consists of the amino acid sequence set forth in SEQ ID NO: 96. In certain embodiments, the linker comprises or consists of the amino acid sequence set forth in SEQ ID NO: 97. In certain embodiments, the linker comprises or consists of the amino acid sequence set forth in SEQ ID NO: 98. In certain embodiments, the linker comprises or consists of the amino acid sequence set forth in SEQ ID NO: 99. In certain embodiments, the linker comprises or consists of the amino acid sequence set forth in SEQ ID NO: 100.

In certain embodiments, the variable regions within the extracellular antigen-binding domain have to be linked one after another such that at the N-terminus of the extracellular antigen-binding domain, a heavy chain variable region (V_{H}) is positioned. In certain embodiments, if the extracellular antigen-binding domain is an scFv, the variable regions are positioned from the N- to the C-terminus: V_{H}-V_{L}.

In certain embodiments, the variable regions within the extracellular antigen-binding domain have to be linked one after another such that at the N-terminus of the extracellular antigen-binding domain, a light chain variable region (V_{L}) is positioned. In certain embodiments, if the extracellular antigen-binding domain is an scFv, the variable regions are positioned from the N- to the C-terminus: V_{L}-V_{H}.

### 5.3.2. Chimeric Antigen Receptor (CAR)

In certain embodiments, the antigen-recognizing receptor is a CAR. CARs are engineered receptors, which graft or confer a specificity of interest onto an immune effector cell. CARs can be used to graft the specificity of a monoclonal antibody onto a T cell; with transfer of their coding sequence facilitated by retroviral vectors.

There are three generations of CARs. “First generation” CARs are typically composed of an extracellular antigen-binding domain (e.g., an scFv), which is fused to a transmembrane domain, which is fused to cytoplasmic/intracellular signaling domain. “First generation” CARs can provide de novo antigen recognition and cause activation of both CD4⁺ and CD8⁺ T cells through their CD3ζ chain signaling domain in a single fusion molecule, independent of HLA-mediated antigen presentation. “Second generation” CARs add intracellular signaling domains from various co-stimulatory molecules (e.g., CD28, 4-1BB, ICOS, OX40) to the cytoplasmic tail of the CAR to provide additional signals to the T cell. “Second generation” CARs comprise those that provide both co-stimulation (e.g., CD28 or 4-1BB) and activation (CD3ζ). “Third generation” CARs comprise those that provide multiple co-stimulation (e.g., CD28 and 4-1B) and activation (CD3ζ). In certain embodiments, the antigen-recognizing receptor is a first-generation CAR. In certain embodiments, the antigen-recognizing receptor is a CAR that does not comprise an intracellular signaling domain of a co-stimulatory molecule or a fragment thereof. In certain embodiments, the antigen-recognizing receptor is a second-generation CAR.

In certain embodiments, the CAR comprises an extracellular antigen-binding domain that specifically binds to CD33, a transmembrane domain, and an intracellular signaling domain.

### 5.3.2.1. Extracellular Antigen-Binding Domain of a CAR

The extracellular antigen-binding domain of the CAR can be any extracellular antigen-binding domain disclosed herein, e.g., in Section 5.3.1.

In certain embodiments, the CAR comprises an extracellular antigen-binding domain disclosed in Section 5.3.1.

In addition, the extracellular antigen-binding domain can comprise a leader or a signal peptide that directs the nascent protein into the endoplasmic reticulum. Signal peptide or leader can be essential if the CAR is to be glycosylated and anchored in the cell membrane. The signal sequence or leader can be a peptide sequence (about 5, about 10, about 15, about 20, about 25, or about 30 amino acids long) present at the N-terminus of newly synthesized proteins that directs their entry to the secretory pathway. In certain embodiments, the signal peptide is covalently joined to the 5′ terminus of the extracellular antigen-binding domain. In certain embodiments, the signal peptide comprises a CD8 polypeptide, e.g., the CAR comprises a truncated CD8 signal peptide. In certain embodiments, the CD8 signal peptide comprises or consists of amino acids 1-18 of SEQ ID NO: 101. An exemplary nucleotide sequence encoding amino acids 1-18 of SEQ ID NO: 101 is set forth in SEQ ID NO: 122, which is provided below.

### ATGGCTCTCCCAGTGACTGCCCTACTGCTTCCCCTAGCGCTTCTCCTGCATGCA [SEQ ID NO: 122]

### 5.3.2.2. Transmembrane Domain of a CAR

In certain embodiments, the transmembrane domain of the CAR comprises a hydrophobic alpha helix that spans at least a portion of the membrane. Different transmembrane domains result in different receptor stability. After antigen recognition, receptors cluster and a signal are transmitted to the cell. In accordance with the presently disclosed subject matter, the transmembrane domain of the CAR can comprise a native or modified transmembrane domain of CD8 or a fragment thereof, a native or modified transmembrane domain of CD28 or a fragment thereof, a native or modified transmembrane domain of CD3ζ or a fragment thereof, a native or modified transmembrane domain of CD4 or a fragment thereof, a native or modified transmembrane domain of 4-1BB or a fragment thereof, a native or modified transmembrane domain of OX40 or a fragment thereof, a native or modified transmembrane domain of ICOS or a fragment thereof, a native or modified transmembrane domain of CD84 or a fragment thereof, a native or modified transmembrane domain of CD166 or a fragment thereof, a native or modified transmembrane domain of CD8a or a fragment thereof, a native or modified transmembrane domain of CD8b or a fragment thereof, a native or modified transmembrane domain of ICAM-1 or a fragment thereof, a native or modified transmembrane domain of CTLA-4 or a fragment thereof, a native or modified transmembrane domain of CD27 or a fragment thereof, a native or modified transmembrane domain of CD40 or a fragment thereof, NKGD2 or a fragment thereof, or a combination thereof.

In certain embodiments, the transmembrane domain of the CAR comprises a CD8 polypeptide (e.g., a transmembrane domain of CD8 or a fragment thereof). In certain embodiments, the transmembrane domain of the CAR comprises a transmembrane domain of human CD8 or a fragment thereof. In certain embodiments, the CD8 polypeptide comprises or consists of an amino acid sequence that is at least about 85%, about 90%, about 95%, about 96%, about 97%, about 98%, about 99% or about 100% homologous or identical to the amino acid sequence having a NCBI Reference No: NP_001139345.1 (SEQ ID NO: 101) or a fragments thereof, and/or may optionally comprise up to one or up to two or up to three conservative amino acid substitutions. In certain embodiments, the CD8 polypeptide comprises or consists of an amino acid sequence that is a consecutive portion of SEQ ID NO: 101, which is at least 20, or at least 30, or at least 40, or at least 50, and up to 235 amino acids in length. In certain embodiments, the CD8 polypeptide comprises or consists of an amino acid sequence of amino acids 1 to 235, 1 to 50, 50 to 100, 100 to 150, 150 to 200, 137 to 209 or 200 to 235 of SEQ ID NO: 101. In certain embodiments, the transmembrane domain of the CAR comprises a CD8 polypeptide comprising or consisting of amino acids 137 to 209 of SEQ ID NO: 101. SEQ ID NO: 101 is provided below.

[SEQ ID NO: 101]
MALPVTALLLPLALLLHAARPSQFRVSPLDRTWNLGETVELKCQVLLSNP
TSGCSWLFQPRGAAASPTFLLYLSQNKPKAAEGLDTQRFSGKRLGDTFVL
TLSDFRRENEGYYFCSALSNSIMYFSHFVPVFLPAKPTTTPAPRPPTPAP
TIASQPLSLRPEACRPAAGGAVHTRGLDFACDIYIWAPLAGTCGVLLLSL
VITLYCNHRNRRRVCKCPRPVVKSGDKPSLSARYV

In certain embodiments, the transmembrane domain of the CAR comprises a transmembrane domain of mouse CD8 or a fragment thereof. In certain embodiments, the CD8 polypeptide comprises or consists of an amino acid sequence that is at least about 85%, about 90%, about 95%, about 96%, about 97%, about 98%, about 99% or about 100% homologous or identical to the amino acid sequence having a NCBI Reference No: AAA92533.1 (SEQ ID NO: 102) or a fragment thereof, and/or may optionally comprise up to one or up to two or up to three conservative amino acid substitutions. In certain embodiments, the CD8 polypeptide comprises or consists of an amino acid sequence that is a consecutive portion of SEQ ID NO: 102, which is at least about 20, or at least about 30, or at least about 40, or at least about 50, or at least about 60, or at least about 70, or at least about 100, or at least about 200, and up to 247 amino acids in length. In certain embodiments, the CD8 polypeptide comprises or consists of an amino acid sequence of amino acids 1 to 247, 1 to 50, 50 to 100, 100 to 150, 150 to 200, 151 to 219, or 200 to 247 of SEQ ID NO: 102. In certain embodiments, the transmembrane domain of the CAR comprises a CD8 polypeptide comprising or consisting of amino acids 151 to 219 of SEQ ID NO: 102. SEQ ID NO: 102 is provided below.

| [SEQ ID No: 102] | |
|---|---|
| 1 | MASPLTRFLS LNLLLMGESI ILGSGEAKPQ APELRIFPKK MDAELGQKVD LVCEVLGSVS |
| 61 | QGCSWLFQNS SSKLPQPTFV VYMASSHNKI TWDEKLNSSK LFSAVRDTNN KYVLTLNKFS |
| 121 | KENEGYYFCS VISNSVMYFS SVVPVLQKVN STTTKPVLRT PSPVHPTGTS QPQRPEDCRP |
| 181 | RGSVKGTGLD FACDIYIWAP LAGICVAPLL SLIITLICYH RSRKRVCKCP RPLVRQEGKP |
| 241 | RPSEKIV |

In certain embodiments, the transmembrane domain of a presently disclosed CAR comprises a CD28 polypeptide (e.g., a transmembrane domain of CD28 or a fragment thereof).

In certain embodiments, the transmembrane domain of the CAR comprises a transmembrane domain of human CD28 or a fragment thereof. In certain embodiments, the CD28 polypeptide comprises or consists of an amino acid sequence that is at least about 85%, about 90%, about 95%, about 96%, about 97%, about 98%, about 99% or 100% homologous or identical to the amino acid sequence having a NCBI Reference No: NP_006130 (SEQ ID No: 103) or a fragment thereof, and/or may optionally comprise up to one or up to two or up to three conservative amino acid substitutions. In non-limiting certain embodiments, the CD28 polypeptide comprises or consists of an amino acid sequence that is a consecutive portion of SEQ ID NO: 103 which is at least 20, or at least 30, or at least 40, or at least 50, and up to 220 amino acids in length. In certain embodiments, the CD28 polypeptide comprises or consists of an amino acid sequence of amino acids 1 to 220, 1 to 50, 50 to 100, 100 to 150, 150 to 200, 153 to 179, or 200 to 220 of SEQ ID NO: 103. In certain embodiments, the transmembrane domain of the CAR comprises a CD28 polypeptide comprising or consisting of amino acids 153 to 179 of SEQ ID NO: 103. SEQ ID NO: 103 is provided below:

| [SEQ ID NO: 103] | |
|---|---|
| 1 | MLRLLLALNL FPSIQVTGNK ILVKQSPMLV AYDNAVNLSC KYSYNLFSRE FRASLHKGLD |
| 61 | SAVEVCVVYG NYSQQLQVYS KTGFNCDGKL GNESVTFYLQ NLYVNQTDIY FCKIEVMYPP |
| 121 | PYLDNEKSNG TIIHVKGKHL CPSPLFPGPS KPFWVLVVVG GVLACYSLLV TVAFIIFWVR |
| 181 | SKRSRLLHSD YMNMTPRRPG PTRKHYQPYA PPRDFAAYRS |

An exemplary nucleotide sequence encoding amino acid 153 to 179 of SEQ ID NO: 103 is set forth in SEQ ID NO: 104, which is provided below.

[SEQ ID NO: 104]
TTTTGGGTGCTGGTGGTGGTTGGTGGAGTCCTGGCTTGCTATAGCTTGCT
AGTAACAGTGGCCTTTATTATTTTCTGGGTG

In certain embodiments, the transmembrane domain of the CAR comprises a CD28 polypeptide (e.g., a transmembrane domain of mouse CD28 or a fragment thereof). In certain embodiments, the CD28 polypeptide comprises or consists of an amino acid sequence that is at least about 85%, about 90%, about 95%, about 96%, about 97%, about 98%, about 99% or 100% homologous or identical to the amino acid sequence having a NCBI Reference No: NP_031668.3 (SEQ ID No: 105) or a fragment thereof, and/or may optionally comprise up to one or up to two or up to three conservative amino acid substitutions. In certain embodiments, the CD28 polypeptide comprises or consists of an amino acid sequence that is a consecutive portion of SEQ ID NO: 105, which is at least 20, or at least 30, or at least 40, or at least 50, and up to 218 amino acids in length. In certain embodiments, the CD28 polypeptide comprises or consists of an amino acid sequence of amino acids 1 to 220, 1 to 50, 50 to 100, 100 to 150, 150 to 200, 151 to 177, or 200 to 218 of SEQ ID NO: 105. In certain embodiments, the transmembrane domain of the CAR comprises a CD28 polypeptide comprising or consisting of amino acids 151 to 177 of SEQ ID NO: 105. SEQ ID NO: 105 is provided below:

| [SEQ ID NO: 105] | |
|---|---|
| 1 | MTLRLLFLAL NFFSVQVTEN KILVKQSPLL VVDSNEVSLS CRYSYNLLAK EFRASLYKGV |
| 61 | NSDVEVCVGN GNFTYQPQFR SNAEFNCDGD FDNETVTFRL WNLHVNHTDI YFCKIEFMYP |
| 121 | PPYLDNERSN GTIIHIKEKH LCHTQSSPKL FWALVVVAGV LFCYGLLVTV ALCVIWTNSR |
| 181 | RNRLLQSDYM NMTPRRPGLT RKPYQPYAPA RDFAAYRP |

In certain non-limiting embodiments, the CAR further comprises a spacer region that links the extracellular antigen-binding domain to the transmembrane domain. The spacer region can be flexible enough to allow the antigen binding domain to orient in different directions to facilitate antigen recognition while preserving the activating activity of the CAR.

In certain embodiments, the hinge/spacer region of the CAR comprises a native or modified hinge region of CD8 or a fragment thereof, a native or modified hinge region of CD28 or a fragment thereof, a native or modified hinge region of CD3ζ or a fragment thereof, a native or modified hinge region of CD40 or a fragment thereof, a native or modified hinge region of 4-1BB or a fragment thereof, a native or modified hinge region of OX40 or a fragment thereof, a native or modified hinge region of CD84 or a fragment thereof, a native or modified hinge region of CD166 or a fragment thereof, a native or modified hinge region of CD8a or a fragment thereof, a native or modified hinge region of CD8b or a fragment thereof, a native or modified hinge region of ICOS or a fragment thereof, a native or modified hinge region of ICAM-1 or a fragment thereof, a native or modified hinge region of CTLA-4 or a fragment thereof, a native or modified hinge region of CD27 or a fragment thereof, a native or modified hinge region of CD40 or a fragment thereof, a native or modified hinge region of NKGD2 or a fragment thereof, a synthetic polypeptide (not based on a protein associated with the immune response), or a combination thereof. The hinge/spacer region can be the hinge region from IgG1, or the CH₂CH₃ region of immunoglobulin and portions of CD3, a portion of a CD8 polypeptide (e.g., a portion of SEQ ID NO: 101 or 102), a portion of a CD28 polypeptide (e.g., a portion of SEQ ID NO: 103 or 105), a variation of any of the foregoing which is at least about 80%, at least about 85%, at least about 90%, at least about 95%, or at least about 100% homologous or identical thereto, or a synthetic spacer sequence.

### 5.3.2.3. Intracellular Signaling Domain of a CAR

In certain embodiments, the CAR comprises an intracellular signaling domain. In certain non-limiting embodiments, the intracellular signaling domain of the CAR comprises a CD3ζ polypeptide.

CD3ζ can activate or stimulate a cell (e.g., a cell of the lymphoid lineage, e.g., a T cell). Wild type (“native”) CD3ζ comprises three functional immunoreceptor tyrosine-based activation motifs (ITAMs), three functional basic-rich stretch (BRS) regions (BRS1, BRS2 and BRS3). CD3ζ transmits an activation signal to the cell (e.g., a cell of the lymphoid lineage, e.g., a T cell) after antigen is bound. The intracellular signaling domain of the CD3ζ-chain is the primary transmitter of signals from endogenous TCRs.

In certain embodiments, the intracellular signaling domain of the CAR comprises a native CD3ζ. In certain embodiments, the CD3ζ polypeptide comprises or consists of an amino acid sequence that is at least about 85%, about 90%, about 95%, about 96%, about 97%, about 98%, about 99% or about 100% homologous or identical to the amino acid sequence having a NCBI Reference No: NP_932170 (SEQ ID NO: 106) or a fragment thereof, and/or may optionally comprise up to one or up to two or up to three conservative amino acid substitutions. In certain non-limiting embodiments, the CD3ζ polypeptide comprises or consists of an amino acid sequence that is a consecutive portion of SEQ ID NO: 106, which is at least 20, or at least 30, or at least 40, or at least 50, and up to 164 amino acids in length. In certain embodiments, the CD3ζ polypeptide comprises or consists of an amino acid sequence of amino acids 1 to 164, 1 to 50, 50 to 100, 52 to 164, 100 to 150, or 150 to 164 of SEQ ID NO: 106. In certain embodiments, the intracellular signaling domain of the CAR comprises a CD3ζ polypeptide comprising or consisting of amino acids 52 to 164 of SEQ ID NO: 106. SEQ ID NO: 106 is provided below:

| [SEQ ID NO: 106] | |
|---|---|
| 1 | MKWKALFTAA ILQAQLPITE AQSFGLLDPK LCYLLDGILF IYGVILTALF LRVKFSRSAD |
| 61 | APAYQQGQNQ LYNELNLGRR EEYDVLDKRR GRDPEMGGKP QRRKNPQEGL YNELQKDKMA |
| 121 | EAYSEIGMKG ERRRGKGHDG LYQGLSTATK DTYDALHMQA LPPR |

In certain embodiments, the intracellular signaling domain of the CAR comprises a CD3 polypeptide comprising or consisting of the amino acid sequence set forth in SEQ ID NO: 107. SEQ ID NO: 107 is provided below.

[SEQ ID NO: 107]
RVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR

An exemplary nucleotide sequence encoding the amino acid sequence of SEQ ID NO: 107 is set forth in SEQ ID NO: 108, which is as provided below.

[SEQ ID NO: 108]
AGAGTGAAGTTCAGCAGGAGCGCAGACGCCCCCGCGTACCAGCAGGGCCA
GAACCAGCTCTATAACGAGCTCAATCTAGGACGAAGAGAGGAGTACGATG
TTTTGGACAAGAGACGTGGCCGGGACCCTGAGATGGGGGGAAAGCCGAGA
AGGAAGAACCCTCAGGAAGGCCTGTACAATGAACTGCAGAAAGATAAGAT
GGCGGAGGCCTACAGTGAGATTGGGATGAAAGGCGAGCGCCGGAGGGGCA
AGGGGCACGATGGCCTTTACCAGGGTCTCAGTACAGCCACCAAGGACACC
TACGACGCCCTTCACATGCAGGCCCTGCCCCCTCGC

In certain embodiments, the intracellular signaling domain of the CAR further comprises at least a co-stimulatory signaling region. In certain embodiments, the co-stimulatory signaling region comprises at least one co-stimulatory molecule or a fragment thereof. In certain embodiments, the co-stimulatory signaling region comprises an intracellular domain of at least one co-stimulatory molecule or a fragment thereof.

As used herein, a “co-stimulatory molecule” refers to a cell surface molecule other than antigen receptor or its ligand that can provide an efficient response of lymphocytes to an antigen. In certain embodiments, a co-stimulatory molecule can provide optimal lymphocyte activation. Non-limiting examples of co-stimulatory molecules include CD28, 4-1BB, OX40, ICOS, DAP-10, CD27, CD40, NKGD2, CD2, FN14, HVEM, LTBR, CD28H, TNFR1, TNFR2, BAFF-R, BCMA, TACI, TROY, RANK, CD40, CD27, CD30, EDAR, XEDAR, GITR, DR6, and NGFR, and combinations thereof. The co-stimulatory molecule can bind to a co-stimulatory ligand, which is a protein expressed on cell surface that upon binding to its receptor produces a co-stimulatory response, i.e., an intracellular response that effects the stimulation provided when an antigen-recognizing receptor (e.g., a chimeric antigen receptor (CAR)) binds to its target antigen. As one example, a 4-1BB ligand (i.e., 4-1BBL) may bind to 4-1BB for providing an intracellular signal that in combination with a CAR signal induces an effector cell function of the CAR⁺ T cell.

In certain embodiments, the intracellular signaling domain of the CAR comprises a co-stimulatory signaling region that comprises a CD28 polypeptide, e.g., an intracellular domain of CD28 or a fragment thereof. In certain embodiments, the intracellular signaling domain of the CAR comprises a co-stimulatory signaling region that comprises a CD28 polypeptide, e.g., an intracellular domain of human CD28 or a fragment thereof. In certain embodiments, the CD28 polypeptide comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence set forth in SEQ ID NO: 103 or a fragment thereof, and/or may optionally comprise up to one or up to two or up to three conservative amino acid substitutions. In certain embodiments, the CD28 polypeptide comprises or consists of an amino acid sequence that is a consecutive portion of SEQ ID NO: 103, which is at least 20, or at least 30, or at least 40, or at least 50, and up to 220 amino acids in length. In certain embodiments, the CD28 polypeptide comprises or consists of an amino acid sequence of amino acids 1 to 220, 1 to 50, 50 to 100, 100 to 150, 114 to 220, 150 to 200, 180 to 220, or 200 to 220 of SEQ ID NO: 103. In certain embodiments, the intracellular signaling domain of the CAR comprises a co-stimulatory signaling region that comprises a CD28 polypeptide comprising or consisting of an amino acid sequence of amino acids 180 to 220 of SEQ ID NO: 103.

An exemplary nucleotide sequence encoding amino acids 180 to 220 of SEQ ID NO: 103 is set forth in SEQ ID NO: 109, which is provided below.

[SEQ ID NO: 109]
AGGAGTAAGAGGAGCAGGCTCCTGCACAGTGACTACATGAACATGACTCC
CCGCCGCCCCGGGCCCACCCGCAAGCATTACCAGCCCTATGCCCCACCAC
GCGACTTCGCAGCCTATCGCTCC

In certain embodiments, the intracellular signaling domain of the CAR comprises a co-stimulatory signaling region that comprises a CD28 polypeptide, e.g., an intracellular domain of mouse CD28 or a fragment thereof. In certain embodiments, the CD28 polypeptide comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence set forth in SEQ ID NO: 104 or a fragment thereof, and/or may optionally comprise up to one or up to two or up to three conservative amino acid substitutions. In non-limiting certain embodiments, the CD28 polypeptide comprises or consists of an amino acid sequence that is a consecutive portion of SEQ ID NO: 104, which is at least about 20, or at least about 30, or at least about 40, or at least about 50, and up to 218 amino acids in length. In certain embodiments, the CD28 polypeptide comprises or consists of an amino acid sequence of amino acids 1 to 218, 1 to 50, 50 to 100, 100 to 150, 150 to 218, 178 to 218, or 200 to 218 of SEQ ID NO: 105. In certain embodiments, the co-stimulatory signaling region of a presently disclosed CAR comprises a CD28 polypeptide that comprises or consists of the amino acids 178 to 218 of SEQ ID NO: 105.

In certain embodiments, the co-stimulatory signaling region of a presently disclosed CAR comprises a CD28 polypeptide comprising a mutated YMNM motif. CD28 is a transmembrane protein that plays a critical role in T cell activation through its function as a costimulatory molecule. CD28 possesses an intracellular domain, which comprises intracellular motifs that are critical for the effective signaling of CD28. In certain embodiments, the CD28 intracellular domain comprises intracellular subdomains (also known as “intracellular motifs”) that regulate signaling pathways post TCR-stimulation. CD28 includes three intracellular motifs: a YMNM motif, and two proline-rick motifs: PRRP motif, and PYAP motif. The CD28 intracellular motifs can serve as docking sites for a number of adaptor molecules that interact with these motifs through their SH2 or SH3 domains. Such interaction transduces downstream signals terminating on transcription factors that regulate gene expression. For example, a native YMNM motif binds to a p85 subunit of a phosphoinositide 3-kinase (PI3K). A native YMNM motif also binds to growth factor receptor-bound protein 2 (Grb2) and/or Grb2-related adaptor protein 2 (GADS). Grb2 binds to Gab1 and Gab2, which in turn can recruit the p85 subunit of a PI3K.

In certain embodiments, a native YMNM motif consists of the amino acid sequence set forth in YMNM (SEQ ID NO: 123). In certain embodiments, a native YMNM motif binds to the p85 subunit of PI3K via a consensus sequence YMxM (SEQ ID NO: 124), wherein x is not an aspartic acid (N). In certain embodiments, a native YMNM motif binds to Grb2 and/or GADs via a consensus sequence YxNx (SEQ ID NO: 125), wherein x is not a methionine (M).

In certain embodiments, the CD28 polypeptide comprising a presently disclosed mutated YMNM motif has reduced recruitment of the p85 subunit of a PI3K as compared to a CD28 molecule comprising a native YMNM motif. In certain embodiments, the p85 subunit of a PI3K does not bind to the mutated YMNM motif, thereby reducing the recruitment of the p85 subunit of a PI3K to the CD28 polypeptide. The mutated YMNM motif that blocks the binding of the p85 subunit of a PI3K retains its binding to Grb2 and/or GADS. Thus, downstream signaling of Grb2/GADS remains intact, e.g., downstream signaling leading to IL-2 secretion remains intact. Such mutated YMNM motif is referred to as “GADS/Grb2-permitting mutant”.

In certain embodiments, the mutated YMNM binds to the p85 subunit of a PI3K, but does not bind to Grb2 and/or GADS. Since the binding of PI3K p85 is retained, the downstream signaling of PI3K retains intact. Since the binding of Grb2/GADS is blocked, the recruitment of PI3K p85 subunit, which is triggered by the binding of Grb2 to Gab1 and Gab2, is reduced or blocked. In addition, the downstream signaling of Grb2/GADS is blocked. Such mutated YMNM motif is referred to as “PI3K-permissive mutant”.

In certain embodiments, the mutated YMNM does not bind to the p85 subunit of a PI3K, and does not bind to Grb2 and/or GADS. Such mutated YMNM motif is referred to as “non-functional mutant”. Non-functional mutants do not provide binding of PI3K, Grb2, or GADS to CD28 at the YMNM motif, but do not preclude these signaling molecules from binding elsewhere in the CD28 molecule.

In certain embodiments, the mutated YMNM retains only one methionine residue of the two methionine residues present in the YMNM motif, i.e. YMxx or YxxM. These motifs potentially modulate signaling via PI3K by limiting how many methionine residues can bind the p85 subunit of PI3K. Such mutated YMNM motif is referred to as “hybrid ‘HEMI’ mutant”.

In certain embodiments, the mutated YMNM motif is a GADS/Grb-2 permitting mutant. In certain embodiments, the mutated YMNM motif consists of the amino acid sequence set forth in YxNx (SEQ ID NO: 125), wherein x is not a methionine (M). In certain embodiments, x is selected from the group consisting of amino acids A, R, N, D, C, E, Q, G, H, I, K, F, P, S, T, W, Y, V, and L. In certain embodiments, the mutated YMNM motif consists of the amino acid sequence set forth in YENV (SEQ ID NO: 126), YSNV (SEQ ID NO: 127), YKNL (SEQ ID NO: 128), YENQ (SEQ ID NO: 129), YKNI (SEQ ID NO: 130), YINQ (SEQ ID NO: 131), YHNK (SEQ ID NO: 132), YVNQ (SEQ ID NO: 133), YLNP (SEQ ID NO: 134), YLNT (SEQ ID NO: 135), YDND (SEQ ID NO: 136), YENI (SEQ ID NO: 137), YENL (SEQ ID NO: 138), YKNQ (SEQ ID NO: 139), YKNV (SEQ ID NO: 140), or YANG (SEQ ID NO: 141). In certain embodiments, the mutated YMNM motif consists of the amino acid sequence set forth in YSNV (SEQ ID NO: 127). In certain embodiments, the mutated YMNM motif consists of the amino acid sequence set forth in YKNI (SEQ ID NO: 130). In certain embodiments, the mutated YMNM motif consists of the amino acid sequence set forth in YENV (SEQ ID NO: 126). In certain embodiments, the mutated YMNM motif consists of the amino acid sequence set forth in YKNL (SEQ ID NO: 128).

In certain embodiments, the mutated YMNM motif is a PI3K-permissive mutant. In certain embodiments, the mutated YMNM motif consists of the amino acid sequence set forth in YMxM (SEQ ID NO: 124), wherein x is not an aspartic acid (N). In certain embodiments, x is selected from the group consisting of amino acids A, R, D, C, E, Q, G, H, I, K, M, F, P, S, T, W, Y, V, and L. In certain embodiments, the mutated YMNM motif consists of the amino acid sequence set forth in YMDM (SEQ ID NO: 142), YMPM (SEQ ID NO: 143), YMRM (SEQ ID NO: 144), or YMSM (SEQ ID NO: 145). In certain embodiments, the mutated YMNM motif consists of the amino acid sequence set forth in YMDM (SEQ ID NO: 142).

In certain embodiments, the mutated YMNM motif consists of the amino acid sequence set forth in YbxM (SEQ ID NO: 146), wherein x is not an aspartic acid (N), and b is not a methionine (M). In certain embodiments, x is selected from the group consisting of amino acids A, R, D, C, E, Q, G, H, I, K, M, F, P, S, T, W, Y, V, and L. In certain embodiments, b is selected from the group consisting of amino acids A, R, N, C, E, Q, G, H, I, K, N, F, P, S, T, W, Y, V, and L. In certain embodiments, the mutated YMNM motif consists of the amino acid sequence set forth in YTHM (SEQ ID NO: 147), YVLM (SEQ ID NO: 148), YIAM (SEQ ID NO: 149), YVEM (SEQ ID NO: 150), YVKM (SEQ ID NO: 151), or YVPM (SEQ ID NO: 152).

In certain embodiments, the mutated YMNM motif consists of the amino acid sequence set forth in YMxb (SEQ ID NO: 153), wherein x is not an aspartic acid (N), and b is not a methionine (M). In certain embodiments, x is selected from the group consisting of amino acids A, R, D, C, E, Q, G, H, I, K, M, F, P, S, T, W, Y, V, and L. In certain embodiments, b is selected from the group consisting of amino acids A, R, N, C, E, Q, G, H, I, K, N, F, P, S, T, W, Y, V, and L. In certain embodiments, the mutated YMNM motif consists of the amino acid sequence set forth in YMAP (SEQ ID NO: 154).

Certain mutated YMNM motifs are described in Mol Cell Proteomics. 2010 November; 9(11):2391-404; Virology. 2015 May; 0: 568-577, both of which are incorporated by reference herein in its entirety.

In certain embodiments, the mutated YMNM motif is a hybrid ‘HEMI’ mutant. In certain embodiments, the mutated YMNM motif consists of the amino acid sequence set forth in YMNx (SEQ ID NO: 155) or YxNM (SEQ ID NO: 156), wherein x is not a methionine (M). In certain embodiments, x is selected from the group consisting of amino acids A, R, N, C, E, Q, G, H, I, K, N, F, P, S, T, W, Y, V, and L. In certain embodiments, the mutated YMNM motif consists of the amino acid sequence set forth in YMNV (SEQ ID NO: 157), YENM (SEQ ID NO: 158), YMNQ (SEQ ID NO: 159), YMNL (SEQ ID NO: 160), or YSNM (SEQ ID NO: 161).

In certain embodiments, the mutated YMNM motif is a non-functional mutant. In certain embodiments, the mutated YMNM motif consists of the amino acid sequence Ybxb (SEQ ID NO: 162), wherein x is not an aspartic acid (N), and b is not a methionine (M). In certain embodiments, x is selected from the group consisting of A, R, D, C, E, Q, G, H, I, K, M, F, P, S, T, W, Y, V, and L. In certain embodiments, b is selected from the group consisting of A, R, N, D, C, E, Q, G, H, I, K, F, P, S, T, W, Y, V, and L. In certain embodiments, the mutated YMNM motif consists of the amino acid sequence set forth in YGGG (SEQ ID NO: 163), YAAA (SEQ ID NO: 164), YFFF (SEQ ID NO: 165), YETV (SEQ ID NO: 166), YQQQ (SEQ ID NO: 167), YHAE (SEQ ID NO: 168), YLDL (SEQ ID NO: 169), YLIP (SEQ ID NO: 170), YLRV (SEQ ID NO: 171), YTAV (SEQ ID NO: 172), or YVHV (SEQ ID NO: 173). In certain embodiments, the mutated YMNM motif consists of the amino acid sequence set forth in YGGG (SEQ ID NO: 163).

In certain embodiments, the intracellular signaling domain of the presently disclosed chimeric receptor comprises a co-stimulatory signaling domain that comprises a CD28 polypeptide comprising a mutated YMNM motif consisting of the amino acid sequence set forth in YENV (SEQ ID NO: 126), wherein the CD28 polypeptide comprises or consists of the amino acid sequence set forth in SEQ ID NO: 174. SEQ ID NO: 174 is provided below.

[SEQ ID NO: 174]
RSKRSRLLHSDYENVTPRRPGPTRKHYQPYAPPRDFAAYRS

In certain embodiments, the intracellular signaling domain of the presently disclosed chimeric receptor comprises a co-stimulatory signaling domain that comprises a CD28 polypeptide comprising a mutated YMNM motif consisting of the amino acid sequence set forth in YKNI (SEQ ID NO: 130), wherein the CD28 polypeptide comprises or consists of the amino acid sequence set forth in SEQ ID NO: 175. SEQ ID NO: 175 is provided below.

[SEQ ID NO: 175]
RSKRSRLLHSDYKNITPRRPGPTRKHYQPYAPPRDFAAYRS

In certain embodiments, the intracellular signaling domain of the presently disclosed chimeric receptor comprises a co-stimulatory signaling domain that comprises a CD28 polypeptide comprising a mutated YMNM motif consisting of the amino acid sequence set forth in YMDM (SEQ ID NO: 142), wherein the CD28 polypeptide comprises or consists of the amino acid sequence set forth in SEQ ID NO: 176. SEQ ID NO: 176 is provided below.

[SEQ ID NO: 176]
RSKRSRLLHSDYMDMTPRRPGPTRKHYQPYAPPRDFAAYRS

In certain embodiments, the intracellular signaling domain of the presently disclosed chimeric receptor comprises a co-stimulatory signaling domain that comprises a CD28 polypeptide comprising a mutated YMNM motif consisting of the amino acid sequence set forth in YGGG (SEQ ID NO: 163), wherein the CD28 polypeptide comprises or consists of the amino acid sequence set forth in SEQ ID NO: 177. SEQ ID NO: 177 is provided below.

[SEQ ID NO: 177]
RSKRSRLLHSDYGGGTPRRPGPTRKHYQPYAPPRDFAAYRS

In certain embodiments, the intracellular signaling domain of the presently disclosed chimeric receptor comprises a co-stimulatory signaling domain that comprises a CD28 polypeptide comprising a mutated YMNM motif consisting of the amino acid sequence set forth in YSNV (SEQ ID NO: 127), wherein the CD28 polypeptide comprises or consists of the amino acid sequence set forth in SEQ ID NO: 178. SEQ ID NO: 178 is provided below.

[SEQ ID NO: 178]
RSKRSRLLHSDYSNVTPRRPGPTRKHYQPYAPPRDFAAYRS

In certain embodiments, the intracellular signaling domain of the presently disclosed CAR comprises a first co-stimulatory signaling domain that comprises a CD28 polypeptide comprising a mutated YMNM motif (as disclosed herein), and a second co-stimulatory signaling domain that comprises an intracellular domain of a co-stimulatory molecule. Additional information regarding CARs including CD28 polypeptide comprising a mutated YMNM motif can be found in International Patent Publication No. WO 2021/158850, which is incorporated by reference in its entirety.

In certain embodiments, the intracellular signaling domain of the CAR comprises a co-stimulatory signaling region that comprises a 4-1BB polypeptide, e.g., an intracellular domain of 4-1BB or a fragment thereof. In certain embodiments, the intracellular signaling domain of the CAR comprises a co-stimulatory signaling region that comprises a 4-1BB polypeptide, e.g., an intracellular domain of human 4-1BB or a fragment thereof. In certain embodiments, the 4-1BB polypeptide comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence having a NCBI Ref. No.: NP_001552 (SEQ ID NO: 110) or a fragment thereof, and/or may optionally comprise up to one or up to two or up to three conservative amino acid substitutions. In non-limiting certain embodiments, the 4-1BB polypeptide comprises or consists of an amino acid sequence that is a consecutive portion of SEQ ID NO: 106, which is at least 20, or at least 30, or at least 40, or at least 50, or at least 100, or at least 150, or at least 150, and up to 255 amino acids in length. In certain embodiments, the 4-1BB polypeptide comprises or consists of an amino acid sequence of amino acids 1 to 255, 1 to 50, 50 to 100, 100 to 150, 150 to 200, or 200 to 255 of SEQ ID NO: 110. In certain embodiments, the intracellular signaling domain of the CAR comprises a co-stimulatory signaling region that comprises a 4-1BB polypeptide comprising or consisting of an amino acid sequence of amino acids 214 to 255 of SEQ ID NO: 110. SEQ ID NO: 110 is provided below.

| [SEQ ID NO: 110] | |
|---|---|
| 1 | MGNSCYNIVA TLLLVLNFER TRSLQDPCSN CPAGTFCDNN RNQICSPCPP NSFSSAGGQR |
| 61 | TCDICRQCKG VFRTRKECSS TSNAECDCTP GFHCLGAGCS MCEQDCKQGQ ELTKKGCKDC |
| 121 | CFGTFNDQKR GICRPWTNCS LDGKSVLVNG TKERDVVCGP SPADLSPGAS SVTPPAPARE |
| 181 | PGHSPQIISF FLALTSTALL FLLFFLTLRF SVVKRGRKKL LYIFKQPFMR PVQTTQEEDG |
| 241 | CSCRFPEEEE GGCEL |

An exemplary nucleotide sequence encoding amino acids 214 to 255 of SEQ ID NO: 110 is set forth in SEQ ID NO: 111, which is provided below.

[SEQ ID NO: 111]
AAACGGGGCAGAAAGAAACTCCTGTATATATTCAAACAACCATTTATGA
GACCAGTACAAACTACTCAAGAGGAAGATGGCTGTAGCTGCCGATTTCC
AGAAGAAGAAGAAGGAGGATGTGAACTG

In certain embodiments, the intracellular signaling domain of the CAR comprises a co-stimulatory signaling region that comprises intracellular domains of two or more co-stimulatory molecules or portions thereof, e.g., an intracellular domain of CD28 or a fragment thereof and an intracellular domain of 4-1BB or a fragment thereof, or an intracellular domain of CD28 or a fragment thereof and an intracellular domain of OX40 or a fragment thereof.

In certain embodiments, a presently disclosed CAR further comprises an inducible promoter, for expressing nucleic acid sequences in human cells. Promoters for use in expressing CAR genes can be a constitutive promoter, such as ubiquitin C (UbiC) promoter.

### 5.3.2.4. Exemplified CARs

In certain embodiments, the CAR is a CD33-targeted CAR. In certain embodiments, the CAR comprises (a) an extracellular antigen-binding domain comprising (i) a V_{H} that comprises a CDR1 comprising the amino acid sequence set forth in SEQ ID NO: 2, a CDR2 comprising the amino acid sequence set forth in SEQ ID NO: 3, and a CDR3 comprising the amino acid sequence set forth in SEQ ID NO: 4, and (ii) a V_{L} that comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 7; (b) a transmembrane domain comprising a CD28 polypeptide (e.g., a transmembrane domain of human CD28 or a fragment thereof), and (c) an intracellular signaling domain comprising (i) a CD3ζ polypeptide, and (ii) a co-stimulatory signaling region comprising a CD28 polypeptide (e.g., an intracellular domain of human CD28 or a fragment thereof). In certain embodiments, the transmembrane domain comprises a CD28 polypeptide that comprises amino acids 153 to 179 of SEQ ID NO: 103. In certain embodiments, the intracellular signaling domain comprises (i) a CD3ζ polypeptide comprising the amino acid sequence set forth in SEQ ID NO: 107, and (ii) a co-stimulatory signaling region comprising a CD28 polypeptide comprising the amino acid sequence set amino acids 180 to 220 of SEQ ID NO: 103. In certain embodiments, the extracellular antigen-binding domain is an scFv that is designated as “3-P14”. In certain embodiments, the V_{H} and V_{L} are linked via a linker comprising or consisting of the amino acid sequence set forth in SEQ ID NO: 96. In certain embodiments, the V_{H} and V_{L} are positioned from the N- to the C-terminus: V_{H}-V_{L}. In certain embodiments, the CAR is designed as “TDI-Y-006_h28z”. In certain embodiments, the CAR comprises the amino acid sequence set forth in SEQ ID NO: 112, which is provided below.

[SEQ ID NO: 112]
EVQLLESGGGLVQPGGSLRLSCAASGFTFSTYAMSWVRQAPGKGLEWVS
AISGRGGSTYYTDSVKGRFTISRDNSKNTVSLQMNSLRAEDTAVYYCAG
RGDYYYYYGMDVWGQGTTVTVSAGGGGSGGGGSGGGGSDIVMTQSPLSS
PVTLGQPASFSCRSSQSLVYSDGNTYLSWLQQRPGQPPRLLIYKISNRF
SGVPDRFSGSGAGTDFTLKISRVEAEDVGVYYCMQSTQFPHTFGQGTKL
EIKEQKLISEEDLAAAIEVMYPPPYLDNEKSNGTIIHVKGKHLCPSPLF
PGPSKPFWVLVVVGGVLACYSLLVTVAFIIFWVRSKRSRLLHSDYMNMT
PRRPGPTRKHYQPYAPPRDFAAYRSRVKESRSADAPAYQQGQNQLYNEL
NLGRREEYDVLDKRRGRDPEMGGKPRRKNPQEGLYNELQKDKMAEAYSE
IGMKGERRRGKGHDGLYQGLSTATKDTYDALHMQALPPR

An exemplary nucleotide sequence encoding the amino acid sequence of SEQ ID NO: 112 is set forth in SEQ ID NO: 113, which is provided below.

[SEQ ID NO: 113]
GAGGTGCAGCTGTTGGAGTCTGGGGGAGGCTTGGTCCAGCCTGGGGGGTCCCTGAGACTCTCCTGTGCAGCCTCTGGATT
CACCTTTAGCACCTATGCCATGAGCTGGGTCCGCCAGGCTCCAGGGAAGGGGCTGGAGTGGGTCTCAGCTATTAGTGGTC
GTGGTGGTAGCACATACTACACAGACTCCGTGAAGGGCCGGTTCACCATCTCCAGAGACAATTCCAAGAACACGGTGTCT
CTGCAAATGAACAGCCTGAGAGCCGAGGACACGGCCGTATATTACTGTGCGGGCCGGGGAGATTACTACTACTACTACGG
TATGGACGTCTGGGGCCAAGGGACCACGGTCACCGTCTCCGCAGGTGGAGGTGGATCAGGTGGAGGTGGATCTGGTGGAG
GTGGATCTGATATTGTGATGACCCAGAGTCCACTCTCCTCACCTGTCACCCTTGGACAGCCGGCCTCCTTCTCCTGCAGG
TCTAGTCAAAGCCTCGTATACAGTGATGGAAACACCTACTTGAGTTGGCTTCAGCAGAGGCCAGGCCAGCCTCCAAGACT
CCTAATTTATAAGATTTCTAACCGGTTCTCTGGGGTCCCAGACAGATTCAGTGGCAGTGGGGCAGGGACAGATTTCACAC
TGAAAATCAGCAGGGTGGAAGCTGAGGATGTCGGGGTTTATTACTGCATGCAATCTACACAATTTCCTCACACTTTTGGC
CAGGGGACCAAGCTGGAGATCAAAGAACAGAAACTGATCTCTGAAGAAGACCTGGCGGCCGCAATTGAAGTTATGTATCC
TCCTCCTTACCTAGACAATGAGAAGAGCAATGGAACCATTATCCATGTGAAAGGGAAACACCTTTGTCCAAGTCCCCTAT
TTCCCGGACCTTCTAAGCCCTTTTGGGTGCTGGTGGTGGTTGGTGGAGTCCTGGCTTGCTATAGCTTGCTAGTAACAGTG
GCCTTTATTATTTTCTGGGTGAGGAGTAAGAGGAGCAGGCTCCTGCACAGTGACTACATGAACATGACTCCCCGCCGCCC
CGGGCCCACCCGCAAGCATTACCAGCCCTATGCCCCACCACGCGACTTCGCAGCCTATCGCTCCAGAGTGAAGTTCAGCA
GGAGCGCAGACGCCCCCGCGTACCAGCAGGGCCAGAACCAGCTCTATAACGAGCTCAATCTAGGACGAAGAGAGGAGTAC
GATGTTTTGGACAAGAGACGTGGCCGGGACCCTGAGATGGGGGGAAAGCCGAGAAGGAAGAACCCTCAGGAAGGCCTGTA
CAATGAACTGCAGAAAGATAAGATGGCGGAGGCCTACAGTGAGATTGGGATGAAAGGCGAGCGCCGGAGGGGCAAGGGGC
ACGATGGCCTTTACCAGGGTCTCAGTACAGCCACCAAGGACACCTACGACGCCCTTCACATGCAGGCCCTGCCCCCTCGC
TAG

In certain embodiments, the CAR is a CD33-targeted CAR. In certain embodiments, the CAR comprises (a) an extracellular antigen-binding domain comprising (i) a V_{H} that comprises a CDR1 comprising the amino acid sequence set forth in SEQ ID NO: 12, a CDR2 comprising the amino acid sequence set forth in SEQ ID NO: 13, and a V_{H} CDR3 comprising the amino acid sequence set forth in SEQ ID NO: 14, and (ii) a V_{L} that comprises a CDR1 comprising the amino acid sequence set forth in SEQ ID NO: 15, a CDR2 comprising the amino acid sequence set forth in SEQ ID NO: 16, and a V_{L} CDR3 comprising the amino acid sequence set forth in SEQ ID NO: 17; (b) a transmembrane domain comprising a CD28 polypeptide (e.g., a transmembrane domain of human CD28 or a portion thereof), and (c) an intracellular signaling domain comprising (i) a CD3ζ polypeptide, and (ii) a co-stimulatory signaling region comprising a CD28 polypeptide (e.g., an intracellular domain of human CD28 or a portion thereof). In certain embodiments, the transmembrane domain comprises a CD28 polypeptide that comprises amino acids 153 to 179 of SEQ ID NO: 103. In certain embodiments, the intracellular signaling domain comprises (i) a CD3ζ polypeptide comprising the amino acid sequence set forth in SEQ ID NO: 107, and (ii) a co-stimulatory signaling region comprising a CD28 polypeptide comprising the amino acid sequence set amino acids 180 to 220 of SEQ ID NO: 103. In certain embodiments, the extracellular antigen-binding domain is an scFv that is designated as “4-B2”. In certain embodiments, the V_{H} and V_{L} are linked via a linker comprising or consisting of the amino acid sequence set forth in SEQ ID NO: 96. In certain embodiments, the V_{H} and V_{L} are positioned from the N- to the C-terminus: V_{H}-V_{L}. In certain embodiments, the CAR is designed as “TDI-Y-007_h28z”. In certain embodiments, the CAR comprises the amino acid sequence set forth in SEQ ID NO: 114, which is provided below.

[SEQ ID NO: 114]
EVHLLESGGGLVQPGGSLRLSCAASGFIFSSNAMSWVRQAPGKGLEWVS
AISGYGGNTYYADSVKGRFTISRDNSKNTLYLQMNSLRAEDTAVYYCAK
WGTYIVGATGDYWGQGTLVTVSSGGGGSGGGGSGGGGSQSALTQPPSAS
GSPGQSVTISCTGTSNDVGGYNYVSWYQQHPGKAPKLLIYEVSKRPSGV
PDRFSGSQSGNTASLTVSGLQAEDEADYYCSSYAGSNNWVFGGGTKLTV
LEQKLISEEDLAAAIEVMYPPPYLDNEKSNGTIIHVKGKHLCPSPLFPG
PSKPFWVLVVVGGVLACYSLLVTVAFIIFWVRSKRSRLLHSDYMNMTPR
RPGPTRKHYQPYAPPRDFAAYRSRVKFSRSADAPAYQQGQNQLYNELNL
GRREEYDVLDKRRGRDPEMGGKPRRKNPQEGLYNELQKDKMAEAYSEIG
MKGERRRGKGHDGLYQGLSTATKDTYDALHMQALPPR

An exemplary nucleotide sequence encoding the amino acid sequence of SEQ ID NO: 114 is set forth in SEQ ID NO: 115, which is provided below.

[SEQ ID NO: 115]
GAGGTGCACCTGTTGGAGTCTGGGGGAGGCTTGGTACAGCCTGGGGGGTCCCTGAGACTCTCCTGTGCAGCCTCTGGATT
CATCTTTAGCAGCAATGCCATGAGCTGGGTCCGCCAGGCTCCAGGGAAGGGACTGGAGTGGGTCTCAGCTATTAGTGGTT
ATGGTGGTAACACATACTACGCAGACTCCGTGAAGGGCCGGTTCACCATCTCCAGAGACAATTCCAAGAACACGCTATAT
CTGCAAATGAACAGCCTGAGAGCCGAGGACACGGCCGTATATTACTGTGCGAAATGGGGGACTTATATAGTGGGAGCTAC
GGGTGACTACTGGGGCCAGGGAACTCTGGTCACCGTCTCCTCAGGTGGAGGTGGATCAGGTGGAGGTGGATCTGGTGGAG
GTGGATCTCAGTCTGCCCTGACTCAGCCTCCCTCCGCGTCCGGGTCTCCTGGACAGTCAGTCACCATCTCCTGCACTGGA
ACCAGCAATGACGTTGGTGGTTATAACTATGTCTCCTGGTACCAACAGCACCCAGGCAAAGCCCCCAAACTCTTGATTTA
TGAGGTCAGTAAGCGGCCCTCAGGGGTCCCTGATCGCTTCTCTGGCTCCCAGTCTGGCAACACGGCCTCCCTGACCGTCT
CTGGGCTCCAGGCTGAGGATGAGGCTGATTATTACTGCAGCTCATATGCAGGCAGCAACAATTGGGTGTTCGGCGGAGGG
ACCAAGCTGACCGTCCTAGAACAGAAACTGATCTCTGAAGAAGACCTGGCGGCCGCAATTGAAGTTATGTATCCTCCTCC
TTACCTAGACAATGAGAAGAGCAATGGAACCATTATCCATGTGAAAGGGAAACACCTTTGTCCAAGTCCCCTATTTCCCG
GACCTTCTAAGCCCTTTTGGGTGCTGGTGGTGGTTGGTGGAGTCCTGGCTTGCTATAGCTTGCTAGTAACAGTGGCCTTT
ATTATTTTCTGGGTGAGGAGTAAGAGGAGCAGGCTCCTGCACAGTGACTACATGAACATGACTCCCCGCCGCCCCGGGCC
CACCCGCAAGCATTACCAGCCCTATGCCCCACCACGCGACTTCGCAGCCTATCGCTCCAGAGTGAAGTTCAGCAGGAGCG
CAGACGCCCCCGCGTACCAGCAGGGCCAGAACCAGCTCTATAACGAGCTCAATCTAGGACGAAGAGAGGAGTACGATGTT
TTGGACAAGAGACGTGGCCGGGACCCTGAGATGGGGGGAAAGCCGAGAAGGAAGAACCCTCAGGAAGGCCTGTACAATGA
ACTGCAGAAAGATAAGATGGCGGAGGCCTACAGTGAGATTGGGATGAAAGGCGAGCGCCGGAGGGGCAAGGGGCACGATG
GCCTTTACCAGGGTCTCAGTACAGCCACCAAGGACACCTACGACGCCCTTCACATGCAGGCCCTGCCCCCTCGCTAG

In certain embodiments, the CAR is a CD33-targeted CAR. In certain embodiments, the CAR comprises (a) an extracellular antigen-binding domain comprising (i) a V_{H} that comprises a CDR1 comprising the amino acid sequence set forth in SEQ ID NO: 22, a CDR2 comprising the amino acid sequence set forth in SEQ ID NO: 23, and a V_{H} CDR3 comprising the amino acid sequence set forth in SEQ ID NO: 24, and (ii) a V_{L} that comprises a CDR1 comprising the amino acid sequence set forth in SEQ ID NO: 25, a CDR2 comprising the amino acid sequence set forth in SEQ ID NO: 26, and a V_{L} CDR3 comprising the amino acid sequence set forth in SEQ ID NO: 27; (b) a transmembrane domain comprising a CD28 polypeptide (e.g., a transmembrane domain of human CD28 or a portion thereof), and (c) an intracellular signaling domain comprising (i) a CD3ζ polypeptide, and (ii) a co-stimulatory signaling region comprising a CD28 polypeptide (e.g., an intracellular domain of human CD28 or a portion thereof). In certain embodiments, the transmembrane domain comprises a CD28 polypeptide that comprises amino acids 153 to 179 of SEQ ID NO: 103. In certain embodiments, the intracellular signaling domain comprises (i) a CD3ζ polypeptide comprising the amino acid sequence set forth in SEQ ID NO: 107, and (ii) a co-stimulatory signaling region comprising a CD28 polypeptide comprising the amino acid sequence set amino acids 180 to 220 of SEQ ID NO: 103. In certain embodiments, the extracellular antigen-binding domain is an scFv that is designated as “1-J19”. In certain embodiments, the V_{H} and V_{L} are linked via a linker comprising or consisting of the amino acid sequence set forth in SEQ ID NO: 96. In certain embodiments, the V_{H} and V_{L} are positioned from the N- to the C-terminus: V_{H}-V_{L}. In certain embodiments, the CAR is designed as “1J19HL_h28z”. In certain embodiments, the CAR comprises the amino acid sequence set forth in SEQ ID NO: 116, which is provided below.

[SEQ ID NO: 116]
QVQLQQSGPGLVKPSQTLSLTCAISGDSVSSNSAAWNWIRQSPSRGLEW
LGRTYFRSKWYNVYAVSVKSRITINPDTSKNQFSLQLNSVTPEDTAVYY
CASEGGSYYDHWGQGTLVTVSSGGGGSGGGGSGGGGSDIQMTQSPSSVS
ASVGDRVTITCRASQGISNWLTWYQQKPGKAPKLLIYAASSLQSGVPSR
FSGSGSGTDFTLTISSLQPEDFATYYCQQADSFPFTFGPGTKVDIKEQK
LISEEDLAAAIEVMYPPPYLDNEKSNGTIIHVKGKHLCPSPLFPGPSKP
FWVLVVVGGVLACYSLLVTVAFIIFWVRSKRSRLLHSDYMNMTPRRPGP
TRKHYQPYAPPRDFAAYRSRVKFSRSADAPAYQQGQNQLYNELNLGRRE
EYDVLDKRRGRDPEMGGKPRRKNPQEGLYNELQKDKMAEAYSEIGMKGE
RRRGKGHDGLYQGLSTATKDTYDALHMQALPPR

An exemplary nucleotide sequence encoding the amino acid sequence of SEQ ID NO: 116 is set forth in SEQ ID NO: 117, which is provided below.

[SEQ ID NO: 117]
CAGGTACAGCTGCAGCAGTCAGGTCCAGGACTGGTGAAGCCCTCGCAGACCCTCTCACTCACCTGTGCCATCTCCGGGGA
CAGTGTCTCTAGCAACAGTGCTGCTTGGAACTGGATCAGGCAGTCCCCATCGAGAGGCCTTGAGTGGCTGGGAAGGACAT
ACTTCAGGTCCAAGTGGTATAATGTTTATGCAGTGTCTGTGAAGAGTCGAATAACCATCAACCCAGACACATCCAAGAAC
CAGTTCTCCCTGCAGCTGAACTCTGTGACTCCCGAGGACACGGCTGTGTATTATTGTGCAAGCGAGGGTGGGAGCTATTA
TGACCACTGGGGCCAGGGAACCCTGGTCACCGTCTCCTCAGGTGGAGGTGGATCAGGTGGAGGTGGATCTGGTGGAGGTG
GATCTGACATCCAGATGACCCAGTCTCCATCTTCCGTGTCTGCATCTGTAGGAGACAGAGTCACCATCACTTGTCGGGCG
AGTCAGGGTATTAGTAATTGGTTAACCTGGTATCAGCAGAAACCAGGGAAAGCCCCTAAGCTCCTGATCTATGCTGCATC
CAGTTTGCAAAGTGGGGTCCCATCAAGGTTCAGCGGCAGTGGATCTGGGACAGATTTCACTCTCACCATCAGCAGCCTGC
AGCCTGAAGATTTTGCAACTTACTATTGTCAACAGGCTGACAGTTTCCCATTCACTTTCGGCCCTGGGACCAAAGTGGAT
ATCAAAGAACAGAAACTGATCTCTGAAGAAGACCTGGCGGCCGCAATTGAAGTTATGTATCCTCCTCCTTACCTAGACAA
TGAGAAGAGCAATGGAACCATTATCCATGTGAAAGGGAAACACCTTTGTCCAAGTCCCCTATTTCCCGGACCTTCTAAGC
CCTTTTGGGTGCTGGTGGTGGTTGGTGGAGTCCTGGCTTGCTATAGCTTGCTAGTAACAGTGGCCTTTATTATTTTCTGG
GTGAGGAGTAAGAGGAGCAGGCTCCTGCACAGTGACTACATGAACATGACTCCCCGCCGCCCCGGGCCCACCCGCAAGCA
TTACCAGCCCTATGCCCCACCACGCGACTTCGCAGCCTATCGCTCCAGAGTGAAGTTCAGCAGGAGCGCAGACGCCCCCG
CGTACCAGCAGGGCCAGAACCAGCTCTATAACGAGCTCAATCTAGGACGAAGAGAGGAGTACGATGTTTTGGACAAGAGA
CGTGGCCGGGACCCTGAGATGGGGGGAAAGCCGAGAAGGAAGAACCCTCAGGAAGGCCTGTACAATGAACTGCAGAAAGA
TAAGATGGCGGAGGCCTACAGTGAGATTGGGATGAAAGGCGAGCGCCGGAGGGGCAAGGGGCACGATGGCCTTTACCAGG
GTCTCAGTACAGCCACCAAGGACACCTACGACGCCCTTCACATGCAGGCCCTGCCCCCTCGCTAG

In certain embodiments, a presently disclosed CAR further comprises an inducible promoter, for expressing nucleotide sequences in human cells. Promoters for use in expressing CAR genes can be a constitutive promoter, such as ubiquitin C (UbiC) promoter.

5.3.3. TCR like Fusion Molecules

In certain embodiments, the antigen-recognizing receptor is a TCR like fusion molecule. Non-limiting examples of TCR fusion molecules include HLA-Independent TCR-based Chimeric Antigen Receptor (also known as “HIT-CAR”, e.g., those disclosed in International Patent Application No. PCT/US19/017525, which is incorporated by reference in its entirety), T cell receptor fusion constructs (TRuCs) (e.g., those disclosed in Baeuerle et al., “Synthetic TRuC receptors engaging the complete T cell receptor for potent anti-tumor response,” Nature Communications volume 10, Article number: 2087 (2019), which is incorporated by reference in its entirety), and T cell antigen coupler (TAC)s (which are chimeric receptors that co-opt the endogenous TCR) (e.g., those disclosed in Helsen et al., “The chimeric TAC receptor co-opts the T cell receptor yielding robust anti-tumor activity without toxicity,” Nature Communications (2018); 9:3049 (2018), which is incorporated by reference in its entirety).

In certain embodiments, the TCR like fusion molecule comprises an antigen binding chain that comprises an extracellular antigen-binding domain and a constant domain, wherein the TCR like fusion molecule binds to an antigen in an HLA-independent manner. In certain embodiments, the constant domain comprises a T cell receptor constant region selected from the group consisting of a native or modified TRAC peptide, a native or modified TRBC peptide, a native or modified TRDC peptide, a native or modified TRGC peptide and any variants or functional fragments thereof. In certain embodiments, the constant domain comprises a native or modified TRAC peptide. In certain embodiments, the constant domain comprises a native or modified TRBC peptide. In certain embodiments, the constant domain is capable of forming a homodimer or a heterodimer with another constant domain. In certain embodiments, the antigen binding chain is capable of associating with a CD3ζ polypeptide. In certain embodiments, the antigen binding chain, upon binding to an antigen, is capable of activating the CD3ζ polypeptide associated to the antigen binding chain. In certain embodiments, the activation of the CD3ζ polypeptide is capable of activating an immunoresponsive cell. In certain embodiments, the TCR like fusion molecule is capable of integrating with a CD3 complex and providing HLA-independent antigen recognition. In certain embodiments, the TCR like fusion molecule replaces an endogenous TCR in a CD3/TCR complex. In certain embodiments, the extracellular antigen-binding domain of the TCR like fusion molecule is capable of dimerizing with another extracellular antigen-binding domain. In certain embodiments, the extracellular antigen-binding domain of the TCR like fusion molecule comprises a ligand for a cell-surface receptor, a receptor for a cell surface ligand, an antigen binding portion of an antibody or a fragment thereof or an antigen binding portion of a TCR. In certain embodiments, the extracellular antigen-binding domain of the TCR like fusion molecule comprises one or two immunoglobulin variable region(s). In certain embodiments, the extracellular antigen-binding domain of the TCR like fusion molecule comprises a heavy chain variable region (V_{H}) of an antibody. In certain embodiments, the extracellular antigen-binding domain of the TCR like fusion molecule comprises a light chain variable region (V_{L}) of an antibody. In certain embodiments, the extracellular antigen-binding domain of the TCR like fusion molecule is capable of dimerizing with another extracellular antigen-binding domain. In certain embodiments, the extracellular antigen-binding domain of the TCR like fusion molecule comprises a V_{H} of an antibody, wherein the V_{H} is capable of dimerizing with another extracellular antigen-binding domain comprising a V_{L} of the antibody and form a fragment variable (Fv). In certain embodiments, the extracellular antigen-binding domain of the TCR like fusion molecule comprises a V_{L} of an antibody, wherein the V_{L} is capable of dimerizing with another extracellular antigen-binding domain comprising a V_{H} of the antibody and form a fragment variable (Fv).

### 5.4. Cells

The presently disclosed subject matter provides cells comprising a presently disclosed CD33-targeted antigen-recognizing receptor (e.g., one disclosed in Section 5.3). In certain embodiments, the cell is selected from the group consisting of cells of lymphoid lineage, cells of myeloid lineage, stem cells from which cells of lymphoid lineage can be derived, and stem cells from which cells of myeloid lineage can be derived. In certain embodiments, the cell is an immunoresponsive cell. In certain embodiments, the immunoresponsive cell is a cell of lymphoid lineage.

In certain embodiments, the cell is a cell of the lymphoid lineage. Cells of the lymphoid lineage can provide production of antibodies, regulation of cellular immune system, detection of foreign agents in the blood, detection of cells foreign to the host, and the like. Non-limiting examples of cells of the lymphoid lineage include T cells, Natural Killer (NK) cells, B cells, dendritic cells, stem cells from which lymphoid cells may be differentiated. In certain embodiments, the stem cell is a pluripotent stem cell (e.g., embryonic stem cell).

In certain embodiments, the cell is a T cell. T cells can be lymphocytes that mature in the thymus and are chiefly responsible for cell-mediated immunity. T cells are involved in the adaptive immune system. The T cells of the presently disclosed subject matter can be any type of T cells, including, but not limited to, helper T cells, cytotoxic T cells, memory T cells (including central memory T cells, stem-cell-like memory T cells (or stem-like memory T cells), and two types of effector memory T cells: e.g., TEM cells and TEMRA cells, Regulatory T cells (also known as suppressor T cells), tumor-infiltrating lymphocyte (TIL), Natural killer T cells, Mucosal associated invariant T cells, and T6 T cells. Cytotoxic T cells (CTL or killer T cells) are a subset of T lymphocytes capable of inducing the death of infected somatic or tumor cells. A patient's own T cells may be genetically modified to target specific antigens through the introduction of an antigen-recognizing receptor, e.g., a CAR. In certain embodiments, the immunoresponsive cell is a T cell. The T cell can be a CD4⁺ T cell or a CD8⁺ T cell. In certain embodiments, the T cell is a CD4⁺ T cell. In certain embodiments, the T cell is a CD8⁺ T cell.

In certain embodiments, the cell is a NK cell. Natural killer (NK) cells can be lymphocytes that are part of cell-mediated immunity and act during the innate immune response. NK cells do not require prior activation in order to perform their cytotoxic effect on target cells.

Types of human lymphocytes of the presently disclosed subject matter include, without limitation, peripheral donor lymphocytes. e.g., those disclosed in Sadelain et al., Nat Rev Cancer (2003); 3:35-45 (disclosing peripheral donor lymphocytes genetically modified to express CARs), in Morgan, R. A., et al. 2006 Science 314:126-129 (disclosing peripheral donor lymphocytes genetically modified to express a full-length tumor antigen-recognizing T cell receptor complex comprising the α and β heterodimer), in Panelli et al., J Immunol (2000); 164:495-504; Panelli et al., J Immunol (2000); 164:4382-4392 (disclosing lymphocyte cultures derived from tumor infiltrating lymphocytes (TILs) in tumor biopsies), and in Dupont et al., Cancer Res (2005); 65:5417-5427; Papanicolaou et al., Blood (2003); 102:2498-2505 (disclosing selectively in vitro-expanded antigen-specific peripheral blood leukocytes employing artificial antigen-presenting cells (AAPCs) or pulsed dendritic cells).

The cells (e.g., T cells) can be autologous, non-autologous (e.g., allogeneic), or derived in vitro from engineered progenitor or stem cells.

The cells of the presently disclosed subject matter can be cells of the myeloid lineage. Non-limiting examples of cells of the myeloid lineage include monocytes, macrophages, neutrophils, dendritic cells, basophils, neutrophils, eosinophils, megakaryocytes, mast cell, erythrocyte, thrombocytes, and stem cells from which myeloid cells may be differentiated. In certain embodiments, the stem cell is a pluripotent stem cell (e.g., an embryonic stem cell or an induced pluripotent stem cell).

In certain embodiments, the presently disclosed cells are capable of modulating the tumor microenvironment. Tumors have a microenvironment that is hostile to the host immune response involving a series of mechanisms by malignant cells to protect themselves from immune recognition and elimination. This “hostile tumor microenvironment” comprises a variety of immune suppressive factors including infiltrating regulatory CD4⁺ T cells (Tregs), myeloid derived suppressor cells (MDSCs), tumor associated macrophages (TAMs), immune suppressive cytokines including TGF-β, and expression of ligands targeted to immune suppressive receptors expressed by activated T cells (CTLA-4 and PD-1). These mechanisms of immune suppression play a role in the maintenance of tolerance and suppressing inappropriate immune responses, however within the tumor microenvironment these mechanisms prevent an effective anti-tumor immune response. Collectively these immune suppressive factors can induce either marked anergy or apoptosis of adoptively transferred CAR modified T cells upon encounter with targeted tumor cells.

In certain embodiments, the cells can be transduced with the presently disclosed CD33-targeted antigen-recognizing receptor such that the cells express the antigen-recognizing receptor.

In certain embodiments, the cell further comprises a soluble single-chain variable fragment (scFv) that binds a polypeptide that has immunosuppressive activity or immunostimulatory activity. In certain embodiments, immunosuppressive activity refers to induction of signal transduction or changes in protein expression in a cell (e.g., an activated immunoresponsive cell) resulting in a decrease in an immune response. Polypeptides known to suppress or decrease an immune response via their binding include CD47, PD-1, CTLA-4, and their corresponding ligands, including SIRPa, PD-L1, PD-L2, B7-1, and B7-2. Such polypeptides are present in the tumor microenvironment and inhibit immune responses to neoplastic cells. In various embodiments, inhibiting, blocking, or antagonizing the interaction of immunosuppressive polypeptides and/or their ligands enhances the immune response of the immunoresponsive cell.

In certain embodiments, immunostimulatory activity refers to induction of signal transduction or changes in protein expression in a cell (e.g., an activated immunoresponsive cell) resulting in an increase in an immune response. Immunostimulatory activity may include pro-inflammatory activity. Polypeptides known to stimulate or increase an immune response via their binding include CD28, OX-40, 4-IBB, and their corresponding ligands, including B7-1, B7-2, OX-40L, and 4-1BBL. Such polypeptides are present in the tumor microenvironment and activate immune responses to neoplastic cells. In various embodiments, promoting, stimulating, or agonizing pro-inflammatory polypeptides and/or their ligands enhances the immune response of the immunoresponsive cell.

Cells comprising an antigen-recognizing receptor (e.g., a CAR) and a soluble scFv that binds a polypeptide that has immunosuppressive activity or immunostimulatory activity are disclosed in International Patent Publication No. WO 2014/134165, which is incorporated by reference in its entirety.

In certain embodiments, the cell further comprises an exogenous CD40L. Cells comprising an antigen-recognizing receptor (e.g., a CAR) and an exogenous CD40L are disclosed in International Patent Publication No. WO 2014/134165.

Furthermore, in certain embodiments, the cell is engineered to express IL-18. In certain embodiments, the cell further comprises an exogenous IL-18 polypeptide. In certain embodiments, the exogenous IL-18 polypeptide comprises or consists of the amino acid sequence set forth in SEQ ID NO: 118, which is provided below.

[SEQ ID NO: 118]
MGYRMQLLSCIALSLALVINSGYFGKLESKLSVIRNLNDQVLFIDQGNR
PLFEDMTDSDCRDNAPRTIFIISMYKDSQPRGMAVTISVKCEKISTLSC
ENKIISFKEMNPPDNIKDTKSDIIFFQRSVPGHDNKMQFESSSYEGYFL
ACEKERDLFKLILKKEDELGDRSIMFTVQNED

In certain embodiments, the cells further comprise a nucleic acid molecule encoding an IL-18 polypeptide. In certain embodiments, the nucleic acid molecule comprises the nucleotide sequence set forth in SEQ ID NO: 119, which is provided below.

[SEQ ID NO: 119]
ATGGGTTACAGGATGCAACTCCTGTCTTGCATTGCACTAAGTCTTGCACTTGTCACAAACAGTGGCTACTTTG
GCAAGCTTGAATCTAAATTATCAGTCATAAGAAATTTGAATGACCAAGTTCTCTTCATTGACCAAGGAAATCG
GCCTCTATTTGAAGATATGACTGATTCTGACTGTAGAGATAATGCACCCCGGACCATATTTATTATAAGTATG
TATAAAGATAGCCAGCCTAGAGGTATGGCTGTAACTATCTCTGTGAAGTGTGAGAAAATTTCAACTCTCTCCT
GTGAGAACAAAATTATTTCCTTTAAGGAAATGAATCCTCCTGATAACATCAAGGATACAAAAAGTGACATCAT
ATTCTTTCAGAGAAGTGTCCCAGGACATGATAATAAGATGCAATTTGAATCTTCATCATACGAAGGATACTTT
CTAGCTTGTGAAAAAGAGAGAGACCTTTTTAAACTCATTTTGAAAAAAGAGGATGAATTGGGGGATAGATCTA
TAATGTTCACTGTTCAAAACGAAGACTAG

Alternatively, in certain embodiments, the cell further comprises a modified promoter/enhancer at an IL-18 gene locus, which can increase IL-18 gene expression, e.g., a constitutive or inducible promoter is placed to drive IL-18 gene expression.

Cells comprising an antigen-recognizing receptor (e.g., a CAR) and engineered to express IL-18, e.g., comprising an exogenous IL-18 polypeptide or a modified promoter/enhancer at an IL-18 gene locus are disclosed in International Patent Publication No. WO2018/027155, which is incorporated by reference in its entirety.

Additionally or alternatively, the cell is engineered to express IL-33. In certain embodiments, the cell further comprises an exogenous IL-33 polypeptide. In certain embodiments, the exogenous IL-33 polypeptide comprises or consists of the amino acid sequence set forth in SEQ ID NO: 120, which is provided below.

[SEQ ID NO: 120]
MYRMQLLSCIALSLALVINSSITGISPITEYLASLSTYNDQSITFALED
ESYEIYVEDLKKDEKKDKVLLSYYESQHPSNESGDGVDGKMLMVTLSPT
KDEWLHANNKEHSVELHKCEKPLPDQAFFVLHNMHSNCVSFECKTDPGV
FIGVKDNHLALIKVDSSENLCTENILEKLSET

In certain embodiments, the cells further comprise a nucleic acid molecule encoding an IL-33 polypeptide. In certain embodiments, the nucleic acid molecule comprises the nucleotide sequence set forth in SEQ ID NO: 121, which is provided below.

[SEQ ID NO: 121]
ATGTACAGGATGCAACTCCTGTCTTGCATTGCACTAAGTCTTGCACTTGTCACAAACAGTAGTATCACAGGAA
TTTCACCTATTACAGAGTATCTTGCTTCTCTAAGCACATACAATGATCAATCCATTACTTTTGCTTTGGAGGA
TGAAAGTTATGAGATATATGTTGAAGACTTGAAAAAAGATGAAAAGAAAGATAAGGTGTTACTGAGTTACTAT
GAGTCTCAACACCCCTCAAATGAATCAGGTGACGGTGTTGATGGTAAGATGTTAATGGTAACCCTGAGTCCTA
CAAAAGACTTCTGGTTGCATGCCAACAACAAGGAACACTCTGTGGAGCTCCATAAGTGTGAAAAACCACTGCC
AGACCAGGCCTTCTTTGTCCTTCATAATATGCACTCCAACTGTGTTTCATTTGAATGCAAGACTGATCCTGGA
GTGTTTATAGGTGTAAAGGATAATCATCTTGCTCTGATTAAAGTAGACTCTTCTGAGAATTTGTGTACTGAAA
ATATCTTGTTTAAGCTCTCTGAAACTTA

Alternatively, in certain embodiments, the cell further comprises a modified promoter/enhancer at an IL-33 gene locus, which can increase IL-33 gene expression, e.g., a constitutive or inducible promoter placed to drive IL-33 gene expression. Cells comprising an antigen-recognizing receptor (e.g., a CAR) and engineered to express IL-33, e.g., comprising an exogenous IL-33 polypeptide or a modified promoter/enhancer at an IL-33 gene locus are disclosed in International Patent Publication No. WO2019/099479, which is incorporated by reference in its entirety.

Additionally or alternatively, the cell is engineered to express IL-36. In certain embodiments, the cell further comprises an exogenous IL-36 polypeptide. In certain embodiments, the cell further comprises a modified promoter/enhancer at an IL-36 gene locus, which can increase IL-36 gene expression, e.g., a constitutive or inducible promoter placed to drive IL-36 gene expression. Cells comprising an antigen-recognizing receptor (e.g., a CAR) and engineered to express IL-36, e.g., comprising an exogenous IL-36 polypeptide or a modified promoter/enhancer at an IL-36 gene locus are disclosed in International Patent Publication No. WO2019/099483, which is incorporated by reference in its entirety.

### 5.5. Nucleic Acid Compositions and Vectors

The presently disclosed subject matter provides nucleic acids encoding the presently disclosed CD33-targeted antigen-recognizing receptors (e.g., those disclosed in Section 5.3). Further provided are nucleic acid compositions comprising the nucleic acids disclosed herein. Also provided are cells comprising such nucleic acid compositions.

In certain embodiments, the nucleic acid composition further comprises a promoter that is operably linked to the presently disclosed CD33-targeted antigen-recognizing receptor.

In certain embodiments, the promoter is endogenous or exogenous. In certain embodiments, the exogenous promoter is selected from an elongation factor (EF)-1 promoter, a cytomegalovirus immediate-early promoter (CMV) promoter, a simian virus 40 early promoter (SV40) promoter, a phosphoglycerate kinase (PGK) promoter, and a metallothionein promoter. In certain embodiments, the promoter is an inducible promoter. In certain embodiment, the inducible promoter is selected from a NFAT transcriptional response element (TRE) promoter, a CD69 promoter, a CD25 promoter, and an IL-2 promoter. The compositions and nucleic acid compositions can be administered to subjects or and/delivered into cells by art-known methods or as described herein. Genetic modification of a cell (e.g., a T cell or a NK cell) can be accomplished by transducing a substantially homogeneous cell composition with a recombinant DNA construct. In certain embodiments, a retroviral vector (e.g., gamma-retroviral vector or lentiviral vector) is employed for the introduction of the DNA construct into the cell. For example, a polynucleotide encoding an antigen-recognizing receptor can be cloned into a retroviral vector and expression can be driven from its endogenous promoter, from the retroviral long terminal repeat, or from a promoter specific for a target cell type of interest. Non-viral vectors may be used as well.

For initial genetic modification of a cell to include a presently disclosed CD33-targeted antigen-recognizing receptor (e.g., a CAR), a retroviral vector can be employed for transduction, however any other suitable viral vector or non-viral delivery system can be used. The antigen-recognizing receptor can be constructed in a single, multicistronic expression cassette, in multiple expression cassettes of a single vector, or in multiple vectors. Examples of elements that create polycistronic expression cassette include, but is not limited to, various viral and non-viral Internal Ribosome Entry Sites (IRES, e.g., FGF-1 IRES, FGF-2 IRES, VEGF IRES, IGF-II IRES, NF-κB IRES, RUNX1 IRES, p53 IRES, hepatitis A IRES, hepatitis C IRES, pestivirus IRES, aphthovirus IRES, picornavirus IRES, poliovirus IRES and encephalomyocarditis virus IRES) and cleavable linkers (e.g., 2A peptides, e.g., P2A, T2A, E2A and F2A peptides). Combinations of retroviral vector and an appropriate packaging line are also suitable, where the capsid proteins will be functional for infecting human cells. Various amphotropic virus-producing cell lines are known, including, but not limited to, PA12 (Miller et al., (1985) Mol Cell Biol (1985); 5:431-437); PA317 (Miller., et al., Mol Cell Biol (1986); 6:2895-2902); and CRIP (Danos et al., Proc Natl Acad Sci USA (1988); 85:6460-6464). Non-amphotropic particles are suitable too, e.g., particles pseudotyped with VSVG, RD114 or GALV envelope and any other known in the art.

Possible methods of transduction also include direct co-culture of the cells with producer cells (Bregni et al., Blood (1992); 80:1418-1422), or culturing with viral supernatant alone or concentrated vector stocks with or without appropriate growth factors and polycations (Xu et al., Exp Hemat (1994); 22:223-230; and Hughes et al. J Clin Invest (1992); 89:1817).

Other transducing viral vectors can be used to modify a cell. In certain embodiments, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). Other viral vectors that can be used include, for example, adenoviral, lentiviral, and adena-associated viral vectors, vaccinia virus, a bovine papilloma virus, or a herpes virus, such as Epstein-Barr Virus (also see, for example, the vectors of Miller, Human Gene Thera (1990); 15-14; Friedman, Science 244:1275-1281, 1989; Eglitis et al., BioTechniques (1988); 6:608-614; Tolstoshev et al., Cur Opin Biotechnol (1990); 1:55-61; Sharp, The Lancet (1991); 337:1277-78; Cornetta et al., Nucleic Acid Research and Molecular Biology 36:311-22, 1987; Anderson, Science (1984); 226:401-409; Moen, Blood Cells 17:407-16, 1991; Miller et al., Biotechnol (1989); 7:980-90; LeGal La Salle et al., Science (1993); 259:988-90; and Johnson, Chest (1995)107:77S-83S). Retroviral vectors are particularly well developed and have been used in clinical settings (Rosenberg et al., N Engl J Med (1990); 323:370, 1990; Anderson et al., U.S. Pat. No. 5,399,346).

Non-viral approaches can also be employed for genetic modification of a cell. For example, a nucleic acid molecule can be introduced into a cell by administering the nucleic acid in the presence of lipofection (Feigner et al., Proc Natl Acad Sci U.S.A. (1987); 84:7413; Ono et al., Neurosci Lett (1990); 17:259; Brigham et al., Am J Med Sci (1989); 298:278; Staubinger et al., Methods in Enzymol (1983); 101:512, Wu et al., J Biol Chem (1988); 263:14621; Wu et al., J Biol Chem (1989); 264:16985), or by micro-injection under surgical conditions (Wolff et al., Science (1990); 247:1465). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Recombinant receptors can also be derived or obtained using transposases or targeted nucleases (e.g. Zinc finger nucleases, meganucleases, or TALE nucleases, CRISPR). Transient expression may be obtained by RNA electroporation.

Any targeted genome editing methods can also be used to deliver a presently disclosed antigen-recognizing receptor to a cell or a subject. In certain embodiments, a CRISPR system is used to deliver a presently disclosed antigen-recognizing receptor disclosed herein. In certain embodiments, zinc-finger nucleases are used to deliver the antigen-recognizing receptor. In certain embodiments, a TALEN system is used to deliver a presently disclosed antigen-recognizing receptor.

Clustered regularly-interspaced short palindromic repeats (CRISPR) system is a genome editing tool discovered in prokaryotic cells. When utilized for genome editing, the system includes Cas9 (a protein able to modify DNA utilizing crRNA as its guide), CRISPR RNA (crRNA, contains the RNA used by Cas9 to guide it to the correct section of host DNA along with a region that binds to tracrRNA (generally in a hairpin loop form) forming an active complex with Cas9), trans-activating crRNA (tracrRNA, binds to crRNA and forms an active complex with Cas9), and an optional section of DNA repair template (DNA that guides the cellular repair process allowing insertion of a specific DNA sequence). CRISPR/Cas9 often employs a plasmid to transfect the target cells. The crRNA needs to be designed for each application as this is the sequence that Cas9 uses to identify and directly bind to the target DNA in a cell. The repair template carrying CAR expression cassette need also be designed for each application, as it must overlap with the sequences on either side of the cut and code for the insertion sequence. Multiple crRNA's and the tracrRNA can be packaged together to form a single-guide RNA (sgRNA). This sgRNA can be joined together with the Cas9 gene and made into a plasmid in order to be transfected into cells.

A zinc-finger nuclease (ZFN) is an artificial restriction enzyme, which is generated by combining a zinc finger DNA-binding domain with a DNA-cleavage domain. A zinc finger domain can be engineered to target specific DNA sequences which allows a zinc-finger nuclease to target desired sequences within genomes. The DNA-binding domains of individual ZFNs typically contain a plurality of individual zinc finger repeats and can each recognize a plurality of basepairs. The most common method to generate new zinc-finger domain is to combine smaller zinc-finger “modules” of known specificity. The most common cleavage domain in ZFNs is the non-specific cleavage domain from the type IIs restriction endonuclease FokI. Using the endogenous homologous recombination (HR) machinery and a homologous DNA template carrying CAR expression cassette, ZFNs can be used to insert the CAR expression cassette into genome. When the targeted sequence is cleaved by ZFNs, the HR machinery searches for homology between the damaged chromosome and the homologous DNA template, and then copies the sequence of the template between the two broken ends of the chromosome, whereby the homologous DNA template is integrated into the genome.

Transcription activator-like effector nucleases (TALEN) are restriction enzymes that can be engineered to cut specific sequences of DNA. TALEN system operates on almost the same principle as ZFNs. They are generated by combining a transcription activator-like effectors DNA-binding domain with a DNA cleavage domain. Transcription activator-like effectors (TALEs) are composed of 33-34 amino acid repeating motifs with two variable positions that have a strong recognition for specific nucleotides. By assembling arrays of these TALEs, the TALE DNA-binding domain can be engineered to bind desired DNA sequence, and thereby guide the nuclease to cut at specific locations in genome. cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element or intron (e.g. the elongation factor 1a enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

5.5.1 Methods of delivering Methods for delivering the genome editing agents/systems can vary depending on the need. In certain embodiments, the components of a selected genome editing method are delivered as DNA constructs in one or more plasmids. In certain embodiments, the components are delivered via viral vectors. Common delivery methods include but is not limited to, electroporation, microinjection, gene gun, impalefection, hydrostatic pressure, continuous infusion, sonication, magnetofection, adeno-associated viruses, envelope protein pseudotyping of viral vectors, replication-competent vectors cis and trans-acting elements, herpes simplex virus, and chemical vehicles (e.g., oligonucleotides, lipoplexes, polymersomes, polyplexes, dendrimers, inorganic Nanoparticles, and cell-penetrating peptides).

In certain embodiments, the delivery methods include use of colloids. As used herein, the term “colloid” refers to systems in which there are two or more phases, with one phase (e.g., the dispersed phase) distributed in the other phase (e.g., the continuous phase). Moreover, at least one of the phases has small dimensions (in the range of about 10-9 to about 10-6 m). Non-limiting examples of colloids encompassed by the presently disclosed subject matter include macromolecule complexes, nanocapsules, microspheres, beads, and lipid-based systems (e.g., micelles, liposomes, and lipid nanoparticles).

In certain embodiments, the delivery methods include use of liposomes. The term “liposome,” as used herein, refers to single- or multi-layered spherical lipid bilayer structures produced from lipids dissolved in organic solvents and then dispersed in aqueous media. Experimentally and therapeutically used for delivering an active pharmaceutical ingredient (e.g., nucleic acid compositions disclosed herein) to cells, liposomes fuse with cell membranes so the contents are transferred into the cytoplasm.

In certain embodiments, the delivery methods include use of lipid nanoparticles. As used herein, the term “lipid nanoparticle” refers to a particle having at least one dimension in the order of nanometers (e.g., from about 1 nm to about 1,000 nm) and including at least one lipid. In certain embodiments, the lipid nanoparticles can include an active pharmaceutical ingredient (e.g., nucleic acid compositions disclosed herein) for delivering to cells. The morphology of the lipid nanoparticles can be different from liposomes. While liposomes are characterized by a lipid bilayer surrounding a hydrophilic core, lipid nanoparticles have an electron-dense core where cationic lipids and/or ionizable lipids are organized into inverted micelles around an active pharmaceutical ingredient (e.g., nucleic acid compositions disclosed herein). Additional information on the morphology and properties of lipid nanoparticles and liposomes can be found in Wilczewska, et al., Pharmacological reports 64, no. 5 (2012): 1020-1037; Eygeris et al., Accounts of Chemical Research 55, no. 1 (2021): 2-12; Zhang et al., Chemical Reviews 121, no. 20 (2021): 12181-12277; and Fan et al., Journal of pharmaceutical and biomedical analysis 192 (2021): 113642.

In certain embodiments, the lipid nanoparticles have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm.

In certain embodiments, the lipid nanoparticles can include a cationic lipid or an ionizable lipid. The term “cationic lipid” refers to lipids including a head group with permanent positive charges. Non-limiting examples of cationic lipids encompassed by the presently disclosed subject matter include 1,2-di-O-octadecenyl-3-trimethylammonium-propane (DOTMA), 1,2-dioleoyl-3-trimethylammonium-propane (DOTAP), 2,3-dioleyloxy-N-[2-(sperminecarboxamido)ethyl]-N,N-dimethyl-1-propanaminium trifluoroacetate (DOSPA), and ethylphosphatidylcholine (ePC).

As used herein, the term “ionizable lipid” refers to lipids that are protonated at low pH and are neutral at physiological pH. The pH-sensitivity of ionizable lipids is particularly beneficial for delivery in vivo (e.g., delivery of nucleic acid compositions disclosed herein), because neutral lipids have less interactions with the anionic membranes of blood cells and, thus, improve the biocompatibility of the lipid nanoparticles. Once trapped in endosomes, ionizable lipids are protonated and promote membrane destabilization to allow the endosomal escape of the nanoparticles. Non-limiting example of ionizable lipids encompassed by the presently disclosed subject matter include tetrakis(8-methylnonyl) 3,3′,3″,3′″-(((methylazanediyl) bis(propane-3,1 diyl))bis (azanetriyl))tetrapropionate; decyl (2-(dioctylammonio)ethyl) phosphate; ((4-hydroxybutyl)azanediyl)bis(hexane-6,1-diyl)bis(2-hexyldecanoate); bis(2-(dodecyldisulfanyl)ethyl) 3,3′-((3-methyl-9-oxo-10-oxa-13,14-dithia-3,6-diazahexacosyl)azanediyl)dipropionate; 1,1′-((2-(4-(2-((2-(bis(2-hydroxydodecyl)amino)ethyl) (2-hydroxydodecyl)amino)ethyl) piperazin-1-yl)ethyl)azanediyl) bis(dodecan-2-ol); cKK-E12, 3,6-bis(4-(bis(2-hydroxydodecyl)amino)butyl)piperazine-2,5-dione; (6Z,9Z,28Z,31Z)-heptatriaconta-6,9,28,31-tetraen-19-yl 4-(dimethylamino) butanoate; hexa(octan-3-yl) 9,9′,9″,9′″,9″″,9′″″-((((benzene-1,3,5-tricarbonyl)yris(azanediyl)) tris (propane-3,1-diyl)) tris(azanetriyl))hexanonanoate; heptadecan-9-yl 8-((2-hydroxyethyl)(6-oxo-6-(undecyloxy)hexyl)amino) octanoate; and (((3,6-dioxopiperazine-2,5-diyl)bis(butane-4, 1-diyl))bis(azanetriyl))tetrakis(ethane-2,1-diyl) (9Z,9′Z,9″Z,9′″Z,12Z,12′Z,12″Z,12′″Z)-tetrakis (octadeca-9,12-dienoate).

Additionally, in certain embodiments, the lipid nanoparticles can include other lipids. For example, but without any limitation, the lipid nanoparticles of the presently disclosed subject matter can include phospholipids, cholesterol, polyethylene glycol (PEG)-functionalized lipids (PEG-lipids). These lipids can improve certain properties of the lipid nanoparticles (e.g., stability, biodistribution, etc.). For example, cholesterol enhances the stability of the lipid nanoparticles by modulating the integrity and rigidity. Non-limiting examples of other lipids present in lipid nanoparticles include cholesterol, DC-cholesterol, β-sitosterol, BHEM-cholesterol, ALC-0159, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoylphosphatidylethanolamine (DSPE), 16-O-monomethyl PE, 16-O-dimethyl PE, 18-1-trans PE, 1-stearioyl-2-oleoyl-phosphatidyethanol amine (SOPE), and 1,2-dielaidoyl-sn-glycero-3-phophoethanolamine (transDOPE).

In certain embodiments, the lipid nanoparticles can include a targeting moiety that binds to a ligand. The use of the targeting moieties allows selective delivery of an active pharmaceutical ingredient (e.g., nucleic acid compositions disclosed herein) to target cells expressing the ligand (e.g., T cells). In certain embodiments, the targeting moiety can be an antibody or antigen-binding fragment thereof that binds to a cell surface receptor. For example, but without any limitation, the targeting domain is an antibody or antigen-binding fragment thereof that binds to a receptor expressed on the surface of a T cell (e.g., CD3, CD4, CD8, CD16, CD40L, CD95, FasL, CTLA-4, OX40, GITR, LAG3, ICOS, and PD-1).

In certain embodiments, the delivery methods are in vivo delivery methods. In certain embodiments, the delivery methods are ex vivo delivery methods.

### 5.6. Polypeptides

The presently disclosed subject matter provides methods for optimizing an amino acid sequence or a nucleotide sequence by producing an alteration in the sequence. Such alterations may include certain mutations, deletions, insertions, or post-translational modifications. The presently disclosed subject matter further includes analogs of any naturally-occurring polypeptides disclosed herein (including, but not limited to, CD33, CD8, CD28, 4-1BB, and CD3ζ,). Analogs can differ from a naturally-occurring polypeptide disclosed herein by amino acid sequence differences, by post-translational modifications, or by both. Analogs can exhibit at least about 85%, about 90%, about 91%, about 92%, about 93%, about 94%, about 95%, about 96%, about 97%, about 98%, about 99% or more homologous or identical to all or part of a naturally-occurring amino, acid sequence of the presently disclosed subject matter. The length of sequence comparison is at least 5, 10, 15 or 20 amino acid residues, e.g., at least 25, 50, or 75 amino acid residues, or more than 100 amino acid residues. Again, in an exemplary approach to determining the degree of identity, a BLAST program may be used, with a probability score between e⁻³ and e⁻¹⁰⁰ indicating a closely related sequence. Modifications include in vivo and in vitro chemical derivatization of polypeptides, e.g., acetylation, carboxylation, phosphorylation, or glycosylation; such modifications may occur during polypeptide synthesis or processing or following treatment with isolated modifying enzymes. Analogs can also differ from the naturally-occurring polypeptides by alterations in primary sequence. These include genetic variants, both natural and induced (for example, resulting from random mutagenesis by irradiation or exposure to ethanemethylsulfate or by site-specific mutagenesis as described in Sambrook, Fritsch and Maniatis, Molecular Cloning: A Laboratory Manual (2d ed.), CSH Press, 1989, or Ausubel et al., supra). Also included are cyclized peptides, molecules, and analogs which contain residues other than L-amino acids, e.g., D-amino acids or non-naturally occurring or synthetic amino acids, e.g., β or γ amino acids.

In addition to full-length polypeptides, the presently disclosed subject matter also provides fragments of any of the polypeptides disclosed herein. As used herein, the term “a fragment” means at least 5, 10, 13, or 15 amino acids. In certain embodiments, a fragment comprises at least 20 contiguous amino acids, at least 30 contiguous amino acids, or at least 50 contiguous amino acids. In certain embodiments, a fragment comprises at least 60 to 80, 100, 200, 300 or more contiguous amino acids. Fragments can be generated by methods known to those skilled in the art or may result from normal protein processing (e.g., removal of amino acids from the nascent polypeptide that are not required for biological activity or removal of amino acids by alternative mRNA splicing or alternative protein processing events).

### 5.7. Formulations and Administration

The presently disclosed subject matter provides compositions comprising the presently disclosed cells. In certain embodiments, the compositions are pharmaceutical compositions further comprising a pharmaceutically acceptable carrier. Compositions comprising the presently disclosed cells can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the genetically modified cells in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the presently disclosed subject matter, however, any vehicle, diluent, or additive used would have to be compatible with the genetically modified cells.

The compositions can be isotonic, i.e., they can have the same osmotic pressure as blood and lacrimal fluid. The desired isotonicity of the compositions may be accomplished using sodium chloride, or other pharmaceutically acceptable agents such as dextrose, boric acid, sodium tartrate, propylene glycol or other inorganic or organic solutes. Sodium chloride can be particularly for buffers containing sodium ions.

Viscosity of the compositions, if desired, can be maintained at the selected level using a pharmaceutically acceptable thickening agent. For example, methylcellulose is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

Compositions comprising the presently disclosed cells can be provided systemically or directly to a subject for treating or ameliorating a disease or disorder. In certain embodiments, the presently disclosed cells or compositions comprising thereof are directly injected into an organ of interest (e.g., an organ affected by a neoplasia). Alternatively, the presently disclosed cells or compositions comprising thereof are provided indirectly to the organ of interest, for example, by administration into the circulatory system (e.g., the tumor vasculature). Expansion and differentiation agents can be provided prior to, during or after administration of the cells or compositions to increase production of cells (e.g., T cells or NK cells) in vitro or in vivo.

The presently disclosed cells can be administered in any physiologically acceptable vehicle, normally intravascularly, although they may also be introduced into bone or other convenient site where the cells may find an appropriate site for regeneration and differentiation (e.g., thymus).

The quantity of cells to be administered can vary for the subject being treated. In certain embodiments, between about 10⁴ and about 10¹⁰, between about 10⁴ and about 10⁷, between about 10¹ and about 10⁷, between about 10¹ and about 10⁹, or between about 10⁶ and about 10⁸ of the presently disclosed cells are administered to a subject. More effective cells may be administered in even smaller numbers. Usually, at least about 1×10⁵ cells will be administered, eventually reaching about 1×10¹⁰ or more. In certain embodiments, at least about 1×10⁵, 5×10⁵, 1×10⁶, about 5×10⁶, about 1×10⁷, about 5×10⁷, about 1×10⁸, or about 5×10⁸ of the presently disclosed cells are administered to a subject. In certain embodiments, about 1×10⁶ of the presently disclosed cells are administered to a subject. The precise determination of what would be considered an effective dose can be based on factors individual to each subject, including their size, age, sex, weight, and condition of the particular subject. Dosages can be readily ascertained by those skilled in the art from this disclosure and the knowledge in the art.

The presently disclosed cells can comprise a purified population of cells. Those skilled in the art can readily determine the percentage of the presently disclosed cells in a population using various well-known methods, such as fluorescence activated cell sorting (FACS). Suitable ranges of purity in populations comprising the presently disclosed immunoresponsive cells are about 50% to about 55%, about 5% to about 60%, and about 65% to about 70%. In certain embodiments, the purity is about 70% to about 75%, about 75% to about 80%, or about 80% to about 85%. In certain embodiments, the purity is about 85% to about 90%, about 90% to about 95%, and about 95% to about 10⁰%. Dosages can be readily adjusted by those skilled in the art (e.g., a decrease in purity may require an increase in dosage). The cells can be introduced by injection, catheter, or the like.

The skilled artisan can readily determine the amount of cells and optional additives, vehicles, and/or carrier in compositions and to be administered in methods. Typically, any additives (in addition to the active cell(s) and/or agent(s)) are present in an amount of 0.001 to 50% (weight) solution in phosphate buffered saline, and the active ingredient is present in the order of micrograms to milligrams, such as about 0.0001 to about 5 wt %, about 0.0001 to about 1 wt %, about 0.0001 to about 0.05 wt % or about 0.001 to about 20 wt %, about 0.01 to about 10 wt %, or about 0.05 to about 5 wt %. For any composition to be administered to an animal or human, the followings can be determined: toxicity such as by determining the lethal dose (LD) and LD50 in a suitable animal model e.g., rodent such as mouse; the dosage of the composition(s), concentration of components therein and timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

In certain embodiments, the composition is a pharmaceutical composition comprising the presently disclosed cells and a pharmaceutically acceptable carrier.

Administration of the compositions can be autologous or heterologous. For example, cells can be obtained from one subject, and administered to the same subject or a different, compatible subject. Peripheral blood derived cells or their progeny (e.g., in vivo, ex vivo or in vitro derived) can be administered. When administering a presently disclosed composition (e.g., a pharmaceutical composition comprising presently disclosed cells), it can be formulated in a unit dosage injectable form (solution, suspension, emulsion).

The presently disclosed cells and compositions can be administered by any method known in the art including, but not limited to, oral administration, intravenous administration, subcutaneous administration, intranodal administration, intratumoral administration, intrathecal administration, intravitreal administration, intrapleural administration, intraosseous administration, intraperitoneal administration, pleural administration, and direct administration to the subject.

Additionally or alternatively, the presently disclosed subject matter also provides compositions comprising lipid nanoparticles (e.g., described in Section 5.5.1) including a nucleic acid or a nucleic acid composition disclosed herein. Compositions comprising the presently disclosed lipid nanoparticles can be conveniently provided as sterile and/or pyrogen-free. Compositions can be prepared to meet the standards of the United States Pharmacopoeia (USP), the European Pharmacopoeia (EP), the British Pharmacopoeia, and/or the International Pharmacopoeia.

Compositions including the presently disclosed lipid nanoparticles can include pharmaceutically acceptable excipients. Non-limiting examples of pharmaceutically acceptable excipients include inert diluents, dispersing agents, granulating agents, surface active agents, emulsifiers, disintegrating agents, binding agents, preservatives, buffering agents, lubricating agents, and/or oils. Furthermore, excipients such as cocoa butter and suppository waxes, coloring agents, coating agents, sweetening, flavoring, and/or perfuming agents can be present in the composition.

In certain embodiments, compositions including the presently disclosed lipid nanoparticles can be prepared as injectable preparations. These injectable preparations can include pharmaceutically acceptable vehicles and solvents including, without any limitation, water, Ringer's solution, U.S.P., isotonic sodium chloride solution, and/or oils (e.g., oleic acid). In certain embodiments, injectable preparations comprising the presently disclosed lipid nanoparticles can include a liquid suspension of crystalline or amorphous material with poor water solubility. Use of these poor water solubility materials allows to slow absorption from subcutaneous or intramuscular injection. Alternatively or additionally, compositions including the presently disclosed lipid nanoparticles can be prepared for rectal or vaginal administration, oral administration, topical and/or transdermal administration, intradermal administration, pulmonary administration, nasal administration, buccal administration, or ophthalmic administration. Additional information on various ways for formulating and preparing pharmaceutical compositions including the presently disclosed lipid nanoparticles can be found in Remington: The Science and Practice of Pharmacy, 22nd Edition, A. R. Gennaro, Lippincott, Williams & Wilkins, Baltimore, Md., 2012.

In certain embodiments, the compositions including the presently disclosed lipid nanoparticles can be formulated for controlled release or sustained release. As used herein, the term “controlled release” refers to a pharmaceutical composition or compound release profile that conforms to a particular pattern of release to effect a therapeutic outcome. As used herein, the term “sustained release” refers to a pharmaceutical composition or compound that conforms to a release rate over a specific period of time. The period of time may include, but is not limited to, hours, days, weeks, months and years.

Compositions comprising the presently disclosed lipid nanoparticles can be provided systemically or directly to a subject for inducing and/or enhancing an immune response to an antigen and/or treating and/or preventing a tumor, e.g., a tumor associated with CD33. In certain embodiments, the presently disclosed lipid nanoparticles or compositions comprising thereof are provided in vivo to immunoresponsive cells. In certain embodiments, the presently disclosed lipid nanoparticles or compositions comprising thereof are directly injected into an organ of interest (e.g., an organ affected by a neoplasia). Alternatively, the presently disclosed lipid nanoparticles or compositions comprising thereof are provided indirectly to the organ of interest, for example, by administration into the circulatory system (e.g., the tumor vasculature). In certain embodiments, the presently disclosed lipid nanoparticles or compositions comprising thereof are provided ex vivo to immunoresponsive cells. Expansion and differentiation agents can be provided prior to, during or after administration of the lipid nanoparticles or compositions to increase production of cells (e.g., T cells or NK cells) ex vivo or in vivo.

The presently disclosed lipid nanoparticles can be administered in any physiologically acceptable vehicle, normally intravascularly, although they may also be introduced into bone or other convenient site where the cells may find an appropriate site for regeneration and differentiation (e.g., thymus).

The quantity of cells to be administered can vary for the subject being treated. In certain embodiments, between about 0.001 mg/kg to about 10 mg/kg, from about 0.005 mg/kg to about 10 mg/kg, from about 0.01 mg/kg to about 10 mg/kg, from about 0.05 mg/kg to about 10 mg/kg, from about 0.1 mg/kg to about 10 mg/kg, from about 1 mg/kg to about 10 mg/kg, from about 2 mg/kg to about 10 mg/kg, from about 5 mg/kg to about 10 mg/kg, from about 0.0001 mg/kg to about 5 mg/kg, from about 0.001 mg/kg to about 5 mg/kg, from about 0.005 mg/kg to about 5 mg/kg, from about 0.01 mg/kg to about 5 mg/kg, from about 0.05 mg/kg to about 5 mg/kg, from about 0.1 mg/kg to about 5 mg/kg, from about 1 mg/kg to about 5 mg/kg, from about 2 mg/kg to about 5 mg/kg, from about 0.0001 mg/kg to about 2.5 mg/kg, from about 0.001 mg/kg to about 2.5 mg/kg, from about 0.005 mg/kg to about 2.5 mg/kg, from about 0.01 mg/kg to about 2.5 mg/kg, from about 0.05 mg/kg to about 2.5 mg/kg, from about 0.1 mg/kg to about 2.5 mg/kg, from about 1 mg/kg to about 2.5 mg/kg, from about 2 mg/kg to about 2.5 mg/kg, from about 0.0001 mg/kg to about 1 mg/kg, from about 0.001 mg/kg to about 1 mg/kg, from about 0.005 mg/kg to about 1 mg/kg, from about 0.01 mg/kg to about 1 mg/kg, from about 0.05 mg/kg to about 1 mg/kg, from about 0.1 mg/kg to about 1 mg/kg, from about 0.0001 mg/kg to about 0.25 mg/kg, from about 0.001 mg/kg to about 0.25 mg/kg, from about 0.005 mg/kg to about 0.25 mg/kg, from about 0.01 mg/kg to about 0.25 mg/kg, from about 0.05 mg/kg to about 0.25 mg/kg, or from about 0.1 mg/kg to about 0.25 mg/kg of the presently disclosed lipid nanoparticles are administered to a subject. In certain embodiments, between about 0.005 mg/kg to about 2.5 mg/kg, from about 0.1 mg/kg to about 1 mg/kg, or from about 0.05 mg/kg to about 1 mg/kg of the presently disclosed cells are administered to a subject. The precise determination of what would be considered an effective dose can be based on factors individual to each subject, including their size, age, sex, weight, and condition of the particular subject. Dosages can be readily ascertained by those skilled in the art from this disclosure and the knowledge in the art. Dosages can be readily adjusted by those skilled in the art (e.g., a decrease in purity may require an increase in dosage).

### 5.8. Methods of Treatment

The presently disclosed subject matter provides methods for inducing and/or increasing an immune response in a subject in need thereof. The presently disclosed cells and compositions comprising thereof can be used in a therapy or a medicament. The presently disclosed subject matter provides various methods of using the cells (e.g., T cells) or compositions comprising thereof. For example, the presently disclosed cells and compositions comprising thereof can be used for reducing tumor burden in a subject. The presently disclosed cell can reduce the number of tumor cells, reduce tumor size, and/or eradicate the tumor in the subject. The presently disclosed cells and compositions comprising thereof can be used for treating and/or preventing a tumor in a subject. The presently disclosed cells and compositions comprising thereof can be used for treating or ameliorating a disease or disorder in a subject. In certain embodiments, the disease or disorder is associated with CD33. In certain embodiments, the disease or disorder is associated with overexpression of CD33. In certain embodiments, the disease or disorder is a tumor. The presently disclosed cells and compositions comprising thereof can be used for prolonging the survival of a subject suffering from a tumor.

Such methods comprise administering the presently disclosed cells or a composition (e.g., a pharmaceutical composition) comprising thereof to achieve the desired effect, e.g., palliation of an existing condition or prevention of recurrence. For treatment, the amount administered is an amount effective in producing the desired effect. An effective amount can be provided in one or a series of administrations. An effective amount can be provided in a bolus or by continuous perfusion.

The presently disclosed subject matter provides various methods of using the cells (e.g., T cells) or compositions comprising thereof. For example, the presently disclosed subject matter provides methods of reducing tumor burden in a subject. In certain embodiments, the method of reducing tumor burden comprises administering the presently disclosed cells or a composition comprising thereof to the subject. The presently disclosed cell can reduce the number of tumor cells, reduce tumor size, and/or eradicate the tumor in the subject.

The presently disclosed subject matter also provides methods of increasing or lengthening survival of a subject having a tumor. In certain embodiments, the method of increasing or lengthening survival of a subject having a tumor or neoplasm comprises administering the presently disclosed immunoresponsive cells or a composition comprising thereof to the subject. The method can reduce or eradicate tumor burden in the subject.

Additionally, the presently disclosed subject matter provides methods for increasing an immune response in a subject, comprising administering the presently disclosed cell or a composition comprising thereof to the subject.

The presently disclosed subject matter further provides methods for treating and/or preventing a tumor in a subject, comprising administering the presently disclosed cells or a composition comprising thereof to the subject.

In certain embodiments, the method comprises administering to a subject in need thereof the presently disclosed cells or compositions comprising thereof. In certain embodiments, the cell is a T cell. The T cell can be a CD4⁺ T cell or a CD8⁺ T cell. In certain embodiments, the T cell is a CD4⁺ T cell.

In certain embodiments, the tumor is cancer. In certain embodiments, the cancer is a hematological cancer or solid tissue cancer. Non-limiting examples of hematological cancer includes acute myeloid leukemia (AML), myelodysplastic syndromes (MDS), acute lymphoblastic leukemia (ALL), chronic lymphocytic leukemia (CLL), myeloproliferative neoplasms (MPNs), and chronic myeloid neoplasms. In certain embodiments, the hematological cancer is AML.

The subjects can have an advanced form of disease, in which case the treatment objective can include mitigation or reversal of disease progression, and/or amelioration of side effects. The subjects can have a history of the condition, for which they have already been treated, in which case the therapeutic objective will typically include a decrease or delay in the risk of recurrence.

As a consequence of surface expression of a presently disclosed CD33-targeted antigen-recognizing receptor, adoptively transferred cells (e.g., immunoresponsive cells, e.g., T cells or NK cells) are endowed with augmented and selective cytolytic activity at the tumor site. Furthermore, subsequent to their localization to tumor or viral infection and their proliferation, the cells turn the tumor or viral infection site into a highly conductive environment for a wide range of immune cells involved in the physiological anti-tumor or antiviral response (tumor infiltrating lymphocytes, NK-, NKT-cells, dendritic cells, and macrophages).

Further modification can be introduced to the CD33-specific CD33-expressing engineered immune cells (e.g., T cells) to avert or minimize the risks of immunological complications (known as “malignant T-cell transformation”), e.g., graft versus-host disease (GvHD). Modification of the engineered immune cells can include engineering a suicide gene into the CD33-specific CAR-expressing T cells. Suitable suicide genes include, but are not limited to, Herpes simplex virus thymidine kinase (hsv-tk), inducible Caspase 9 Suicide gene (iCasp-9), and a truncated human epidermal growth factor receptor (EGFRt) polypeptide. In certain embodiments, the suicide gene is an EGFRt polypeptide. The EGFRt polypeptide can enable T cell elimination by administering anti-EGFR monoclonal antibody (e.g., cetuximab). EGFRt can be covalently joined to the C-terminus of the intracellular domain of the CD33-specific CAR. The suicide gene can be included within the vector comprising nucleic acids encoding the presently disclosed CD33-specific CARs. The incorporation of a suicide gene into the a presently disclosed CD33-specific CAR gives an added level of safety with the ability to eliminate the majority of CAR T cells within a very short time period. A presently disclosed engineered immune cell (e.g., a T cell) incorporated with a suicide gene can be pre-emptively eliminated at a given time point post CAR T cell infusion, or eradicated at the earliest signs of toxicity.

### 5.9. Kits

The presently disclosed subject matter provides kits for or ameliorating a disease or disorder in a subject, inducing and/or enhancing an immune response in a subject, treating and/or preventing a tumor in a subject, reducing tumor burden in a subject, and/or increasing or lengthening survival of a subject having a tumor in a subject. In certain embodiments, the kit comprises the presently disclosed cells or a composition comprising thereof. In certain embodiments, the kit comprises a sterile container; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments. In certain non-limiting embodiments, the kit includes a nucleic acid molecule encoding a presently disclosed CD33-targeted antigen-recognizing receptor (e.g., a CAR).

If desired, the cells and/or nucleic acid molecules are provided together with instructions for administering the cells or nucleic acid molecules to a subject having or at risk of developing a disease or disorder (e.g., a tumor). The instructions generally include information about the use of the composition for the treatment and/or prevention of a tumor. In certain embodiments, the instructions include at least one of the following: description of the therapeutic agent; dosage schedule and administration for treatment or prevention of a tumor or neoplasm; precautions; warnings; indications; counter-indications; over-dosage information; adverse reactions; animal pharmacology; clinical studies; and/or references. The instructions may be printed directly on the container (when present), or as a label applied to the container, or as a separate sheet, pamphlet, card, or folder supplied in or with the container.

### 5.10. Exemplary Embodiments

A1. In certain non-limiting embodiments, the presently disclosed subject matter provides an antigen-recognizing receptor, comprising an extracellular antigen-binding domain, a transmembrane domain, and an intracellular signaling domain, wherein the extracellular antigen-binding domain specifically binds to CD33, wherein the extracellular antigen-binding domain comprises a heavy chain variable region and a light chain variable region, wherein the heavy chain variable region comprises:
(a) a CDR1 comprising the amino acid sequence set forth in SEQ ID NO: 2 or a conservative modification thereof, a CDR2 comprising the amino acid sequence set forth in SEQ ID NO: 3 or a conservative modification thereof, and a CDR3 comprising the amino acid sequence set forth in SEQ ID NO: 4 or a conservative modification thereof;
(b) a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof;
(c) a CDR1 comprising the amino acid sequence set forth in SEQ ID NO: 22 or a conservative modification thereof, a CDR2 comprising the amino acid sequence set forth in SEQ ID NO: 23 or a conservative modification thereof, and a CDR3 comprising the amino acid sequence set forth in SEQ ID NO: 24 or a conservative modification thereof;
(d) a CDR1 comprising the amino acid sequence set forth in SEQ ID NO: 32 or a conservative modification thereof, a CDR2 comprising the amino acid sequence set forth in SEQ ID NO: 33 or a conservative modification thereof, and a CDR3 comprising the amino acid sequence set forth in SEQ ID NO: 34 or a conservative modification thereof;
(e) a CDR1 comprising the amino acid sequence set forth in SEQ ID NO: 37 or a conservative modification thereof, a CDR2 comprising the amino acid sequence set forth in SEQ ID NO: 38 or a conservative modification thereof, and a CDR3 comprising the amino acid sequence set forth in SEQ ID NO: 39 or a conservative modification thereof;
(f) a CDR1 comprising the amino acid sequence set forth in SEQ ID NO: 47 or a conservative modification thereof, a CDR2 comprising the amino acid sequence set forth in SEQ ID NO: 38 or a conservative modification thereof, and a CDR3 comprising the amino acid sequence set forth in SEQ ID NO: 48 or a conservative modification thereof;
(g) a CDR1 comprising the amino acid sequence set forth in SEQ ID NO: 56 or a conservative modification thereof, a CDR2 comprising the amino acid sequence set forth in SEQ ID NO: 57 or a conservative modification thereof, and a CDR3 comprising the amino acid sequence set forth in SEQ ID NO: 58 or a conservative modification thereof;
(h) a CDR1 comprising the amino acid sequence set forth in SEQ ID NO: 66 or a conservative modification thereof, a CDR2 comprising the amino acid sequence set forth in SEQ ID NO: 67 or a conservative modification thereof, and a CDR3 comprising the amino acid sequence set forth in SEQ ID NO: 68 or a conservative modification thereof;
(i) a CDR1 comprising the amino acid sequence set forth in SEQ ID NO: 76 or a conservative modification thereof, a CDR2 comprising the amino acid sequence set forth in SEQ ID NO: 57 or a conservative modification thereof, and a CDR3 comprising the amino acid sequence set forth in SEQ ID NO: 77 or a conservative modification thereof; or
(j) a CDR1 comprising the amino acid sequence set forth in SEQ ID NO: 85 or a conservative modification thereof, a CDR2 comprising the amino acid sequence set forth in SEQ ID NO: 86 or a conservative modification thereof, and a CDR3 comprising the amino acid sequence set forth in SEQ ID NO: 87 or a conservative modification thereof.
A2. The foregoing antigen-recognizing receptor of A1, wherein the extracellular antigen-binding domain is a single-chain variable fragment (scFv).
A3. The foregoing antigen-recognizing receptor of A2, wherein the extracellular antigen-binding domain is a human scFv.
A4. The foregoing antigen-recognizing receptor of A1, wherein the extracellular antigen-binding domain is a Fab, which is optionally crosslinked.
A5. The foregoing antigen-recognizing receptor of A1, wherein the extracellular antigen-binding domain is a F(ab)₂.
A6. The foregoing antigen-recognizing receptor of any one of A2-A5, wherein one or more of the scFv, Fab and F(ab)₂ are comprised in a fusion protein with a heterologous sequence to form the extracellular antigen-binding domain.
A7. The foregoing antigen-recognizing receptor of any one of A1-A6, wherein the heavy chain variable region comprises:
(a) a CDR1 comprising the amino acid sequence set forth in SEQ ID NO: 2 or a conservative modification thereof, a CDR2 comprising the amino acid sequence set forth in SEQ ID NO: 3 or a conservative modification thereof, and a CDR3 comprising the amino acid sequence set forth in SEQ ID NO: 4 or a conservative modification thereof;
(b) a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof; or
(c) a CDR1 comprising the amino acid sequence set forth in SEQ ID NO: 22 or a conservative modification thereof, a CDR2 comprising the amino acid sequence set forth in SEQ ID NO: 23 or a conservative modification thereof, and a CDR3 comprising the amino acid sequence set forth in SEQ ID NO: 24 or a conservative modification thereof.
A8. The foregoing antigen-recognizing receptor of any one of A1-A7, wherein the light chain variable region comprises:
(a) a CDR1 comprising the amino acid sequence set forth in SEQ ID NO: 5 or a conservative modification thereof, a CDR2 comprising the amino acid sequence set forth in SEQ ID NO: 6 or a conservative modification thereof, and a CDR3 comprising the amino acid sequence set forth in SEQ ID NO: 7 or a conservative modification thereof;
(b) a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising SEQ ID NO: 17 or a conservative modification thereof;
(c) a CDR1 comprising the amino acid sequence set forth in SEQ ID NO: 25 or a conservative modification thereof, a CDR2 comprising the amino acid sequence set forth in SEQ ID NO: 26 or a conservative modification thereof, and a CDR3 comprising the amino acid sequence set forth in SEQ ID NO: 27 or a conservative modification thereof;
(d) a CDR1 comprising the amino acid sequence set forth in SEQ ID NO: 40 or a conservative modification thereof, a CDR2 comprising SEQ ID NO: 41 or a conservative modification thereof, and a CDR3 comprising the amino acid sequence set forth in SEQ ID NO: 42 or a conservative modification thereof;
(e) a CDR1 comprising the amino acid sequence set forth in SEQ ID NO: 49 or a conservative modification thereof, a CDR2 comprising the amino acid sequence set forth in SEQ ID NO: 50 or a conservative modification thereof, and a CDR3 comprising the amino acid sequence set forth in SEQ ID NO: 51 or a conservative modification thereof;
(f) a CDR1 comprising the amino acid sequence set forth in SEQ ID NO: 59 or a conservative modification thereof, a CDR2 comprising the amino acid sequence set forth in SEQ ID NO: 60 or a conservative modification thereof, and a CDR3 comprising the amino acid sequence set forth in SEQ ID NO: 61 or a conservative modification thereof;
(g) a CDR1 comprising the amino acid sequence set forth in SEQ ID NO: 69 or a conservative modification thereof, a CDR2 comprising the amino acid sequence set forth in SEQ ID NO: 70 or a conservative modification thereof, and a CDR3 comprising the amino acid sequence set forth in SEQ ID NO: 71 or a conservative modification thereof;
(h) a CDR1 comprising the amino acid sequence set forth in SEQ ID NO: 78 or a conservative modification thereof, a CDR2 comprising the amino acid sequence set forth in SEQ ID NO: 79 or a conservative modification thereof, and a CDR3 comprising the amino acid sequence set forth in SEQ ID NO: 80 or a conservative modification thereof; or
(i) a CDR1 comprising the amino acid sequence set forth in SEQ ID NO: 5 or a conservative modification thereof, a CDR2 comprising the amino acid sequence set forth in SEQ ID NO: 41 or a conservative modification thereof, and a CDR3 comprising the amino acid sequence set forth in SEQ ID NO: 42 or a conservative modification thereof.
A9. The foregoing antigen-recognizing receptor of any one of A1-A8, wherein the light chain variable region comprises:
(a) a CDR1 comprising the amino acid sequence set forth in SEQ ID NO: 5 or a conservative modification thereof, a CDR2 comprising the amino acid sequence set forth in SEQ ID NO: 6 or a conservative modification thereof, and a CDR3 comprising the amino acid sequence set forth in SEQ ID NO: 7 or a conservative modification thereof;
(b) a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising SEQ ID NO: 17 or a conservative modification thereof; or
(c) a CDR1 comprising the amino acid sequence set forth in SEQ ID NO: 25 or a conservative modification thereof, a CDR2 comprising the amino acid sequence set forth in SEQ ID NO: 26 or a conservative modification thereof, and a CDR3 comprising the amino acid sequence set forth in SEQ ID NO: 27 or a conservative modification thereof.
A10. The foregoing antigen-recognizing receptor of any one of A1-A9, wherein:
(a) the heavy chain variable region comprises a CDR1 comprising the amino acid sequence set forth in SEQ ID NO: 2, a CDR2 comprising the amino acid sequence set forth in SEQ ID NO: 3, and a CDR3 comprising the amino acid sequence set forth in SEQ ID NO: 4; the light chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 7;
(b) the heavy chain variable region comprises a CDR1 comprising the amino acid sequence set forth in SEQ ID NO: 12, a CDR2 comprising an amino acid sequence set forth in SEQ ID NO: 13, and a CDR3 comprising the amino acid sequence set forth in SEQ ID NO: 14; the light chain variable region comprises a CDR1 comprising the amino acid sequence set forth in SEQ ID NO: 15, and a CDR2 comprising the amino acid sequence set forth in SEQ ID NO: 16, and a CDR3 comprising the amino acid sequence set forth in SEQ ID NO: 17;
(c) the heavy chain variable region comprises a CDR1 comprising the amino acid sequence set forth in SEQ ID NO: 22, a CDR2 comprising the amino acid sequence set forth in SEQ ID NO: 23, and a CDR3 comprising the amino acid sequence set forth in SEQ ID NO: 24 the light chain variable region comprises a CDR1 comprising the amino acid sequence set forth in SEQ ID NO: 25, a CDR2 comprising the amino acid sequence set forth in SEQ ID NO: 26, and a CDR3 comprising the amino acid sequence set forth in SEQ ID NO: 27;
(d) the heavy chain variable region comprises a CDR1 comprising the amino acid sequence set forth in SEQ ID NO: 32, a CDR2 comprising the amino acid sequence set forth in SEQ ID NO: 33, and a CDR3 comprising the amino acid sequence set forth in SEQ ID NO: 34 the light chain variable region comprises a CDR1 comprising the amino acid sequence set forth in SEQ ID NO: 25, a CDR2 comprising the amino acid sequence set forth in SEQ ID NO: 26, and a CDR3 comprising the amino acid sequence set forth in SEQ ID NO: 27;
(e) the heavy chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 39; the light chain variable region comprises a CDR1 comprising the amino acid sequence set forth in SEQ ID NO: 40, a CDR2 comprising the amino acid sequence set forth in SEQ ID NO: 41, and a CDR3 comprising the amino acid sequence set forth in SEQ ID NO: 42;
(f) the heavy chain variable region comprises a CDR1 comprising the amino acid sequence set forth in SEQ ID NO: 47, a CDR2 comprising the amino acid sequence set forth in SEQ ID NO: 38, and a CDR3 comprising the amino acid sequence set forth in SEQ ID NO: 48; the light chain variable region comprises a CDR1 comprising the amino acid sequence set forth in SEQ ID NO: 49, a CDR2 comprising the amino acid sequence set forth in SEQ ID NO: 50, and a CDR3 comprising the amino acid sequence set forth in SEQ ID NO: 51;
(g) the heavy chain variable region comprises a CDR1 comprising the amino acid sequence set forth in SEQ ID NO: 56, a CDR2 comprising the amino acid sequence set forth in SEQ ID NO: 57, and a CDR3 comprising the amino acid sequence set forth in SEQ ID NO: 58; the light chain variable region comprises a CDR1 comprising the amino acid sequence set forth in SEQ ID NO: 59, a CDR2 comprising the amino acid sequence set forth in SEQ ID NO: 60, and a CDR3 comprising the amino acid sequence set forth in SEQ ID NO: 61;
(h) the heavy chain variable region comprises a CDR1 comprising the amino acid sequence set forth in SEQ ID NO: 66, a CDR2 comprising the amino acid sequence set forth in SEQ ID NO: 67, and a CDR3 comprising the amino acid sequence set forth in SEQ ID NO: 68; the light chain variable region comprises a CDR1 comprising the amino acid sequence set forth in SEQ ID NO: 69, a CDR2 comprising the amino acid sequence set forth in SEQ ID NO: 70, and a CDR3 comprising the amino acid sequence set forth in SEQ ID NO: 71;
(i) the heavy chain variable region comprises a CDR1 comprising the amino acid sequence set forth in SEQ ID NO: 76, a CDR2 comprising the amino acid sequence set forth in SEQ ID NO: 57, and a CDR3 comprising the amino acid sequence set forth in SEQ ID NO: 77; the light chain variable region comprises a CDR1 comprising the amino acid sequence set forth in SEQ ID NO: a CDR2 comprising the amino acid sequence set forth in SEQ ID NO: 79, and a CDR3 comprising the amino acid sequence set forth in SEQ ID NO: 80; or
(j) the heavy chain variable region comprises a CDR1 comprising the amino acid sequence set forth in SEQ ID NO: 85, a CDR2 comprising the amino acid sequence set forth in SEQ ID NO: 86, and a CDR3 comprising the amino acid sequence set forth in SEQ ID NO: 87; the light chain variable region comprises a CDR1 comprising the amino acid sequence set forth in SEQ ID NO: 5, a CDR2 comprising the amino acid sequence set forth in SEQ ID NO: 41, and a CDR3 comprising the amino acid sequence set forth in SEQ ID NO: 42.

All. The foregoing antigen-recognizing receptor of any one of A1-A10, wherein:
(a) the heavy chain variable region comprises a CDR1 comprising the amino acid sequence set forth in SEQ ID NO: 2, a CDR2 comprising the amino acid sequence set forth in SEQ ID NO: 3, and a CDR3 comprising the amino acid sequence set forth in SEQ ID NO: 4; the light chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 7;
(b) the heavy chain variable region comprises a CDR1 comprising the amino acid sequence set forth in SEQ ID NO: 12, a CDR2 comprising an amino acid sequence set forth in SEQ ID NO: 13, and a CDR3 comprising the amino acid sequence set forth in SEQ ID NO: 14; the light chain variable region comprises a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17; or
(c) the heavy chain variable region comprises a CDR1 comprising the amino acid sequence set forth in SEQ ID NO: 22, a CDR2 comprising the amino acid sequence set forth in SEQ ID NO: 23, and a CDR3 comprising the amino acid sequence set forth in SEQ ID NO: 24; the light chain variable region comprises a CDR1 comprising the amino acid sequence set forth in SEQ ID NO: 25, a CDR2 comprising the amino acid sequence set forth in SEQ ID NO: 26, and a CDR3 comprising the amino acid sequence set forth in SEQ ID NO: 27.
A12. The foregoing antigen-recognizing receptor of any one A1-A11, wherein the heavy chain variable region comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence set forth in SEQ ID NO: 8, SEQ ID NO: 18, SEQ ID NO: 28, SEQ ID NO: 35, SEQ ID NO: 43, SEQ ID NO: 52, SEQ ID NO: 62, SEQ ID NO: 72, SEQ ID NO: 81, or SEQ ID NO: 88.
A13. The foregoing antigen-recognizing receptor of any one of A1-A12, wherein the heavy chain variable region comprises the amino acid sequence set forth in SEQ ID NO: 8, SEQ ID NO: 18, SEQ ID NO: 28, SEQ ID NO: 35, SEQ ID NO: 43, SEQ ID NO: 52, SEQ ID NO: 62, SEQ ID NO: 72, SEQ ID NO: 81, or SEQ ID NO: 88.
A14. The foregoing antigen-recognizing receptor of any one of A1-A10, wherein the heavy chain variable region comprises the amino acid sequence set forth in SEQ ID NO: 8, SEQ ID NO: 18, or SEQ ID NO: 28.
A15. The foregoing antigen-recognizing receptor of any one of A1-A14, wherein the light chain variable region comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence set forth in SEQ ID NO: 9, SEQ ID NO: 19, SEQ ID NO: 29, SEQ ID NO: 44, SEQ ID NO: 53, SEQ ID NO: 63, SEQ ID NO: 73, SEQ ID NO: 82, SEQ ID NO: 89, or SEQ ID NO: 92.
A16. The foregoing antigen-recognizing receptor of any one of A1-A15, wherein the light chain variable region comprises the amino acid sequence set forth in SEQ ID NO: 9, SEQ ID NO: 19, SEQ ID NO: 29, SEQ ID NO: 44, SEQ ID NO: 53, SEQ ID NO: 63, SEQ ID NO: 73, SEQ ID NO: 82, SEQ ID NO: 89, or SEQ ID NO: 92.
A17. The foregoing antigen-recognizing receptor of any one of A1-A16, wherein the light chain variable region comprises the amino acid sequence set forth in SEQ ID NO: 9, SEQ ID NO: 19, or SEQ ID NO: 29.
A18. The foregoing antigen-recognizing receptor of any one of A1-A17, wherein:
(a) the heavy chain variable region comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence selected set forth in SEQ ID NO: 8, SEQ ID NO: 18, SEQ ID NO: 28, SEQ ID NO: 35, SEQ ID NO: 43, SEQ ID NO: 52, SEQ ID NO: 62, SEQ ID NO: 72, SEQ ID NO: 81, or SEQ ID NO: 88; and
(b) the light chain variable region comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence set forth in SEQ ID NO: 9, SEQ ID NO: 19, SEQ ID NO: 29, SEQ ID NO: 44, SEQ ID NO: 53, SEQ ID NO: 63, SEQ ID NO: 73, SEQ ID NO: 82, SEQ ID NO: 89, or SEQ ID NO: 92.
A19. The foregoing antigen-recognizing receptor of any one of A1-A18, wherein:
(a) the heavy chain variable region comprises the amino acid sequence set forth in SEQ ID NO: 8, SEQ ID NO: 18, SEQ ID NO: 28, SEQ ID NO: 35, SEQ ID NO: 43, SEQ ID NO: 52, SEQ ID NO: 62, SEQ ID NO: 72, SEQ ID NO: 81, or SEQ ID NO: 88; and
(b) the light chain variable region comprises the amino acid sequence set forth in SEQ ID NO: 9, SEQ ID NO: 19, SEQ ID NO: 29, SEQ ID NO: 44, SEQ ID NO: 53, SEQ ID NO: 63, SEQ ID NO: 73, SEQ ID NO: 82, SEQ ID NO: 89, or SEQ ID NO: 92.
A20. The foregoing antigen-recognizing receptor of any one of A1-A19, wherein:
(a) the heavy chain variable region comprises the amino acid sequence set forth in SEQ ID NO: 8, SEQ ID NO: 18, or SEQ ID NO: 28; and
(b) the light chain variable region comprises the amino acid sequence set forth in SEQ ID NO: 9, SEQ ID NO: 19, or SEQ ID NO: 29.
A21. The foregoing antigen-recognizing receptor of any one of A1-A20, wherein:
(a) the heavy chain variable region comprises the amino acid sequence set forth in SEQ ID NO: 8, and the light chain variable region comprises the amino acid sequence set forth in SEQ ID NO: 9;
(b) the heavy chain variable region comprises the amino acid sequence set forth in SEQ ID NO: 18, and the light chain variable region comprises the amino acid sequence set forth in SEQ ID NO: 19;
(c) the heavy chain variable region comprises the amino acid sequence set forth in SEQ ID NO: 28, and the light chain variable region comprises the amino acid sequence set forth in SEQ ID NO: 29;
(d) the heavy chain variable region comprises the amino acid sequence set forth in SEQ ID NO: 35, and the light chain variable region comprises the amino acid sequence set forth in SEQ ID NO: 29;
(e) the heavy chain variable region comprises the amino acid sequence set forth in SEQ ID NO: 43, and the light chain variable region comprises the amino acid sequence set forth in SEQ ID NO: 44;
(f) the heavy chain variable region comprises the amino acid sequence set forth in SEQ ID NO: 52, and the light chain variable region comprises the amino acid sequence set forth in SEQ ID NO: 53;
(g) the heavy chain variable region comprises the amino acid sequence set forth in SEQ ID NO: 62, and the light chain variable region comprises the amino acid sequence set forth in SEQ ID NO: 63;
(h) the heavy chain variable region comprises the amino acid sequence set forth in SEQ ID NO: 72, and the light chain variable region comprises the amino acid sequence set forth in SEQ ID NO: 73;
(i) the heavy chain variable region comprises the amino acid sequence set forth in SEQ ID NO: 81, and the light chain variable region comprises the amino acid sequence set forth in SEQ ID NO: 82;
(j) the heavy chain variable region comprises the amino acid sequence set forth in SEQ ID NO: 88, and the light chain variable region comprises the amino acid sequence set forth in SEQ ID NO: 89; or
(k) the heavy chain variable region comprises the amino acid sequence set forth in SEQ ID NO: 8, and the light chain variable region comprises the amino acid sequence set forth in SEQ ID NO: 93.
A22. The antigen-recognizing receptor of any one of A1-A21, wherein:
(a) the heavy chain variable region comprises the amino acid sequence set forth in SEQ ID NO: 8, and the light chain variable region comprises the amino acid sequence set forth in SEQ ID NO: 9;
(b) the heavy chain variable region comprises the amino acid sequence set forth in SEQ ID NO: 18, and the light chain variable region comprises the amino acid sequence set forth in SEQ ID NO: 19; or
(c) the heavy chain variable region comprises the amino acid sequence set forth in SEQ ID NO: 28, and the light chain variable region comprises the amino acid sequence set forth in SEQ ID NO: 29.
A23. The foregoing antigen-recognizing receptor of any one of A1-A22, wherein the extracellular antigen-binding domain comprises a linker between the heavy chain variable region and the light chain variable region.
A24. The foregoing antigen-recognizing receptor of A23, wherein the linker comprises or consists of the amino acid sequence set forth in SEQ ID NO: 95, SEQ ID NO: 96, SEQ ID NO: 97, SEQ ID NO: 98, SEQ ID NO: 99, or SEQ ID NO: 100.
A25. The foregoing antigen-recognizing receptor of any one of A1-A24, wherein a signal peptide is covalently joined to the 5′ terminus of the extracellular antigen-binding domain.
A26. The foregoing antigen-recognizing receptor of any one of A1-A25, wherein the transmembrane domain comprises a CD8 polypeptide, a CD28 polypeptide, a CD3ζ polypeptide, a CD4 polypeptide, a 4-1BB polypeptide, an OX40 polypeptide, an ICOS polypeptide, a CTLA-4 polypeptide, a PD-1 polypeptide, a LAG-3 polypeptide, a 2B4 polypeptide, a BTLA polypeptide, or a combination thereof.
A27. The foregoing antigen-recognizing receptor of any one of A1-A26, wherein the intracellular signaling domain comprises a CD3ζ polypeptide.
A28. The foregoing antigen-recognizing receptor of any one of A1-A27, wherein the intracellular signaling domain further comprises at least one co-stimulatory signaling region.
A29. The foregoing antigen-recognizing receptor of A28, wherein the at least one co-stimulatory signaling region comprises a CD28 polypeptide, a 4-1BB polypeptide, an OX40 polypeptide, an ICOS polypeptide, a DAP-10 polypeptide, or a combination thereof.
A30. The foregoing antigen-recognizing receptor of any one of A1-A29, wherein the antigen-recognizing receptor is a chimeric antigen receptor (CAR), or a T-cell like fusion protein.
A31. The foregoing antigen-recognizing receptor of any one of A1-A30, wherein the antigen-recognizing receptor is a CAR.
A32. The foregoing antigen-recognizing receptor of any one of A1-A31, wherein the antigen-recognizing receptor is recombinantly expressed.
A33. The foregoing antigen-recognizing receptor of any one of A1-A32, wherein the antigen-recognizing receptor is expressed from a vector.
A34. The foregoing antigen-recognizing receptor of A33, wherein the vector is a γ-retroviral vector.
B1. In certain non-limiting embodiments, the presently disclosed subject matter provides a cell comprising the antigen-recognizing receptor of any one of A1-A34.
B2. The foregoing cell of B1, wherein the cell is transduced with the antigen-recognizing receptor.
B3. The foregoing cell of B1 or B2, wherein the antigen-recognizing receptor is constitutively expressed on the surface of the cell.
B4. The foregoing cell of any one of B1-B3, wherein the cell is an immunoresponsive cell.
B5. The foregoing cell of any one of B1-B4, wherein the cell is a cell of the lymphoid lineage or a cell of the myeloid lineage.
B6. The foregoing cell of any one of B1-B5, wherein the cell is selected from the group consisting of a T cell, a Natural Killer (NK) cell, and a stem cell from which a lymphoid cell may be differentiated.
B7. The foregoing cell of any one of B1-B6, wherein the cell is a T cell.
B8. The foregoing cell of B6 or B7, wherein the T cell is a cytotoxic T lymphocyte (CTL) or a regulatory T cell.
B9. The foregoing cell of B6, wherein the stem cell is a pluripotent stem cell.
B10. The foregoing cell of B9, wherein the pluripotent stem cell is an embryoid stem cell or an induced pluripotent stem cell.
C. In certain non-limiting embodiments, the presently disclosed subject matter provides a nucleic acid encoding the antigen-recognizing receptor of any one of A1-A34.
D1. In certain non-limiting embodiments, the presently disclosed subject matter provides a vector comprising the nucleic acid of C.
D2. The foregoing vector of claim D1, wherein the vector is a γ-retroviral vector.
E1. In certain non-limiting embodiments, the presently disclosed subject matter provides a host cell expressing the nucleic acid of C.
E2. The foregoing host cell of E1, wherein the host cell is a T cell.
F1. In certain non-limiting embodiments, the presently disclosed subject matter provides a composition comprising the cell of any one of B1-B10.
F2. The foregoing composition of F1, which is a pharmaceutical composition further comprising a pharmaceutically acceptable carrier.
G. In certain non-limiting embodiments, the presently disclosed subject matter provides a lipid nanoparticle comprising the nucleic acid of C.
H1. In certain non-limiting embodiments, the presently disclosed subject matter provides a composition comprising the lipid nanoparticle of G.
H2. The foregoing composition of H1, which is a pharmaceutical composition further comprising a pharmaceutically acceptable carrier.
I1. In certain non-limiting embodiments, the presently disclosed subject matter provides a method of treating or ameliorating a disease or disorder in a subject, comprising administering to the subject the cell of any one of B1-B10, or the composition of any one of claims F1, F2, H1, or H2.
I2. The foregoing method of I1, wherein the disease or disorder is a tumor.
J1. In certain non-limiting embodiments, the presently disclosed subject matter provides a method of reducing tumor burden in a subject, comprising administering to the subject the cell of any one of B1-B10, or the composition of any one F1, F2, H1, or H2.
J2. The foregoing method of J1, wherein the method reduces the number of the tumor cells, reduces the tumor size, and/or eradicates the tumor in the subject.
K. In certain non-limiting embodiments, the presently disclosed subject matter provides a method of treating and/or preventing a tumor in a subject, comprising administering to the subject the cell of any one of B1-B10, or the composition of any one of F1, F2, H1, or H2.
L1. In certain non-limiting embodiments, the presently disclosed subject matter provides a method of increasing or lengthening survival of a subject having a tumor, comprising administering to the subject the cell of any one of claims B1-B10, or the composition of any one of claims F1, F2, H1, or H2.
L2. The foregoing method of L1, wherein the method reduces or eradicates tumor burden in the subject.
L3. The foregoing method of any one of I2-L2, wherein the tumor is cancer.
L4. The foregoing method of any one of I2-L3, wherein the tumor is hematological cancer or solid tissue cancer.
L5. The foregoing method of any one I2-L4, wherein the tumor is selected from the group consisting of acute myeloid leukemia (AML), myelodysplastic syndromes (MDS), acute lymphoblastic leukemia (ALL), chronic lymphocytic leukemia (CLL), myeloproliferative neoplasms (MPNs), and chronic myeloid neoplasms.
L6. The foregoing method of L5, wherein the tumor is acute myeloid leukemia (AML).
L7. The foregoing method of any one of 12-L6, wherein the subject is a human.
M1. In certain non-limiting embodiments, the presently disclosed subject matter provides a kit for treating or ameliorating a disease or disorder in a subject, reducing tumor burden in a subject, treating and/or preventing a tumor in a subject, and/or increasing or lengthening survival of a subject having a tumor, comprising the cell of any one B1-B10, the nucleic acid of C, the lipid nanoparticle of G, or the composition of any one of claims F1, F2, H1, or H2.
M2. The foregoing kit of claim M1, wherein the kit further comprises written instructions for using the cell or composition for treating or ameliorating a disease or disorder in a subject, reducing tumor burden in a subject, treating and/or preventing a tumor in a subject, and/or increasing or lengthening survival of a subject having a tumor.
N. In certain non-limiting embodiments, the presently disclosed subject matter provides a method for producing a CD33-targeted antigen-recognizing receptor of any one of claims A1-A34, comprising introducing into the cell a nucleic acid that encodes the antigen-recognizing receptor.
O1. In certain non-limiting embodiments, the presently disclosed subject matter provides the cell of any one of B1-B10 or the composition of F1, F2, H1, or H2 for use in treating or ameliorating a disease.
O2. The foregoing cell or the foregoing composition for use in O1, wherein the disease or disorder is a tumor.
O3. The foregoing cell or the foregoing composition for use in O2, wherein the tumor is cancer.
O4. The foregoing cell or the foregoing composition for use in any one 01-03, wherein the disease or disorder is selected from the group consisting of neuroendocrine tumors of the lung, extrapulmonary neuroendocrine carcinomas, melanoma, neuroendocrine prostate cancer, breast cancer, neuroendocrine tumors of the gastrointestinal tract, pancreatic cancer, medullary thyroid cancer, small cell bladder cancer, ovarian small cell carcinoma, low-grade glioma, glioblastoma and neuroblastoma.
O5. The foregoing cell or the foregoing composition for use in O4, wherein the neuroendocrine tumors of the lung are selected from the group consisting of pulmonary neuroendocrine cancer, large cell neuroendocrine carcinoma, and small-cell lung cancer.
O6. The foregoing cell or the foregoing composition for use in O5, wherein the tumor is small-cell lung cancer.
O7. The foregoing cell or the foregoing composition for use in any one of 01-06, wherein the subject is a human.

### 6. EXAMPLES

The practice of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides disclosed herein, and, as such, may be considered in making and practicing the presently disclosed subject matter. Particularly useful techniques for particular embodiments will be discussed in the sections that follow.

The following examples are put forth so as to provide those of ordinary skill in the art with a complete disclosure and description of how to make and use the antibodies, multi-specific antibodies, compositions comprising thereof, screening, and therapeutic methods of the presently disclosed subject matter, and are not intended to limit the scope of what the inventors regard as their presently disclosed subject matter. It is understood that various other embodiments may be practiced, given the general description provided above.

### Example 1-Design of CAR Constructs and Cell Surface Expression

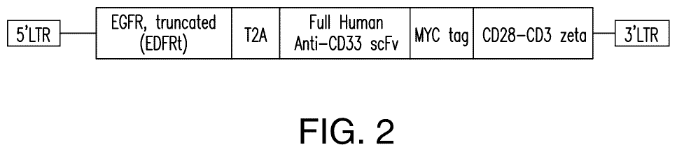

Variable regions for candidate mAbs with unique sequences were formatted as scFvs. Two antibodies (3P14 and 4B2) were IgC2 specific, while the other 5 (1A20, 1J19, 1P13, 1P23 and 2N3) targeted the IgV domain. These scFvs were cloned into a second-generation CAR vector containing a CD8 leader sequence, truncated EGFR (EGFRt) as transduction efficiency marker, a T2A cleavage signal, the candidate antibody scFvs, a Myc tag for CAR detection, a CD28 transmembrane segment and the CD28/CD3ζ T cell signaling domains flanked by 5′ and 3′ long terminal repeats (LTR) as shown in FIG. 2. All scFvs were tested in V_{L}-V_{H} and V_{H}-V_{L} orientation connected by a 15-amino-acid glycine serine linker set forth in SEQ ID NO: 96. The final construct was cloned into a retroviral vector SFG14. Antibody H195 (lintuzumab) was included as a reference control. The sequences of the variable regions of lintuzumab were derived from the patent literature.

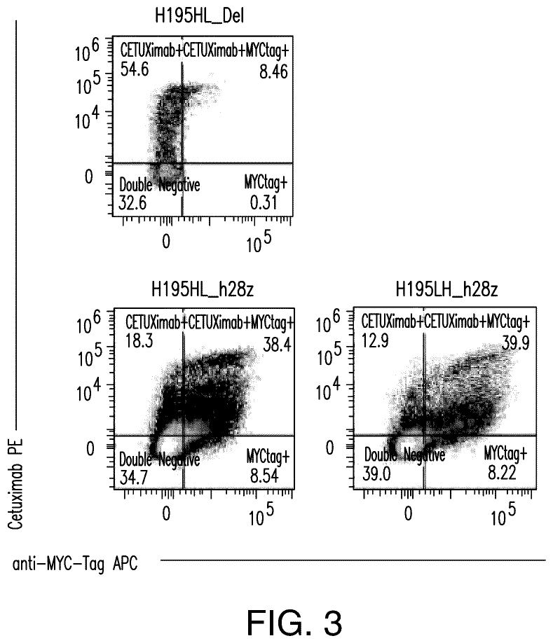

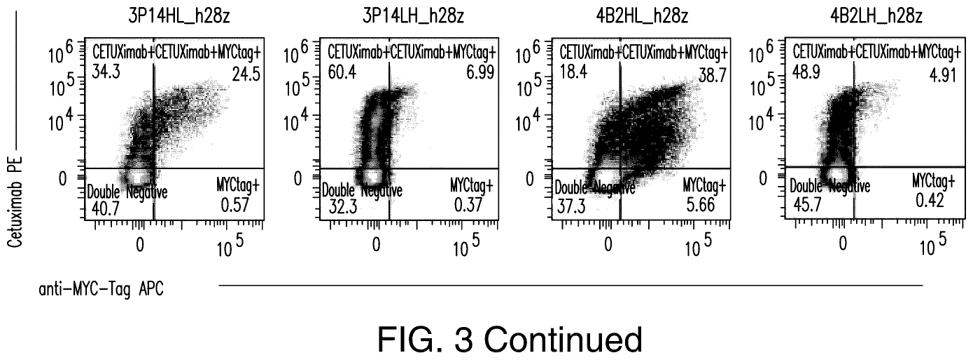

Retrovirus production was carried out using packaging cells. Peripheral blood mononuclear cells (PBMCs) were isolated from various donors and activated using standard methods. Activated T cells were transduced with virus supernatants produced with different CARs on day 3 and 4 post-activation. Cells were expanded and tested by flow cytometry using anti-EGFR and anti-Myc antibodies to monitor transduction efficiency and CAR expression, respectively. Overall, a greater than 60% transduction efficiency was achieved in various donors with CAR constructs. Among the two CARs targeting a membrane-proximal epitope of CD33, both 3P14 and 4B2 showed good surface expression in the V_{H}-V_{L} orientation only (see FIG. 3). The surface expression of the 5 CARs targeting a membrane-distal epitope varied from low to medium (data not shown).

### Example 2—In Vitro Cytotoxicity of CD33-CAR T Cells

The cytolytic capacity of CAR-engineered human T cells was assessed by killing assays using U937 or OCi-AML3 AML cell lines with a GFP-firefly luciferase reporter. CAR T cells were co-cultured with 1×10⁴ target cells at various effector to target ratios in triplicates in 96-well plates. Target cells were plated with non-signaling control CAR T cells at the same cell densities to determine maximal luciferase expression as a reference. Bioluminescence was measured 24 hours later, and percent lysis was determined as a percentage of killing observed with a non-functioning CAR T cell.

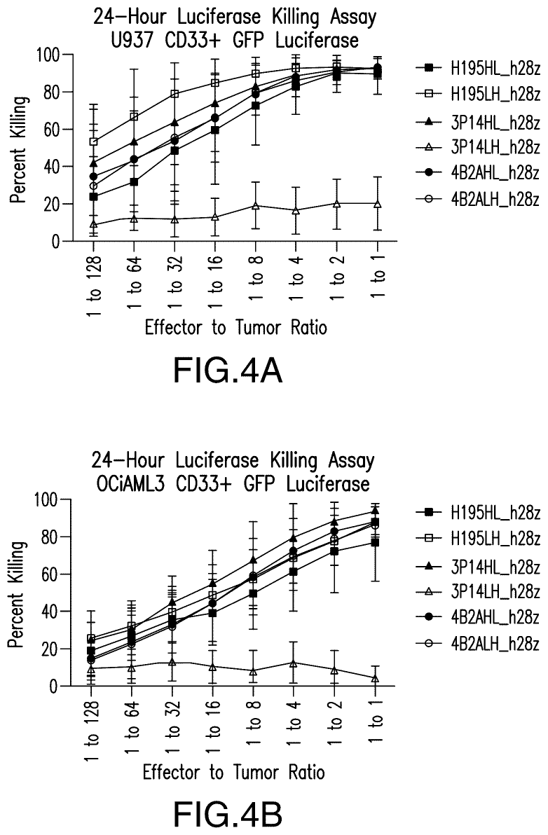

CARs with antibodies targeting the membrane-proximal IgC2 domain of CD33 were assessed on U937 (CD33high) and OCi-AML3 (CD33low) cell lines. 3P14 showed killing only in V_{H}-V_{L}orientation. This is consistent with a lack of surface expression of this scFv in vL-vH orientation. However, surprisingly, CARs with 4B2 showed comparable killing in both orientations even though vL-vH showed weak surface expression. Overall, both CARs showed a greater than 60% killing activity when compared to inactive CAR. The killing potency on both CD33high (U937) and CD33low (OCi-AML3) expressing cells was comparable although slightly different with low E/T ratio (see FIGS. 4A and 4B).

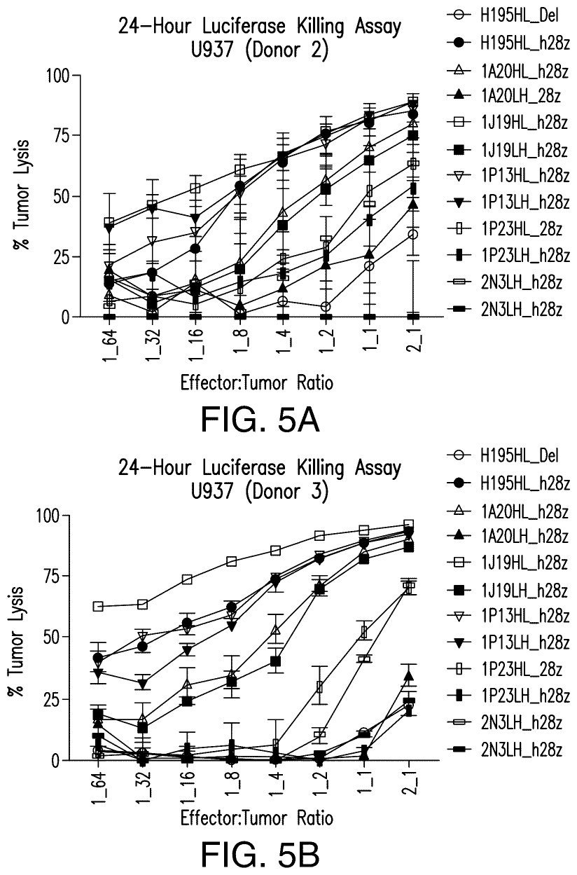

The cytotoxicity assessment of the CARs with the 5 antibodies targeting the membrane-distal IgV domain of CD33 was carried out in U937 with two different donors. One clone (1J19) showed stronger killing than the reference CAR made with the H195 (lintuzumab) antibody (see FIGS. 5A and 5B).

Based on these results, two membrane-proximal clones 3P14 and 4B2 in V_{H}-V_{L} scFv orientation (designated as “TDI-Y-006” and “TDI-Y-007”, respectively) and 1J19 were selected for further evaluation.

### Example 3—Functional Characterization of CD33-CAR T Cells

### Cytotoxicity.

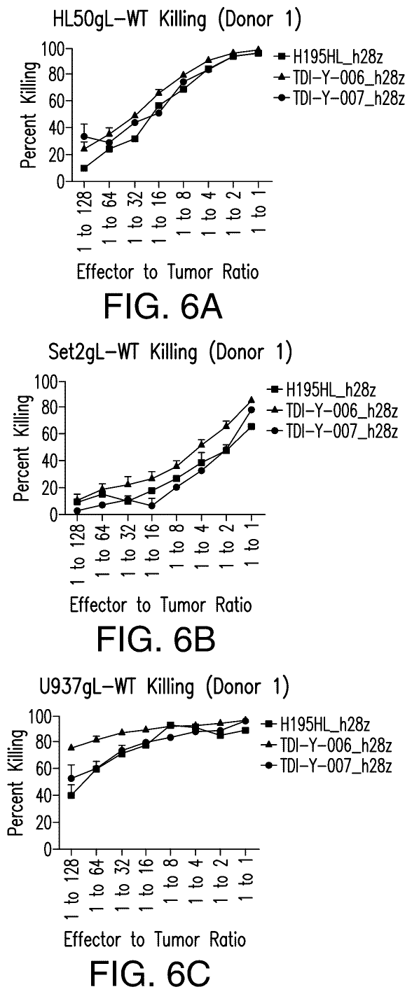

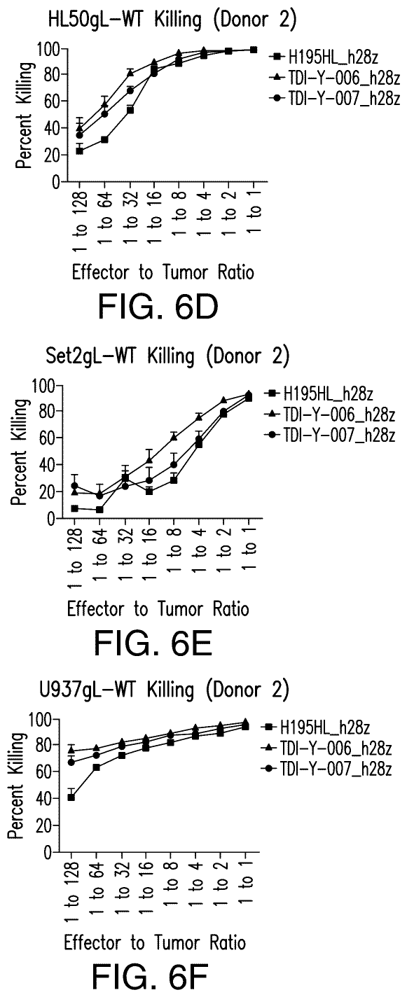

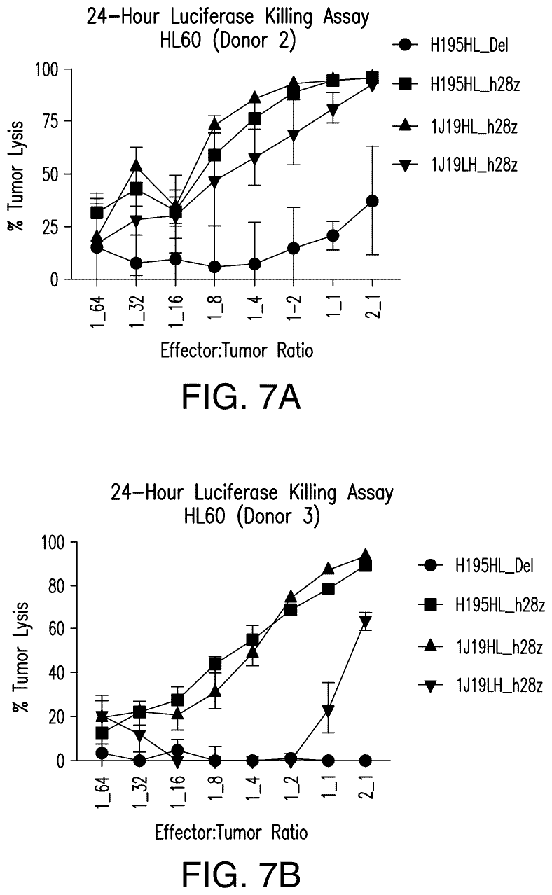

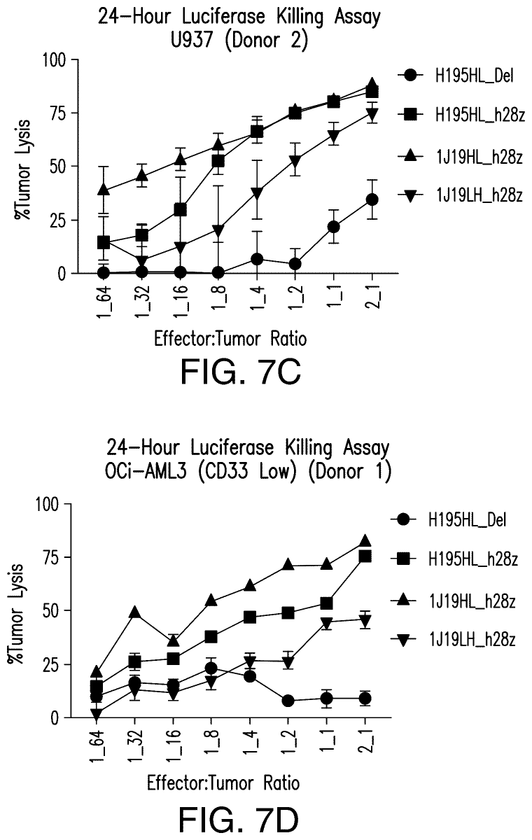

To assess the cytotoxicity of TDI-Y-006 and TDI-Y-007 CARs in vitro, CAR T cells were co-cultured with CD33-expressing human AML target lines. Three AML cell lines (U937, HL60 and Set2) were used. Based on the data of Example 2, only V_{H}-V_{L} orientation was selected for further characterization. A reference CAR with H195 in V_{H}-V_{L} orientation was included in all experiments. TDI-Y-006 and TDI-Y-007 CARs potently lysed various types of CD33-expressing AML target cells when engineered in T cells from donor 1 (see FIGS. 6A-6C) or donor 2 (see FIGS. 6D-6F). Overall, more than 80% killing was observed at the highest effector to target ratio. These data are consistent with the earlier evaluation of these clones during lead selection phase. 1J19 CAR lysed CD33-expressing target cells equally well when tested with different donors (see FIGS. 7A-7D). All three CAR T cells showed superior killing compared to the H195 reference CAR and a clear ranking in potency in the order of TDI-Y-006, TDI-Y-007, 1J19, and H195 was observed.

### Proliferation and Cytokine Profile.

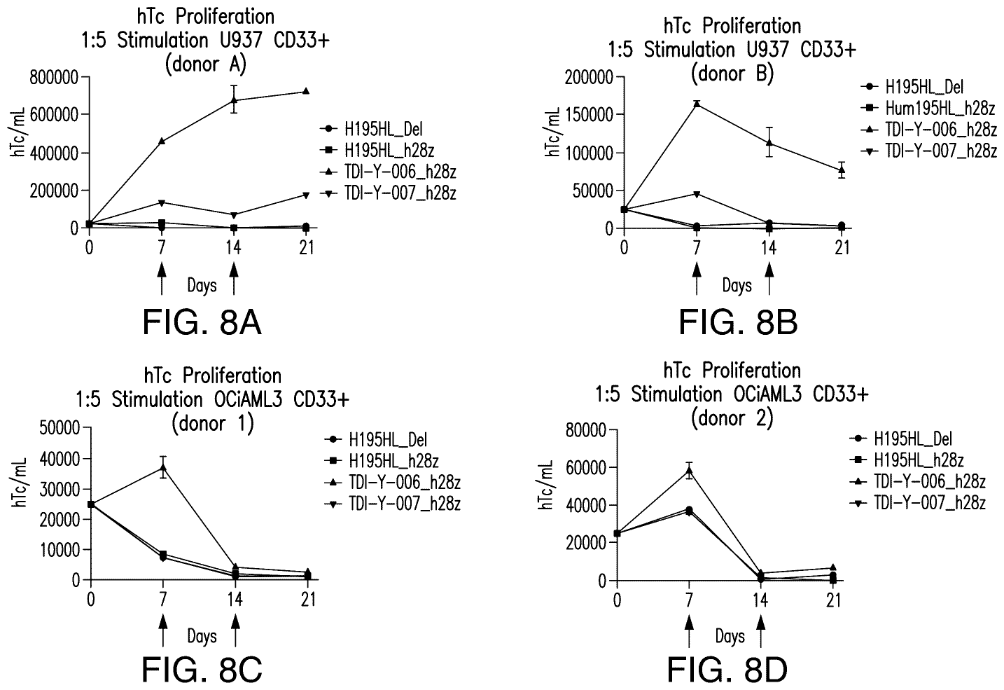

The in vitro T cell proliferation and cytokine activation profile of TDI-Y-006 and TDI-Y-007 CAR T were evaluated by co-culturing target and effector cells and measuring proliferation of T cells and cytokine levels. Briefly, CAR T cells from two different donors were co-cultured with U937 or OCi-AML3 AML cells at 1:5 tumor to CAR T cell ratio. Every seven days, flow cytometry was used to detect the remaining tumor and CAR T cells and then further stimulated with tumor at a 1:5 tumor to CAR T cell ratio. On U937 cells (CD33^{high}), TDI-Y-006 CAR T cells from both donors showed very strong proliferation followed by TDI-Y-007, which had moderate but still significant stimulation when compared to H195 reference CAR T. Similar results were observed with OCi-AML3 cells, with slight variation among two donors. In all these settings, H195 reference CAR T cells had background levels of proliferation only (see FIGS. 8A-8D). Taken together, these data suggest that TDI-Y-006 CAR T, and to a lesser extent TDI-Y-007 CAR T, are more potent than the H195 reference CAR T cell and exhibit superior proliferation when they bind to target cells.

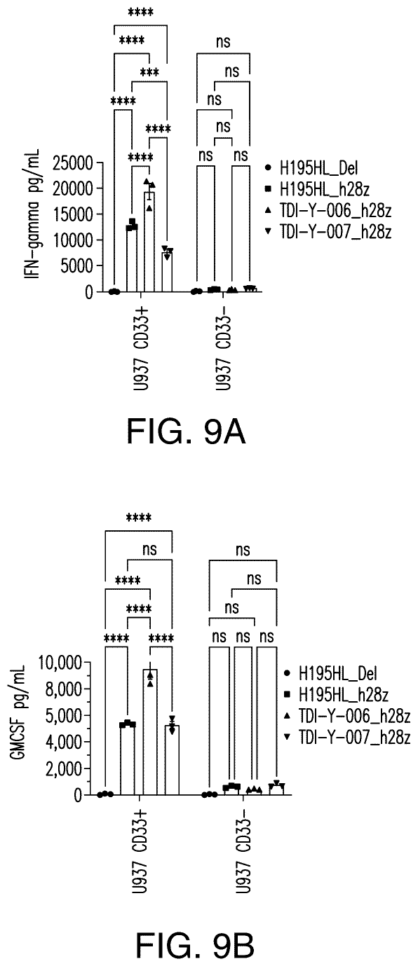

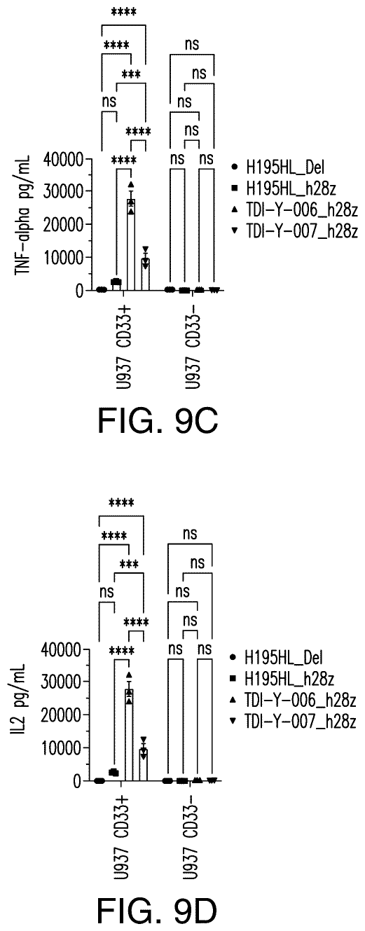

A panel of four cytokine markers (IL-2, GMCSF, IFN-gamma and TNF-alpha) was evaluated to assess the potency of each CAR. TDI-Y-006 CAR T cells derived from three healthy donors secreted high levels of IL-2, GMCSF, IFN-gamma and TNF-alpha in the presence of CD33-expressing U937 target cells at an E:T of 1:1 (40,000 CAR T cells: 40,000 U937 cells). FIGS. 9A-9D show representative data from one donor, while a consistent cytokine profile was seen across all three donors. The levels of cytokine secretion induced by TDI-Y-007 CAR T cells were 30% to 60% lower than the TDI-Y-006 CAR T depending on the donor. The level of cytokine secretion by the H195 reference CAR T was similar to TDI-Y-007 or further reduced depending on the donor. These data indicate that TDI-Y-006 and TDI-Y-007 CAR T induce strong cytokine production upon target cell engagement. The stronger effect seen for TDI-Y-006 compared to TDI-Y-007 is consistent with its superior proliferation ability and may correlate with the different affinities of the CARs for CD33 on cells.

The production and secretion of these cytokines likely enhances in vitro proliferation of the high-affinity TDI-Y-006 CAR T to a greater extent than the low-affinity TDI-Y-007 CAR T and the H195 reference CAR T. Even though H195 CAR T have a high affinity for the target, the cytokine secretion levels are generally lower than for the lead CARs, especially TDI-Y-006. This is likely due to the fact that the epitopes of TDI-Y-006 and TDI-Y-007, located in the membrane-proximal IgC2 domain. Binding of the CAR to a region of CD33 close to the membrane of the target cells may allow for the establishment of a stronger immune synapse and hence result in potent CAR T. Importantly, IL2 secretion was consistently higher in TDI-Y-006, followed by TDI-Y-007, indicating a potential for proliferation in the context of antigen-positive cells.

### In Vivo Efficacy: AML Xenograft Mouse Model.

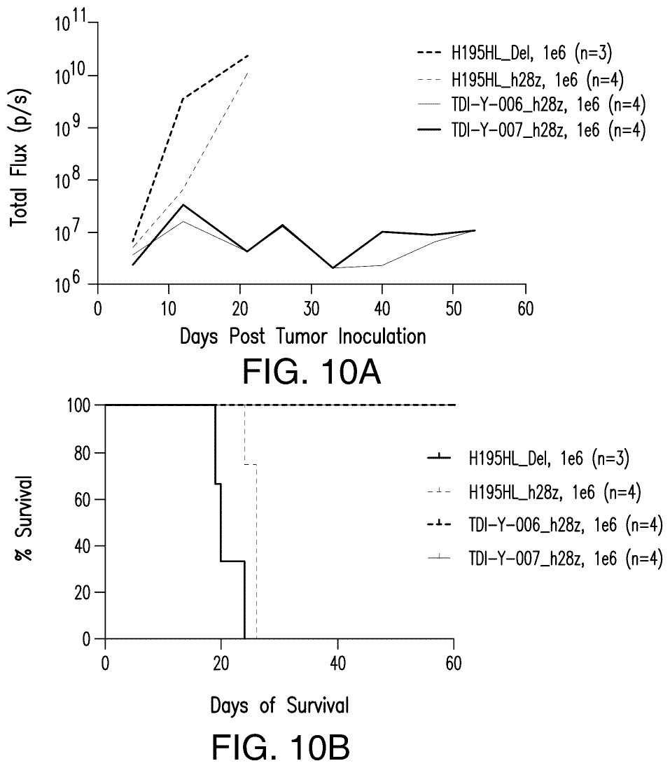

The in vivo efficacy of TDI-Y-006 and TDI-Y-007 CARs was assessed by injecting NCG mice with U937 tumor cells intravenously (IV), and infused with 1×10⁶ CAR T cells three days later. Mouse survival and overall tumor burden was tracked with bioluminescent imaging of the GFP-firefly luciferase-expressing tumor cells. Sustained tumor control was observed with the infusion of TDI-Y-006 and TDI-Y-007 CAR T cells (see FIG. 10A), whereas CAR T with the H195 reference antibody only minimally delayed tumor growth compared to a non-signaling H195 control. In addition to promoting consistent tumor regression, TDI-Y-006 and TDI-Y-007 CAR T cells substantially enhanced survival of tumor-bearing mice compared to H195 reference CAR T (see FIG. 10B).

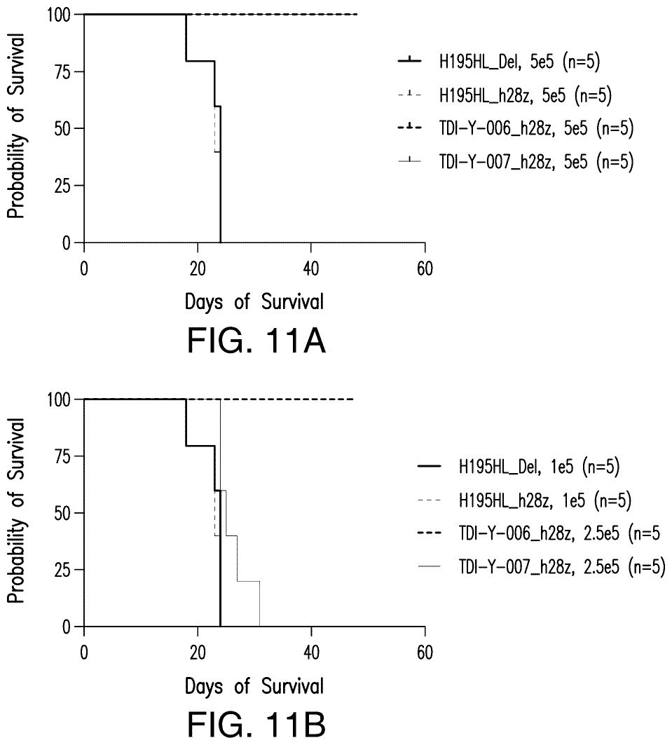

To further validate the in vivo efficacy of the lead CAR T cells, NCG mice were inoculated with U937 cells and treated three days later with reduced doses of TDI-Y-006 and TDI-Y-007 CAR T cells. Both CAR T cells exhibited comparable survival benefit over non-signaling or signaling H195 T cells at 5×10⁵ doses (see FIGS. 11A and 11B). Interestingly, a more subtle difference among TDI-Y-006 and TDI-Y-007 was seen when T cells were used at 2.5×10⁵ where TDI-Y-006 provided long-term survival to mice at this lower CAR T cell dose.

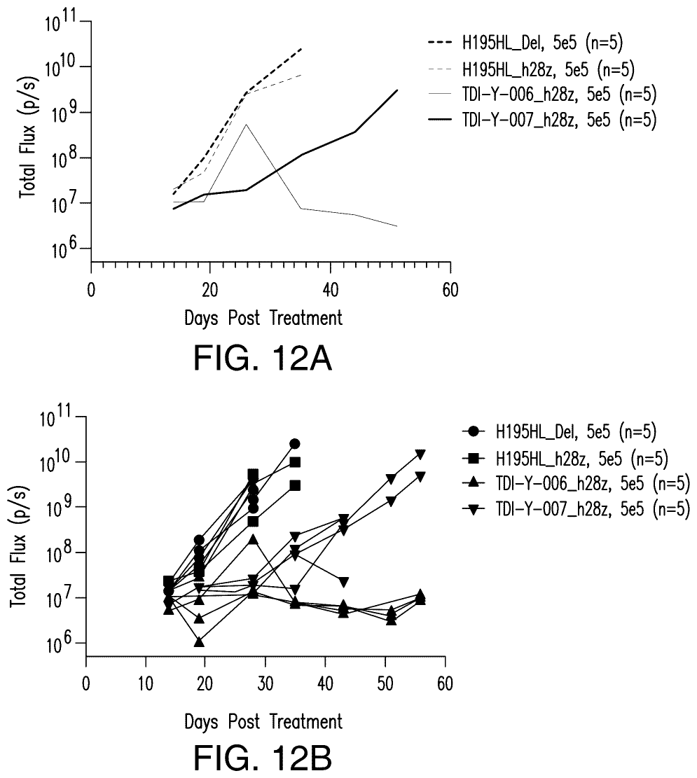

To recapitulate these results in a tumor model with cells expressing low levels of CD33, NCG mice were inoculated with OCi-AML3 tumor cells IV and infused with 5×10⁵ CAR T cells three days later. Mouse survival and overall tumor burden was tracked with bioluminescent imaging of the OCi-AML3 tumor cells, which were engineered to express GFP-firefly luciferase. TDI-Y-006 and TDI-Y-007 CAR T cells enhanced survival of tumor-bearing mice compared to the H195 reference CAR T cells. As seen in previous experiments, TDI-Y-006 CAR T cells showed the strongest tumor control by cohort averaged (see FIG. 12A) or individual animal imaging (see FIG. 12B).

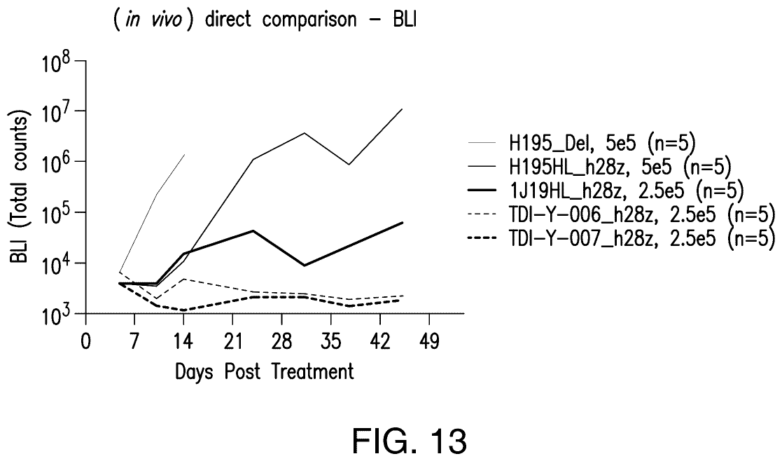

In order to assess the in vivo potency and ranking of two lead and one backup 1J19 CAR, a direct comparison was carried out using U937 xenograft. As shown in FIG. 13, a clear ranking in the order of TDI-Y-006, TDI-Y-007, 1J19 and H195 was observed. This was consistent with in vitro killing data and across multiple donors.

### In Vivo Efficacy: Patient-Derived Xenograft Mouse Model.

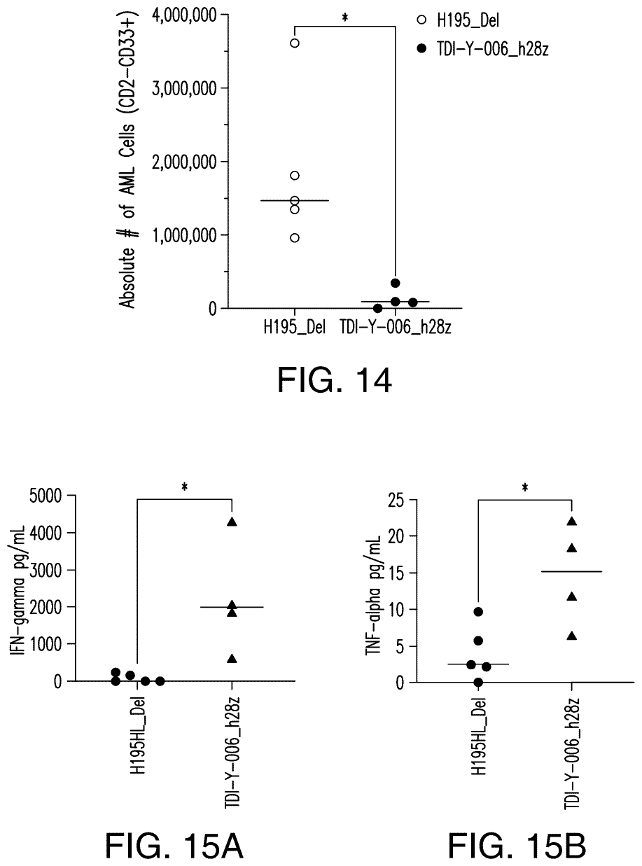

TDI-Y-006 CAR T cells, which showed the strongest efficacy in the AML cell line xenograft models described above, were further evaluated in an in vivo patient-derived xenograft (PDX) model. NSGS mice were sub-lethally irradiated with 250 cGy and infused intravenously with PBMCs derived from AML patients with peripheral blast. Tumor cells were allowed to grow for 10 days, at which point tumor engraftment was verified by flow cytometry staining of human CD45 in peripheral blood. Mice were infused with allogeneic CAR T cells 14 days after tumor inoculation. Seven days after T cell infusion, significantly less hCD45+, CD2−, CD33+ AML cells were present in the bone marrow of the TDI-Y-006 CAR T cells treated mice compared to mice treated with non-signaling control CAR T cells (see FIG. 14).

Furthermore, significantly elevated levels of the pro-inflammatory cytokines IFN-gamma and TNF-alpha were found in the serum of the mice treated with TDI-Y-006 CAR T cells (see FIGS. 15A and 15B), indicating strong T cell activation and proliferation in this model as well.

### Toxicity.

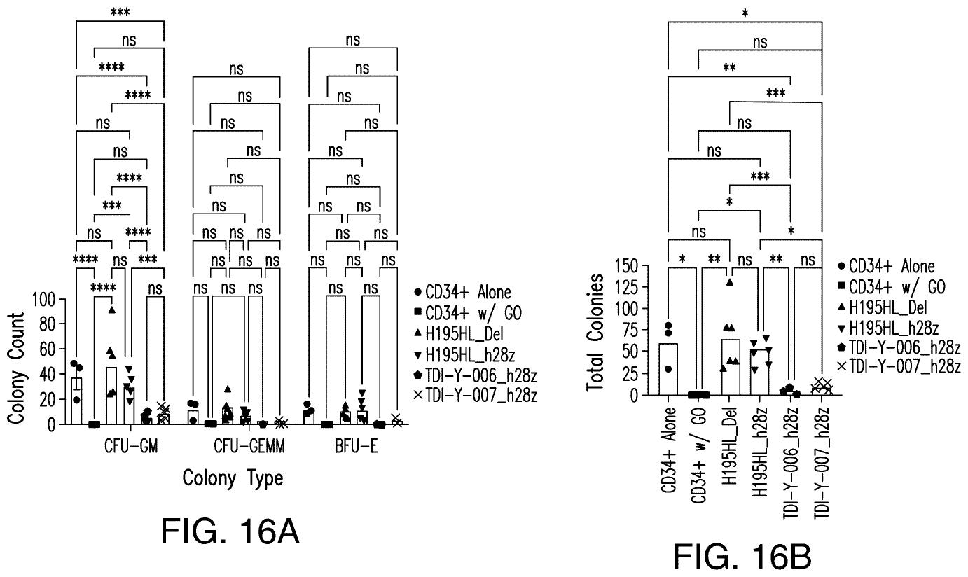

The hematopoietic stem cell killing capacity of the CD33 targeting CAR T cells in vitro was evaluated using a colony-forming unit (CFU) assay. Quantified by total colony count, TDI-Y-006 and TDI-Y-007 CAR T cells lysed CD34+ HSCs and was comparable to gemtuzumab ozogamicin (GO), which was used as positive control (see FIGS. 16A and 16B). These data suggest that like any other CD33-based therapy, these CAR T cells will most likely affect HSCs, as these cells express CD33. Therefore, their clinical use would require a rescue allogeneic stem cell transplant. This capacity of TDI-Y-006 and TDI-Y-007 to ablate HSCs could in fact be exploited to develop chemotherapy-free transplant regimens, as these CAR T cells will allow for engraftment of allogeneic cells without the need for bone marrow preparation with harsh cytotoxic agents. Furthermore, different dosing of CAR T cells could be employed to avoid targeting of antigen low population.

### Conclusions.

In summary, TDI-Y-006 and TDI-Y-007 are novel anti-CD33 antibodies which target membrane-proximal domain of antigen. These targets have shown superior in vitro and in vivo efficacy in CAR T format and outperformed an existing reference CAR Disease-relevant mouse models resulted in complete tumor eradication with substantial survival benefits. A summary of the pharmacological properties is provided in Table 12 below.

| | | |
|---|---|---|
| Parameter | TDI-Y-006 | TDI-Y-007 |

Molecular and Cellular Selectivity

| | | |
|---|---|---|
| Binding to NALM6 (CD33 negative | No binding at 100 | No binding at 100 |
| cell line) | μg/mL | μg/mL |

In vitro Potency-CAR T format

| | | |
|---|---|---|
| Cytotoxicity of CD33 High AML | >75% cell death | >65% cell death |
| cells (U937) | | |
| Cytotoxicity of CD33 low AML cells | >60% cell death | >50% cell death |
| (OCi-AML3) | | |

In vivo Efficacy-Mouse Models

| | | |
|---|---|---|
| Efficacy in U937 intravenous xenograft | Complete eradication | Complete eradication |
| model | of tumor implant and | of tumor implant and |
| | survival benefits over | survival benefits over |
| | 60 days post-xenograft | 60 days post-xenograft |
| Efficacy in AML patient-derived | Substantial decrease in | Not tested |
| peripheral blast xenograft | implanted cells, | |
| | significant elevated | |
| | levels of cytokine | |
| | markers of T cell | |
| | potency | |

In conclusion, these novel molecules have a great potential to be used as next generation AML CAR T therapies as a single agent or in combination with other antigens for complimentary approaches.

### Example 4—Developing a Membrane-Proximal CD33-Targeting CAR T Cell

The present example analyzes differences between the lintuzumab-CD28/CD3Z (H195HLh28Z) platform and a presently disclosed antigen-recognizing receptor that targets the membrane-proximal IgC domain of CD33 (CD33-IgC). The present example demonstrates that high-affinity CD33-IgC-targeting CAR T cells had greater proliferative capabilities and increased polyfunctionality in vitro compared to H195HLh28Z, leading to improved survival and tumor control in xenograft models of high and low antigen density and of high tumor burden. Surprisingly, low-affinity CD33 IgC-targeting CAR T cells demonstrated superior functionality compared to high-affinity, membrane-distal-targeting H195HLh28Z. Collectively, these data support targeting CD33-IgC to markedly enhance the efficacy of CAR T cells for AML treatment.

### Methods

### Animal Models.

For xenogeneic studies, NOD-PrkdC^{em26Cd52}Il2rg^{em26CD22}/NjuCrl, Coisogenic Immunodeficient (NCG) mice were purchased from Charles River and subsequently bred and housed under specific-pathogen-free (SPF) conditions in the animal facility. For all experiments, 6- to 12-week-old mice were used. For CD33-high tumor studies, 5×10⁴ U937 AML cells expressing green florescent protein (GFP)-firefly luciferase were inoculated on day 0. For CD33-low tumor studies, 5×10⁵ OCiAML3 AML cells expressing GFP-firefly luciferase were inoculated on day 0. In both models, mice were treated with varying doses of CAR T cells 3 days after tumor inoculation. For patient-derived AML xenograft animal studies, NCG mice were inoculated with 1×10⁶primary AML blasts and treated with 1×10⁶ CAR T cells on day 14.

### Cell Lines.

Two-hundred-ninety-three Glv9-packaging cells were maintained in DMEM (Dulbecco's Modified Eagle Medium) with high-glucose supplemented with 10% heat-inactivated FBS (fetal bovine serum) nonessential amino acids (Atlanta Biological Flowery Branch), 2 mM L-glutamine (Invitrogen), and 1% penicillin/streptomycin (Invitrogen). The U937 human acute leukemia line, the OCiAML3 human acute myeloid leukemia line, and the HL60 human acute myeloid leukemia line were modified to express GFP-firefly luciferase to detect tumor in vitro and in vivo by luminescence. All tumor cell lines were maintained in RPMI-1640 medium supplemented with 10% heat-inactivated FBS nonessential amino acids (Atlanta Biological Flowery Branch), 10 mM HEPES (hydroxyethyl piperazineethanesulfonic acid, Invitrogen), 2 mM L-glutamine (Invitrogen), 1% penicillin/streptomycin (Invitrogen), and 11 mM glucose (Invitrogen). Tumor lines were sorted by fluorescence-activated cell sorting (FACS) based on high expression of GFP. Cell lines were routinely tested for potential mycoplasma contamination.

Selection of scFv.

Antibody generation and characterization studies were carried out by the Tri-Institutional Therapeutics Discovery Institute (TriI-TDI) utilizing AlivaMab® transgenic mice (Ablexis) with extracellular domain of CD33 recombinant proteins either sourced commercially or produced in-house. Recombinant human CD33 protein was sourced commercially from R&D Systems and Acro Biosystems. Mouse and cynomolgus monkey CD33 proteins were purchased from Sino Biological. In addition, human CD33-IgC domain (residue 140-259) with a terminal mouse IgG1 Fc or 6×His tag was produced in-house. Immunizations and screenings were carried out at LakePharma (Belmont, CA). Multiple cohorts of AlivaMab® mice were immunized with recombinant proteins, with one cohort receiving CD33-IgC. Serum was collected on days 17, 24, 28, and 31, and the immune response was analyzed by enzyme-linked immunosorbent assay (ELISA) using recombinant human full-length CD33-6×His and CD33 IgC2-6×His proteins. Hybridoma were generated by electrofusion, and the supernatants were screened by ELISA on recombinant proteins as well as by flow cytometry on 3T3 cells overexpressing either full-length CD33 or CD33-IgC alone and AML cell lines with endogenous CD33 expression. Subcloning was performed by limited dilution, and sequencing of the top 10 lead candidates was carried out using standard IgG primers recommended by Ablexis.

### Binding Selectivity and Affinity.

Lead and reference antibodies were produced recombinantly with a human IgG1 constant region. Selectivity was tested using His tag recombinant human full-length CD33 or CD33-IgC, mouse full-length CD33, and cynomolgus full-length CD33 proteins. Recombinant proteins at 5 μg/mL were captured by pre-blocked Ni-NTA plates. Candidate antibodies were added at 10 μg/mL in triplicate and detected using horseradish peroxidase (HRP)-conjugated anti-human Fc antibody. To assess the binding to cell-surface-bound CD33, 3T3 cells overexpressing CD33, as well as the U937 AML cell line, were used. U937 CD33-knockout (CD33KO) or NALM6 cells were included as negative controls. NALM6, U937 CD33⁺, and CD33KO cells were blocked with human IgG Fc for 20 minutes on ice. The recombinant antibodies were then serially diluted starting at 100 μg/mL concentration and added for 30 minutes on ice. Alexa Fluor 647-conjugated goat anti-human F(ab′)₂ was added to cells for 30 minutes on ice, washed and analyzed by flow cytometry, and normalized to secondary-only staining (MFI ratio). EC₅₀ values were determined by non-linear regression. The binding affinity and epitope binning to human CD33 protein was measured by biolayer interferometry (BLI) using an Octet Red96e. All experiments were carried out using kinetic buffer (PBS pH 7.4, 0.01% BSA, 0.002% Tween-20). For affinity measurements, the antibodies were captured by an anti-huFc biosensor, and a 7-point, 2-fold dilution series of huCD33-His was used as analyte. The data were processed by double reference subtraction, and response curves were globally fit to a 1:1 Langmuir binding model.

### Epitope Binning.

For epitope binning, human CD33-His was captured by an anti-penta-His biosensor. A 3×3 matrix with 3P14, 4B2A, and H195 was tested. A buffer-only reference biosensor was used to determine the overall capture level for each antibody. After pre-binding the antibodies at saturation levels, the biosensors were dipped into antibody solutions to assess competition.

### Generation of Retroviral Constructs.

Constructs were cloned into the SFG gammaretroviral vector with human signaling domains. Retroviral producer cell lines were generated by using CaPO4 (Promega) according to the manufacturer's instructions to transiently transfect gpg29 fibroblasts (H29) with retroviral constructs encoding the CAR. Supernatant from the H29 cells was used to transduce 293Glv9 to produce stable retroviral producer cell lines.

### T Cell Isolation and Retroviral Transduction.

Briefly, peripheral blood mononuclear cells (PBMCs) were isolated from healthy donor peripheral blood or leukopacks (New York Blood Center). Following red blood cell lysis with ACK (Ammonium-Chloride-Potassium) Lysing Buffer (Lonza), human T cells were isolated from PBMCs (StemCell Technologies) and subsequently activated with 100 IU/mL of IL-2 and Dynabeads Human T-Activator CD3/CD28 at a bead:cell ratio of 1:5 (Thermo Fisher Scientific). Forty-eight hours after initial expansion, T cells were spinoculated with viral supernatant collected from 293Glv9 packaging cells on RetroNectin-coated plates on 2 consecutive days (Takara Clontech). Transduction efficiency was determined by flow cytometric analysis. All experiments were normalized for CAR+ viable cells.

### Flow Cytometry.

Flow cytometric analyses were performed using 10-color Gallios B43618 (Beckman Coulter, Indianapolis, IN) and 14-color Attune NxT (Thermo Fisher Scientific) instruments. Data were analyzed using FlowJo (Tree Star). Flow cytometry was used to determine transduction efficiency of transduced cells following staining with phycoerythrin (PE) conjugated cetuximab antibody and Myc-tag (9B11, Alexa Fluor 647, Cell Signaling). DAPI (0.5 mg/mL, Sigma Aldrich) or 7-amino-actinomycin D (0.05 mg/mL, BioLegend) and PO-PRO-1 Iodine (Invitrogen) staining were used to exclude dead cells in all experiments. The following anti-human antibodies were used for flow cytometry: anti-CD4 (RPA-T4), anti-CD8 (SK1), anti-62L (DREG-56), anti-CD45RA (HI100), anti-CD2 (TS1/8), anti-CD371 (50C1), anti-CD45 (2D1), anti-CD33 (WM53), and anti-CD123 (6H6). The following anti-mouse antibodies were used for flow cytometry: anti-CD45 (2D1). All antibodies were purchased from BioLegend, BD Biosciences, Invitrogen, or eBioscience.

### In Vitro Colony Forming Unit Assay.

PBMCs were isolated from healthy umbilical cord blood units (New York Blood Center). Following red blood cell lysis with ACK Lysing Buffer (Lonza), CD34⁺ stem cells were isolated and subsequently expanded in Serum-Free Expansion Medium (SFEM) II and 1.0 μM UM171(StemCell Technologies). Expanded CD34⁺ progenitors and CAR T cells were co-cultured at 1:1 cell ratio for approximately 48 hours. Treated human CD34⁺ progenitors were seeded at a density of 3,333 cells/condition into cytokine-supplemented methylcellulose medium (MethoCult H4435; STEMCELL Technologies) on 6-well plates. Each condition was cultured in triplicate. Colonies were propagated in culture and scored at day 10 and counts were averaged across replicates.

### Cytotoxicity Assays.

The cytolytic capacity of CAR-modified human T cells was assessed through a luciferase-killing assay. CAR T cells were co-cultured with 1×10⁴ target cells, U937-gfpLuc⁺ or OCiAML3-gfpLuc⁺ tumor cells, at various effector-to-target ratios in quadruplicates in white-walled 96-well plates (Thermo Scientific) in a total volume of 200 μL of cell media. Target cells were plated with non-signaling control CAR T cells at the same cell densities to determine maximal luciferase expression as a reference (max signal). Twenty-four hours later, 15 μg D-Luciferin (Gold Biotechnology) dissolved in 50 μL PBS was added to each well. Emitted luminescence of each sample (sample signal) was detected in a Spark plate reader (Tecan) and measured using SparkControl software (Tecan). Percent lysis was determined as [100-(sample signal/average max signal)]×100. For long-term killing assays, CAR T cells and tumor cells were co-cultured at an effector-to-tumor ratio of 1:40 in duplicates in white-walled 96-well plates (Thermo Scientific) in a total volume of 200 μL of cell media, and imaged over the course of 138 hours with the Sartorius IncuCyte S3. Tumor cell killing was measured as total green count over time.

### Proliferation Assays.

CAR T cells were co-cultured with U937-gfpLuc⁺ tumor cells or OCiAML3-gfpLuc⁺ tumor cells at 1:5 CAR T:tumor cell ratio. Following 7 days, flow cytometry was used to detect tumor and T cells following staining with fluorescently labeled cetuximab. CAR T cells were re-stimulated with fresh tumor cells at the same 1:5 CAR T:tumor cell ratio. The procedure above was repeated weekly for 21 days after initial stimulation.

### In Vitro Cytokine Secretion Analysis.

To measure in vitro T cell cytokine production, CAR T cells were co-cultured in a 1:1 ratio for 24 hours with antigen positive tumor cells or antigen negative tumor cells in a 96-well round-bottom plate. 24 hours later, the supernatant fluid was collected and analyzed for cytokines on a Luminex IS100 instrument. Luminex FlexMap3D system, Luminex xPONENT 4.2, and 12-plex Human panel (Millipore) were used to detect cytokines.

Intracellular Flow Cytometry for Polyfunctionality. To measure intracellular T cell cytokine and granzyme B production, CAR T cells were co-cultured at a 1:5 with U937-gfpLuc⁺ tumor cells. Approximately 16 hours later, a 500× Protein Transport Inhibitor cocktail was added to cell culture (eBiosciences), and 6 hours later cells were collected, stained with Fixable Yellow Dead stain (ThermoFisher Scientific), and fixed and permeabilized with BD Cytofix/Cytoperm Fixation and Permeabilization Solution (BD Biosciences) as per the manufacturer's suggested protocol. Cells were counted and stained with the following human antibodies: cetuximab conjugated in-house with (phycoerythrin) PE, anti-CD4 (RPA-T4), anti-CD8 (SKI), anti-IL2 (MQ1-17H12), anti-TNF-alpha (MAb11), anti-IFN-gamma (4S.B3), and anti-granzyme B (QA16A02).

### Multiparametric Flow Cytometric Analysis.

Cells were washed with PBS, resuspended in PBS at a concentration of 3.0×10⁶/mL, and incubated with Human TruStain FcX Fc receptor blocking solution (BioLegend) and Live/DEAD Fixable Blue Dead Cell Stain (Invitrogen) according to the manufacturers' specifications for 20 minutes at room temperature, protected from light. The cells were then washed once in Flow Wash Buffer (FWB; RPMI 1640, no phenol red+4% FBS+0.01% sodium-azide). Cells were incubated with the antibody mix for 20 minutes at room temperature in the dark in 100 μl staining volume in the presence of Super Bright Staining Buffer (eBiosciences), washed twice in FWB, resuspended in 0.5% paraformaldehyde/PBS, and immediately acquired using a Cytek Aurora 5L flow cytometer (Cytek). Analysis was performed with FlowJo v10.8.0.

### In Vivo Experiments.

For CD33-high expressing tumor studies, NCG mice were inoculated via tail vein with 5×10⁴ U937-gfpLuc⁺ tumor cells on day 0. On day 3, mice were blindly randomized into different treatment cohorts with CAR T cells treated with 5×10⁴ to 5×10⁵ CAR T cells via tail vein. For CD33-low expressing tumor studies, NCG mice were inoculated via tail vein with 5×10⁵ O CiAML3-gfpLuc⁺ tumor cells on day 0. On day 3, mice were blindly randomized into different treatment cohorts with CAR T cells treated with 5×10⁵ CAR T cells via tail vein. Both U937-gfpLuc⁺ and OCiAML3-gfpLuc⁺ tumor cells produced very even tumor burdens and no mice were excluded prior to treatment. Day 0 bioluminescence was assigned to be 1×10⁷. Tumor burden was measured weekly using bioluminescence imaging using the Xenogen IVIS Imaging system (Xenogen) with Living Image software (Xenogen). For the AML60B patient-derived xenograft model, viably cryopreserved primary AML specimens were obtained via Institutional Review Board-approved research protocols of MSK. NCG mice were inoculated with 1×10⁶ cells on day 0. On day 14, mice were randomized into different treatment cohorts and subsequently treated with 1×10⁶ CAR T cells via tail vein. On day 28, bone marrow aspirations were performed, and tumor cells were quantified by flow cytometry.

### Statistical Analysis.

All statistical analyses were performed using GraphPad Prism software (GraphPad). Data points represent biological replicates and are shown as the mean±standard error of the mean (SEM) as indicated in the figure legends. Statistical significance was determined by paired t-test, one-way ANOVA, or two-way ANOVA as indicated in the figure legends. The log-rank (Mantel-Cox) test was used to determine statistical significance for overall survival in mouse survival experiments. Significance was indicated with *, P<0.05; **, P<0.01; ***, P<0.001; and ****, P<0.0001.

### Results.

### CD33-IgC Immunization is Necessary for Discovery of Site-Specific Binders.

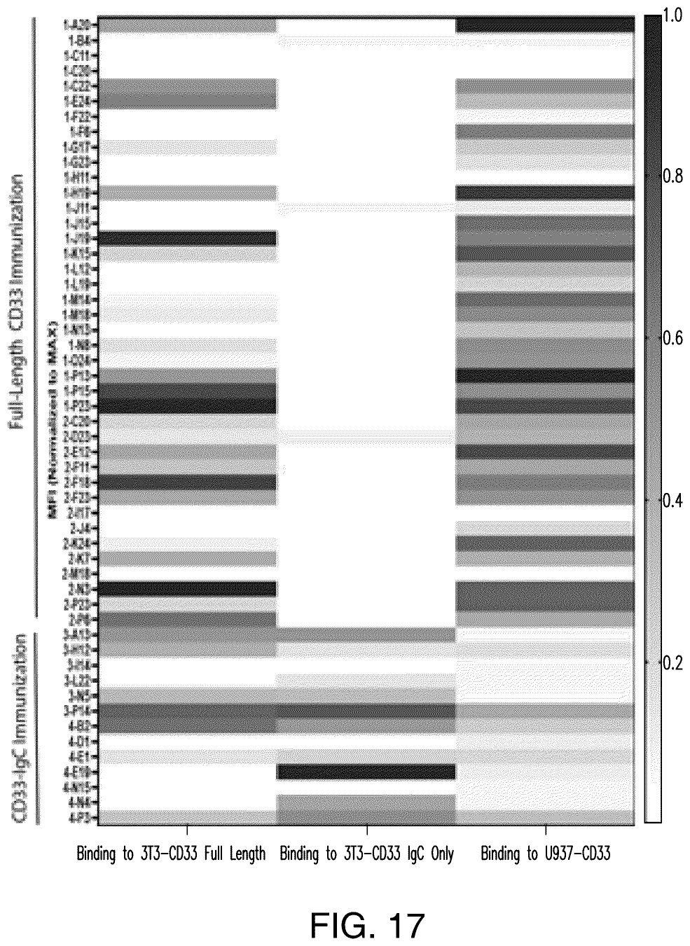

To generate CD33-IgC-specific antibodies, mice were immunized with either full-length CD33 or CD33-IgC recombinant proteins, as it was unknown whether the distal IgV domain would be critical to retain an IgC conformation necessary for immunological recognition. Hybridomas were generated and 53 hits were identified by ELISA using recombinant human CD33. On-cell binding was assessed by flow cytometry on 3T3 cells expressing either full-length CD33 or CD33-IgC, as well as U937 (AML cell line) with endogenous CD33 expression (FIG. 17). The specificity of binders was tested by screening CD33-negative lines such as U937 with CD33 knocked-out using CRISPR/Cas9 or NALM6 (CD33-negative cell line). Interestingly, all CD33-IgC domain-specific antibodies exhibiting consistent binding to various cell lines were derived from the CD33-IgC immunized cohort only. From this group, 3 hybridomas were selected and sequenced, resulting in the identification of 2 unique clones, 3P14 and 4B2A.

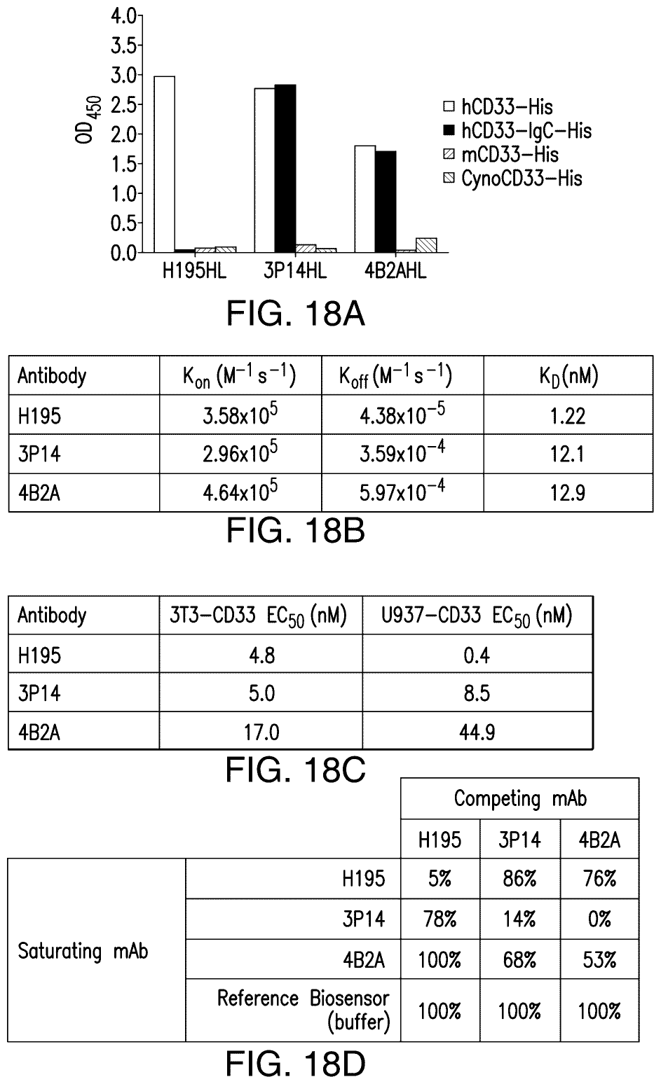

Next, it was confirmed the binding specificity and affinity of 3P14, 4B2A, and a reference CD33 IgV-specific monoclonal antibody (mAb), lintuzumab (H195). Antibodies were recombinantly expressed as human IgG1 sub-class. All mAbs bound full-length human CD33, but not cynomolgus or mouse orthologs, and as expected only 3P14 and 4B2A bound human CD33-IgC (FIG. 18A).

Utilizing biolayer interferometry (BLI), all mAbs had similar binding affinities for recombinant full-length CD33, with similar dissociation constants (K_{D}) and on-rates, but with a log lower off-rate for H195 compared with 3P14 and 4B2A (FIG. 18A). Given that mAbs could have varying affinities for soluble versus cell-bound antigen, their binding to cell-surface CD33 was measured utilizing 3T3 cells over-expressing the antigen (3T3-CD33) as well as a relevant AML tumor cell line, U937, which has endogenous expression of CD33. The hierarchy of affinity, from highest to lowest, was H195>3P14>4B2A, with the most pronounced differences noted for U937 cells (FIGS. 18B and 18C).

Next, epitope binning with immobilized CD33 was used to determine if 3P14 and 4B2A bound to similar epitopes, and if their differing affinities influenced binding of one antibody over another. It was observed that, when 3P14 was the saturating mAb, 4B2A was unable to demonstrate binding (0%), and when 4B2A was the saturating mAb, 3P14 was able to bind (68%); no binding competition was observed between H195 and either 3P14 or 4B2A (FIG. 18D), indicating that while 3P14 and 4B2A may have overlapping epitopes, both differed from membrane-distal H195.

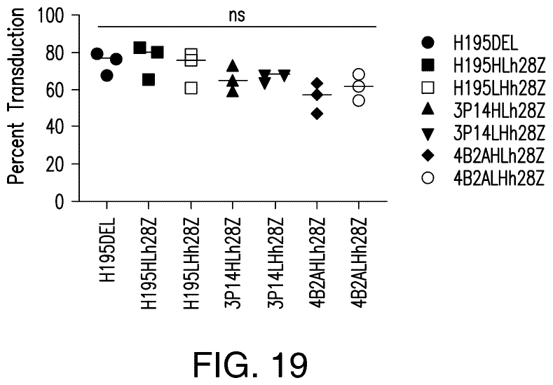

Next, single-chain variable fragments (scFvs) of H195, 3P14, and 4B2A were generated in both the VH-VL (HL) and VL-VH (LH) orientations, and cloned these into a bicistronic vector containing a truncated epidermal growth factor receptor tag (EGFRt), and a Myc-tag in line with human CD28/CD3Z (h28Z) signaling motifs (FIG. 2). For consistency, transduction was assessed by EGFR positivity, which was similar across all constructs (FIG. 19). Myc-tag staining confirmed that the HL formats more reliably demonstrated cell-surface CAR expression (FIG. 3).

These data show that CD33-IgC immunization is critical for raising binders to this domain, and that 3P14 binds with a higher affinity to CD33-IgC and likely recognizes a similar epitope to 4B2A, given the capacity of the former to displace the latter during epitope binning. These data also suggest that, because the HL scFv orientation more consistently demonstrates CAR surface expression of our candidate binders, it is the preferred format for preclinical testing.

### CAR T Cells Targeting CD33-IgC Demonstrate Enhanced Functionality In Vitro.

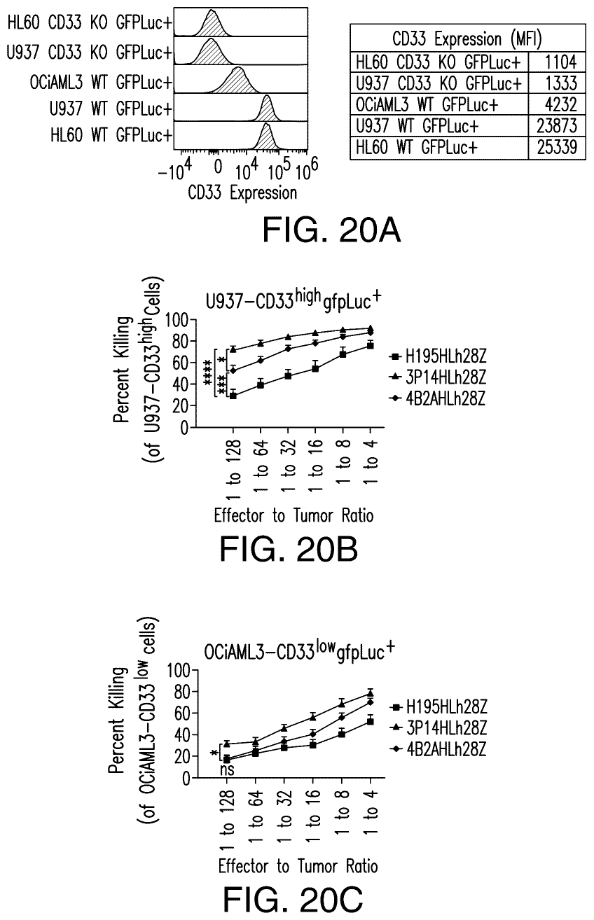

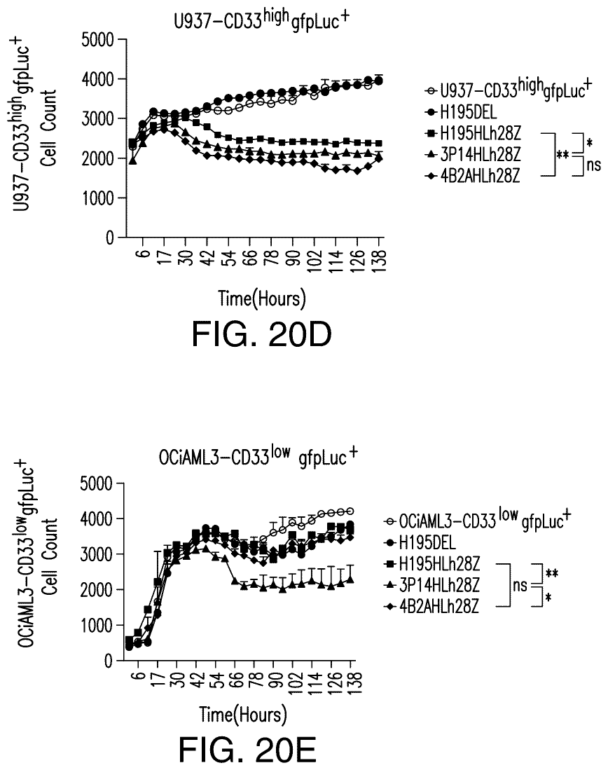

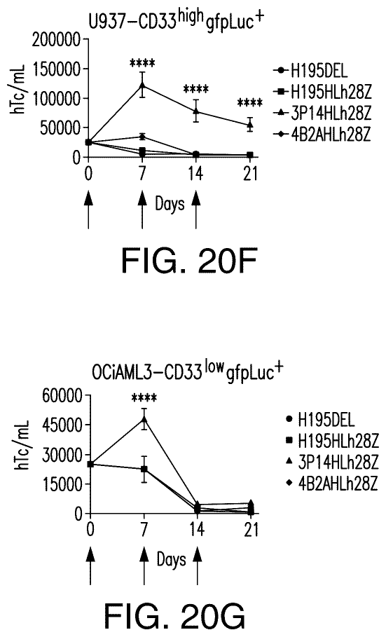

Low surface antigen density and excessive tumor burden are major mechanisms of resistance and treatment failure, leading to impaired tumor control and reduced effector expansion of CAR T cells (Majzner et al., Cancer Discov. 10, 702-723 (2020); Spiegel et al., Nat. Med. 27, 1419-1431 (2021); Locke et al., Blood Adv 4, 4898-4911 (2020)). The CT or TT genotypes of the rs12459419 C>T SNP are present in ˜50% of patients with AML, resulting in low CD33 surface expression on malignant cells. Utilizing flow cytometry, CD33 expression levels were determined on 3 cell lines with s12459419 C>T SNP genotype reported: HL60 (CC), U937 (CT), and OCiAML3 (TT) (Godwin et al., Leukemia 35, 2496-2507 (2021)). While HL60 and U937 cell lines expressed equivalent CD33 expression, U937 was selected to characterize a high-expressing CD33 model (U937-CD33^{high}) and OCiAML3 to characterize a low-expressing CD33 model (OCiAML3-CD33^{low}) given the reported genotypes (FIG. 20A). Target lines were transduced with a GFP-Firefly luciferase fused gene (gfpLuc) to generate U937-CD33^{high} gfpLuc⁺ and OCiAML3-CD33^{low}gfpLuc⁺ for in vitro tracking (data not shown).

Next, the capacity of CD33-IgC-specific CAR T cells to kill tumor cells with high and low CD33 antigen density was determined. In 24-hour killing assays at varying effector-to-target (E:T) ratios (1:4 to 1:128), 3P14HLh28Z and 4B2AHLh28Z showed increased dose-dependent killing of U937-CD33^{high} at low E:T ratios compared to H195HLh28Z, but only 3P14HLh28Z demonstrated enhanced cytotoxicity against OCiAML3-CD331^{low} cells at the same low ratio (FIGS. 20B and 20C). In 6-day long-term killing assays utilizing E:T ratios of 1:40, 3P14HLh28Z and 4B2AHLh28Z showed superior tumor control of U937-CD33^{high}gfpLuc⁺ compared to H195HLh28Z (FIG. 20D), while in the setting of OCiAML3-CD33^{low}gfpLuc⁺, 3P14HLh28Z showed superior killing compared to both 4B2AHLh28Z and H195HLh28Z (FIG. 20E).

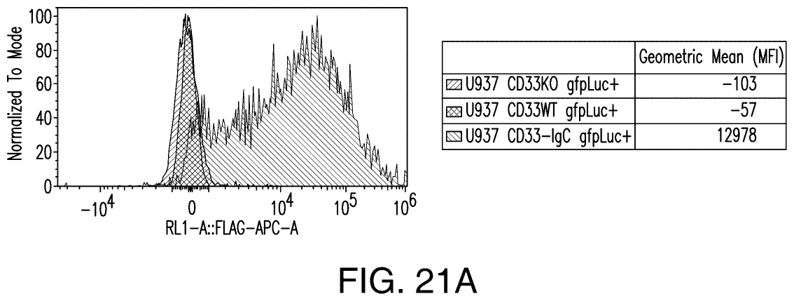

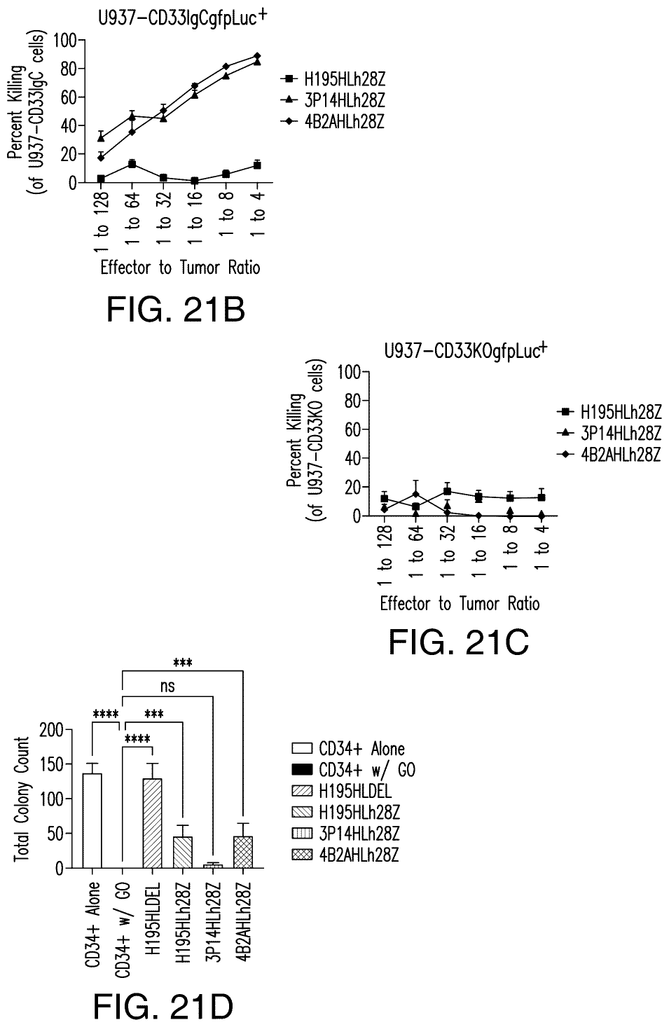

To validate CD33-IgC specificity of 3P14HLh28Z and 4B2AHLh28Z, U937-CD33^{KO}gfpLuc⁺ cells were transduced with a retroviral vector encoding a modified FLAG-tagged CD33-IgC, and surface expression was confirmed by flow cytometry (FIG. 21A). Compared to H195HLh28Z, both 3P14HLh28Z and 4B2AHLh28Z lysed target cells in a dose-dependent manner (FIG. 21B). Importantly, no CAR T cell lysed the negative control U937-CD33^{KO}gfpLuc⁺ (FIG. 21C). Given the reported expression of low levels of CD33 on HSCs, a colony-forming unit (CFU) assay was used to determine if 3P14HLh28Z and 4B2AHLh28Z could detect and eliminate these cells. Complete ablation of colonies was observed with only 3P14HLh28Z and high-dose gemtuzumab ozogamicin (GO), which served as a positive control, and incomplete ablation of colonies with either 4B2AHLh28Z or H195HLh28Z (FIG. 21D).

Given the relevance of CAR T cell expansion to clinical efficacy, the proliferative capacity of 3P14HLh28Z and 4B2AHLh28Z was tested in the context of recursive stimulation with high and low CD33 antigen density targets. 3P14HLh28Z had increased expansion and persistence compared to other CAR T cells when co-cultured with U937-CD33^{high}gfpLuc⁺ (FIG. 20F) and OCiAML3-CD33^{low}gfpLuc⁺ cells (FIG. 20G), although the magnitude of expansion and persistence was dampened in the latter, as expected given low antigen density.

In summary, these data show that the high-affinity CD33-IgC-specific 3P14HLh28Z is superior functionally to low-affinity CD33-IgC-specific 4B2AHLh28Z and high-affinity CD33-IgV-specific H195HLh28Z in the setting of high-tumor burden, as assessed by recursive stimulation or low-antigen-density encounters. Furthermore, low-affinity, membrane-proximal 4B2HLh28Z exhibited superior CAR T activity compared with high-affinity, membrane-distal H195HLh28Z, underscoring the importance of antigen target proximity in the context of CD33 CAR T cells.

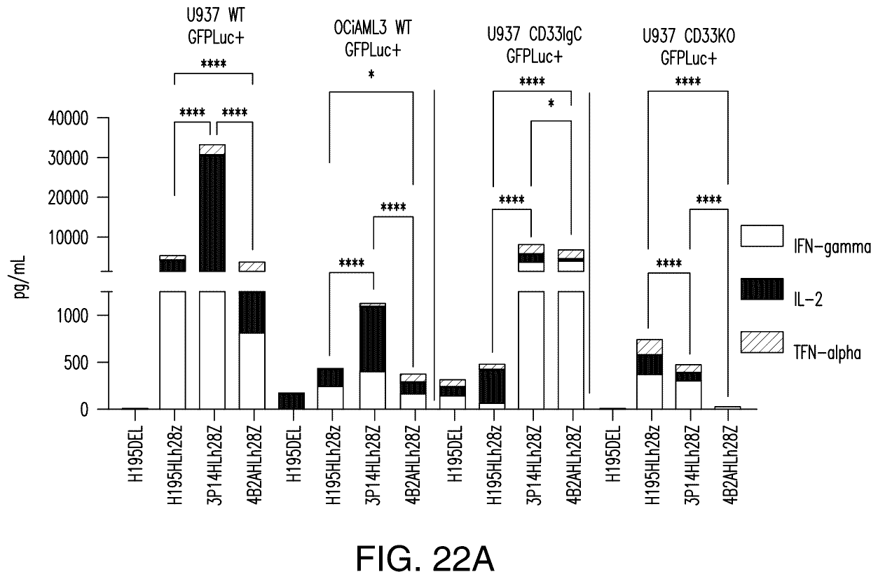

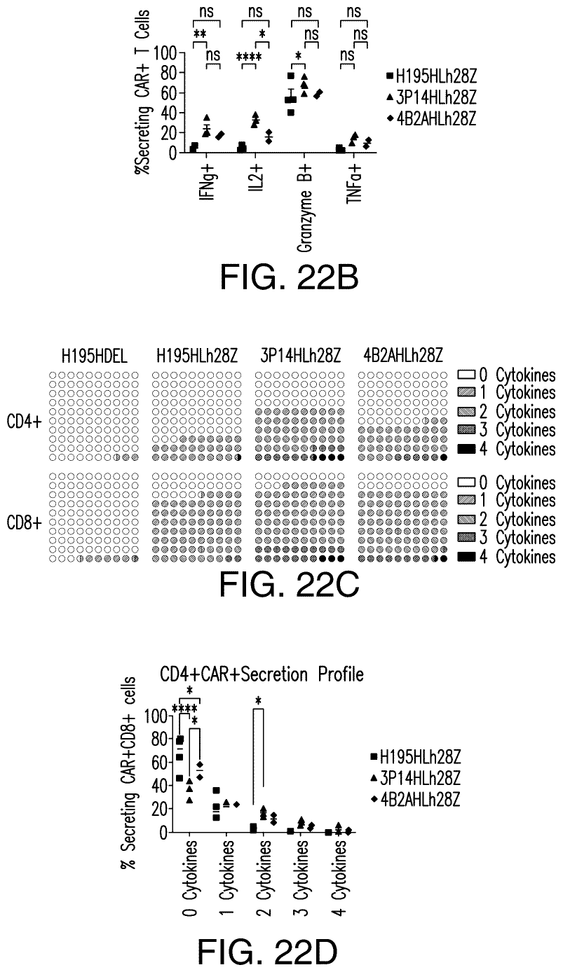

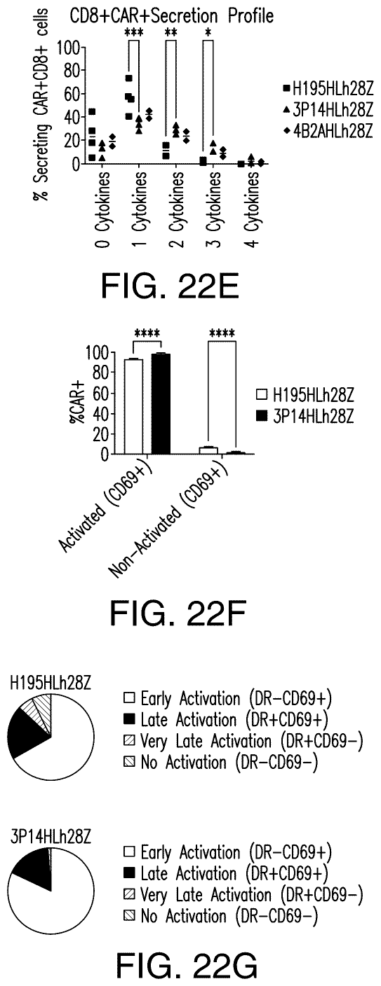

CAR T cells targeting CD33-IgC with high affinity are polyfunctional. To identify the mechanism of enhanced functionality of 3P14HLh28Z, 24-hour in vitro cytokine production was assessed by co-culturing CAR T cells with U937-CD33^{high}gfpLuc⁺ or OCiAML3-CD33^{low}gfpLuc⁺. 3P14HLh28Z secreted elevated levels of Tc1/Th1 cytokines in a target-specific manner as compared to both 4B2AHLh28Z and H195HLh28Z (FIG. 22A). Given the higher cumulative cytokine production by 3P14HLh28Z, the role of polyfunctionality, which has been associated with enhanced clinical responses, was determined (Rossi et al., Blood 132, 804-814 (2018)). After 24 hours of CAR T cell and U937-CD33^{high}gfpLuc⁺ co-culture, at an E:T ratio of 1:5, intracellular flow cytometry showed increased levels of IFN-γ, IL-2, and Granzyme B with 3P14HLh28Z versus H195HLh28Z (FIG. 22B). Furthermore, 3P14HLh28Z had fewer non-secreting CD4⁺ cells, and more CD4⁺ and CD8⁺ cells secreting at least two factors as compared to H195HLh28Z. Conversely, 4B2AHLh28Z showed a secretion pattern in between 3P14HLh28Z and H195HLh28Z (FIGS. 22C-22E).

Given the prior observation of peak CAR T cell proliferation at 7 days after U937-CD33^{high}gfpLuc⁺ encounter (FIG. 22F), these CAR T cells were further characterized at this time point utilizing multi-parametric flow cytometry. It was observed an increased number of 3P14HLh28Z cells showing an immunophenotype consistent with activation, based on CD69 and HLA-DR (DR) positivity (FIGS. 22F and 22G).

Collectively, these data suggest that 3P14HLh28Z cells display a polyfunctional soluble factor secreting profile on antigen encounter and demonstrate high levels of activation after antigen encounter.

### CAR T Cells Targeting CD33-IgC are Effective in Xenograft Models of AML.

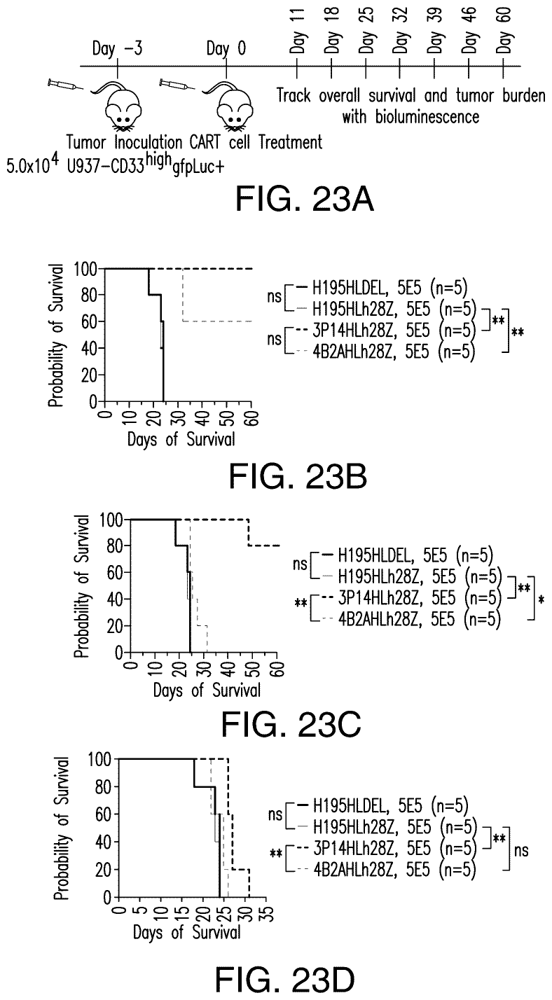

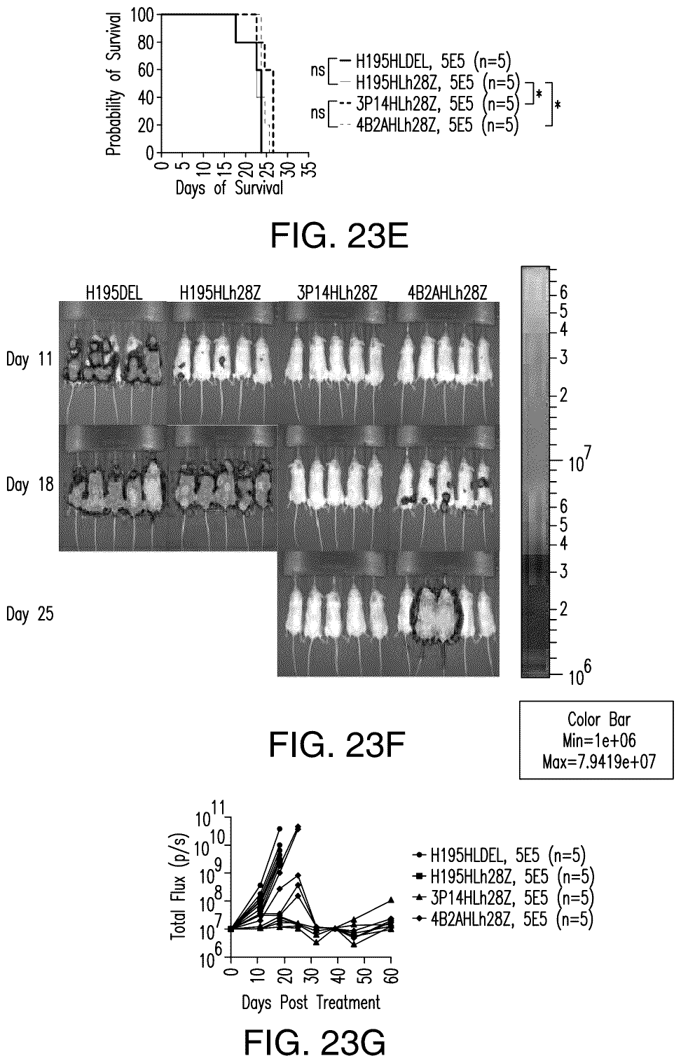

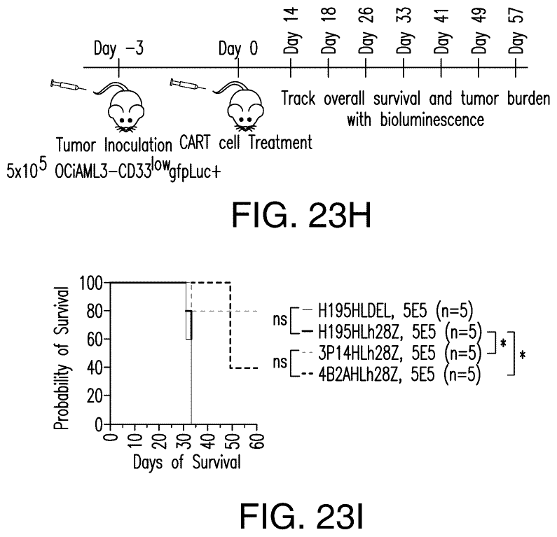

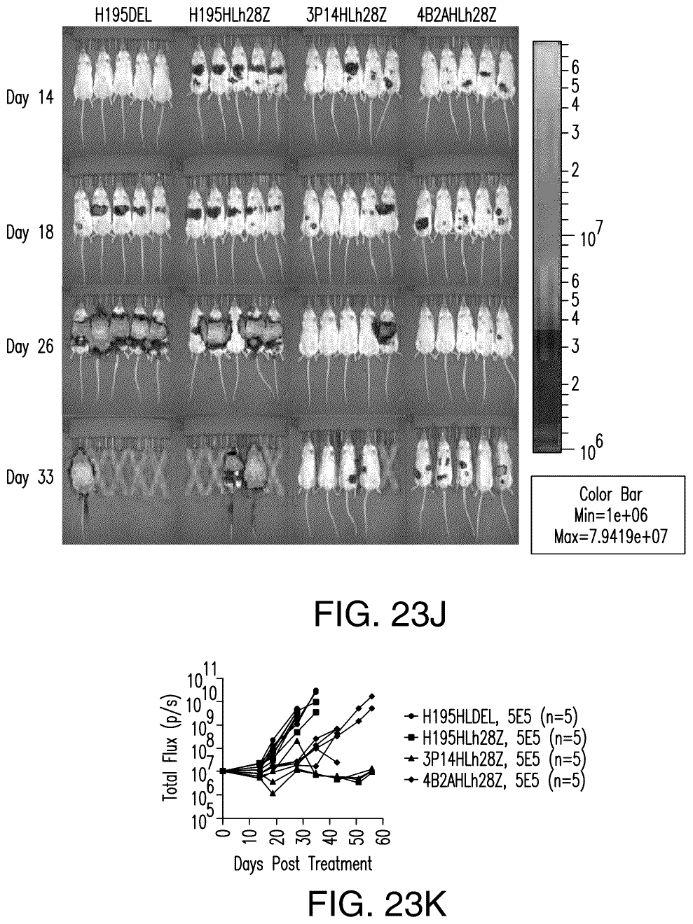

CD33-directed CAR T cells must demonstrate efficacy at low cell numbers and in the setting of low-antigen-density AML. To determine whether 3P14HLh28Z and 4B2AHLh28Z could control disease in these settings, NCG mice were inoculated with U937-CD33^{high}gfpLuc⁺ and treated with varying low doses of CAR T cells (5×10⁵, 2.5×10⁵, 1×10⁵, and 5×10⁴) 3 days later, tracking bioluminescence (BLI) and survival (FIG. 23A). CD33-IgC-directed CAR T cells demonstrated superior tumor control and improved survival in a dose-dependent manner as compared to H195HLh28Z, while 3P14HLh28Z demonstrated rapid tumor control and prolonged tumor-free states, leading to improved survival at all dose levels (FIGS. 23B-23G). The capacity of these CAR T cells to control tumor growth in the setting of low-antigen-density tumor was assessed by utilizing NCG mice engrafted with OCiAML3-CD331^{low}gfpLuc⁺ (FIG. 23H). In this setting, 3P14HLh28Z again demonstrated superior tumor control and improved survival as compared to 4B2AHLh28Z, which in turn showed improved survival over H195HLh28Z (FIG. 23I-23K).

Collectively, these data suggest 3P14HLh28Z cells have enhanced functionality in both high- and low-antigen-density xenograft models, allowing for long-term survival.

### CAR T Cells Targeting CD33-IgC are Effective in High-Tumor Burden AML Patient-Derived Xenografts.

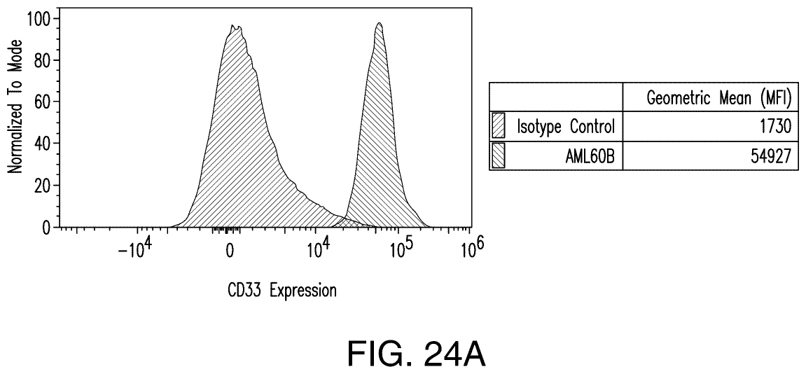

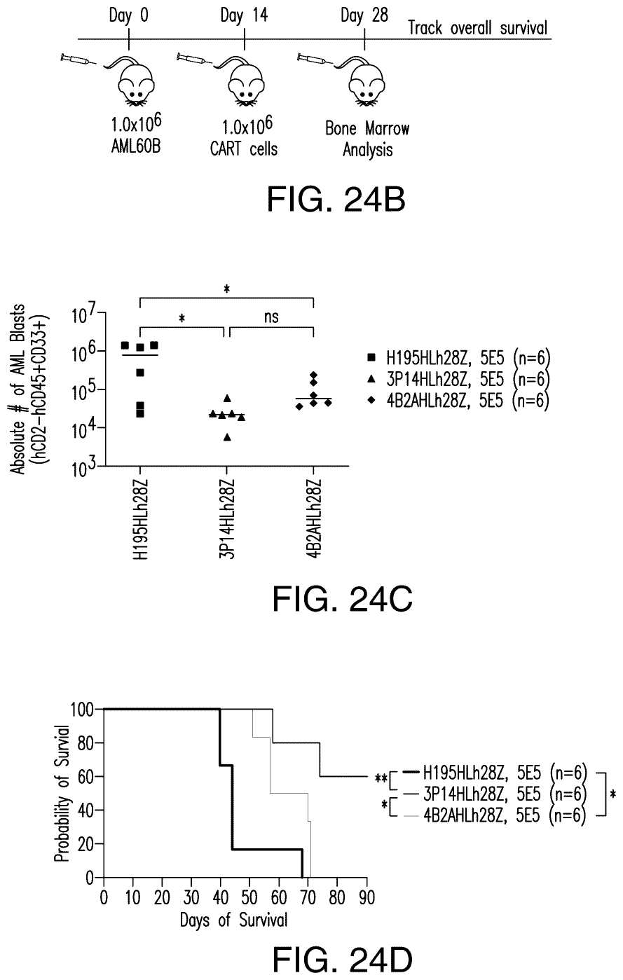

To demonstrate translational relevance, an in vivo model of high tumor burden was established utilizing peripheral blasts from a patient with R/R CD33⁺ AML (AML60B; FIG. 24A). AML60B can engraft without conditioning irradiation, allowing for increased tumor burden over time and delayed CAR T cell treatment. Fourteen days after NCG mice were inoculated, they were randomized and treated with allogeneic H195h28Z, 3P14HLh28Z or 4B2AHLh28Z CAR T cells (FIG. 24B). Compared to H195HLh28Z, 3P14HLh28Z and 4B2AHLh28Z conferred superior tumor control as evaluated on day 28 bone marrow aspiration (FIG. 24C), with only 3P14HLh28Z-treated mice achieving long-term survival in a majority of mice (FIG. 24D).

Taken together, these data demonstrate that 3P14HLh28Z provides superior tumor control, leading to long-term survival in vivo in the setting of high tumor burden established from R/R primary AML.

### Discussion

Despite the introduction of novel agents to the AML therapeutic armamentarium, this myeloid malignancy continues to have a very dismal prognosis; there is a critical need for potent interventions such as adoptive T cell therapy. CAR T cells have revolutionized the treatment of B-cell malignancies but have yet to show efficacy in myeloid disease.

Recently, it was described a phase I trial (NCT03126864) utilizing autologous CD33-directed 4-1BB/CD3Z CAR T cells for the treatment of adult patients with R/R AML (Tambaro et al., Leukemia 35, 3282-3286 (2021)). The CAR T cell product was successfully manufactured and administered to only 3 of 10 enrolled patients, in part due to lymphopenia and inadequate numbers of starting T cells obtained at the apheresis step. None of these 3 treated patients had observable anti-leukemia response at the first dose level. This study showed that AML-directed CAR T cells must be capable of robust proliferation and killing at low effector-to-tumor ratios, as many patients with R/R AML have lymphopenia and high disease burden of circulating peripheral blasts. Another study recently described an extensive preclinical screen of CD33 IgV-directed CAR T cells derived from lintuzumab and gemtuzumab, integrating either CD28/CD3Z or 4-1BB/CD3Z costimulatory domains (Qin et al., J Immunother Cancer 9 (2021)). This study reported the lintuzumab-CD28/CD3Z platform as the best-in-class CAR for the treatment of AML. The presently disclosed subject matter hypothesized that the efficacy of CD33-directed CAR T cells could be improved by targeting the membrane-proximal IgC domain of CD33, allowing for more robust T cell functionality and improved tumor control. It was demonstrated that the potency of CD33 CAR T cells can be markedly increased by utilizing scFvs that target CD33-IgC with high affinity. Surprisingly, raising human binders to membrane-proximal epitopes was dependent on IgC immunization, as full-length CD33 immunization always led to the generation of IgV-specific antibodies, suggesting that IgV is an immunodominant epitope. These data suggest that selecting scFvs targeting membrane-proximal epitopes with high affinity via domain-specific immunization can enhance CAR T cells' efficacy against a target.

### Embodiments of the Presently Disclosed Subject Matter

From the foregoing description, it will be apparent that variations and modifications may be made to the presently disclosed subject matter to adopt it to various usages and conditions. Such embodiments are also within the scope of the following claims.

The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. An antigen-recognizing receptor, comprising an extracellular antigen-binding domain, a transmembrane domain, and an intracellular signaling domain, wherein the extracellular antigen-binding domain specifically binds to CD33, wherein the extracellular antigen-binding domain comprises a heavy chain variable region and a light chain variable region, wherein:
(a) the heavy chain variable region comprises a CDR1 comprising the amino acid sequence set forth in SEQ ID NO: 2, a CDR2 comprising the amino acid sequence set forth in SEQ ID NO: 3, and a CDR3 comprising the amino acid sequence set forth in SEQ ID NO: 4; the light chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 7;
(b) the heavy chain variable region comprises a CDR1 comprising the amino acid sequence set forth in SEQ ID NO: 12, a CDR2 comprising an amino acid sequence set forth in SEQ ID NO: 13, and a CDR3 comprising the amino acid sequence set forth in SEQ ID NO: 14; the light chain variable region comprises a CDR1 comprising the amino acid sequence set forth in SEQ ID NO: 15, and a CDR2 comprising the amino acid sequence set forth in SEQ ID NO: 16, and a CDR3 comprising the amino acid sequence set forth in SEQ ID NO: 17;
(c) the heavy chain variable region comprises a CDR1 comprising the amino acid sequence set forth in SEQ ID NO: 22, a CDR2 comprising the amino acid sequence set forth in SEQ ID NO: 23, and a CDR3 comprising the amino acid sequence set forth in SEQ ID NO: 24 the light chain variable region comprises a CDR1 comprising the amino acid sequence set forth in SEQ ID NO: 25, a CDR2 comprising the amino acid sequence set forth in SEQ ID NO: 26, and a CDR3 comprising the amino acid sequence set forth in SEQ ID NO: 27;
(d) the heavy chain variable region comprises a CDR1 comprising the amino acid sequence set forth in SEQ ID NO: 32, a CDR2 comprising the amino acid sequence set forth in SEQ ID NO: 33, and a CDR3 comprising the amino acid sequence set forth in SEQ ID NO: 34 the light chain variable region comprises a CDR1 comprising the amino acid sequence set forth in SEQ ID NO: 25, a CDR2 comprising the amino acid sequence set forth in SEQ ID NO: 26, and a CDR3 comprising the amino acid sequence set forth in SEQ ID NO: 27;
(e) the heavy chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 39; the light chain variable region comprises a CDR1 comprising the amino acid sequence set forth in SEQ ID NO: 40, a CDR2 comprising the amino acid sequence set forth in SEQ ID NO: 41, and a CDR3 comprising the amino acid sequence set forth in SEQ ID NO: 42;
(f) the heavy chain variable region comprises a CDR1 comprising the amino acid sequence set forth in SEQ ID NO: 47, a CDR2 comprising the amino acid sequence set forth in SEQ ID NO: 38, and a CDR3 comprising the amino acid sequence set forth in SEQ ID NO: 48; the light chain variable region comprises a CDR1 comprising the amino acid sequence set forth in SEQ ID NO: 49, a CDR2 comprising the amino acid sequence set forth in SEQ ID NO: 50, and a CDR3 comprising the amino acid sequence set forth in SEQ ID NO: 51;
(g) the heavy chain variable region comprises a CDR1 comprising the amino acid sequence set forth in SEQ ID NO: 56, a CDR2 comprising the amino acid sequence set forth in SEQ ID NO: 57, and a CDR3 comprising the amino acid sequence set forth in SEQ ID NO: 58; the light chain variable region comprises a CDR1 comprising the amino acid sequence set forth in SEQ ID NO: 59, a CDR2 comprising the amino acid sequence set forth in SEQ ID NO: 60, and a CDR3 comprising the amino acid sequence set forth in SEQ ID NO: 61;
(h) the heavy chain variable region comprises a CDR1 comprising the amino acid sequence set forth in SEQ ID NO: 66, a CDR2 comprising the amino acid sequence set forth in SEQ ID NO: 67, and a CDR3 comprising the amino acid sequence set forth in SEQ ID NO: 68; the light chain variable region comprises a CDR1 comprising the amino acid sequence set forth in SEQ ID NO: 69, a CDR2 comprising the amino acid sequence set forth in SEQ ID NO: 70, and a CDR3 comprising the amino acid sequence set forth in SEQ ID NO: 71;
(i) the heavy chain variable region comprises a CDR1 comprising the amino acid sequence set forth in SEQ ID NO: 76, a CDR2 comprising the amino acid sequence set forth in SEQ ID NO: 57, and a CDR3 comprising the amino acid sequence set forth in SEQ ID NO: 77; the light chain variable region comprises a CDR1 comprising the amino acid sequence set forth in SEQ ID NO: a CDR2 comprising the amino acid sequence set forth in SEQ ID NO: 79, and a CDR3 comprising the amino acid sequence set forth in SEQ ID NO: 80; or
(j) the heavy chain variable region comprises a CDR1 comprising the amino acid sequence set forth in SEQ ID NO: 85, a CDR2 comprising the amino acid sequence set forth in SEQ ID NO: 86, and a CDR3 comprising the amino acid sequence set forth in SEQ ID NO: 87; the light chain variable region comprises a CDR1 comprising the amino acid sequence set forth in SEQ ID NO: 5, a CDR2 comprising the amino acid sequence set forth in SEQ ID NO: 41, and a CDR3 comprising the amino acid sequence set forth in SEQ ID NO: 42.

2. The antigen-recognizing receptor of claim 1, wherein the extracellular antigen-binding domain is a single-chain variable fragment (scFv), a Fab, or a F(ab)₂.

3. The antigen-recognizing receptor of claim 2, wherein one or more of the scFv, Fab and F(ab)₂ are comprised in a fusion protein with a heterologous sequence to form the extracellular antigen-binding domain.

4. The antigen-recognizing receptor of claim 1, wherein
(a) the heavy chain variable region comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence selected set forth in SEQ ID NO: 8, SEQ ID NO: 18, SEQ ID NO: 28, SEQ ID NO: 35, SEQ ID NO: 43, SEQ ID NO: 52, SEQ ID NO: 62, SEQ ID NO: 72, SEQ ID NO: 81, or SEQ ID NO: 88; and
(b) the light chain variable region comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence set forth in SEQ ID NO: 9, SEQ ID NO: 19, SEQ ID NO: 29, SEQ ID NO: 44, SEQ ID NO: 53, SEQ ID NO: 63, SEQ ID NO: 73, SEQ ID NO: 82, SEQ ID NO: 89, or SEQ ID NO: 92.

5. The antigen-recognizing receptor of claim 4, wherein
(a) the heavy chain variable region comprises the amino acid sequence set forth in SEQ ID NO: 8, SEQ ID NO: 18, SEQ ID NO: 28, SEQ ID NO: 35, SEQ ID NO: 43, SEQ ID NO: 52, SEQ ID NO: 62, SEQ ID NO: 72, SEQ ID NO: 81, or SEQ ID NO: 88; and
(b) the light chain variable region comprises the amino acid sequence set forth in SEQ ID NO: 9, SEQ ID NO: 19, SEQ ID NO: 29, SEQ ID NO: 44, SEQ ID NO: 53, SEQ ID NO: 63, SEQ ID NO: 73, SEQ ID NO: 82, SEQ ID NO: 89, or SEQ ID NO: 92.

6. The antigen-recognizing receptor of claim 1, wherein
(a) the heavy chain variable region comprises the amino acid sequence set forth in SEQ ID NO: 8, and the light chain variable region comprises the amino acid sequence set forth in SEQ ID NO: 9;
(b) the heavy chain variable region comprises the amino acid sequence set forth in SEQ ID NO: 18, and the light chain variable region comprises the amino acid sequence set forth in SEQ ID NO: 19;
(c) the heavy chain variable region comprises the amino acid sequence set forth in SEQ ID NO: 28, and the light chain variable region comprises the amino acid sequence set forth in SEQ ID NO: 29;
(d) the heavy chain variable region comprises the amino acid sequence set forth in SEQ ID NO: 35, and the light chain variable region comprises the amino acid sequence set forth in SEQ ID NO: 29;
(e) the heavy chain variable region comprises the amino acid sequence set forth in SEQ ID NO: 43, and the light chain variable region comprises the amino acid sequence set forth in SEQ ID NO: 44;
(f) the heavy chain variable region comprises the amino acid sequence set forth in SEQ ID NO: 52, and the light chain variable region comprises the amino acid sequence set forth in SEQ ID NO: 53;
(g) the heavy chain variable region comprises the amino acid sequence set forth in SEQ ID NO: 62, and the light chain variable region comprises the amino acid sequence set forth in SEQ ID NO: 63;
(h) the heavy chain variable region comprises the amino acid sequence set forth in SEQ ID NO: 72, and the light chain variable region comprises the amino acid sequence set forth in SEQ ID NO: 73;
(i) the heavy chain variable region comprises the amino acid sequence set forth in SEQ ID NO: 81, and the light chain variable region comprises the amino acid sequence set forth in SEQ ID NO: 82;
(j) the heavy chain variable region comprises the amino acid sequence set forth in SEQ ID NO: 88, and the light chain variable region comprises the amino acid sequence set forth in SEQ ID NO: 89; or
(k) the heavy chain variable region comprises the amino acid sequence set forth in SEQ ID NO: 8, and the light chain variable region comprises the amino acid sequence set forth in SEQ ID NO: 93.

7. The antigen-recognizing receptor of claim 1, wherein the extracellular antigen-binding domain comprises a linker between the heavy chain variable region and the light chain variable region.

8. The antigen-recognizing receptor of claim 1, wherein a signal peptide is covalently joined to the 5′ terminus of the extracellular antigen-binding domain.

9. The antigen-recognizing receptor of claim 1, wherein the transmembrane domain comprises a CD8 polypeptide, a CD28 polypeptide, a CD3ζ polypeptide, a CD4 polypeptide, a 4-1BB polypeptide, an OX40 polypeptide, an ICOS polypeptide, a CTLA-4 polypeptide, a PD-1 polypeptide, a LAG-3 polypeptide, a 2B4 polypeptide, a BTLA polypeptide, or a combination thereof.

10. The antigen-recognizing receptor of claim 1, wherein the intracellular signaling domain comprises a CD3ζ polypeptide.

11. The antigen-recognizing receptor of claim 1, wherein the intracellular signaling domain further comprises at least one co-stimulatory signaling region.

12. The antigen-recognizing receptor of claim 11, wherein the at least one co-stimulatory signaling region comprises a CD28 polypeptide, a 4-1BB polypeptide, an OX40 polypeptide, an ICOS polypeptide, a DAP-10 polypeptide, or a combination thereof.

13. The antigen-recognizing receptor of claim 1, wherein the antigen-recognizing receptor is a chimeric antigen receptor (CAR), or a T-cell like fusion protein.

14. The antigen-recognizing receptor of claim 1, wherein the antigen-recognizing receptor is recombinantly expressed or expressed from a vector.

15. A cell comprising the antigen-recognizing receptor of claim 1.

16. The cell of claim 15, wherein the antigen-recognizing receptor is constitutively expressed on the surface of the cell.

17. The cell of claim 15, wherein the cell is an immunoresponsive cell, a cell of the lymphoid lineage, or a cell of the myeloid lineage.

18. The cell of claim 15, wherein the cell is selected from the group consisting of a T cell, a Natural Killer (NK) cell, and a stem cell from which a lymphoid cell may be differentiated.

19. The cell of claim 18, wherein the cell is a T cell.

20. The cell of claim 19, wherein the T cell is a cytotoxic T lymphocyte (CTL) or a regulatory T cell.

21. The cell of claim 18, wherein the stem cell is a pluripotent stem cell, an embryoid stem cell, or an induced pluripotent stem cell.

22. A nucleic acid encoding the antigen-recognizing receptor of claim 1.

23. A vector comprising the nucleic acid of any one of claim 22.

24. A host cell expressing the nucleic acid of claim 22.

25. A composition comprising the cell of claim 15.

26. A lipid nanoparticle comprising the nucleic acid of claim 22.

27. A composition comprising the lipid nanoparticle of claim 26.

28. A method of treating or ameliorating a disease or disorder in a subject, reducing tumor burden in a subject, treating and/or preventing a tumor in a subject, and/or increasing or lengthening survival of a subject having a tumor comprising administering to the subject the cell of claim 15.

29. The method of claim 28, wherein the tumor is hematological cancer or solid tissue cancer.

30. The method of claim 28, wherein the tumor is selected from the group consisting of acute myeloid leukemia (AML), myelodysplastic syndromes (MDS), acute lymphoblastic leukemia (ALL), chronic lymphocytic leukemia (CLL), myeloproliferative neoplasms (MPNs), and chronic myeloid neoplasms.

31. The method of claim 30, wherein the tumor is acute myeloid leukemia (AML).

32. The method of claim 28, wherein the subject is a human.

33. A kit for treating or ameliorating a disease or disorder in a subject, reducing tumor burden in a subject, treating and/or preventing a tumor in a subject, and/or increasing or lengthening survival of a subject having a tumor, comprising the cell of claim 15.

34. A method for producing a CD33-targeted antigen-recognizing receptor of any one of claim 1, comprising introducing into the cell a nucleic acid that encodes the antigen-recognizing receptor.


SEQUENCE LISTING (178 sequences)

SEQ ID NO: 1 - protein, 364 aa, Homo sapiens
MPLLLLLPLLWAGALAMDPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGAIGGAGVTALLALCLCLIFFIVKTHRRKAARTAVGRNDTHPTTGSASPKHQKKSKLHGPTETSSCSGAAPTVEMDEELHYASLNFHGMNPSKDTSTEYSEVRTQ

SEQ ID NO: 2 - protein, 8 aa, synthetic construct
GFTFSTYA

SEQ ID NO: 3 - protein, 8 aa, synthetic construct
ISGRGGST

SEQ ID NO: 4 - protein, 14 aa, synthetic construct
AGRGDYYYYYGMDV

SEQ ID NO: 5 - protein, 11 aa, synthetic construct
QSLVYSDGNTY

SEQ ID NO: 6 - skipped

SEQ ID NO: 7 - protein, 9 aa, synthetic construct
MQSTQFPHT

SEQ ID NO: 8 - protein, 121 aa, synthetic construct
EVQLLESGGGLVQPGGSLRLSCAASGFTFSTYAMSWVRQAPGKGLEWVSAISGRGGSTYYTDSVKGRFTISRDNSKNTVSLQMNSLRAEDTAVYYCAGRGDYYYYYGMDVWGQGTTVTVSA

SEQ ID NO: 9 - protein, 112 aa, synthetic construct
DIVMTQSPLSSPVTLGQPASFSCRSSQSLVYSDGNTYLSWLQQRPGQPPRLLIYKISNRFSGVPDRFSGSGAGTDFTLKISRVEAEDVGVYYCMQSTQFPHTFGQGTKLEIK

SEQ ID NO: 10 - DNA, 363 bp, synthetic construct
  source 1..363: mol_type = other DNA
gaggtgcagctgttggagtctgggggaggcttggtccagcctggggggtccctgagactctcctgtgcagcctctggattcacctttagcacctatgccatgagctgggtccgccaggctccagggaaggggctggagtgggtctcagctattagtggtcgtggtggtagcacatactacacagactccgtgaagggccggttcaccatctccagagacaattccaagaacacggtgtctctgcaaatgaacagcctgagagccgaggacacggccgtatattactgtgcgggccggggagattactactactactacggtatggacgtctggggccaagggaccacggtcaccgtctccgca

SEQ ID NO: 11 - DNA, 336 bp, synthetic construct
  source 1..336: mol_type = other DNA
gatattgtgatgacccagagtccactctcctcacctgtcacccttggacagccggcctccttctcctgcaggtctagtcaaagcctcgtatacagtgatggaaacacctacttgagttggcttcagcagaggccaggccagcctccaagactcctaatttataagatttctaaccggttctctggggtcccagacagattcagtggcagtggggcagggacagatttcacactgaaaatcagcagggtggaagctgaggatgtcggggtttattactgcatgcaatctacacaatttcctcacacttttggccaggggaccaagctggagatcaaa

SEQ ID NO: 12 - protein, 8 aa, synthetic construct
GFIFSSNA

SEQ ID NO: 13 - protein, 8 aa, synthetic construct
ISGYGGNT

SEQ ID NO: 14 - protein, 14 aa, synthetic construct
AKWGTYIVGATGDY

SEQ ID NO: 15 - protein, 9 aa, synthetic construct
SNDVGGYNY

SEQ ID NO: 16 - skipped

SEQ ID NO: 17 - protein, 10 aa, synthetic construct
SSYAGSNNWV

SEQ ID NO: 18 - protein, 121 aa, synthetic construct
EVHLLESGGGLVQPGGSLRLSCAASGFIFSSNAMSWVRQAPGKGLEWVSAISGYGGNTYYADSVKGRFTISRDNSKNTLYLQMNSLRAEDTAVYYCAKWGTYIVGATGDYWGQGTLVTVSS

SEQ ID NO: 19 - protein, 110 aa, synthetic construct
QSALTQPPSASGSPGQSVTISCTGTSNDVGGYNYVSWYQQHPGKAPKLLIYEVSKRPSGVPDRFSGSQSGNTASLTVSGLQAEDEADYYCSSYAGSNNWVFGGGTKLTVL

SEQ ID NO: 20 - DNA, 363 bp, synthetic construct
  source 1..363: mol_type = other DNA
gaggtgcacctgttggagtctgggggaggcttggtacagcctggggggtccctgagactctcctgtgcagcctctggattcatctttagcagcaatgccatgagctgggtccgccaggctccagggaagggactggagtgggtctcagctattagtggttatggtggtaacacatactacgcagactccgtgaagggccggttcaccatctccagagacaattccaagaacacgctatatctgcaaatgaacagcctgagagccgaggacacggccgtatattactgtgcgaaatgggggacttatatagtgggagctacgggtgactactggggccagggaactctggtcaccgtctcctca

SEQ ID NO: 21 - DNA, 330 bp, synthetic construct
  source 1..330: mol_type = other DNA
cagtctgccctgactcagcctccctccgcgtccgggtctcctggacagtcagtcaccatctcctgcactggaaccagcaatgacgttggtggttataactatgtctcctggtaccaacagcacccaggcaaagcccccaaactcttgatttatgaggtcagtaagcggccctcaggggtccctgatcgcttctctggctcccagtctggcaacacggcctccctgaccgtctctgggctccaggctgaggatgaggctgattattactgcagctcatatgcaggcagcaacaattgggtgttcggcggagggaccaagctgaccgtccta

SEQ ID NO: 22 - protein, 10 aa, synthetic construct
GDSVSSNSAA

SEQ ID NO: 23 - protein, 9 aa, synthetic construct
TYFRSKWYN

SEQ ID NO: 24 - protein, 10 aa, synthetic construct
ASEGGSYYDH

SEQ ID NO: 25 - protein, 6 aa, synthetic construct
QGISNW

SEQ ID NO: 26 - skipped

SEQ ID NO: 27 - protein, 9 aa, synthetic construct
QQADSFPFT

SEQ ID NO: 28 - protein, 120 aa, synthetic construct
QVQLQQSGPGLVKPSQTLSLTCAISGDSVSSNSAAWNWIRQSPSRGLEWLGRTYFRSKWYNVYAVSVKSRITINPDTSKNQFSLQLNSVTPEDTAVYYCASEGGSYYDHWGQGTLVTVSS

SEQ ID NO: 29 - protein, 107 aa, synthetic construct
DIQMTQSPSSVSASVGDRVTITCRASQGISNWLTWYQQKPGKAPKLLIYAASSLQSGVPSRFSGSGSGTDFTLTISSLQPEDFATYYCQQADSFPFTFGPGTKVDIK

SEQ ID NO: 30 - DNA, 360 bp, synthetic construct
  source 1..360: mol_type = other DNA
caggtacagctgcagcagtcaggtccaggactggtgaagccctcgcagaccctctcactcacctgtgccatctccggggacagtgtctctagcaacagtgctgcttggaactggatcaggcagtccccatcgagaggccttgagtggctgggaaggacatacttcaggtccaagtggtataatgtttatgcagtgtctgtgaagagtcgaataaccatcaacccagacacatccaagaaccagttctccctgcagctgaactctgtgactcccgaggacacggctgtgtattattgtgcaagcgagggtgggagctattatgaccactggggccagggaaccctggtcaccgtctcctca

SEQ ID NO: 31 - DNA, 321 bp, synthetic construct
  source 1..321: mol_type = other DNA
gacatccagatgacccagtctccatcttccgtgtctgcatctgtaggagacagagtcaccatcacttgtcgggcgagtcagggtattagtaattggttaacctggtatcagcagaaaccagggaaagcccctaagctcctgatctatgctgcatccagtttgcaaagtggggtcccatcaaggttcagcggcagtggatctgggacagatttcactctcaccatcagcagcctgcagcctgaagattttgcaacttactattgtcaacaggctgacagtttcccattcactttcggccctgggaccaaagtggatatcaaa

SEQ ID NO: 32 - protein, 10 aa, synthetic construct
GFSLSTSGMC

SEQ ID NO: 33 - protein, 7 aa, synthetic construct
IDWDDDK

SEQ ID NO: 34 - protein, 14 aa, synthetic construct
ARTPYSGSYNWFDP

SEQ ID NO: 35 - protein, 122 aa, synthetic construct
QVTLRESGPALVKPTQTLTLTCTFSGFSLSTSGMCVSWIRQPPGKALEWLALIDWDDDKYYSTSLKTRLTISKDTSKNQVVLTMTNMDPVDTATYYCARTPYSGSYNWFDPWGQGTLVTVSS

SEQ ID NO: 36 - DNA, 366 bp, synthetic construct
  source 1..366: mol_type = other DNA
caggtcaccttgagggagtctggtcctgcgctggtgaaacccacacagaccctcacactgacctgcaccttctctgggttctcactcagcactagtggaatgtgtgtgagctggatccgtcagcccccagggaaggccctggagtggcttgcactcattgattgggatgatgataaatactacagcacatctctgaagaccaggctcaccatctccaaggacacctccaaaaaccaggtggtccttacaatgaccaacatggaccctgtggacacagccacgtattattgtgcacggaccccctatagtgggagctacaactggttcgacccctggggccagggaaccctggtcaccgtctcctca

SEQ ID NO: 37 - protein, 8 aa, synthetic construct
GFTFSTYG

SEQ ID NO: 38 - protein, 8 aa, synthetic construct
ISYDGSNK

SEQ ID NO: 39 - protein, 10 aa, synthetic construct
ARGRVGTLDY

SEQ ID NO: 40 - protein, 11 aa, synthetic construct
RGLVYSDVNTN

SEQ ID NO: 41 - skipped

SEQ ID NO: 42 - protein, 9 aa, synthetic construct
MQGTHWPWT

SEQ ID NO: 43 - protein, 117 aa, synthetic construct
QVQLVESGGGVVQPGRSLRLSCAASGFTFSTYGMHWVRQAPGKGLEWVAVISYDGSNKYHGDAVKGRFTISRDNSKNTLYLQMNSLRAEDTAVYYCARGRVGTLDYWGQGTLVTVSS

SEQ ID NO: 44 - protein, 112 aa, synthetic construct
DVVMTQSPLSLPVSLGQPASISCRSSRGLVYSDVNTNLNWFQQRPGQSPRRLIYKVSNRDSGVPDRFSGSGSGTDFTLKISRVEAEDVGVYYCMQGTHWPWTFGQGTKVEIK

SEQ ID NO: 45 - DNA, 351 bp, synthetic construct
  source 1..351: mol_type = other DNA
caggtgcagctggtggagtctgggggaggcgtggtccagcctgggaggtccctgagactctcctgtgcagcctctggatttaccttcagtacctatggcatgcactgggtccgccaggctccaggcaaggggctggagtgggtggcagtcatatcatatgatggaagtaataaatatcatggagacgccgtgaagggccggttcaccatctccagagacaattccaagaacacgctgtatctgcaaatgaacagcctgagagctgaggacacggctgtgtattactgtgcgagggggagagtgggaactcttgactattggggccagggaaccctggtcaccgtctcctca

SEQ ID NO: 46 - DNA, 336 bp, synthetic construct
  source 1..336: mol_type = other DNA
gatgttgtgatgactcagtctccactctccctgcccgtcagccttggacagccggcctccatctcctgcaggtctagtcgaggcctcgtatacagtgatgtaaacaccaacttgaattggtttcagcagaggccaggccaatctccaaggcgcctaatttataaggtttctaaccgggactctggggtcccagacagattcagcggcagtgggtcaggcactgatttcacactgaaaatcagcagggtggaggctgaggatgttggggtttattactgcatgcaaggtacacactggccttggacgttcggccaagggaccaaggtggaaatcaaa

SEQ ID NO: 47 - protein, 8 aa, synthetic construct
GFTFNSYG

SEQ ID NO: 48 - protein, 16 aa, synthetic construct
ARDNYDSSGYNWYFDL

SEQ ID NO: 49 - protein, 6 aa, synthetic construct
SLRSYY

SEQ ID NO: 50 - skipped

SEQ ID NO: 51 - protein, 11 aa, synthetic construct
NSRDSSGNLWV

SEQ ID NO: 52 - protein, 123 aa, synthetic construct
QVQLVESGGGVVQPGRSLRLSCAASGFTFNSYGMHWVRQAPGKGLEWVAIISYDGSNKYYADSVKGRFTISRDNSKNTLYLQMNSLRAEDTAVYYCARDNYDSSGYNWYFDLWGRGTLVTVSS

SEQ ID NO: 53 - protein, 108 aa, synthetic construct
SSELTQDPAVSVALGQTVRITCQGDSLRSYYASWYQQKPGQAPVLVIYGKNNRPSGIPDRFSGSSSGNTASLTITGAQAEDEADYYCNSRDSSGNLWVFGGGTKLTVL

SEQ ID NO: 54 - DNA, 369 bp, synthetic construct
  source 1..369: mol_type = other DNA
caggtgcagctggtggagtctgggggaggcgtggtccagcctgggaggtccctgagactctcctgtgcagcctctggattcaccttcaacagctatggcatgcactgggtccgccaggctccaggcaaggggctggagtgggtggcaattatatcatatgatggaagtaataaatactatgcagactccgtgaagggccgattcaccatctccagagacaattccaagaacacgctgtatctgcaaatgaacagcctgagagctgaggacacggctgtgtattattgtgcgagagataattatgatagtagtggttataactggtacttcgatctctggggccgtggcaccctggtcactgtctcctca

SEQ ID NO: 55 - DNA, 324 bp, synthetic construct
  source 1..324: mol_type = other DNA
tcttctgagctgactcaggaccctgctgtgtctgtggccttgggacagacagtcaggatcacatgccaaggagacagcctcagaagctattatgcaagctggtaccagcagaagccaggacaggcccctgtacttgtcatctatggtaaaaacaaccggccctcagggatcccagaccgattctctggctccagctcaggaaacacagcttccttgaccatcactggggctcaggcggaagatgaggctgactattactgtaactcccgggacagcagtggtaacctctgggtgttcggcggagggaccaagctgaccgtccta

SEQ ID NO: 56 - protein, 8 aa, synthetic construct
GDSISSYY

SEQ ID NO: 57 - protein, 7 aa, synthetic construct
IYTSGNT

SEQ ID NO: 58 - protein, 14 aa, synthetic construct
ARDGDNRDSDAFDI

SEQ ID NO: 59 - protein, 7 aa, synthetic construct
QNISSSY

SEQ ID NO: 60 - skipped

SEQ ID NO: 61 - protein, 9 aa, synthetic construct
QQYGSSPLT

SEQ ID NO: 62 - protein, 120 aa, synthetic construct
QVQLQESGPGLVKPSETLSLTCTVSGDSISSYYWSWIRQPAGKGLEWIGRIYTSGNTNYNPSLKSRVTMSVDKSKNQFSLKLRSVTAADTAVYYCARDGDNRDSDAFDIWGQGTMVTVSS

SEQ ID NO: 63 - protein, 102 aa, synthetic construct
SPGTLSLSPGERATLSCRASQNISSSYLAWCQQKPGQAPRLFIYGTSRRATGIPDRFSGSGSGTDFTLTISRLEPEDFAVYYCQQYGSSPLTFGGGTKVEIK

SEQ ID NO: 64 - DNA, 360 bp, synthetic construct
  source 1..360: mol_type = other DNA
caggtgcagctgcaggagtcgggcccaggactggtgaagccttcggagaccctgtccctcacctgcactgtctctggtgactccatcagtagttattactggagctggatccggcagcccgccgggaagggactggagtggattggacgtatctataccagtgggaacaccaactacaacccctccctcaagagtcgagtcaccatgtcagtagacaagtccaagaaccagttctccctgaagctgaggtctgtgaccgccgcggacacggccgtgtattactgtgcgagagatggtgataaccgggactctgatgcttttgatatctggggccaagggacaatggtcaccgtctcttca

SEQ ID NO: 65 - DNA, 324 bp, synthetic construct
  source 1..324: mol_type = other DNA
gaaattgtgttgacgcagtctccaggcaccctgtctttgtctccaggggaaagagccaccctctcctgcagggccagtcagaatattagcagcagctacttagcctggtgccagcagaaacctggccaggctcccaggctcttcatctatggtacatcccgcagggccactggcatcccagacaggttcagtggcagtgggtctgggacagacttcactctcaccatcagcagactggagcctgaagattttgcagtgtattactgtcagcagtatggtagttcacctctcactttcggcggagggaccaaggtggagatcaaa

SEQ ID NO: 66 - protein, 8 aa, synthetic construct
GFSFTSHW

SEQ ID NO: 67 - protein, 8 aa, synthetic construct
IYPGDSDT

SEQ ID NO: 68 - protein, 15 aa, synthetic construct
ARHETGNGYSYGMDV

SEQ ID NO: 69 - protein, 11 aa, synthetic construct
QSLLHSNGYNY

SEQ ID NO: 70 - skipped

SEQ ID NO: 71 - protein, 9 aa, synthetic construct
MQALQPPLT

SEQ ID NO: 72 - protein, 122 aa, synthetic construct
EVQLVQSGADVKKPGESLKISCKGSGFSFTSHWIAWVRQMPGKGLEWMGIIYPGDSDTRYSPSLQGRVTISADKSISTAYLQWSSLKASDTAMYFCARHETGNGYSYGMDVWGQGTTVTVSS

SEQ ID NO: 73 - protein, 112 aa, synthetic construct
DIVMTQSPLSLPVTPGEPASISCRSSQSLLHSNGYNYLDWYLQKPGQSPQLLFSLGSNRASGVPDRFSGSGSGTDFSLKISRVEAEDVGLYYCMQALQPPLTFGGGTKVEIK

SEQ ID NO: 74 - DNA, 366 bp, synthetic construct
  source 1..366: mol_type = other DNA
gaggtgcagctggtgcagtctggagcagacgtgaaaaagcccggggagtctctgaagatctcctgtaagggttctggattcagttttaccagccactggatcgcctgggtgcgccagatgcccgggaaaggcctggagtggatggggataatctatcctggtgactctgataccagatacagcccgtccttacaaggccgggtcaccatctcagccgacaagtccatcagcaccgcctacctgcagtggagcagcctgaaggcctcggacaccgccatgtatttctgtgcgaggcatgaaactgggaatggctactcctacggaatggacgtctggggccaagggaccacggtcaccgtctcctca

SEQ ID NO: 75 - DNA, 336 bp, synthetic construct
  source 1..336: mol_type = other DNA
gatattgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgttctctttgggttctaatcgggcctccggggtccctgacaggttcagtggcagtggatcaggcacagatttttcactgaaaatcagcagagtggaggctgaggatgttgggctttattactgcatgcaagctctacaacctccgctcactttcggcggagggaccaaggtggagatcaaa

SEQ ID NO: 76 - protein, 8 aa, synthetic construct
GGSISTYY

SEQ ID NO: 77 - protein, 14 aa, synthetic construct
ARDGDNRNSDAFDI

SEQ ID NO: 78 - protein, 9 aa, synthetic construct
SSNIGAGYD

SEQ ID NO: 79 - skipped

SEQ ID NO: 80 - protein, 11 aa, synthetic construct
QSYDRSLSGWV

SEQ ID NO: 81 - protein, 120 aa, synthetic construct
QVQLQESGPGLVKPSETLSLTCTVSGGSISTYYWSWIRQPAGKGLEWIGRIYTSGNTNYNPSLKSRVTMSVDTSKNQLSLKLRSVTAADTAVYYCARDGDNRNSDAFDIWGQGTMVTVSS

SEQ ID NO: 82 - protein, 111 aa, synthetic construct
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDRSLSGWVFGGGTKLTVL

SEQ ID NO: 83 - DNA, 360 bp, synthetic construct
  source 1..360: mol_type = other DNA
caggtgcagctgcaggagtcgggcccaggactggtgaagccttcggagaccctgtccctcacctgcactgtctctggtggctccatcagtacttattactggagctggatccggcagcccgccgggaagggactggagtggattgggcgtatctataccagtgggaacaccaactacaacccctccctcaagagtcgagtcaccatgtcagtagacacgtccaagaatcagctctccctgaagctgaggtctgtgaccgccgcggacacggccgtgtattactgtgcgagagatggggataaccggaactctgatgcttttgatatttggggccaagggacaatggtcaccgtctcttca

SEQ ID NO: 84 - DNA, 333 bp, synthetic construct
  source 1..333: mol_type = other DNA
cagtctgtgctgacgcagccgccctcagtgtctggggccccagggcagagggtcaccatctcctgcactgggagcagctccaacatcggggcaggttatgatgtacactggtaccagcagcttccaggaacagcccccaaactcctcatctatggtaacagcaatcggccctcaggggtccctgaccgattctctggctccaagtctggcacctcagcctccctggccatcactgggctccaggctgaggatgaggctgattattactgccagtcctatgaccgcagcctgagtggttgggtgttcggcggagggaccaagctgaccgtccta

SEQ ID NO: 85 - protein, 8 aa, synthetic construct
GFTFSAYP

SEQ ID NO: 86 - protein, 8 aa, synthetic construct
ISYDGSNN

SEQ ID NO: 87 - protein, 10 aa, synthetic construct
ARGKVGTLDF

SEQ ID NO: 88 - protein, 117 aa, synthetic construct
QIQLVESGGGVVQPGRSLRLSCAASGFTFSAYPMHWVRQAPGKGLEWVAIISYDGSNNYYADSVKGRFTISRDNSKNTMYLQINSLRAEDTGVYYCARGKVGTLDFWGQGTLVTVSS

SEQ ID NO: 89 - protein, 112 aa, synthetic construct
DVVLTQSPLSLPVTLGQPASISCRSSQSLVYSDGNTYLNWFQQRPGQSPRRLIYKVSNRDSGVPDRFSGSGSGTDFTLKISRVEAEDVGVYYCMQGTHWPWTFGQGTKVEIK

SEQ ID NO: 90 - DNA, 351 bp, synthetic construct
  source 1..351: mol_type = other DNA
cagatacagctggtggagtctgggggaggcgtggtccagcctgggaggtccctgagactctcctgtgcagcctctggattcaccttcagtgcctatcccatgcactgggtccgccaggctccaggcaaggggctggagtgggtggcaattatatcatatgatggaagtaataactactatgcagactccgtgaagggccgattcaccatctccagagacaattccaagaacacgatgtatctgcaaattaacagcctgagagctgaggacacgggtgtgtattactgtgcgcgagggaaagtgggaacccttgacttctggggccagggaaccctggtcaccgtctcctca

SEQ ID NO: 91 - DNA, 336 bp, synthetic construct
  source 1..336: mol_type = other DNA
gatgttgtgctgactcagtctccactctccctgcccgtcacccttggacagccggcctccatctcctgcaggtctagtcaaagcctcgtatacagtgatggaaacacctacttgaattggtttcagcagaggccaggccaatctccaaggcgcctaatttataaggtttctaaccgggactctggggtcccagacagattcagcggcagtgggtcaggcactgatttcacactgaaaatcagcagggtggaggctgaggatgttggggtttattactgcatgcaaggtacacactggccgtggacgttcggccaagggaccaaggtggaaatcaaa

SEQ ID NO: 92 - protein, 112 aa, synthetic construct
DIVMTQSPLSSPVTLGQPASISCRSSQSLVYSDGNTYLSWLQQRPGQPPRLLIYKISNRFSGVPDRFSGSGAGTDFTLKISRVEAEDVGVYYCMQSTQFPHTFGQGTKLEIK

SEQ ID NO: 93 - DNA, 363 bp, synthetic construct
  source 1..363: mol_type = other DNA
gaggtgcagctgttggagtctgggggaggcttggtccagcctggggggtccctgagactctcctgtgcagcctctggattcacctttagcacctatgccatgagctgggtccgccaggctccagggaaggggctggagtgggtctcagctattagtggtcgtggtggtagcacatactacacagactccgtgaagggccggttcaccatctccagagacaattccaagaatacggtgtctctgcaaatgaacagcctgagagccgaggacacggccgtatattactgtgcgggccggggagattactactactactacggtatggacgtctggggccaagggaccacggtcaccgtctccgca

SEQ ID NO: 94 - DNA, 336 bp, synthetic construct
  source 1..336: mol_type = other DNA
gatattgtgatgacccagagtccactctcctcacctgtcacccttggacagccggcctccatctcctgcaggtctagtcaaagcctcgtatacagtgatggaaacacctacttgagttggcttcagcagaggccaggccagcctccaagactcctaatttataagatttctaaccggttctctggggtcccagacagattcagtggcagtggggcagggacagatttcacactgaaaatcagcagggtggaagctgaggatgtcggggtttattactgcatgcaatctacacaatttcctcacacttttggccaggggaccaagctggagatcaaa

SEQ ID NO: 95 - protein, 19 aa, synthetic construct
GGGGSGGGGSGGGSGGGGS

SEQ ID NO: 96 - protein, 15 aa, synthetic construct
GGGGSGGGGSGGGGS

SEQ ID NO: 97 - protein, 24 aa, synthetic construct
GGGGSGGGGSGGGGSGGGSGGGGS

SEQ ID NO: 98 - protein, 29 aa, synthetic construct
GGGGSGGGGSGGGGSGGGGSGGGSGGGGS

SEQ ID NO: 99 - protein, 5 aa, synthetic construct
GGGGS

SEQ ID NO: 100 - protein, 10 aa, synthetic construct
GGGGSGGGGS

SEQ ID NO: 101 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 102 - protein, 247 aa, Mus musculus
MASPLTRFLSLNLLLMGESIILGSGEAKPQAPELRIFPKKMDAELGQKVDLVCEVLGSVSQGCSWLFQNSSSKLPQPTFVVYMASSHNKITWDEKLNSSKLFSAVRDTNNKYVLTLNKFSKENEGYYFCSVISNSVMYFSSVVPVLQKVNSTTTKPVLRTPSPVHPTGTSQPQRPEDCRPRGSVKGTGLDFACDIYIWAPLAGICVAPLLSLIITLICYHRSRKRVCKCPRPLVRQEGKPRPSEKIV

SEQ ID NO: 103 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 104 - DNA, 81 bp, synthetic construct
  source 1..81: mol_type = other DNA
ttttgggtgctggtggtggttggtggagtcctggcttgctatagcttgctagtaacagtggcctttattattttctgggtg

SEQ ID NO: 105 - protein, 218 aa, Mus musculus
MTLRLLFLALNFFSVQVTENKILVKQSPLLVVDSNEVSLSCRYSYNLLAKEFRASLYKGVNSDVEVCVGNGNFTYQPQFRSNAEFNCDGDFDNETVTFRLWNLHVNHTDIYFCKIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRP

SEQ ID NO: 106 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 107 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 108 - DNA, 336 bp, synthetic construct
  source 1..336: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 109 - DNA, 123 bp, synthetic construct
  source 1..123: mol_type = other DNA
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 110 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 111 - DNA, 126 bp, synthetic construct
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 112 - protein, 480 aa, synthetic construct
EVQLLESGGGLVQPGGSLRLSCAASGFTFSTYAMSWVRQAPGKGLEWVSAISGRGGSTYYTDSVKGRFTISRDNSKNTVSLQMNSLRAEDTAVYYCAGRGDYYYYYGMDVWGQGTTVTVSAGGGGSGGGGSGGGGSDIVMTQSPLSSPVTLGQPASFSCRSSQSLVYSDGNTYLSWLQQRPGQPPRLLIYKISNRFSGVPDRFSGSGAGTDFTLKISRVEAEDVGVYYCMQSTQFPHTFGQGTKLEIKEQKLISEEDLAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 113 - DNA, 1443 bp, synthetic construct
  source 1..1443: mol_type = other DNA
gaggtgcagctgttggagtctgggggaggcttggtccagcctggggggtccctgagactctcctgtgcagcctctggattcacctttagcacctatgccatgagctgggtccgccaggctccagggaaggggctggagtgggtctcagctattagtggtcgtggtggtagcacatactacacagactccgtgaagggccggttcaccatctccagagacaattccaagaacacggtgtctctgcaaatgaacagcctgagagccgaggacacggccgtatattactgtgcgggccggggagattactactactactacggtatggacgtctggggccaagggaccacggtcaccgtctccgcaggtggaggtggatcaggtggaggtggatctggtggaggtggatctgatattgtgatgacccagagtccactctcctcacctgtcacccttggacagccggcctccttctcctgcaggtctagtcaaagcctcgtatacagtgatggaaacacctacttgagttggcttcagcagaggccaggccagcctccaagactcctaatttataagatttctaaccggttctctggggtcccagacagattcagtggcagtggggcagggacagatttcacactgaaaatcagcagggtggaagctgaggatgtcggggtttattactgcatgcaatctacacaatttcctcacacttttggccaggggaccaagctggagatcaaagaacagaaactgatctctgaagaagacctggcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctag

SEQ ID NO: 114 - protein, 478 aa, synthetic construct
EVHLLESGGGLVQPGGSLRLSCAASGFIFSSNAMSWVRQAPGKGLEWVSAISGYGGNTYYADSVKGRFTISRDNSKNTLYLQMNSLRAEDTAVYYCAKWGTYIVGATGDYWGQGTLVTVSSGGGGSGGGGSGGGGSQSALTQPPSASGSPGQSVTISCTGTSNDVGGYNYVSWYQQHPGKAPKLLIYEVSKRPSGVPDRFSGSQSGNTASLTVSGLQAEDEADYYCSSYAGSNNWVFGGGTKLTVLEQKLISEEDLAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 115 - DNA, 1437 bp, synthetic construct
  source 1..1437: mol_type = other DNA
gaggtgcacctgttggagtctgggggaggcttggtacagcctggggggtccctgagactctcctgtgcagcctctggattcatctttagcagcaatgccatgagctgggtccgccaggctccagggaagggactggagtgggtctcagctattagtggttatggtggtaacacatactacgcagactccgtgaagggccggttcaccatctccagagacaattccaagaacacgctatatctgcaaatgaacagcctgagagccgaggacacggccgtatattactgtgcgaaatgggggacttatatagtgggagctacgggtgactactggggccagggaactctggtcaccgtctcctcaggtggaggtggatcaggtggaggtggatctggtggaggtggatctcagtctgccctgactcagcctccctccgcgtccgggtctcctggacagtcagtcaccatctcctgcactggaaccagcaatgacgttggtggttataactatgtctcctggtaccaacagcacccaggcaaagcccccaaactcttgatttatgaggtcagtaagcggccctcaggggtccctgatcgcttctctggctcccagtctggcaacacggcctccctgaccgtctctgggctccaggctgaggatgaggctgattattactgcagctcatatgcaggcagcaacaattgggtgttcggcggagggaccaagctgaccgtcctagaacagaaactgatctctgaagaagacctggcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctag

SEQ ID NO: 116 - protein, 474 aa, synthetic construct
QVQLQQSGPGLVKPSQTLSLTCAISGDSVSSNSAAWNWIRQSPSRGLEWLGRTYFRSKWYNVYAVSVKSRITINPDTSKNQFSLQLNSVTPEDTAVYYCASEGGSYYDHWGQGTLVTVSSGGGGSGGGGSGGGGSDIQMTQSPSSVSASVGDRVTITCRASQGISNWLTWYQQKPGKAPKLLIYAASSLQSGVPSRFSGSGSGTDFTLTISSLQPEDFATYYCQQADSFPFTFGPGTKVDIKEQKLISEEDLAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 117 - DNA, 1425 bp, synthetic construct
  source 1..1425: mol_type = other DNA
caggtacagctgcagcagtcaggtccaggactggtgaagccctcgcagaccctctcactcacctgtgccatctccggggacagtgtctctagcaacagtgctgcttggaactggatcaggcagtccccatcgagaggccttgagtggctgggaaggacatacttcaggtccaagtggtataatgtttatgcagtgtctgtgaagagtcgaataaccatcaacccagacacatccaagaaccagttctccctgcagctgaactctgtgactcccgaggacacggctgtgtattattgtgcaagcgagggtgggagctattatgaccactggggccagggaaccctggtcaccgtctcctcaggtggaggtggatcaggtggaggtggatctggtggaggtggatctgacatccagatgacccagtctccatcttccgtgtctgcatctgtaggagacagagtcaccatcacttgtcgggcgagtcagggtattagtaattggttaacctggtatcagcagaaaccagggaaagcccctaagctcctgatctatgctgcatccagtttgcaaagtggggtcccatcaaggttcagcggcagtggatctgggacagatttcactctcaccatcagcagcctgcagcctgaagattttgcaacttactattgtcaacaggctgacagtttcccattcactttcggccctgggaccaaagtggatatcaaagaacagaaactgatctctgaagaagacctggcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctag

SEQ ID NO: 118 - protein, 179 aa, synthetic construct
MGYRMQLLSCIALSLALVTNSGYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 119 - DNA, 540 bp, synthetic construct
  source 1..540: mol_type = other DNA
atgggttacaggatgcaactcctgtcttgcattgcactaagtcttgcacttgtcacaaacagtggctactttggcaagcttgaatctaaattatcagtcataagaaatttgaatgaccaagttctcttcattgaccaaggaaatcggcctctatttgaagatatgactgattctgactgtagagataatgcaccccggaccatatttattataagtatgtataaagatagccagcctagaggtatggctgtaactatctctgtgaagtgtgagaaaatttcaactctctcctgtgagaacaaaattatttcctttaaggaaatgaatcctcctgataacatcaaggatacaaaaagtgacatcatattctttcagagaagtgtcccaggacatgataataagatgcaatttgaatcttcatcatacgaaggatactttctagcttgtgaaaaagagagagacctttttaaactcattttgaaaaaagaggatgaattgggggatagatctataatgttcactgttcaaaacgaagactag

SEQ ID NO: 120 - protein, 179 aa, synthetic construct
MYRMQLLSCIALSLALVTNSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 121 - DNA, 540 bp, synthetic construct
  source 1..540: mol_type = other DNA
atgtacaggatgcaactcctgtcttgcattgcactaagtcttgcacttgtcacaaacagtagtatcacaggaatttcacctattacagagtatcttgcttctctaagcacatacaatgatcaatccattacttttgctttggaggatgaaagttatgagatatatgttgaagacttgaaaaaagatgaaaagaaagataaggtgttactgagttactatgagtctcaacacccctcaaatgaatcaggtgacggtgttgatggtaagatgttaatggtaaccctgagtcctacaaaagacttctggttgcatgccaacaacaaggaacactctgtggagctccataagtgtgaaaaaccactgccagaccaggccttctttgtccttcataatatgcactccaactgtgtttcatttgaatgcaagactgatcctggagtgtttataggtgtaaaggataatcatcttgctctgattaaagtagactcttctgagaatttgtgtactgaaaatatcttgtttaagctctctgaaacttag

SEQ ID NO: 122 - DNA, 54 bp, synthetic construct
  source 1..54: mol_type = other DNA
atggctctcccagtgactgccctactgcttcccctagcgcttctcctgcatgca

SEQ ID NO: 123 - protein, 4 aa, synthetic construct
YMNM

SEQ ID NO: 124-125 - skipped (2 entries)

SEQ ID NO: 126 - protein, 4 aa, synthetic construct
YENV

SEQ ID NO: 127 - protein, 4 aa, synthetic construct
YSNV

SEQ ID NO: 128 - protein, 4 aa, synthetic construct
YKNL

SEQ ID NO: 129 - protein, 4 aa, synthetic construct
YENQ

SEQ ID NO: 130 - protein, 4 aa, synthetic construct
YKNI

SEQ ID NO: 131 - protein, 4 aa, synthetic construct
YINQ

SEQ ID NO: 132 - protein, 4 aa, synthetic construct
YHNK

SEQ ID NO: 133 - protein, 4 aa, synthetic construct
YVNQ

SEQ ID NO: 134 - protein, 4 aa, synthetic construct
YLNP

SEQ ID NO: 135 - protein, 4 aa, synthetic construct
YLNT

SEQ ID NO: 136 - protein, 4 aa, synthetic construct
YDND

SEQ ID NO: 137 - protein, 4 aa, synthetic construct
YENI

SEQ ID NO: 138 - protein, 4 aa, synthetic construct
YENL

SEQ ID NO: 139 - protein, 4 aa, synthetic construct
YKNQ

SEQ ID NO: 140 - protein, 4 aa, synthetic construct
YKNV

SEQ ID NO: 141 - protein, 4 aa, synthetic construct
YANG

SEQ ID NO: 142 - protein, 4 aa, synthetic construct
YMDM

SEQ ID NO: 143 - protein, 4 aa, synthetic construct
YMPM

SEQ ID NO: 144 - protein, 4 aa, synthetic construct
YMRM

SEQ ID NO: 145 - protein, 4 aa, synthetic construct
YMSM

SEQ ID NO: 146 - skipped

SEQ ID NO: 147 - protein, 4 aa, synthetic construct
YTHM

SEQ ID NO: 148 - protein, 4 aa, synthetic construct
YVLM

SEQ ID NO: 149 - protein, 4 aa, synthetic construct
YIAM

SEQ ID NO: 150 - protein, 4 aa, synthetic construct
YVEM

SEQ ID NO: 151 - protein, 4 aa, synthetic construct
YVKM

SEQ ID NO: 152 - protein, 4 aa, synthetic construct
YVPM

SEQ ID NO: 153 - skipped

SEQ ID NO: 154 - protein, 4 aa, synthetic construct
YMAP

SEQ ID NO: 155-156 - skipped (2 entries)

SEQ ID NO: 157 - protein, 4 aa, synthetic construct
YMNV

SEQ ID NO: 158 - protein, 4 aa, synthetic construct
YENM

SEQ ID NO: 159 - protein, 4 aa, synthetic construct
YMNQ

SEQ ID NO: 160 - protein, 4 aa, synthetic construct
YMNL

SEQ ID NO: 161 - protein, 4 aa, synthetic construct
YSNM

SEQ ID NO: 162 - skipped

SEQ ID NO: 163 - protein, 4 aa, synthetic construct
YGGG

SEQ ID NO: 164 - protein, 4 aa, synthetic construct
YAAA

SEQ ID NO: 165 - protein, 4 aa, synthetic construct
YFFF

SEQ ID NO: 166 - protein, 4 aa, synthetic construct
YETV

SEQ ID NO: 167 - protein, 4 aa, synthetic construct
YQQQ

SEQ ID NO: 168 - protein, 4 aa, synthetic construct
YHAE

SEQ ID NO: 169 - protein, 4 aa, synthetic construct
YLDL

SEQ ID NO: 170 - protein, 4 aa, synthetic construct
YLIP

SEQ ID NO: 171 - protein, 4 aa, synthetic construct
YLRV

SEQ ID NO: 172 - protein, 4 aa, synthetic construct
YTAV

SEQ ID NO: 173 - protein, 4 aa, synthetic construct
YVHV

SEQ ID NO: 174 - protein, 41 aa, synthetic construct
RSKRSRLLHSDYENVTPRRPGPTRKHYQPYAPPRDFAAYRS

SEQ ID NO: 175 - protein, 41 aa, synthetic construct
RSKRSRLLHSDYKNITPRRPGPTRKHYQPYAPPRDFAAYRS

SEQ ID NO: 176 - protein, 41 aa, synthetic construct
RSKRSRLLHSDYMDMTPRRPGPTRKHYQPYAPPRDFAAYRS

SEQ ID NO: 177 - protein, 41 aa, synthetic construct
RSKRSRLLHSDYGGGTPRRPGPTRKHYQPYAPPRDFAAYRS

SEQ ID NO: 178 - protein, 41 aa, synthetic construct
RSKRSRLLHSDYSNVTPRRPGPTRKHYQPYAPPRDFAAYRS